SEQUENCE LISTING (3 sequences)

SEQ ID NO: 1 - DNA, 19 bp, Artificial Sequence
  PCR Primer
ctgtgccttgggtggcttt

SEQ ID NO: 2 - DNA, 24 bp, Artificial Sequence
  PCR Primer
aaggaaagaagtcagaaggcaaaa

SEQ ID NO: 3 - DNA, 34 bp, Artificial Sequence
  PCR Probe
agctccaaattctttataagggtcgatgtccatg

(19) United States
(12) Patent Application Publication
(10) Pub. No.: US 2023/0075856 A1
(43) Pub. Date: Mar. 9, 2023

(54) CYCLIC SULFAMIDE COMPOUNDS FOR TREATMENT OF HBV

(71) Applicant: Assembly Biosciences, Inc., South San Francisco, CA (US)

(72) Inventors: Michael Walker, South San Francisco, CA (US); Leping Li, Carmel, IN (US); Simon Nicolas Haydar, South San Francisco, CA (US)

(21) Appl. No.: 17/274,055

(22) PCT Filed: Sep. 5, 2019
(86) PCT No.: PCT/US2019/049730

§ 371 Date: Mar. 5, 2021

Related U.S. Application Data
(60) Provisional application No. 62/727,274, filed on Sep. 5, 2018.

(51) Int. Cl. C07D 417/12; C07D 417/14; C07D 285/18; A61P 31/14

(52) U.S. Cl. CPC C07D 417/12; A61P 31/14; C07D 285/18; C07D 417/14

(57) ABSTRACT

The present disclosure provides, in part, cyclic sulfamide compounds, and pharmaceutical compositions thereof, useful for disruption of HBV core protein assembly, and methods of treating Hepatitis B (HBV) infection.

### RELATED APPLICATION

This application claims priority to and the benefit of U.S. Provisional Application No. 62/727,274, filed Sep. 5, 2018, the entire contents of which are incorporated by reference herein.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleotide analogs (e.g., entecavir) that suppress the virus while the patient remains on treatment, but do not eliminate the infection, even after many years of treatment. Once a patient starts taking nucleotide analogs, most must continue taking them or risk the possibility of a life threatening immune response due to viral rebound. Further, nucleotide therapy may lead to the emergence of antiviral drug resistance.

The only FDA approved alternative to nucleotide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients are likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

### SUMMARY

The present disclosure provides, in part, cyclic sulfamide compounds and pharmaceutical compositions thereof, useful for disruption of HBV core protein assembly, and methods of treating HBV infections.

In one aspect, the disclosure provides compounds of Formula I:

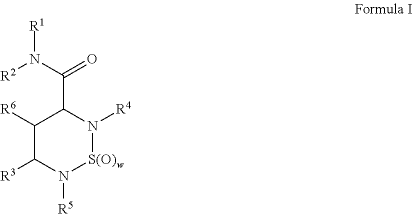

or a pharmaceutically acceptable salt thereof, where the variables are described in the detailed description.

In another aspect, the disclosure provides pharmaceutical compositions comprising aa compound of Formula I, or a pharmaceutically acceptable salt thereof, and a pharmaceutically acceptable excipient.

In another aspect, the disclosure provides a method of treating an HBV infection in a subject in need thereof, comprising: administering to the subject a therapeutically effective amount of compound of Formula I, or a pharmaceutically acceptable salt thereof.

In another aspect, the disclosure provides a method of treating an HBV infection in a subject in need thereof, comprising: administering to the subject a pharmaceutical composition comprising a therapeutically effective amount of a compound of Formula I, or a pharmaceutically acceptable salt thereof, and a pharmaceutically acceptable excipient.

### BRIEF DESCRIPTION OF DRAWINGS

FIG. 1 is the crystal structure of HBV-CSU-016-Isomer-I as described herein.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “alkenyl” as used herein refers to an unsaturated straight or branched hydrocarbon having at least one carbon-carbon double bond. Exemplary alkenyl groups include, but are not limited to, a straight or branched group of 2-6 carbon atoms, referred to herein as C₂₋₆alkenyl. Exemplary alkenyl groups include, but are not limited to, vinyl, allyl, butenyl, pentenyl, etc.

The term “alkoxy” as used herein refers to a straight or branched alkyl group attached to oxygen (i.e., alkyl-O—). Exemplary alkoxy groups include, but are not limited to, alkoxy groups of 1-6 or 1-4 carbon atoms, referred to herein as C₁₋₆alkoxy and C₁₋₄alkoxy, respectively. Exemplary alkoxy groups include, but are not limited to methoxy, ethoxy, isopropoxy, etc.

The term “alkoxyalkyl” as used herein refers to an alkyl group substituted with an alkoxy group (i.e., alkoxy-alkyl- or alkyl-O-alkyl-). Examples include, but are not limited to, CH₃CH₂OCH₂—, CH₃OCH₂CH₂— and CH₃OCH₂—.

The term “alkyl” as used herein refers to a saturated straight or branched hydrocarbon. Exemplary alkyl groups include, but are not limited to, straight or branched hydrocarbons of 1-6 or 1-4 carbon atoms, referred to herein as C₁₋₆alkyl and C₁₋₄alkyl, respectively. Exemplary alkyl groups include, but are not limited to, methyl, ethyl, n-propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, t-butyl, n-pentyl, isopentyl, neopentyl, n-hexyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6 carbon atoms, referred to herein as C₂₋₆alkynyl. Exemplary alkynyl groups include, but are not limited to, ethynyl, propynyl, butynyl, pentynyl, hexynyl, methylpropynyl, etc.

The term “carbonyl” as used herein refers to the biradical —C(O)—.

The term “cyano” as used herein refers to the radical —CN.

The term “cycloalkyl” as used herein refers to a saturated monocyclic hydrocarbon group of, for example, 3-6 carbons, referred to herein as C₃-6cycloalkyl, or bicyclic hydrocarbon ring structure of, for example, 8-12 carbons, referred to herein as C₈₋₁₂cycloalkyl. For bicyclic cycloalkyl groups, the two rings may be attached through the same or different carbons. Exemplary monocyclic cycloalkyl groups include, but are not limited to, cyclohexyl, cyclopentyl, cyclopentenyl, cyclobutyl and cyclopropyl. Exemplary bicyclic cycloalkyl groups include, but are not limited to, spiro[2.5]octanyl, spiro[3.5]nonanyl, bicyclo[2.2.2]octanyl, bicyclo[4.1.0]heptanyl, octahydropentalenyl, bicyclo[4.2.0]octanyl, bicyclo[1.1.1]pentanyl, bicyclo[2.2.1]heptanyl, and bicyclo[2.2.2]octanyl.

The term “cycloalkenyl” as used herein refers to a partially unsaturated monocyclic hydrocarbon group of, for example, 3-7 carbons, referred to herein as C₄₋₇cycloalkenyl, or bicyclic hydrocarbon ring structure of, for example, 8-12 carbons, referred to herein as C₈₋₁₂cycloalkenyl. For bicyclic cycloalkenyl groups: 1) either one or both rings contain one or more double bonds and 2) the two rings may be attached through the same or different ring carbons. Exemplary monocyclic cycloalkenyl groups include, but are not limited to, cyclopropenyl, cyclobutenyl, cyclopentenyl, cyclohexenyl and cycloheptenyl. Exemplary bicyclic cycloalkenyl groups include, but are not limited to, spiro[2.5]oct-5-enyl, spiro[2.5]oct-4-enyl, spiro[3.5]non-5-enyl, spiro[3.5]non-6-enyl, bicyclo[4.1.0]hept-3-enyl, bicyclo[4.1.0]hept-2-enyl, and bicyclo[2.2.2]oct-2-enyl.

The term “carbocyclyl” as used herein refers to a bicyclic ring system formed by fusing a phenyl ring to a C₃₋₆cycloalkyl, C₄₋₇cycloalkenyl, 4-7 membered monocyclic heterocycloalkyl or 4-7 membered monocyclic heterocycloalkenyl ring. Where possible, the rings may be linked to the adjacent radical though carbon or nitrogen. Examples of heterocyclyls include, but are not limited to 2,3-dihydro-1H-indenyl, 1,2,3,4-tetrahydronaphthalene, isochromanyl, and 1H-indenyl, and 2H-quinolinyl.

The terms “halo” or “halogen” as used herein refer to F, Cl, Br or I.

The term “haloalkyl” as used herein refers to an alkyl group substituted with one or more halogen atoms. For example, haloC₁₋₆alkyl refers to a straight or branched alkyl group of 1-6 carbon atoms substituted with one or more halogen atoms. Examples include but are not limited to —CH₂F, —CHCl₂, —CF₃, —CH₂CF₃, —CF₂CH₃, —CCl₂CF₃ and —CF₂CF₃.

The term “haloalkoxy” as used herein refers to an alkoxy group substituted with one or more halogen atoms. Examples include, but are not limited to, CCl₃O—, CF₃O—, CF₃CH₂O—, and CF₃CF₂O—.

The terms “heteroaryl” as used herein refers to a monocyclic aromatic 5-6 membered ring system or bicyclic aromatic 8-12 membered ring system containing one or more independently selected heteroatoms, for example one to four heteroatoms, such as nitrogen, oxygen and sulfur. Where possible, the heteroaryl ring may be linked to the adjacent radical though carbon or nitrogen. Examples of 5-6 membered monocyclic heteroaryls include, but are not limited to, franyl, thiophenyl (also referred to as thienyl), pyrrolyl, thiazolyl, oxazolyl, isothiazolyl, isoxazolyl, imidazolyl, pyrazolyl, 1H-1,2,3-triazolyl, 2H-1,2,3-triazolyl, 1,2,4-triazolyl, pyridinyl, pyridazinyl, pyrimidinyl, pyrazinyl, 1,3,5-triazinyl, 1,2,4-triazinyl, 1,2,3-triazinyl, 1,2,4-oxadiazolyl, 1,3,4-oxadiazolyl, 1,2,5-oxadiazolyl, 1,2,4-thiadiazolyl, 1,3,4-thiadiazolyl, 1,2,5-thiadiazolyl and tetrazolyl. Examples of 8-12 membered bicyclic heteroaryls include, but are not limited to, benzofranyl, isobenzofuranyl, benzo[b]thiophenyl, benzo[c]thiophenyl, indolyl, isoindolyl, benzo[d]isoxazolyl, benzo[c]isoxazolyl, benzo[d]oxazolyl, benzo[d]isothiazolyl, benzo[c]isothiazolyl, benzo[d]thiazolyl, indazolyl, benzo[d]imidazolyl, benzo[d]imidazolyl, and benzo[d][1,2,3]triazolyl.

The term “heterocycloalkyl” refers to a saturated monocyclic 3-7 membered ring system or bicyclic 8-12 membered ring system containing one or more independently selected heteroatoms, such as nitrogen, oxygen, and sulfur (including its oxidation states: S, S(O) and SO₂). Where possible, “heterocycloalkyl” rings may be linked to the adjacent radical through carbon or nitrogen. Examples of 4-7 membered monocyclic “heterocycloalkyl” groups include, but are not limited to, aziridinyl, oxiranyl, thiiranyl 1,1-dioxide, oxetanyl, azetidinyl, thietanyl 1,1-dioxide, pyrrolidinyl, tetrahydrofuranyl, piperidinyl, tetrahydro-2H-pyranyl, morpholinyl, thiomorpholinyl, and piperazinyl. Examples of bicyclic 8-12 membered heterocycloalkyl groups include, but are not limited to, 1,4-dioxaspiro[4.5]decanyl and 1,5-dioxaspiro[5.5]undecanyl.

The term “heterocycloalkenyl” refers to a partially unsaturated monocyclic 3-7 membered ring system or bicyclic 8-12 membered ring system containing one, two or three independently selected heteroatoms, such as nitrogen, oxygen, and sulfur (including its oxidation states: S, S(O) or SO₂). Where possible, heterocycloalkenyl rings may be linked to the adjacent radical through carbon or nitrogen. For bicyclic heterocycloalkenyl groups: 1) either one or both rings contain one or more double bonds and 2) the two rings may be attached through the same or different ring atoms. Examples of 4-7 membered monocyclic heterocycloalkenyl groups include, but are not limited to 2,3-dihydro-1H-pyrrolyl, 2,5-dihydro-1H-pyrrolyl, 4,5-dihydro-1H-pyrazolyl, 2,3-dihydro-1H-pyrazolyl, 4,5-dihydro-1H-imidazolyl, 2,3-dihydro-1H-imidazolyl, 2,3-dihydrothiophenyl, 2,5-dihydrothiophenyl, 4,5-dihydrothiazolyl, 2,3-dihydrothiazolyl, 4,5-dihydroisothiazolyl, 2,3-dihydroisothiazolyl, 2,3-dihydrofuranyl, 2,5-dihydrofuranyl, 4,5-dihydrooxazolyl, 2,3-dihydrooxazolyl, 4,5-dihydroisoxazolyl, 2,3-dihydroisoxazolyl, 3,4-dihydropyridinyl, 2,3-dihydropyridinyl, 2,3,4,5-tetrahydropyridinyl, 1,6-dihydropyridazinyl, 4,5-dihydropyridazinyl, 3,4,5,6-tetrahydropyridazinyl, 4,5-dihydropyrimidinyl, 1,2,5,6-tetrahydropyrimidinyl, 1,2-dihydropyrimidinyl, 1,2-dihydropyrazinyl, 2,3-dihydropyrazinyl, 1,2,3,6-tetrahydropyrazinyl, 4H-1,4-oxazinyl, 3,4-dihydro-2H-1,4-oxazinyl, 4H-1,4-thiazinyl, and 3,4-dihydro-2H-1,4-thiazinyl. Examples of 8-12 membered heterocycloalkyl groups include, but are not limited to 6,7-dihydroindolyl, 4,5-dihydroindolyl, 7,8-dihydroimidazo[1,2-a]pyridinyl, 5,6-dihydroimidazo[1,2-a]pyridinyl, 4,5-dihydrobenzo[d]imidazolyl, 6,7-dihydro-1H-indazolyl, 4,5-dihydro-1H-indazolyl, 4,5-dihydropyrazolo[1,5-a]pyridinyl, and 6,7-dihydropyrazolo[1,5-a]pyridinyl.

The term “heterocyclyl” as used herein refers to a bicyclic ring system formed by fusing a monocyclic aromatic 5-6 membered heteroaryl ring to a C₃₋₆cycloalkyl, C₄₋₇cycloalkenyl, 4-7 membered monocyclic heterocycloalkyl or 4-7 membered monocyclic heterocycloalkenyl ring. Where possible, the rings may be linked to the adjacent radical though carbon or nitrogen. Examples of heterocyclyls include, but are not limited to 6,7,8,9-tetrahydro-5H-[1,2,4]triazolo[4,3-a]azepine, 5,6,8,9-tetrahydro-[1,2,4]triazolo[4,3-d][1,4]oxazepane, 6,7-dihydro-5H,9H-[1,2,4]triazolo[3,4-c][1,4]oxazepane, 5,6,8,9-tetrahydro-712-[1,2,4]triazolo[4,3-d][1,4]diazepine, 8,9-dihydro-5H-[1,2,4]triazolo[4,3-a]azepine, 6,9-dihydro-5H-[1,2,4]triazolo[4,3-a]azepine, 5,6,7,8-tetrahydro-[1,2,4]triazolo[4,3-a]pyridine, 5,6-dihydro-8H-[1,2,4]triazolo[3,4-c][1,4]oxazine, 5,6,7,8-tetrahydroimidazo[1,2-a]pyridine, and 5H,9H-[1,2,4]triazolo[3,4-c][1,4]oxazepine.

The terms “hydroxy” and “hydroxyl” as used herein refers to the radical —OH.

The term “hydroxyalkyl” as used herein refers to an alkyl group substituted with one or more hydroxy groups. Examples include, but are not limited to, HOCH₂—, HOCH₂CH₂—, CH₃CH(OH)CH₂— and HOCH₂CH(OH)CH₂—.

The term “hydroxyalkoxy” as used herein refers to an alkoxy group substituted with one or more hydroxy groups. Examples include but are not limited to HOCH₂O—, HOCH₂CH₂O—, CH₃CH(OH)CH₂O— and HOCH₂CH(OH)CH₂O—.

The term “R^{a}R^{b}N—C₁₋₆alkyl-,” as used herein refers to an alkyl group substituted with a R^{a}R^{b}N— group, as defined herein. Examples include but are not limited to NH₂CH₂—, NH(CH₃)CH₂—, N(CH₃)₂CH₂CH₂— and CH₃CH(NH₂)CH₂—.

The term “R^{a}R^{b}N—C₁₋₆alkoxy,” as used herein refers to an alkoxy group substituted with one or more R^{a}R^{b}N— groups, as defined herein. Examples include but are not limited to NH₂CH₂—, NH(CH₃)CH₂O—, N(CH₃)₂CH₂CH₂O— and CH₃CH(NH₂)CH₂O—.

The term “oxo” as used herein refers to the radical ═O.

As used herein, when a bicyclic ring is shown with a floating point of attachment and/or floating substituents, for example as in

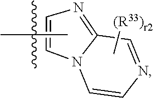

it signifies that the bicyclic ring can be attached via a carbon atom on either ring, and that the substituents (e.g., the R³³ group(s)) can be independently attached to either or both rings.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the disclosure is desirably a mammal in which treatment of HBV infection is desired.

The term “modulation” includes antagonism (e.g., inhibition), agonism, partial antagonism and/or partial agonism.

The term “Pharmaceutically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g. mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

The term “treating” includes any effect, e.g., lessening, reducing, modulating, or eliminating, via disruption of HBV core protein assembly, that results in the improvement of the disease. “Disruption” includes inhibition of HBV viral assembly and infection.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(f)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol ═ denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaemo, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al. Nature Reviews Drug Discovery 2008, 7, 255).

### I. Cyclic Sulfamide Compounds

In one aspect, the disclosure provides a compound of Formula I:

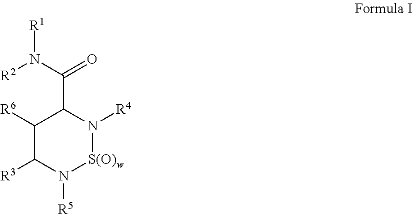

or a pharmaceutically acceptable salt thereof, wherein:

R¹ is a phenyl, naphthyl or heteroaryl, wherein: the phenyl, naphthyl or heteroaryl is optionally substituted with one, two, or three independently selected R³² groups;

R² is hydrogen or C₁₋₆alkyl;

R³ is -L-R⁷;

L is C₁₋₆alkylene;

R⁷ is a 5-6 membered monocyclic heteroaryl or 8-12 membered bicyclic heteroaryl selected from the group consisting of:

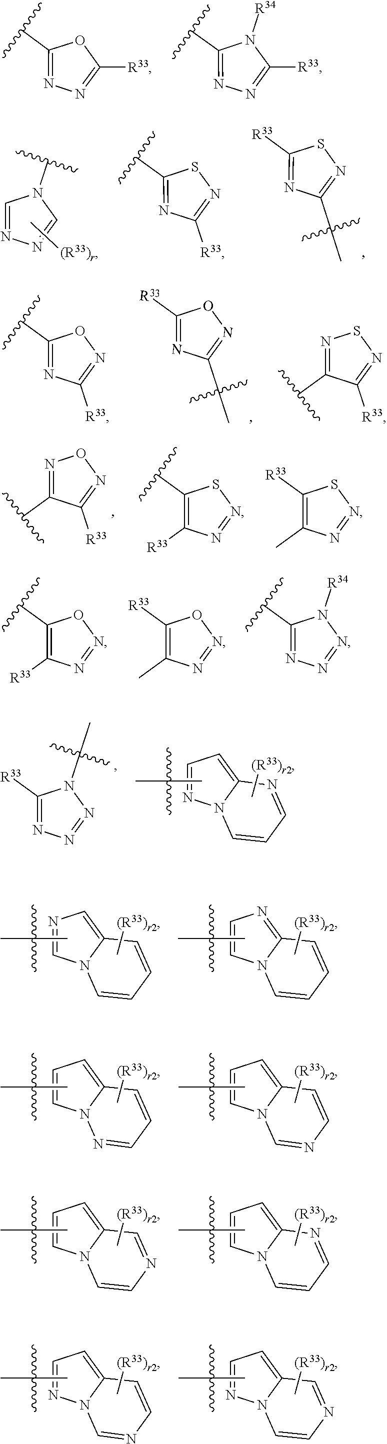

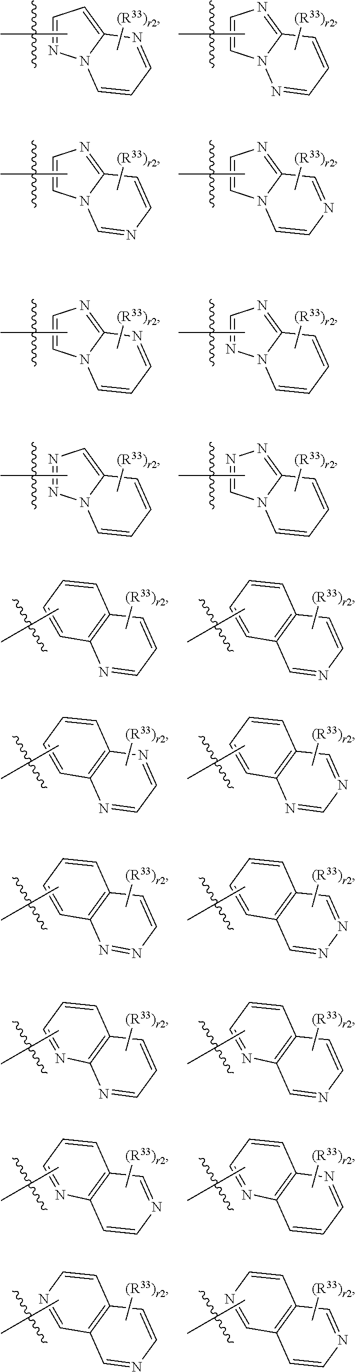

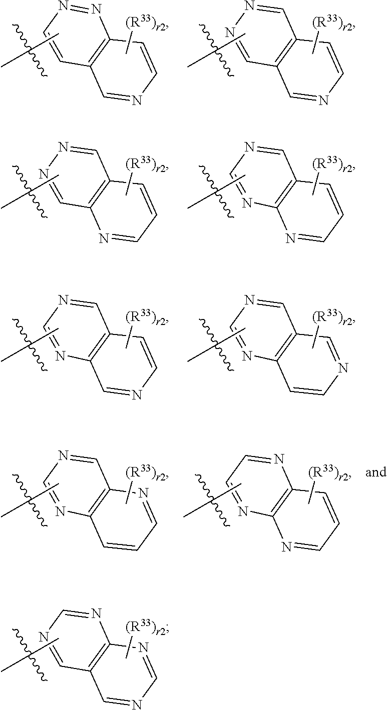

R⁴ is hydrogen or C₁₋₆alkyl optionally substituted with one, two, or three substituents independently selected from the group consisting of halogen, —OH, —CN, —S(O)_{q}—C₁₋₆alkyl, —NR^{a}R^{b}, —NR^{c}—S(O)ₜ—C₁₋₆alkyl, —S(O)—NR^{a}R^{b}, C₂₋₆alkenyl, C₂₋₆alkynyl, haloC₁₋₆alkyl, C₁₋₆alkoxy, haloC₁₋₆alkoxy, —C(O)NR^{a}R^{b}, —C(O)—C₁₋₆alkyl, formyl, —C(O)OH, a-C(O)O—C₁₋₆alkyl, benzyloxy, C₁₋₆alkoxyphenyl, pyrrolidinyl, morpholinyl, tetrahydrofuranyl and triazolyl;

R⁵ is hydrogen or C₁₋₆alkyl optionally substituted with one, two or three substituents independently selected from the group consisting of halogen, —OH, C₁₋₆alkoxy, —NR^{a}R^{b}, and R^{a}R^{b}N—C₁₋₆alkyl;

R⁶ is hydrogen or C₁₋₆alkyl;

R^{7a} is a phenyl or heteroaryl, wherein: the phenyl or heteroaryl is optionally substituted with one, two or three independently selected R³² groups;

R³² is halo, —OH, —CN, —NO₂, oxo, hydrazino, formyl, azido, silyl, siloxy, —S(O)_{q}—C₁₋₆alkyl, —NR^{a}R^{b}, —NR^{c}—S(O)ₜ—C₁₋₆alkyl, —S(O)—NR^{a}R^{b}, C₁₋₆alkyl, C₂₋₆alkenyl, C₂₋₆alkynyl, C₃₋₆cycloalkyl, haloC₁₋₆alkyl, hydroxyC₁₋₆alkyl, R^{a}R^{b}N—C₁₋₆alkyl-, C₁₋₆alkoxy, haloC₁₋₆alkoxy, hydroxyC₁₋₆alkoxy-, R^{a}R^{b}N—C₁₋₆alkoxy-, C₁₋₆alkoxyC₁₋₆alkyl, —C(O)NR^{a}R^{b}, —C(O)—C₁₋₆alkyl, —C(O)OH, or —C(O)O—C₁₋₆alkyl;

R³³ is independently selected for each occurrence from the group consisting of R³² and R^{7a};

R³⁴ is hydrogen or C₁₋₄alkyl;

R^{a} and R^{b} are independently selected for each occurrence from the group consisting of hydrogen and C₁₋₆alkyl; or

R^{a} and R^{b} may be taken together with the nitrogen to which R^{a} and R^{b} are attached to form:

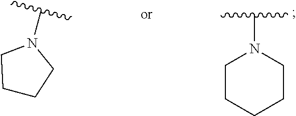 [SMILES: *N1CCCC1 ; *N1CCCCC1]

R^{c} is independently selected for each occurrence from the group consisting of hydrogen and C₁₋₆alkyl;

for each occurrence, q is independently 0, 1 or 2;

for each occurrence, t is independently 1 or 2;

r is 0, 1 or 2;

r2 is 0, 1, 2 or 3; and

w is 0, 1 or 2.

In another aspect, the disclosure provides a compound of Formula I:

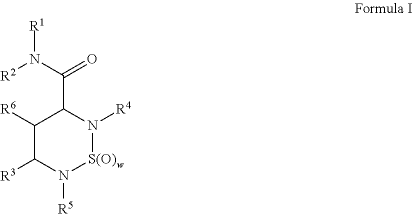

or a pharmaceutically acceptable salt thereof, wherein:

R¹ is a phenyl, naphthyl or heteroaryl, wherein: the phenyl, naphthyl or heteroaryl is optionally substituted with one, two, or three independently selected R³² groups;

R² is hydrogen or C₁₋₆alkyl;

R³ is -L-R⁷;

L is C₁₋₆alkylene, C₁₋₆alkenylene, or C₁₋₆alkynylene;

R⁷ is hydrogen, cycloalkyl, cycloalkenyl, carbocyclyl, heterocycloalkyl, heterocycloalkenyl, heterocyclyl, phenyl or heteroaryl, wherein: the cycloalkyl, cycloalkenyl, carbocyclyl, heterocycloalkyl, heterocycloalkenyl, heterocyclyl, phenyl or heteroaryl is optionally substituted with one, two or three substituents independently selected from the group consisting of R³², R³⁴ and R^{7a};

R^{7a} is a phenyl or heteroaryl, wherein: the phenyl or heteroaryl is optionally substituted with one, two or three independently selected R³² groups;

R⁴ is hydrogen or C₁₋₆alkyl optionally substituted with one, two, or three substituents independently selected from the group consisting of halogen, —OH, —CN, —S(O)_{q}—C₁₋₆alkyl, —NR^{a}R^{b}, —NR^{c}—S(O)ₜ—C₁₋₆alkyl, —S(O)—NR^{a}R^{b}, C₂₋₆alkenyl, C₂₋₆alkynyl, haloC₁₋₆alkyl, C₁₋₆alkoxy, haloC₁₋₆alkoxy, —C(O)NR^{a}R^{b}, —C(O)—C₁₋₆alkyl, formyl, —C(O)OH, a-C(O)O—C₁₋₆alkyl, benzyloxy, C₁₋₆alkoxyphenyl, pyrrolidinyl, morpholinyl, tetrahydrofuranyl and triazolyl;

R⁵ is hydrogen or C₁₋₆alkyl optionally substituted with one, two or three substituents independently selected from the group consisting of halogen, —OH, C₁₋₆alkoxy, —NR^{a}R^{b}, and R^{a}R^{b}N—C₁₋₆alkyl;

R⁶ is hydrogen or C₁₋₆alkyl;

R³² is halo, —OH, —CN, —NO₂, oxo, hydrazino, formyl, azido, silyl, siloxy, —S(O)_{q}—C₁₋₆alkyl, —NR^{a}R^{b}, —NR^{c}—S(O)ₜ—C₁₋₆alkyl, —S(O)ₜ—NR^{a}R^{b}, C₁₋₆alkyl, C₂₋₆alkenyl, C₂₋₆alkynyl, C₃₋₆cycloalkyl, haloC₁₋₆alkyl, hydroxyC₁₋₆alkyl, R^{a}R^{b}N—C₁₋₆alkyl-, C₁₋₆alkoxy, haloC₁₋₆alkoxy, hydroxyC₁₋₆alkoxy-, R^{a}R^{b}N—C₁₋₆alkoxy-, C₁₋₆alkoxyC₁₋₆alkyl, —C(O)NR^{a}R^{b}, —C(O)—C₁₋₆alkyl, —C(O)OH, or —C(O)O—C₁₋₆alkyl;

R³⁴ is hydrogen or C₁₋₄alkyl;

R^{a} and R^{b} are independently selected for each occurrence from the group consisting of hydrogen and C₁₋₆alkyl; or

R^{a} and R^{b} may be taken together with the nitrogen to which R^{a} and R^{b} are attached to form:

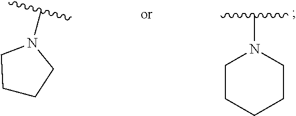 [SMILES: *N1CCCC1 ; *N1CCCCC1]

R^{c} is independently selected for each occurrence from the group consisting of hydrogen and C₁₋₆alkyl;

for each occurrence, q is independently 0, 1 or 2;

for each occurrence, t is independently 1 or 2; and

w is 0, 1 or 2;

with the proviso that when L is C₁₋₆alkylene, R⁷ is C₈₋₁₂cycloalkyl, C₈₋₁₂cycloalkenyl, carbocyclyl, heterocycloalkyl, heterocycloalkenyl, or heterocyclyl wherein: the C₈₋₁₂cycloalkyl, C₈₋₁₂cycloalkenyl, carbocyclyl, heterocycloalkyl, heterocycloalkenyl, or heterocyclyl is optionally substituted with one, two or three substituents independently selected from the group consisting of R³², R³⁴ and R^{7a}.

In another aspect, the disclosure provides a compound of Formula I:

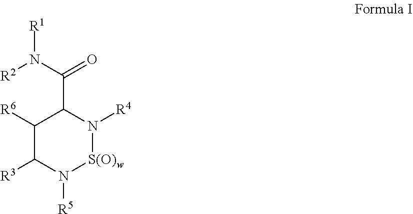

or a pharmaceutically acceptable salt thereof, wherein:

R¹ is a phenyl, naphthyl or heteroaryl, wherein: the phenyl, naphthyl or heteroaryl is optionally substituted with one, two, or three independently selected R³² groups;

R² is hydrogen or C₁₋₆alkyl;

R³ is -L-R⁷;

L is —C(O)—, —C(O)C₁₋₆alkyl-, —C₁₋₆alkylC(O)—, —C(O)NR^{c}—, —NR^{c}C(O)—, —C(O)NR^{c}C₁₋₆alkyl-, —C(O)NR^{c}CHR^{d}—, —NR^{c}C(O)C₁₋₆alkyl-, —C₁₋₆alkylC(O)NR^{c}—, or —C₁₋₆alkylNR^{c}C(O)—;

R⁷ is hydrogen, cycloalkyl, cycloalkenyl, carbocyclyl, heterocycloalkyl, heterocycloalkenyl, heterocyclyl, phenyl or heteroaryl, wherein: the cycloalkyl, cycloalkenyl, carbocyclyl. heterocycloalkyl, heterocycloalkenyl, heterocyclyl, phenyl or heteroaryl is optionally substituted with one, two or three substituents independently selected from the group consisting of R³², R³⁴ and -L²-R^{a};

L² is a bond, —C₁₋₆alkyl-, —O—, —OC₁₋₆alkyl- or —C(O)OC₁₋₆alkyl-;

R^{7a} is a cycloalkyl, heterocycloalkyl, phenyl or heteroaryl, wherein: the cycloalkyl, heterocycloalkyl, phenyl or heteroaryl is optionally substituted with one, two or three groups independently selected from R³², R³⁴ and -L³-R^{7b};

L³ is a bond, —C₁₋₆alkyl-, —O—, —OC₁₋₆alkyl- or —C(O)OC₁₋₆alkyl-;

R^{7b} is a cycloalkyl, heterocycloalkyl, phenyl or heteroaryl, wherein: the cycloalkyl, heterocycloalkyl, phenyl or heteroaryl is optionally substituted with one, two or three groups independently selected from R³²;

R⁴ is hydrogen or C₁₋₆alkyl optionally substituted with one, two, or three substituents independently selected from the group consisting of halogen, —OH, —CN, —S(O)_{q}—C₁₋₆alkyl, —NR^{a}R^{b}, —NR^{c}—S(O)ₜ—C₁₋₆alkyl, —S(O)—NR^{a}R^{b}, C₂₋₆alkenyl, C₂₋₆alkynyl, haloC₁₋₆alkyl, C₁₋₆alkoxy, haloC₁₋₆alkoxy, —C(O)NR^{a}R^{b}, —C(O)—C₁₋₆alkyl, formyl, —C(O)OH, a-C(O)O—C₁₋₆alkyl, benzyloxy, C₁₋₄alkoxyphenyl, pyrrolidinyl, morpholinyl, tetrahydrofuranyl and triazolyl;

R⁵ is hydrogen or C₁₋₆alkyl optionally substituted with one, two or three substituents independently selected from the group consisting of halogen, —OH, C₁₋₆alkoxy, —NR^{a}R^{b}, and R^{a}R^{b}N—C₁₋₆alkyl;

R⁶ is hydrogen or C₁₋₆alkyl;

R³² is halo, —OH, —CN, —NO₂, oxo, hydrazino, formyl, azido, silyl, siloxy, —S(O)_{q}—C₁₋₆alkyl, —NR^{a}R^{b}, —NR^{c}—S(O)—C₁₋₆alkyl, —S(O)ₜ—NR^{a}R^{b}, C₁₋₆alkyl, C₂₋₆alkenyl, C₂₋₆alkynyl, C₃₋₆cycloalkyl, haloC₁₋₆alkyl, hydroxyC₁₋₆alkyl, R^{a}R^{b}N—C₁₋₆alkyl-, C₁₋₆alkoxy, haloC₁₋₆alkoxy, hydroxyC₁₋₆alkoxy-, R^{a}R^{b}N—C₁₋₆alkoxy-, C₁₋₆alkoxyC₁₋₆alkyl, —C(O)NR^{a}R^{b}, —C(O)—C₁₋₆alkyl, —C(O)OH, or —C(O)O—C₁₋₆alkyl;

R³⁴ is hydrogen or C₁₋₆alkyl;

R^{a} and R^{b} are independently selected for each occurrence from the group consisting of hydrogen and C₁₋₆alkyl; or

R^{a} and R^{b} may be taken together with the nitrogen to which R^{a} and R^{b} are attached to form:

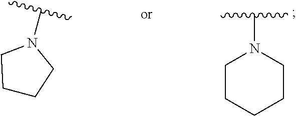 [SMILES: *N1CCCC1 ; *N1CCCCC1]

R^{c} is independently selected for each occurrence from the group consisting of hydrogen and C₁₋₆alkyl;

R^{d} is phenyl optionally substituted with one, two or three independently selected R³² groups;

for each occurrence, q is independently 0, 1 or 2;

for each occurrence, t is independently 1 or 2; and

w is 0, 1 or 2.

In another aspect, the disclosure provides a compound of Formula I:

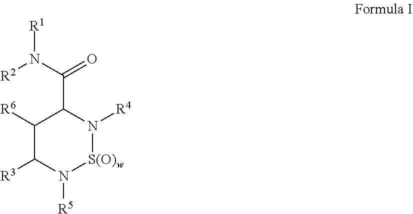

or a pharmaceutically acceptable salt thereof, wherein:

R¹ is a phenyl, naphthyl or heteroaryl, wherein: the phenyl, naphthyl or heteroaryl is optionally substituted with one, two, or three independently selected R³² groups;

R² is hydrogen or C₁₋₆alkyl;

R³ is -L-R⁷;

L is —C(O)—, —C(O)C₁₋₆alkyl-, —C₁₋₆alkylC(O)—, —C(O)NR^{c}—, —NR^{c}C(O)—, —C(O)NR^{c}C₁₋₆alkyl-, —NR^{c}C(O)C₁₋₆alkyl-, —C₁₋₆alkylC(O)NR^{c}—, or —C₁₋₆alkylNR^{c}C(O)—;

R⁷ is hydrogen, cycloalkyl, cycloalkenyl, carbocyclyl, heterocycloalkyl, heterocycloalkenyl, heterocyclyl, phenyl or heteroaryl, wherein: the cycloalkyl, cycloalkenyl, carbocyclyl, heterocycloalkyl, heterocycloalkenyl, heterocyclyl, phenyl or heteroaryl is optionally substituted with one, two or three substituents independently selected from the group consisting of R³², R³⁴ and R^{7a};

R^{7a} is a phenyl or heteroaryl, wherein: the phenyl or heteroaryl is optionally substituted with one, two or three independently selected R³² groups;

R⁴ is hydrogen or C₁₋₆alkyl optionally substituted with one, two, or three substituents independently selected from the group consisting of halogen, —OH, —CN, —S(O)_{q}—C₁₋₆alkyl, —NR^{a}R^{b}, —NR^{c}—S(O)ₜ—C₁₋₆alkyl, —S(O)—NR^{a}R^{b}, C₂₋₆alkenyl, C₂₋₆alkynyl, haloC₁₋₆alkyl, C₁₋₆alkoxy, haloC₁₋₆alkoxy, —C(O)NR^{a}R^{b}, —C(O)—C₁₋₆alkyl, formyl, —C(O)OH, a-C(O)O—C₁₋₆alkyl, benzyloxy, C₁₋₄alkoxyphenyl, pyrrolidinyl, morpholinyl, tetrahydrofuranyl and triazolyl;

R⁵ is hydrogen or C₁₋₆alkyl optionally substituted with one, two or three substituents independently selected from the group consisting of halogen, —OH, C₁₋₅alkoxy, —NR^{a}R^{b}, and R^{a}R^{b}N—C₁₋₆alkyl;

R⁶ is hydrogen or C₁₋₆alkyl;

R³² is halo, —OH, —CN, —NO₂, oxo, hydrazino, formyl, azido, silyl, siloxy, —S(O)_{q}—C₁₋₆alkyl, —NR^{a}R^{b}, —NR^{c}—S(O)ₜ—C₁₋₆alkyl, —S(O)—NR^{a}R^{b}, C₁₋₆alkyl, C₂₋₆alkenyl, C₂₋₆alkynyl, C₃₋₆cycloalkyl, haloC₁₋₆alkyl, hydroxyC₁₋₆alkyl, R^{a}R^{b}N—C₁₋₆alkyl-, C₁₋₆alkoxy, haloC₁₋₆alkoxy, hydroxyC₁₋₆alkoxy-, R^{a}R^{b}N—C₁₋₆alkoxy-, C₁₋₆alkoxyC₁₋₆alkyl, —C(O)NR^{a}R^{b}, —C(O)—C₁₋₆alkyl, —C(O)OH, or —C(O)O—C₁₋₆alkyl;

R³⁴ is hydrogen or C₁₋₄alkyl;

R^{a} and R^{b} are independently selected for each occurrence from the group consisting of hydrogen and C₁₋₆alkyl; or

R^{a} and R^{b} may be taken together with the nitrogen to which R^{a} and R^{b} are attached to form:

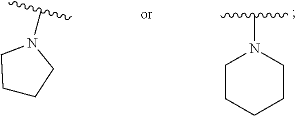 [SMILES: *N1CCCC1 ; *N1CCCCC1]

R^{c} is independently selected for each occurrence from the group consisting of hydrogen and C₁₋₆alkyl;

for each occurrence, q is independently 0, 1 or 2;

for each occurrence, t is independently 1 or 2; and

w is 0, 1 or 2.

In certain embodiments, the compound of Formula I is a compound of Formula II or III:

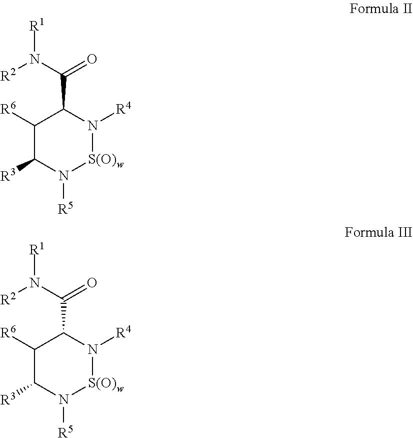

or a pharmaceutically acceptable salt thereof.

In certain embodiments, the compound of Formula I is a compound of Formula II:

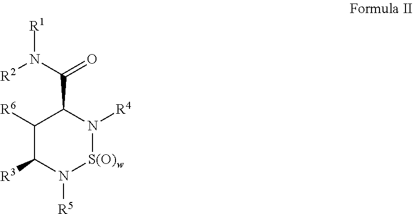

or a pharmaceutically acceptable salt thereof.

In certain embodiments, the compound of Formula I is a compound of Formula III:

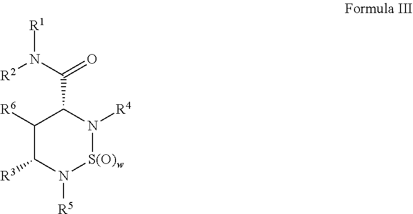

or a pharmaceutically acceptable salt thereof.

In certain embodiments, the compound of Formula I is a compound of Formula IV or V:

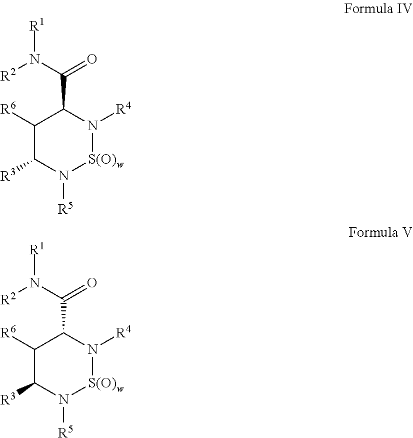

or a pharmaceutically acceptable salt thereof.

In certain embodiments, the compound of Formula I is a compound of Formula IV:

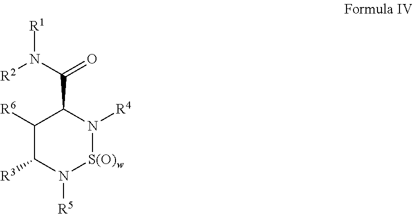

or a pharmaceutically acceptable salt thereof.

In certain embodiments, the compound of Formula I is a compound of Formula V:

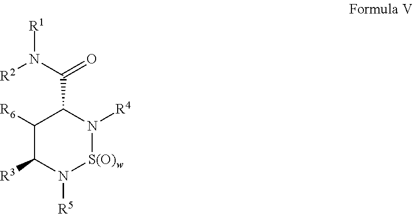

or a pharmaceutically acceptable salt thereof.

In certain embodiments, w is 0.

In certain embodiments, w is 1.

In certain embodiments, w is 2.

In certain embodiments, L is —C(O)—, —C(O)C₁₋₆alkyl- or —C₁₋₆alkylC(O)—.

In certain embodiments, L is —C₁₋₆alkylC(O)NR^{c}— or —C₁₋₆alkylNR^{c}C(O)—.

In certain embodiments, L is —C₁₋₆alkylC(O)NH— or —C₁₋₆alkylNHC(O)—.

In certain embodiments, L is —C(O)NR^{c}—, —C(O)NR^{c}C₁₋₆alkyl- or —NR^{c}C(O)C₁₋₆alkyl-.

In certain embodiments, L is —C(O)NR^{c}—.

In certain embodiments, L is —C(O)NH—.

In certain embodiments, —C(O)NR^{c}C₁₋₆alkyl-.

In certain embodiments, —C(O)NHC₁₋₆alkyl-.

In certain embodiments, R¹ is a phenyl optionally substituted with one, two, or three independently selected R³² groups.

In certain embodiments, R¹ is

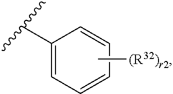

wherein:

R³² is independently selected for each occurrence from the group consisting of hydrogen, halo, cyano, C₁₋₆alkyl and C₁₋₆haloalkyl; and

r2 is 0, 1, 2 or 3.

In certain embodiments, R¹ is

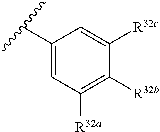

wherein: R^{32a}, R^{32b} and R^{32c} are independently selected from the group consisting of hydrogen, cynano, F, Cl and Br.

In certain embodiments, R¹ is

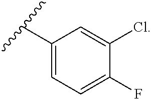 [SMILES: *c1ccc(F)c(Cl)c1]

In certain embodiments, R¹ is a heteroaryl optionally substituted with one, two, or three independently selected R³² groups.

In certain embodiments, R¹ is a 5-6 membered monocyclic heteroaryl optionally substituted with one, two, or three independently selected R³² groups.

In certain embodiments, R¹ is a 5-6 membered monocyclic heteroaryl optionally substituted with one, two, or three independently selected R³² groups; wherein:

the 5-6 membered monocyclic heteroaryl is selected from the group consisting of: furanyl, thienyl, pyrrolyl, thiazolyl, oxazolyl, isothiazolyl, isoxazolyl, imidazolyl, pyrazolyl, 1H-1,2,3-triazolyl, 2H-1,2,3-triazolyl, 1,2,4-triazolyl, pyridinyl, pyridazinyl, pyrimidinyl, pyrazinyl, 1,3,5-triazinyl, 1,2,4-triazinyl, 1,2,3-triazinyl, 1,2,4-oxadiazolyl, 1,3,4-oxadiazolyl, 1,2,5-oxadiazolyl, 1,2,4-thiadiazolyl, 1,3,4-thiadiazolyl and 1,2,5-thiadiazolyl.

In certain embodiments, R¹ is a pyridyl optionally substituted with one, two, or three substituents independently selected from the group consisting of halo, cyano, C₁₋₆alkyl and C₁₋₆haloalkyl.

In certain embodiments, R¹ is a 8-12 membered bicyclic heteroaryl optionally substituted with one, two, or three independently selected R³² groups.

In certain embodiments, R¹ is a 8-12 membered bicyclic heteroaryl optionally substituted with one, two, or three independently selected R³² groups, wherein:

the 8-12 membered bicyclic heteroaryl is selected from the group consisting of: benzofuranyl, isobenzofuranyl, benzo[b]thiophenyl, benzo[c]thiophenyl, indolyl, isoindolyl, benzo[d]isoxazolyl, benzo[c]isoxazolyl, benzo[d]oxazolyl, benzo[d]isothiazolyl, benzo[c]isothiazolyl, benzo[d]thiazolyl, indazolyl, benzo[d]imidazolyl, benzo[d]imidazolyl, and benzo[d][1,2,3]triazolyl.

In certain embodiments, R² is hydrogen or methyl.

In certain embodiments, R² is hydrogen.

In certain embodiments, R⁴ is hydrogen, C₁₋₆alkyl, C₂₋₆alkenyl or C₂₋₆alkynyl, wherein: the C₁₋₄alkyl, C₂₋₆alkenyl or C₂₋₆alkynyl is optionally substituted with hydroxy, cyano, C₁₋₄alkoxy, haloC₁₋₄alkoxy, methylsulfonyl, diethylamino, carboxy, carbamoyl, benzyloxy, formyl, methoxyphenyl, pyrrolidinyl, morpholinyl, tetrahydrofuranyl or triazolyl.

In certain embodiments, R⁴ is hydrogen or C₁₋₆alkyl optionally substituted with C₁₋₆alkoxy, —NR^{a}R^{b}, C₂₋₆alkenyl, —OH, —COOH or C₁₋₆haloalkoxy.

In certain embodiments, R⁴ is C₁₋₆alkyl optionally substituted with C₁₋₆alkoxy, —NR^{a}R^{b}, C₂₋₆alkenyl, —OH, —COOH or C₁₋₆haloalkoxy.

In certain embodiments, R⁴ is —CH₂CH₂OCH₃.

In certain embodiments, R⁴ is methyl.

In certain embodiments, R⁵ is hydrogen, C₁₋₄alkyl, C₁₋₄alkoxy, or R^{a}R^{b}N—C₁₋₄alkyl-.

In certain embodiments, R⁵ is hydrogen, methyl, methoxymethyl-, methoxyethyl- or dimethylaminoethyl-.

In certain embodiments, R⁵ is hydrogen or methyl.

In certain embodiments, R⁵ is hydrogen.

In certain embodiments, R⁶ is hydrogen or C₁₋₆alkyl.

In certain embodiments, R⁶ is hydrogen.

In certain embodiments, R⁷ hydrogen.

In certain embodiments, R⁷ is a cycloalkyl, cycloalkenyl or carbocyclyl, wherein: the cycloalkyl, cycloalkenyl or carbocyclyl is optionally substituted with one, two or three substituents independently selected from the group consisting of R³², R³⁴ and R^{7a}.

In certain embodiments, R⁷ is a cycloalkyl optionally substituted with one, two or three substituents independently selected from the group consisting of R³², R³⁴ and R^{7a}.

In certain embodiments, R⁷ is a cycloalkenyl optionally substituted with one, two or three substituents independently selected from the group consisting of R³², R³⁴ and R^{7a}.

In certain embodiments, R⁷ is a carbocyclyl optionally substituted with one, two or three substituents independently selected from the group consisting of R³², R³⁴ and R^{7a}.

In certain embodiments, R⁷ is a heterocyclkoalkyl, heterocyclkoalkenyl or heterocyclyl, wherein: the heterocyclkoalkyl, heterocyclkoalkenyl or heterocyclyl is optionally substituted with one, two or three substituents independently selected from the group consisting of R³², R³⁴ and R^{7a}.

In certain embodiments, R⁷ is a heterocyclkoalkyl optionally substituted with one, two or three substituents independently selected from the group consisting of R³², R³⁴ and R^{7a}.

In certain embodiments, R⁷ is a 4-7 membered monocyclic heterocyclkoalkyl optionally substituted with one, two or three substituents independently selected from the group consisting of R³², R³⁴ and R^{7a}, wherein:

the 4-7 membered monocyclic heterocyclkoalkyl is selected form the group consisting of: aziridinyl, oxiranyl, thiiranyl 1,1-dioxide, oxetanyl, azetidinyl, thietanyl 1,1-dioxide, pyrrolidinyl, tetrahydrofuranyl, piperidinyl, tetrahydro-2H-pyranyl, morpholinyl, thiomorpholinyl and piperazinyl.

In certain embodiments, R⁷ is a heterocyclkoalkenyl optionally substituted with one, two or three substituents independently selected from the group consisting of R³², R³⁴ and R^{7a}.

In certain embodiments, R⁷ is a heterocyclyl optionally substituted with one, two or three substituents independently selected from the group consisting of R³², R³⁴ and R^{7a}.

In certain embodiments, R⁷ is a phenyl optionally substituted with one, two or three substituents independently selected from the group consisting of R³², R³⁴ and R^{7a}.

In certain embodiments, R⁷ is a heteroaryl optionally substituted with one, two or three substituents independently selected from the group consisting of R³², R³⁴ and R^{7a}.

In certain embodiments, R⁷ is a 5-6 membered monocyclic heteroaryl optionally substituted with one, two or three substituents independently selected from the group consisting of R³², R³⁴ and R^{7a}.

In certain embodiments, R⁷ is a 5-6 membered monocyclic heteroaryl optionally substituted with one, two or three substituents independently selected from the group consisting of R³², R³⁴ and R^{7a}, wherein:

the 5-6 membered monocyclic heteroaryl is selected from the group consisting of: furanyl, thienyl, pyrrolyl, thiazolyl, oxazolyl, isothiazolyl, isoxazolyl, imidazolyl, pyrazolyl, 1H-1,2,3-triazolyl, 2H-1,2,3-triazolyl, 1,2,4-triazolyl, pyridinyl, pyridazinyl, pyrimidinyl, pyrazinyl, 1,3,5-triazinyl, 1,2,4-triazinyl, 1,2,3-triazinyl, 1,2,4-oxadiazolyl, 1,3,4-oxadiazolyl, 1,2,5-oxadiazolyl, 1,2,4-thiadiazolyl, 1,3,4-thiadiazolyl and 1,2,5-thiadiazolyl.

In certain embodiments, R⁷ is a 8-12 membered bicyclic heteroaryl optionally substituted with one, two or three substituents substituents independently selected from the group consisting of R³², R³⁴ and R^{7a}.

In certain embodiments, R⁷ is a 8-12 membered bicyclic heteroaryl optionally substituted with one, two or three substituents independently selected from the group consisting of R³², R³⁴ and R^{7a}, wherein:

the 8-12 membered bicyclic heteroaryl is selected from the group consisting of: benzofuranyl, isobenzofuranyl, benzo[b]thiophenyl, benzo[c]thiophenyl, indolyl, isoindolyl, benzo[d]isoxazolyl, benzo[c]isoxazolyl, benzo[d]oxazolyl, benzo[d]isothiazolyl, benzo[c]isothiazolyl, benzo[d]thiazolyl, indazolyl, benzo[d]imidazolyl, benzo[d]imidazolyl, and benzo[d][1,2,3]triazolyl.

In certain embodiments, R^{7a} is a phenyl optionally substituted with one, two or three independently selected R³² groups.

In certain embodiments, R^{7a} is a heteroaryl optionally substituted with one, two or three independently selected R³² groups.

In certain embodiments, R^{7a} is a 5-6 membered monocyclic heteroaryl optionally substituted with one, two or three independently selected R³² groups.

In certain embodiments, R⁷ is a 8-12 membered bicyclic heteroaryl selected from the group consisting of:

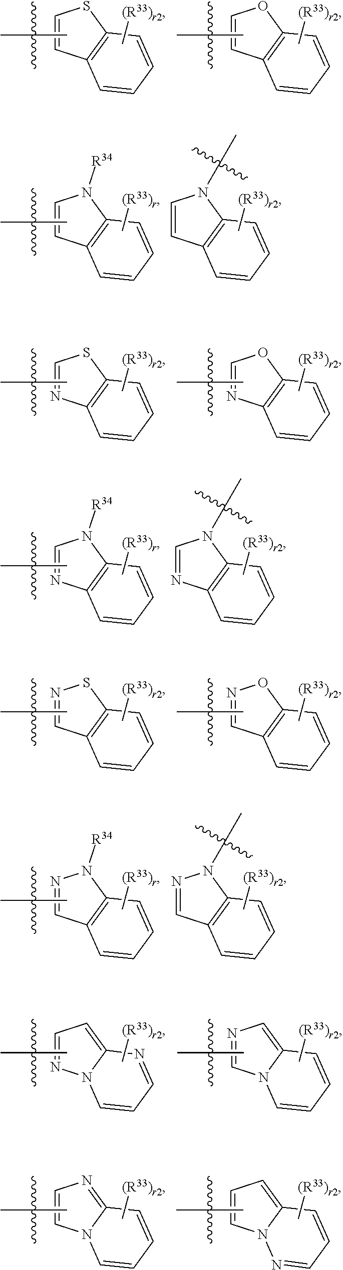

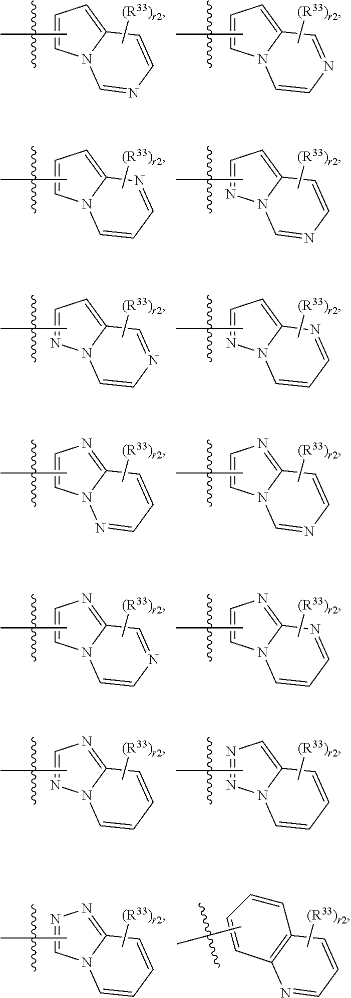

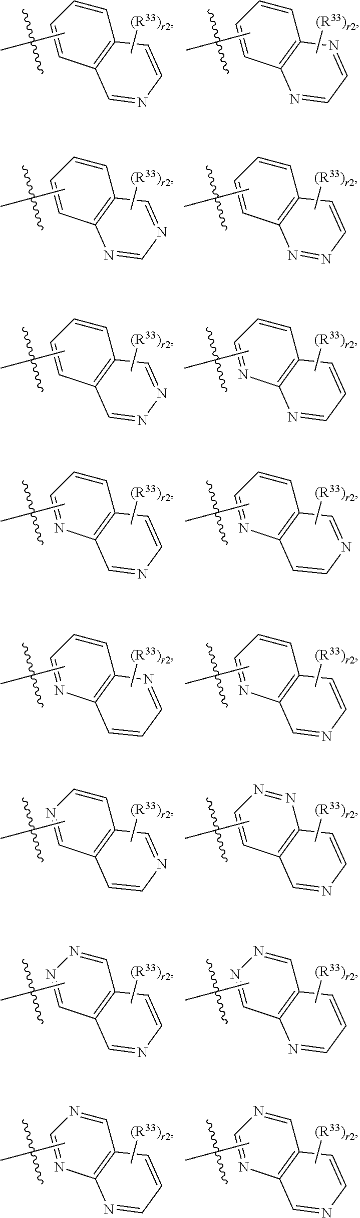

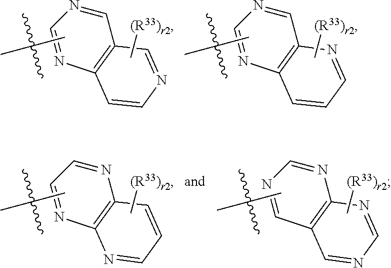

R³³ is independently selected for each occurrence from the group consisting of R³² and R^{7a};

R³⁴ is hydrogen or C₁₋₄alkyl;

r is 0, 1 or 2; and

r2 is 0, 1, 2 or 3.

In certain embodiments, R⁷ is a 5-6 membered monocyclic heteroaryl selected from the group consisting of:

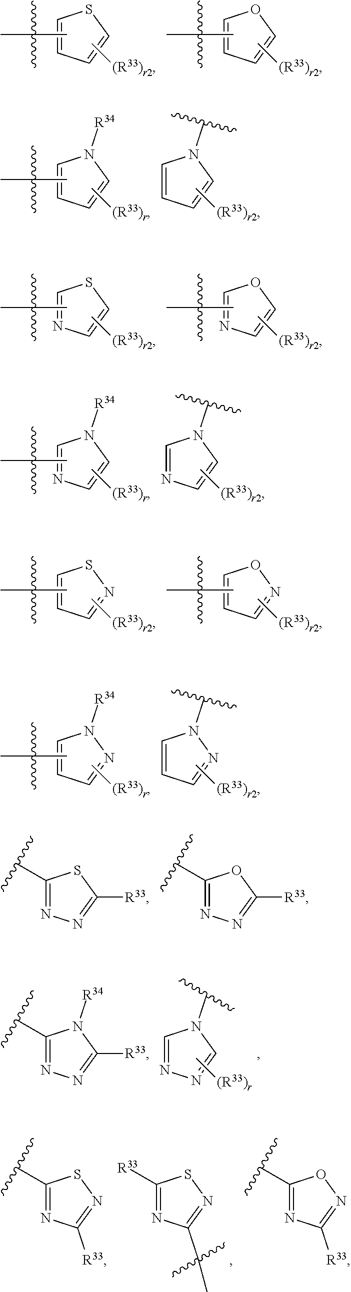

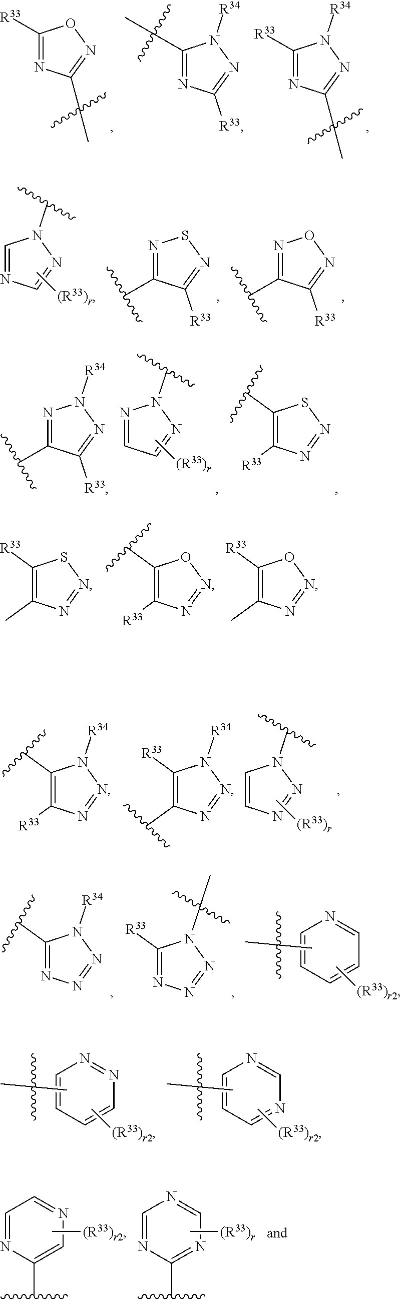

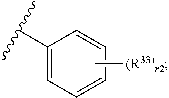

R³³ is independently selected for each occurrence from the group consisting of R³² and R^{7a};

R³⁴ is hydrogen or C₁₋₄alkyl;

r is 0, 1 or 2; and

r2 is 0, 1, 2 or 3.

In certain embodiments, R⁷ is

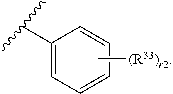

In certain embodiments, R⁷ is

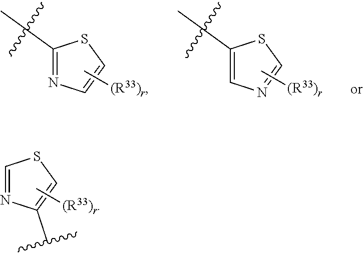

In certain embodiments, R⁷ is

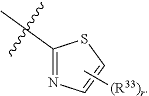

In certain embodiments, R⁷ is

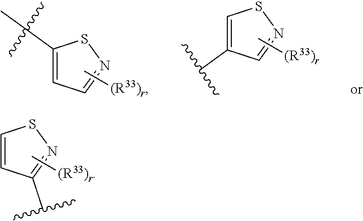

In certain embodiments, R⁷ is

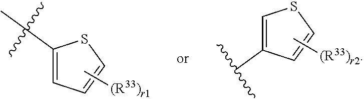

In certain embodiments, R⁷ is

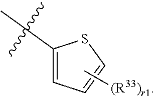

In certain embodiments, R⁷ is

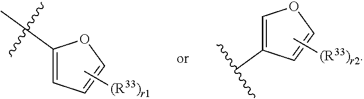

In certain embodiments, R⁷ is

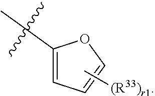

In certain embodiments, R² and R⁶ are hydrogen.

In certain embodiments, R² and R⁶ are hydrogen, and w is 2.

In certain embodiments, R², R⁵ and R⁶ are hydrogen.

In certain embodiments, R², R⁵ and R⁶ are hydrogen, and w is 2.

In certain embodiments, R², R⁵ and R⁶ are hydrogen, and R⁴ is methyl.

In certain embodiments, R², R⁵ and R⁶ are hydrogen, R⁴ is methyl, and w is 2.

In certain embodiments, R¹ is 3-chloro-4-fluorophenyl, R² is hydrogen, and R⁶ is hydrogen.

In certain embodiments, R¹ is 3-chloro-4-fluorophenyl, R² is hydrogen, R⁶ is hydrogen, and w is 2.

In certain embodiments, R¹ is 3-chloro-4-fluorophenyl, R² is hydrogen, R⁵ is hydrogen, and R⁶ is hydrogen.

In certain embodiments, R¹ is 3-chloro-4-fluorophenyl, R² is hydrogen, R⁵ is hydrogen, R⁶ is hydrogen, and w is 2.

In certain embodiments, R¹ is 3-chloro-4-fluorophenyl, R² is hydrogen, R⁵ is hydrogen, R⁶ is hydrogen, and R⁴ is methyl.

In certain embodiments, R¹ is 3-chloro-4-fluourophenyl, R² is hydrogen, R⁵ is hydrogen, R⁶ is hydrogen, R⁴ is methyl, and w is 2.

It will be appreciated that all chemically allowable combinations of the embodiments described above, and elsewhere in this disclosure, are envisioned as further embodiments of the invention.

### II. Pharmaceutical Compositions and Kits

In another aspect, the disclosure provides pharmaceutical compositions comprising a compound of Formula I, or a pharmaceutically acceptable salt thereof, and a pharmaceutically acceptable excipient. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

In another aspect, the disclosure provides a pharmaceutical composition comprises a compound of Table 2 or 3, or a pharmaceutically acceptable salt and/or stereoisomer thereof.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methylacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by a e.g. a consumer in need of HBV infection treatment. Such kits include a suitable dosage form such as those described above and instructions describing the method of using such dosage form to mediate, reduce or prevent HBV infection. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### III. Methods

In a further aspect, a method for treating a hepatitis B infection in a patient in need thereof is provided, comprising administering to a subject or patient an effective amount of a disclosed compound, and/or administering a first disclosed compound and optionally, an additional, different disclosed compound(s). In another embodiment, a method for treating a hepatitis B infection in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

For use in accordance with this aspect, the appropriate dosage is expected to vary depending on, for example, the particular compound employed, the mode of administration, and the nature and severity of the infection to be treated as well as the specific infection to be treated and is within the purview of the treating physician. Usually, an indicated administration dose may be in the range between about 0.1 to about 1000 μg/kg body weight. In some cases, the administration dose of the compound may be less than 400 μg/kg body weight. In other cases, the administration dose may be less than 200 μg/kg body weight. In yet other cases, the administration dose may be in the range between about 0.1 to about 100 μg/kg body weight. The dose may be conveniently administered once daily, or in divided doses up to, for example, four times a day or in sustained release form.

A compound of the present disclosure may be administered by any conventional route, in particular: enterally, topically, orally, nasally, e.g. in the form of tablets or capsules, via suppositories, or parenterally, e.g. in the form of injectable solutions or suspensions, for intravenous, intra-muscular, sub-cutaneous, or intra-peritoneal injection. Suitable formulations and pharmaceutical compositions will include those formulated in a conventional manner using one or more physiologically acceptable carriers or excipients, and any of those known and commercially available and currently employed in the clinical setting. Thus, the compounds may be formulated for oral, buccal, topical, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound(s) over an extended period. For buccal administration the compositions may take the form of tablets or lozenges formulated in a conventional manner known to the skilled artisan.

A disclosed compound may also be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the compound may be in powder form for constitution with a suitable vehicle, e.g. sterile pyrogen-free water, before use. Compounds may also be formulated for rectal administration as suppositories or retention enemas, e.g. containing conventional suppository bases such as cocoa butter or other glycerides.

Also contemplated herein are methods and compositions that include a second active agent, or administering a second active agent. For example, in addition to being infected with HBV, a subject or patient can further have HBV infection-related co-morbidities, i.e., diseases and other adverse health conditions associated with, exacerbated by, or precipitated by being infected with HBV. Contemplated herein are disclosed compounds in combination with at least one other agent that has previously been shown to treat these HBV-infection-related conditions.

In some cases, a disclosed compound may be administered as part of a combination therapy in conjunction with one or more antivirals. Example antivirals include nucleoside analogs, interferon α, and other assembly effectors, for instance heteroaryldihydropyrimidines (HAPs) such as methyl 4-(2-chloro-4-fluorophenyl)-6-methyl-2-(pyridin-2-yl)-1,4-dihydropyrimidine-5-carboxylate (HAP-1). For example, provided herein is a method of treating a patient suffering from hepatitis B infection comprising administering to the patient a first amount of a disclosed compound and a second amount of an antiviral, or other anti HBV agent, for example a second amount of a second compound selected from the group consisting of: a HBV capsid assembly promoter (for example, GLS4, BAY 41-4109, AT-130, DVR-23 (e.g., as depicted below),

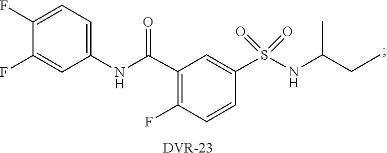

NVR 3-778, NVR1221 (by code); and N890 (as depicted below):

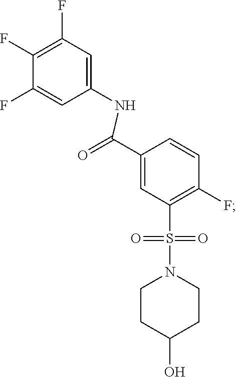 [SMILES: O=C(Nc1cc(F)c(F)c(F)c1)c1ccc(F)c(S(=O)(=O)N2CCC(O)CC2)c1]

other CpAMs such as those disclosed in the following patent applications hereby incorporated by reference: WO2014037480, WO2014184328, WO2013006394, WO2014089296, WO2014106019, WO2013102655, WO2014184350, WO2014184365, WO2014161888, WO2014131847, WO2014033176, WO2014033167, and WO2014033170; Nucleoside analogs interfering with viral polymerase, such as entecavir (Baraclude), Lamivudine, (Epivir-HBV), Telbivudine (Tyzeka, Sebivo), Adefovir dipivoxil (Hepsera), Tenofovir (Viread), Tenofovir alafenamide funarate (TAF), prodrugs of tenofavir (e.g. AGX-1009), L-FMAU (Clevudine), LB80380 (Besifovir) and:

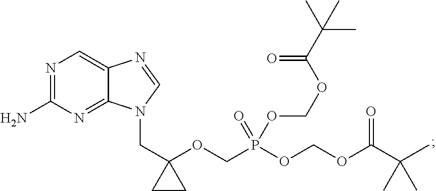 [SMILES: CC(C)(C)C(=O)OCOP(=O)(COC1(Cn2cnc3cnc(N)nc32)CC1)OCOC(=O)C(C)(C)C]

viral entry inhibitors such as Myrcludex B and related lipopeptide derivatives; HBsAg secretion inhibitors such as REP 9AC′ and related nucleic acid-based amphipathic polymers, HBF-0529 (PBHBV-001), PBHBV-2-15 as depicted below:

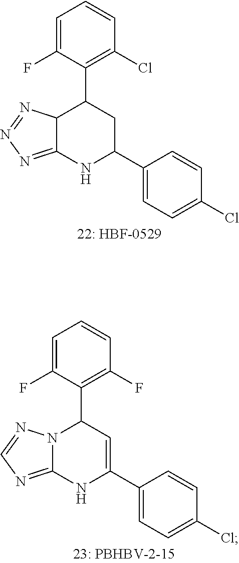 [SMILES: Fc1cccc(Cl)c1C1CC(c2ccc(Cl)cc2)NC2=NN=NC21 ; Fc1cccc(F)c1C1C=C(c2ccc(Cl)cc2)Nc2ncnn21]

and BM601 as depicted below:

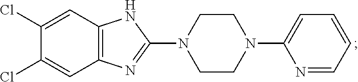 [SMILES: Clc1cc2nc(N3CCN(c4ccccn4)CC3)[nH]c2cc1Cl]

disruptors of nucleocapsid formation or integrity such as NZ-4/W28F:

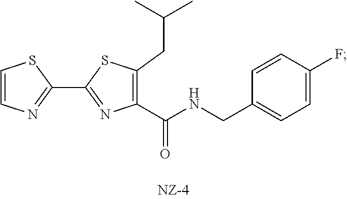 [SMILES: CC(C)Cc1sc(-c2nccs2)nc1C(=O)NCc1ccc(F)cc1]

cccDNA formation inhibitors such as BSBI-25, CCC-0346, CCC-0975 (as depicted below):

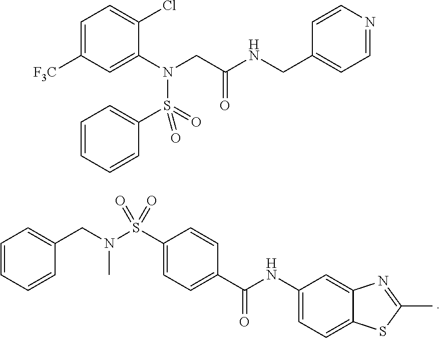

HBc directed transbodies such as those described in Wang Y, et al, Transbody against hepatitis B virus core protein inhibits hepatitis B virus replication in vitro, Int. Immunopharmacol (2014), located at //dx.doi.org/10.1016/j.intimp.2015.01.028; antiviral core protein mutant (such as Cp183-V124W and related mutations as described in WO/2013/010069, WO2014/074906, each incorporated by reference); inhibitors of HBx-interactions such as RNAi, antisense and nucleic acid based polymers targeting HBV RNA, e.g., RNAi (for example ALN-HBV, ARC-520, TKM-HBV, ddRNAi), antisense (ISIS-HBV), or nucleic acid based polymer: (REP 2139-Ca); immunostimulants such as Interferon alpha 2a (Roferon), Intron A (interferon alpha 2b), Pegasys (peginterferon alpha 2a), Pegylated IFN 2b, IFN lambda 1a and PEG IFN lambda 1a, Wellferon, Roferon, Infergen, lymphotoxin beta agonists such as CBE11 and BS1); Non-Interferon Immune enhancers such as Thymosin alpha-1 (Zadaxin) and Interleukin-7 (CYT107); TLR-7/9 agonists such as GS-9620, CYT003, Resiquimod; Cyclophilin Inhibitors such as NVP018; OCB-030; SCY-635; Alisporivir; NIM811 and related cyclosporine analogs; vaccines such as GS-4774, TG1050, Core antigen vaccine; SMAC mimetics such as birinapant and other IAP-antagonists; Epigenetic modulators such as KMT inhibitors (EZH1/2, G9a, SETD7, Suv39 inhibitors), PRMT inhibitors, HDAC inhibitors, SIRT agonists, HAT inhibitors, WD antagonists (e.g. OICR-9429), PARP inhibitors, APE inhibitors, DNMT inhibitors, LSD1 inhibitors, JMJD HDM inhibitors, and Bromodomain antagonists; kinase inhibitors such as TKB1 antagonists, PLK1 inhibitors, SRPK inhibitors, CDK2 inhibitors, ATM & ATR kinase inhibitors; STING Agonists; Ribavirin; N-acetyl cysteine; November-205 (BAM205); Nitazoxanide (Alinia), Tizoxanide; SB 9200 Small Molecule Nucleic Acid Hybrid (SMNH); DV-601; Arbidol; FXR agonists (such as GW 4064 and Fexaramin); antibodies, therapeutic proteins, gene therapy, and biologics directed against viral components or interacting host proteins.

In some embodiments, the disclosure provides a method of treating a hepatitis B infection in a patient in need thereof, comprising administering a first compound selected from any one of the disclosed compounds, and one or more other HBV agents each selected from the group consisting of HBV capsid assembly promoters, HBF viral polymerase interfering nucleosides, viral entry inhibitors, HBsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a hepatitis B infection in a patient in need thereof, comprising administering an amount of a disclosed compound, and administering another HBV capsid assembly promoter.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of a disclosed compound first, for example one or more days or weeks prior to initiation of administration of the antiviral. Moreover, additional drugs may be given in conjunction with the above combination therapy.

In another embodiment, a disclosed compound may be conjugated (e.g., covalently bound directly or through molecular linker to a free carbon, nitrogen (e.g. an amino group), or oxygen (e.g. an active ester) of a disclosed compound), with a detection moiety, for e.g., a fluorophore moiety (such a moiety may for example re-emit a certain light frequency upon binding to a virus and/or upon photon excitation). Contemplated fluorophores include AlexaFluor® 488 (Invitrogen) and BODIPY FL (Invitrogen), as well as fluorescein, rhodamine, cyanine, indocarbocyanine, anthraquinones, fluorescent proteins, aminocoumarin, methoxycoumarin, hydroxycoumarin, Cy2, Cy3, and the like. Such disclosed compounds conjugated to a detection moiety may be used in e.g. a method for detecting HBV or biological pathways of HBV infection, e.g., in vitro or in vivo; and/or methods of assessing new compounds for biological activity.

### IV. Examples

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Abbreviations

DCM Dichloromethane
EtOAC Ethyl acetate
MeOH Methanol
DMSO Dimethyl sulfoxide
NMO N-Methylmorpholine N-oxide
LiHMDS Lithium bis(trimethylsilyl)amide
p-TSA p-Toluenesulfonic acid
DMF N,N-Dimethylformamide
THF Tetrahydrofuran
TLC Thin-layer chromatography
LCMS Liquid chromatography-mass spectrometry
HPLC High performance liquid chromatography

### Synthetic Methods

The compounds described herein can be prepared by various methods based on the teachings contained herein and synthetic procedures known in the art. The variables shown in the synthetic schemes are distinct from and should not be confused with the variables in the claims or the rest of the specification. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

Methods useful for the preparation of compounds of this invention are illustrated in the following schemes. In Scheme I, an appropriately substituted methyl ketone (I-1) can be reacted with a bis-ester of oxalic acid (I-2) in the presence of a suitable base such as, EtONa, t-BuOK, LiHMDS or LDA to form diketoester I-3. Synthesis of the corresponding 2H-1,2,6-thiadiazine, 1,1-dioxide derivative, I-4, can be accomplished by condensing intermediate I-2 with sulfuric diamide (H4N202S) under acidic conditions (eg. HCl) similar to those described in Bioorganic & Medicinal Chemistry Letters, (2007), 7, 7480. This intermediate can be reacted with an appropriate alcohol (R⁴OH) under Mitsunobu reaction conditions (Mitsunobu, O. et al. Bulletin of the Chemical Society of Japan, (1967). 40, 935) or an alkyl halide (R⁴X where X=I, Br or Cl) in the presence of a base (eg. NaOH, KOH, K₂CO₃, NaH, LiHMDS, NaHMDS) to selectively form intermediate I-5. Intermediate I-5 can be taken on to the final product (I-9) by a number of different pathways. In one of these pathways, intermediate I-5 is treated under hydrolytic conditions (eg. NaOH or Et₃N/H₂O) to form carboxylic acid I-6. This intermediate can be treated with a reducing agent such as NaBH₄ to form I-8. Alternatively, I-8 can be formed by the hydrogenation of I-6 using a Ni, Pd, Pt, Ru, Rh or Ir based-catalyst and H2 or a hydrogen-donating reagent (eg. N₂H₄, H₂N₂, dihydronaphthalene, dihydroanthracene, isopropanol, formic acid, H₂O). Following this, I-8 is coupled with an appropriate amine (R¹R²NH) using an amide bond forming reagent (eg. DCC, PyBOP, PyBrop, TDBTU, HATU) and base (eg. Et₃N, iPr₂NEt) to yield the final product. In an alternative pathway, ester I-5 can be directly converted to amide I-7 using an appropriate amine (R¹R²NH) and a reagent such as Me₃Al, which mediates ester-amide exchange. Intermediate I-7 can be reduced to form the final product, I-9, using methods similar to those described for the conversion of I-6 to I-8.

It will be understood by one skilled in the art that the 3,5-disubstituted 1,2,6-thiadiazinane 1,1, dioxide I-9 can exist as two different configurational isomers referred to as cis or trans depending on whether substituents at the 3- and 5-positions lie on the same- or opposite-face of the ring, respectively. In the current invention the 3,5-cis stereoisomer can be selectively produced by the reduction or hydrogenation reactions of I-6 to form I-8 or by the reduction of I-7 to form I-9.

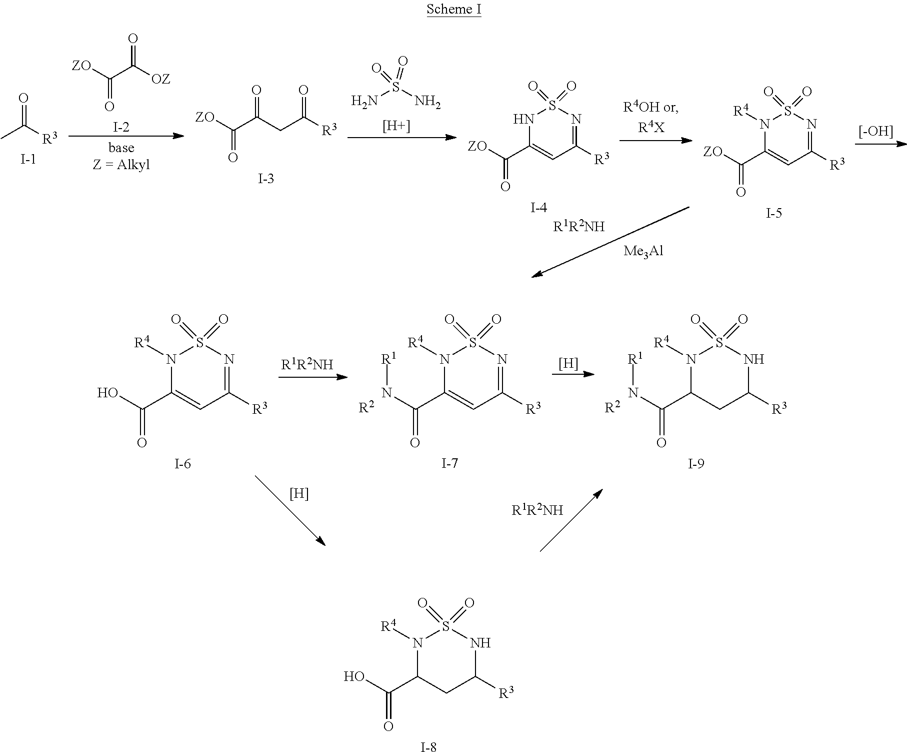

It will be appreciated by one skilled in the art that the cis-isomer of I-9 can exist as a mixture of enantiomers, 3R,5S and 3S,5R. The individual enantiomers can be derived from racemic I-8 or isolated from racemic I-9 according to the methods illustrated in Scheme II. In one method, intermediate I-8 can be resolved using chiral chromatography or selective salt-formation and crystallization using an enantiomerically pure chiral amine. The purified enantiomers, II-1a and II-1b can then be individually converted to the corresponding amides II-2a and II-2b. Alternatively, racemic I-8 can be converted to racemic I-9 which can then be separated into its individual enantiomers II-2a and II-2b using chiral chromatography. The isolated enantiomers are referred to as Isomer I and Isomer II based on their order of elution from the chiral column regardless of the absolute stereochemistry. The first eluting isomer is designated as Isomer I and the second is referred to as Isomer II.

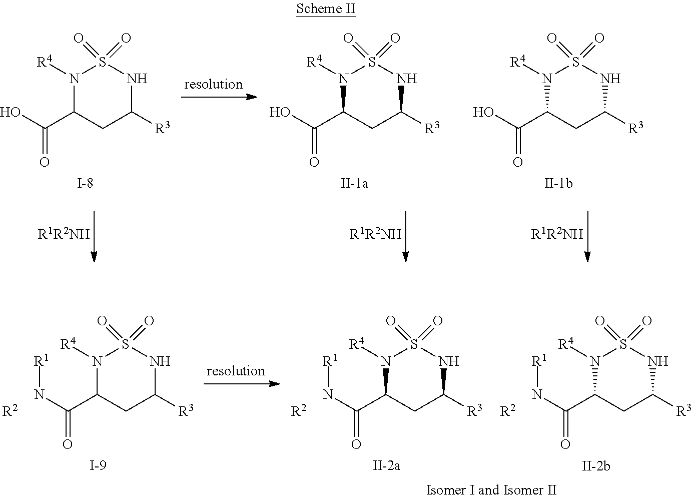

Scheme III illustrates the direct synthesis of enantiomers II-2a or II-2b from prochiral intermediate I-7. For example, this can be accomplished by asymmetric transfer hydrogenation, as described in Accounts of Chemical Research (1997) 30, 97 or Angewandte Chemie International Edition (1998) 12, 41.

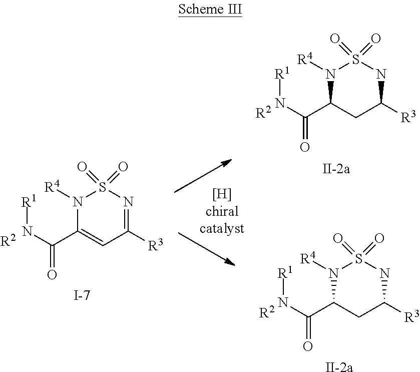

Certain compounds of this invention can be produced according to the methods shown in Scheme IV. Advanced intermediate IV-6 can be synthesized using the methods described in Scheme I. Intermediate IV-6 can then be treated with a suitable oxidizing reagent such as KMnO₄ to form carboxylic acid IV-7. This intermediate can be coupled to an amine, R⁷R^{c}NH, under the conditions described previously to provide racemic compound IV-8. The individual enantiomers, IV-9a and IV-9b can be isolated by chiral chromatography.

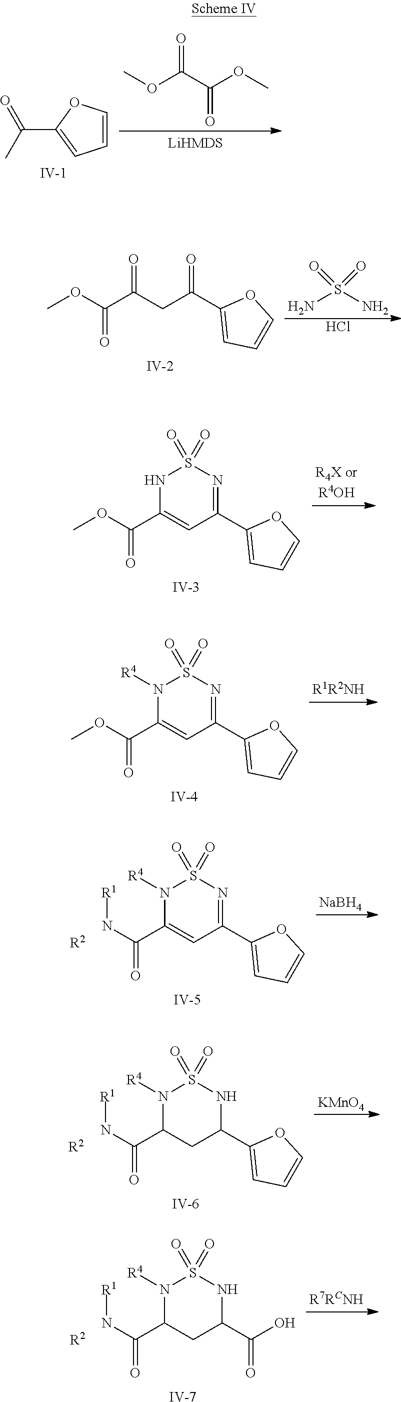

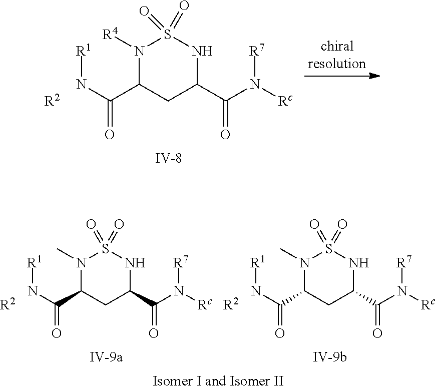

In Scheme V, intermediate V-2 can be synthesized by coupling IV-7 with amine V-1 under standard amide bond forming conditions. Compound V-2 can then be derivatized using Y—X (X=H, halide Zn, Mg, B(OH)₂, B(OZ)₂, SnZ₃, or SiZ₃ (Z=alkyl or aryl)) under palladium, nickel, copper, platinum, iron or cobalt catalysis to yield V-3.

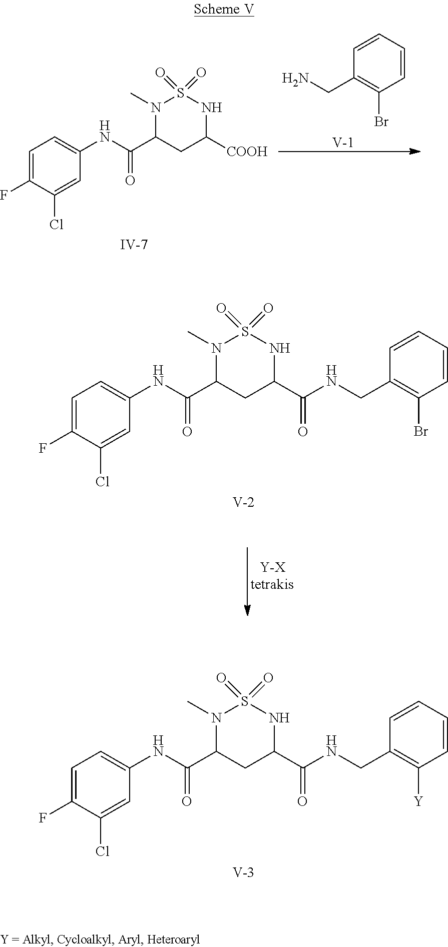

### Methods for Chiral Separation

Isolation of the individual enantiomers of compounds can be accomplished using one or more of the following chromatography methods, Separation Method A, Separation Method B and Separation Method C described below. In the following examples, the compound eluting first is referred to as Isomer I, while the second eluting compound is referred to as Isomer II.

### Separation Method A

Column: YMC chiral Amylose-SA, 250 mm×20 mm, 5 micron

### Mobile Phase:

A: n-Hexane+0.1% DEA

### B: DCM:MeOH (1:1)

### Isocratic: 30-90% B

Flow rate: 18 mL/Min

### Separation Method B

Column: DIACEL CHIRALPACK-IA, 250 mm×20 mm, 5 micron

### Mobile Phase:

A: n-Hexane+0.1% DEA

### B: DCM:MeOH (1:1)

Gradient: Hold 50% B till 4 min then 100% B at 5 min & hold up to 15 min
Flow rate: 18 mL/Min

### Separation Method C

Column: CHIRALPACK-IA, 250 mm×30 mm, 5 micron

### Mobile Phase:

A: n-Hexane+0.1% DEA

### B: DCM:MeOH (1:1)

### Isocratic: 30-90% B

Flow rate: 30 mL/Min

### Chiral Purity Determination

Analysis of the level of chiral purity of the compounds can be evaluated using one or more the chromatography methods Chiral Purity Method A, Chiral Purity Method B and Chiral Purity Method C described below.

### Chiral Purity Method A

Column: YMC chiral Amylose-SA, 250 mm×4.6 mm, 5 micron

### Mobile Phase:

A: n-Hexane+0.1% DEA

### B: DCM:MeOH (1:1)

### Isocratic: 30-90% B

Flow rate: 1 mL/Min

### Chiral Purity Method B

Column: YMC chiral art cellulose-SC, 250 mm×4.6 mm, 5 micron

### Mobile Phase:

A: n-Hexane+0.1% DEA

### B: DCM:MeOH (1:1)

### Isocratic: 30-90% B

Flow rate: 1 mL/Min

### Chiral Purity Method C

Column: CHIRALPACK-IA, 250 mm×4.6 mm, 5 micron

### Mobile Phase:

A: n-Hexane+0.1% DEA

### B: DCM:MeOH (1:1)

### Isocratic: 30-90% B

Flow rate: 30 mL/Min

### General Synthetic Methods

### Method A

To a stirred solution of 1 equivalent of methyl ketone (I-1), in dry THF (10 volumes per gram of methyl ketone) at −78° C. under an Argon atmosphere, was added lithium hexamethyldisilazide (1M in THF, 1.3 eq.). The mixture was stirred for 1 h, following which dimethyl oxalate (1.5 eq.) dissolved in dry THF (5 volumes per gram of dimethyl oxalate) was added dropwise and the resulting reaction mixture stirred at room temperature overnight. After completion, the mixture was concentrated under reduced pressure. The residue was diluted with water and the resulting was collected by filtration. The solid was washed with ethyl acetate followed by diethyl ether and dried under reduced pressure. The resulting diketoester (I-3) was used in the next step without further purification.

### Method B

In a sealable tube, a stirred solution consisting of 1 equivalent of the 2, 4-diketoester, I-3 and 1 equivalent of sulfuric diamide in MeOH (10 volumes per gram of 2,4-diketoester) was purged with HCl gas for 2 h at 0° C. The tube was sealed, and the reaction stirred at 80° C. 24 h. After completion, the reaction mixture was cooled to 0° C. and the resulting precipitated solid was filtered, washed with water, cold methanol then dried in vacuo to afford 2H-1,2,6-thiadiazine 1,1-dioxide, I-4.

### Method C

In a round bottom flask fitted with reflux condenser, 2, 4-diketoester, I-3, (1 eq.) and sulfuric diamide (1 eq.) was taken up in 4 N methanolic HCl (10 volumes per gram of I-3). The resulting reaction mixture was stirred at 60° C. for 16 h after which was cooled to 0° C., to form a precipitate. The precipitated solid was filtered, washed with water followed by diethyl ether and dried in vacuo to afford 2H-1,2,6-thiadiazine 1,1-dioxide, I-4.

### Method D

To a stirred solution of 2H-1,2,6-thiadiazine 1,1-dioxide, I-4 (1 eq.) in dry DMF (8 volumes per gram of 2H-1,2,6-thiadiazine 1,1-dioxide, I-4) at 0° C. under an atmosphere of Ar, NaH (60% w/w in mineral oil, 1.5 eq.) was added and the resulting mixture stirred at 0° C. 45 min. Mel (1.1 eq.) was added slowly and the resulting reaction mixture stirred at room temperature for 12 h. After completion, the reaction mixture was diluted with ice cold water; to afford a solid which was collected by filtration. The solid was washed with diethyl ether and dried in vacuo to yield 2-methyl-2H-1,2,6-thiadiazine 1,1-dioxide, I-5, after silica gel column chromatography.

### Method E

To a stirred solution of 2H-1,2,6-thiadiazine 1,1-dioxide, I-4 (1 eq.) in dry THF (4 volumes per gram of 2H-1,2,6-thiadiazine 1,1-dioxide, I-4) at 0° C. under an Ar atmosphere was added triphenyl phosphine (2 eq.) and methanol (10 eq.). The solution was stirred at 0° C. for 15 min. To this solution was added diethyl azodicarboxylate or diisopropyl azodicarboxylate (2 eq.) and the resulting reaction mixture was stirred at RT for 16 h. After completion, the reaction mixture was concentrated under vacuum and the resulting residue taken up in diethyl ether. The suspension was stirred for 30 min. and the solid isolated by filtration. The solid was stirred in methanol for 30 min., filtered and dried in vacuo to afford 2-methyl-2H-1,2,6-thiadiazine 1,1-dioxide, I-5. This intermediate could be further purified by column chromatography.

### Method F

To a stirred solution of HNR¹R² (3 eq.) in dichloromethane or toluene at 0° C. under Ar atmosphere, was added AlMe₃ (2M in toluene, 3 eq.) and the reaction mixture was stirred at 0° C. for 10 min. The reaction was allowed to warm to RT and stirring continued for 1 h. To this solution, was added 2-methyl-2H-1,2,6-thiadiazine 1,1-dioxide, I-5 (1 eq.), at 0° C. under Ar atmosphere. The resulting mixture was heated to refluxed and stirred overnight. After completion, the reaction mixture was cooled to 0° C. then slowly quenched by the addition of 1N HCl. The mixture was extracted with dichloromethane and the combined organic layers were collected, dried over anhydrous sodium sulphate and concentrated in vacuo. The crude compound was purified by silica gel column chromatography followed by trituration with diethyl ether to afford intermediate I-7.

### Method G

To a solution of 2-methyl-2H-1,2,6-thiadiazine 1,1-dioxide, I-5 (1 eq.) in 1:1 CH₃CN:H₂O (10 volumes per gram of 2-methyl-2H-1,2,6-thiadiazine 1,1-dioxide, I-5) at 0° C. was added triethylamine (5 eq.) and the resulting reaction mixture was stirred until a clear solution was observed (4-6 h). After completion, the mixture was concentrated under reduced pressure, and the resulting residue treated with 6N HCl followed by extraction with ethyl acetate. The combined organic layers were dried over anhydrous sodium sulphate, filtered and concentrated under reduced pressure to afford carboxylic acid I-6 which was used in the next step after trituration with diethyl ether. To a stirred solution of carboxylic acid, I-6 (1 eq.) in CH₂Cl₂ or DMF (10 volumes per gram of I-6) at 0° C. was added diisopropylethylamine (2 eq.). After stirring for 15 min, 1-[bis(dimethylamino)methylene]-1H-1,2,3-triazolo[4,5-b]pyridinium-3-oxide hexafluorophosphate (2 eq.), was added and stirring continued for 15 min after which HNR¹R² (1.2 eq.) was added. The reaction mixture was then stirred at room temperature overnight. After completion, the reaction mixture was diluted with ice cold water and extracted with CH₂Cl₂. The combined organic layers were dried over anhydrous sodium sulphate and concentrated under reduced pressure to afford the crude product. The crude compound was taken up in methanol (10 volumes per gram of crude product), stirred for 15 min., filtered and dried under reduced pressure to yield compound desired I-7.

### Method H

To a stirred solution of compound intermediate I-7 (1 eq.) in EtOH/MeOH at 0° C. under Ar atmosphere was added NaBH₄ (2 eq.) and the reaction stirred at room temperature for 1-2 h. After completion, the reaction mixture was concentrated in vacuo, the residue obtained was diluted with water and extracted using ethyl acetate. The combined organic layers were collected, dried over anhydrous sodium sulphate, filtered, concentrated in vacuo and purified by silica gel column chromatography to afford the I-9.

### Method I

A mixture of a bromine substituted compound (1 eq.), boronic acid/boronate ester (1 eq.) in 1, 4-dioxane, and 2.5 eq. of 2M solution of potassium phosphate, was purged with Ar for 15 min, after which tetrakistriphenyl phosphine palladium (0.06 eq.) was added and the reaction stirred at 90° C. overnight. After completion, the reaction mixture was filtered through Celite and evaporated to dryness. The residue was taken up in ethyl acetate, washed with water, followed by brine, then dried over anhydrous sodium sulfate and the solvent removed under reduced pressure. The crude product was purified by column chromatography or preparative HPLC to afford final product.

### Intermediates 1-3

The compounds in Table 1 were synthesized according to the method listed in the column titled Method.

TABLE 1

| Inter- | Meth- | |
|---|---|---|
| mediate | od | Structure, |
| 1 | A | 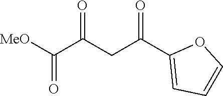 [SMILES: COC(=O)C(=O)CC(=O)c1ccco1] |
| | | Methyl 4-(furan-2-yl)-2,4-dioxobutanoate. LCMS |
| | | Calculated for C₉H₈O₅: 196.04; Found: 197 (M + 1) |
| 2 | C | 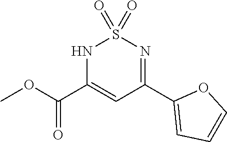 [SMILES: COC(=O)C1=CC(c2ccco2)=NS(=O)(=O)N1] |
| | | Methyl 5-(furan-2-yl)-2H-1,2,6-thiadiazine-3-carboxylate |
| | | 1,1-dioxide. ¹H NMR (DMSO-d₆, 400 MHz): δ 8.03 (s, |
| | | 1H), 7.49 (d, J = 4.0 Hz, 1H), 7.19-7.05 (m, |
| | | 1H), 6.83 (s, 1H), 6.77-6.75 (m, 1H), 3.86 (s, 3H); |
| | | LCMS Calculated for C₉H₈N₂O₅S: |
| | | 256.02. Found 256.9 (M + 1). |
| 3 | E | 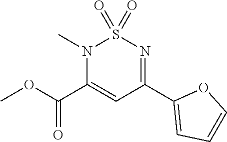 [SMILES: COC(=O)C1=CC(c2ccco2)=NS(=O)(=O)N1C] |
| | | Methyl 5-(furan-2-yl)-2-methyl-2H-1,2,6-thiadiazine-3- |
| | | carboxylate 1,1-dioxide. ¹H NMR (DMSO-d₆, 400 MHz): δ |
| | | 8.17 (s, 1H), 7.75 (d, J = 3.6 Hz, 1H), 7.75 (d, J = 1.2 Hz, |
| | | 1H), 6.86-6.85 (m, 1H), 3.93 (s, 3H), 3.51 (s, 3H); LCMS |
| | | Calculated for C₁₀H₁₀N₂O₅S; 270.03. Found 270.9 (M + 1). |

### Stereochemistry of the Examples

The absolute stereochemistry of all other sets of enantiomers were assigned based on the crystal structure determination of HBV-CSU-016 Isomer I. In each case one of the stereoisomers of the pair was significantly more active activity and was assigned the same absolute stereochemistry (3S,5R) as HBV-CSU-016-Isomer 1.

The synthesis of HBV-CSU-016 Isomer I is illustrated in Scheme VI. Advanced intermediate VI-6 was synthesized using the general synthetic methods provided above and listed in the Scheme. HBV-CSU-016 Isomer I was isolated by chiral chromatography.

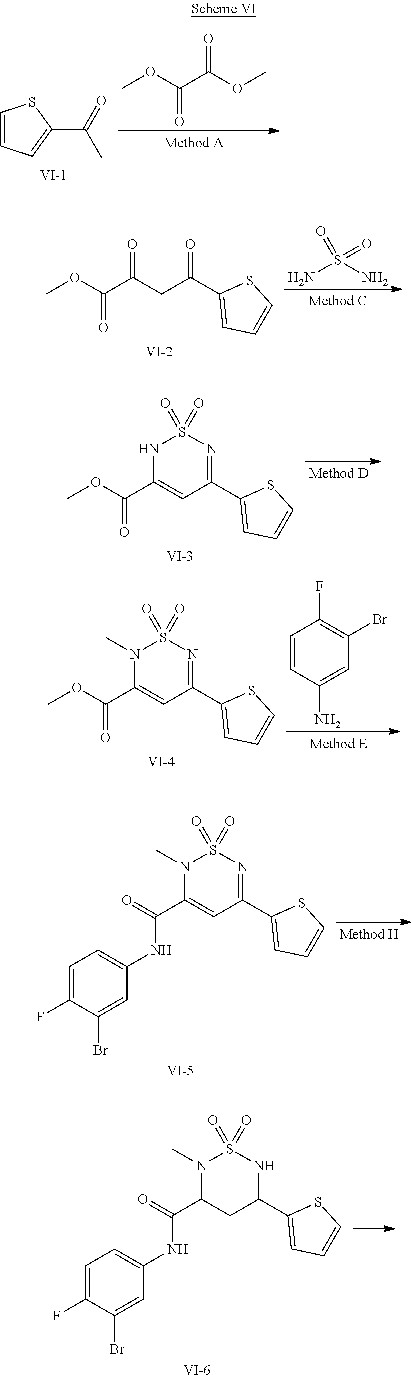

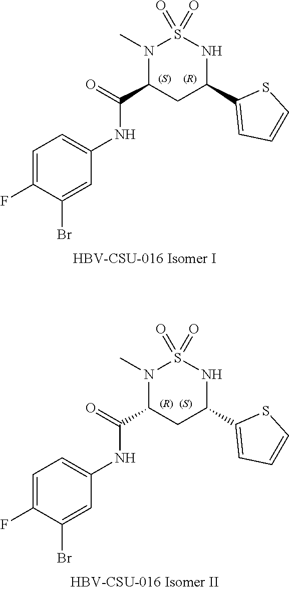

### Intermediate VI-2

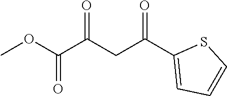

Methyl 2, 4-dioxo-4-(thiophen-2-yl)butanoate. TLC: 10% MeOH/DCM (R_{f}. 0.1); ¹H NMR (DMSO-d₆, 400 MHz): δ 7.68 (d, J=5.2 Hz, 1H), 7.61 (d, J=4.4 Hz, 1H), 7.10 (t, J=5.2 Hz, 1H), 6.34 (s, 1H), 3.69 (s, 3H); LCMS Calculated for C₉H₈O₄S: 212.01; Observed: 212.95 (M+1).

### Intermediate VI-3

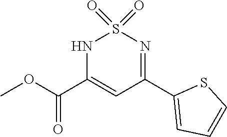 [SMILES: COC(=O)C1=CC(c2cccs2)=NS(=O)(=O)N1]

Methyl 5-(thiophen-2-yl)-2H-1,2,6-thiadiazine-3-carboxylate 1,1-dioxide. TLC: 20% MeOH/DCM (R_{f}. 0.1); ¹H NMR (DMSO-d₆, 400 MHz): δ 11.50 (br.s, 1H), 8.06 (d, J=4.0 Hz, 1H), 7.93 (d, J=5.2 Hz, 1H), 7.23 (t, J=4.0 Hz, 1H), 6.99 (s, 1H), 3.87 (s, 3H); LCMS Calculated for C₉H₈N₂O₄S₂: 271.99; LCMS observed: 272.85 (M+1).

### Intermediate VI-4

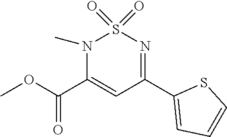 [SMILES: COC(=O)C1=CC(c2cccs2)=NS(=O)(=O)N1C]

Methyl 2-methyl-5-(thiophen-2-yl)-2H-1,2,6-thiadiazine-3-carboxylate 1,1-dioxide. TLC: 40% EtOAc/hexanes (R_{f}. 0.4); ¹H NMR (DMSO-d₆, 400 MHz): δ 8.23 (d, J=4.0 Hz, 1H), 8.10 (d, J=4.8 Hz, 1H), 7.32-7.30 (m, 2H), 3.94 (s, 3H), 3.50 (s, 3H); LCMS Calculated for C₁₀H₁₀N₂O₄S₂: 286.01; LCMS observed: 286.94 (M+1).

### Intermediate VI-5

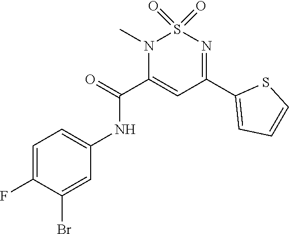 [SMILES: CN1C(C(=O)Nc2ccc(F)c(Br)c2)=CC(c2cccs2)=NS1(=O)=O]

N-(3-Bromo-4-fluorophenyl)-2-methyl-5-(thiophen-2-yl)-2H-1,2,6-thiadiazine-3-carboxamide 1,1-dioxide. ¹H-NMR (DMSO-d6, 400 MHz): δ 11.29 (s, 1H), 8.25 (d, J=3.6 Hz, 1H), 8.11-8.09 (m, 2H), 7.71-7.67 (m, 1H), 7.47 (t, J=8.0 Hz, 1H), 7.34-7.32 (m, 1H), 7.19 (s, 1H), 3.45 (s, 3H). LCMS Calculated for C₁₅H₁₁BrFN₃O₃S₂: 442.94; LCMS observed: 445.65 (M+2)

### Intermediate VI-6

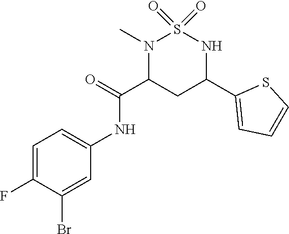 [SMILES: CN1C(C(=O)Nc2ccc(F)c(Br)c2)CC(c2cccs2)NS1(=O)=O]

N-(3-Bromo-4-fluorophenyl)-2-methyl-5-(thiophen-2-yl)-1,2,6-thiadiazinane-3-carboxamide 1,1-dioxide. ¹H-NMR (DMSO-d₆, 400 MHz): δ 10.55 (s, 1H), 8.10 (dd, J=6.4, 2.6 Hz, 1H), 7.71-7.54 (m, 2H), 7.52 (d, J=5.3 Hz, 1H), 7.37 (t, J=8.8 Hz, 1H), 7.15-7.14 (m, 1H), 7.03-7.01 (m, 1H), 4.85-4.74 (m, 1H), 4.30 (dd, J=11.7, 3.0 Hz, 1H), 2.61 (s, 3H), 2.29-2.08 (m, 2H). LCMS Calculated for C₁₅H₁₅BrFN₃O₃S₂: 446.97; LCMS observed: 449.90 (M+1)

### HBV-CSU-016 Isomer I

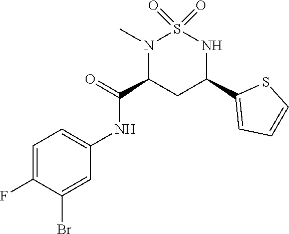 [SMILES: CN1[C@H](C(=O)Nc2ccc(F)c(Br)c2)C[C@H](c2cccs2)NS1(=O)=O]

(3S,5R)—N-(3-Bromo-4-fluorophenyl)-2-methyl-5-(thiophen-2-yl)-1,2,6-thiadiazinane-3-carboxamide 1,1-dioxide. ¹H-NMR (DMSO-d₆, 400 MHz): δ 10.55 (s, 1H), 8.09-8.06 (m, 1H), 7.68-7.66 (m, 1H), 7.62-7.58 (m, 1H), 7.51-7.49 (m, 1H), 7.36 (t, J=8.8 Hz, 1H), 7.14-7.13 (m, 1H), 7.02-7.00 (m, 1H), 4.80-4.76 (m, 1H), 4.29-4.25 (m, 1H), 2.60 (s, 3H), 2.31-2.08 (m, 2H). LCMS Calculated for C₁₅H₁₅BrFN₃O₃S₂: 446.97; LCMS observed: 449.90 (M+1)

### Single Crystal X-Ray Structure of HBV-CSU-016 Isomer I

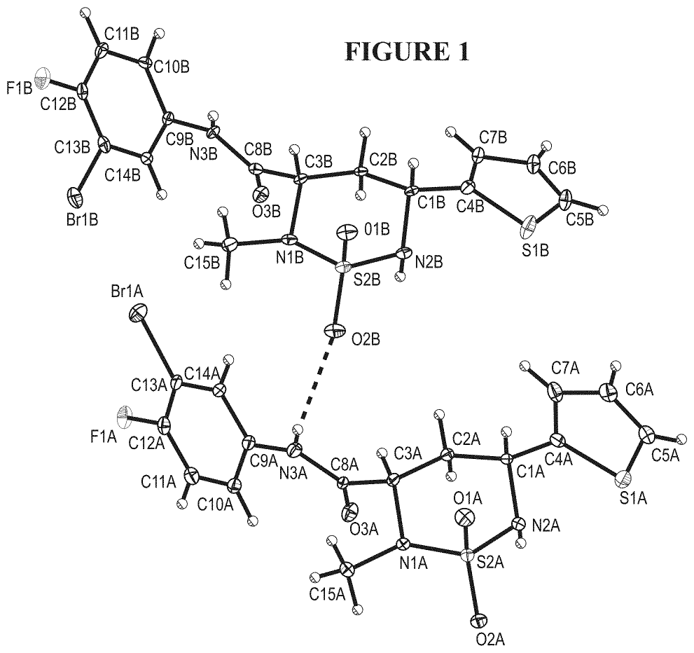

The Crystal structure of HBV-CSU-016-Isomer-I is shown in FIG. 1. Displacement ellipsoids are drawn at the 30% probability level and H atoms are shown as small spheres of arbitrary radii. Dashed line indicates hydrogen bond. The Crystal data and structure refinement for HBV-CSU-016-Isomer-I are as follows:

| Identification code | SAP-MA1703-08(isomer-1) | |
|---|---|---|
| | (IICT file code: KA84_0 m) | |
| Empirical formula | C₁₅H₁₅BrF N₃O₃S₂ | |
| Formula weight | 448.33 | |
| Temperature | 100(2) K | |
| Wavelength | 0.71073 Å | |
| Crystal system | Monoclinic | |
| Space group | P2₁ | |
| Unit cell dimensions | a = 5.001(6) Å | α = 90°. |
| | b = 25.63(3) Å | β = 94.240(19)°. |
| | c = 13.390(16) Å | γ = 90°. |

| | |
|---|---|
| Volume | 1712(4) Å³ |
| Z | 4 |
| Density (calculated) | 1.740 Mg/m³ |
| Absorption coefficient | 2.676 mm⁻¹ |
| F(000) | 904 |
| Crystal size | 0.310 × 0.240 × 0.190 mm³ |
| θ range for data collection | 2.830 to 28.374°. |
| Index ranges | −6 <= h <= 6, −34 <= k <= 34, −17 <= 1 <= 17 |
| Reflections collected | 50333 |
| Independent reflections | 8467 [R(int) = 0.0361] |
| Completeness to θ = 25.242° | 99.8% |
| Refinement method | Full-matrix least-squares on F² |
| Data/restraints/parameters | 8467/1/469 |
| Goodness-of-fit on F² | 1.022 |
| Final R indices [I > 2σ(I)] | R1 = 0.0265, wR2 = 0.0596 |
| R indices (all data) | R1 = 0.0313, wR2 = 0.0610 |
| Absolute structure parameter | 0.034(2) |
| Largest diff. peak and hole | 0.311 and −0.513 e.Å⁻³ |
| Measurement | Broker D8 QUEST PHOTON-100 Detector |
| Software Used | SHELXTL-PLUS |

The absolute stereochemistry at each chiral center was assigned using the PLATON computer application as described by A. L. Spek in J. APPL. CRYST. 36, 7-13, 2003. The designated chiral centers are:
C(1A) Chiral: R
C(1B) Chiral: R
C(3A) Chiral: S
C(3B) Chiral: S
   The absolute stereochemistry of all other sets of enantiomers were assigned based on this crystal structure determination. In each case only one of the stereoisomers of the pair had significant activity and the active isomer was assigned the same stereochemistry as HBV-CSU-016-Isomer-1.

### Intermediate 4

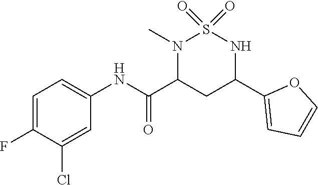 [SMILES: CN1C(C(=O)Nc2ccc(F)c(Cl)c2)CC(c2ccco2)NS1(=O)=O]

N-(3-Chloro-4-fluorophenyl)-5-(furan-2-yl)-2-methyl-1,2,6-thiadiaznane-3-carboxamide 1,1-dioxide. The title compound was synthesized from N-(3-chloro-4-fluorophenyl)-5-(furan-2-yl)-2-methyl-2H-1,2,6-thiadiazine-3-carboxamide 1,1-dioxide (intermediate 7) via Method H provided 5.7 g (70.54%, yield) of product as a white solid. TLC: 50% EtOAc/hexanes (R_{f}. 0.7); ¹H NMR (DMSO-d₆, 400 MHz): δ 10.55 (s, 1H), 7.97 (dd, J=6.9, 2.6 Hz, 1H), 7.66 (s, 1H), 7.63-7.51 (m, 2H), 7.40 (t, J=9.1 Hz, 1H), 6.46-6.45 (m, 2H), 4.65-4.64 (m, 1H), 4.30-4.26 (m, 1H), 2.60 (s, 3H), 2.23-2.02 (m, 2H); LCMS Calculated for C₁₅H₁₅ClFN₃O₄S; 387.05. Found: 387.90 (M+1).

### Intermediate 5

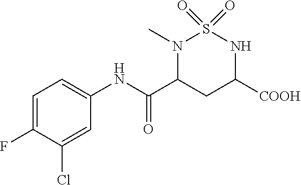 [SMILES: CN1C(C(=O)Nc2ccc(F)c(Cl)c2)CC(C(=O)O)NS1(=O)=O]

Cis-5-((3-Chloro-4-fluorophenyl)carbamoyl)-6-methyl-1,2,6-thiadiazinane-3-carboxylic acid 1,1-dioxide To a stirred solution of N-(3-chloro-4-fluorophenyl)-5-(furan-2-yl)-2-methyl-1,2,6-thiadiazinane-3-carboxamide 1,1-dioxide (5.6 g, 14.43 mmol) in acetone:water (1:1,200 mL), was added KMnO₄ (15.96 g, 101.01 mmol) (exotherm observed) slowly and the mixture heated at 60° C. for 3 h. Following this, the reaction mixture was cooled to ambient temperature and isopropyl alcohol (100 mL) added. This mixture was stirred for 18 h and then filtered through a Celite pad and washed with isopropyl alcohol. The filtrate was evaporated, the residue was dissolved in 1N NaOH and the solution was washed with diethyl ether. The basic layer was acidified with 1N HCl and solid NaCl was added. The resulting suspension was extracted with ethyl acetate. The organic extracts were collected, dried over anhydrous sodium sulphate and concentrated in vacuo. The solid was washed with CH₂Cl₂ and dried in vacuo to afford 3 grams of the title compound (57.03%) as a white solid. ¹H NMR (400 MHz, DMSO-d6): δ 13.00 (br.s, 1H), 10.52 (s, 1H), 7.95 (dd, J=6.8, 2.4 Hz, 1H), 7.57-7.52 (m, 1H), 7.41-7.36 (m, 2H), 4.25-4.21 (m, 1H), 4.15-4.10 (m, 1H), 2.55 (s, 3H), 2.07-2.03 (m, 1H), 1.96-1.86 (m, 1H). LCMS Calculated for C₁₂H₁₃ClFN₃O₅S; 365.02. Found; 366 (M+1.)

### EXAMPLES

The compounds in Table 2 were synthesized from cis-5-((3-chloro-4-fluorophenyl)carbamoyl)-6-methyl-1,2,6-thiadiazinane-3-carboxylic acid 1,1-dioxide, intermediate 5, and readily available amine reactants using the procedure similar to that described in Method G. Isomers I and II can be isolated from the racemic parent via the chiral separation methods

TABLE 2

| Example ID | Structure | NMR and LCMS |
|---|---|---|
| 1006 | 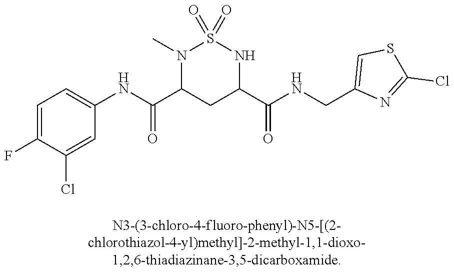 [SMILES: CN1C(C(=O)Nc2ccc(F)c(Cl)c2)CC(C(=O)NCc2csc(Cl)n2)NS1(=O)=O] | ¹H NMR (400 MHz, DMSO-d6): δ 10.49 (s, 1H), 8.63 (t, J = 5.6 Hz, 1H), 7.92-7.89 (m, 1H), 7.52- 7.48 (m, 1H),7.42 (s, 1H), 7.37-7.33(m, 2H), 4.29 (t, J = 5.6 Hz, 2H), 4.17-4.13 (m, 2H), 2.54 (s, 3H), 1.98-1.89 (m, 2H). LCMS calcd for C₁₆H₁₆Cl₂FN₅O₄S₂. Found; 496 (M + 1). |
| 1007 | 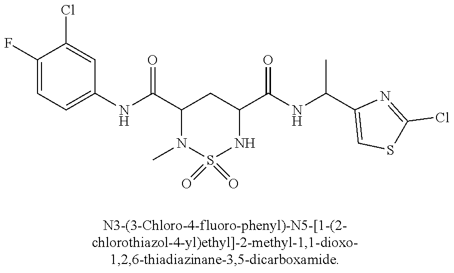 [SMILES: CC(NC(=O)C1CC(C(=O)Nc2ccc(F)c(Cl)c2)N(C)S(=O)(=O)N1)c1csc(Cl)n1] | ¹H NMR (400 MHz, DMSO-d6): δ 10.53 (s, 1H), 8.56 (t, J = 9.3 Hz, 1H), 7.95 (dd, J = 6.9, 2.6 Hz, 1H), 7.59-7.46 (m, 1H), 7.45-7.35 (m, 2H), 7.30-7.28 (m, 1H), 5.03-4.95 (m, 1H), 4.22- 4.14 (m, 2H), 2.57 (s, 3H), 1.99-1.94 (m, 2H), 1.40 (t, J = 7.0 Hz, 3H). LCMS calcd for; C₁₇H₁₈Cl₂FN₅O₄S₂. Found; 509.9 (M + 1). |
| 1009 | 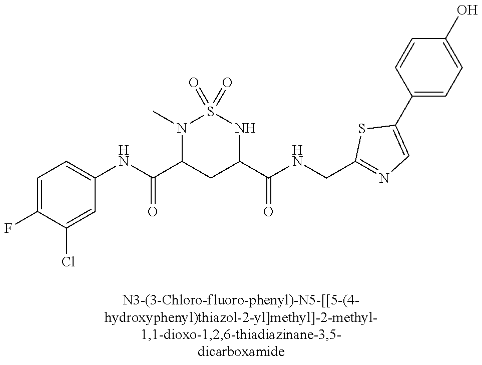 [SMILES: CN1C(C(=O)Nc2ccc(F)c(Cl)c2)CC(C(=O)NCc2ncc(-c3ccc(O)cc3)s2)NS1(=O)=O] | .¹H NMR (400 MHz, DMSO-d6): δ 10.53 (s, 1H), 9.74 (s, 1H), 9.02-9.00 (m, 1H), 7.97-7.94 (m, 1H), 7.87 (s, 1H), 7.56-7.54 (m, 1H), 7.44-7.37 (m, 4H), 6.80 (d, J = 8.4 Hz, 2H), 4.57- 4.52 (m, 2H), 4.25-4.15 (m, 2H), 2.58 (s, 3H), 1.99-1.94 (m, 2H). LCMS calcd for ; C₂₂H₂₁ClFN₅O₅S₂. Found; 554.05 (M + 1). |
| 1010 | 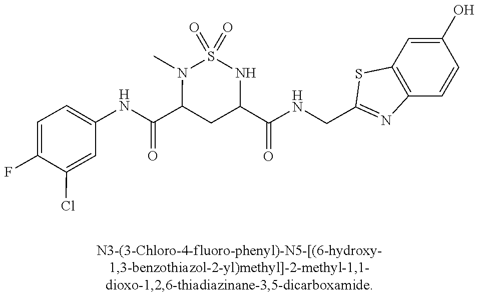 [SMILES: CN1C(C(=O)Nc2ccc(F)c(Cl)c2)CC(C(=O)NCc2nc3ccc(O)cc3s2)NS1(=O)=O] | ¹H NMR (400 MHz, DMSO-d6): δ 10.54 (s, 1H), 9.74 (s, 1H), 9.06 (t, J = 6.1 Hz, 1H), 7.96 (dd, J = 6.9, 2.6 Hz, 1H), 7.72 (d, J = 8.8 Hz, 1H), 7.57- 7.54 (m, 1H), 7.44-7.37 (m, 2H), 7.34-7.32 (m, 1H), 6.94-6.92 (m, 1H), 4.73-4.53 (m, 2H), 4.28-41.3 (m, 2H), 2.58 (s, 3H), 2.04-1.93 (m, 2H). LCMS calcd for; C₂₀H₁₉ClFN₅O₅S₂. Found; 528.07 (M + 1). |
| 1011 | 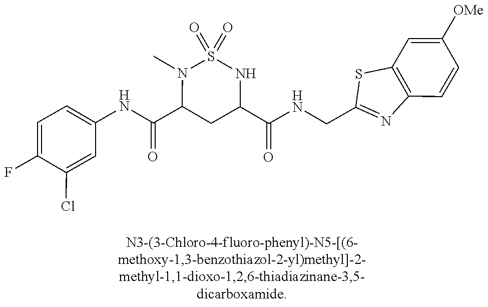 [SMILES: COc1ccc2nc(CNC(=O)C3CC(C(=O)Nc4ccc(F)c(Cl)c4)N(C)S(=O)(=O)N3)sc2c1] | ¹H NMR (400 MHz, DMSO-d6): δ 10.54 (s, 1H), 9.09 (t, J = 6.0 Hz, 1H), 7.96 (dd, J = 6.8, 2.6 Hz, 1H), 7.82 (d, J = 9.0 Hz, 1H), 7.64 (d, J = 2.6 Hz, 1H), 7.57-7.53 (m, 1H), 7.48-7.35 (m, 2H), 7.08 (dd, J = 9.0, 2.6 Hz, 1H), 4.76-4.56 (m, 2H), 4.30-4.13 (m, 2H), 3.81 (s, 3H), 2.58 (s, 3H), 2.06-194 (m, 2H). LCMS calcd for; C₂₁H₂₁ClFN₅O₅S₂. Found: 542 (M + 1). |
| 1011- | N3-(3-Chloro-4-fluoro-phenyl)-N5-[(6- | ¹H NMR (400 MHz, DMSO-d6): δ |
| ISOMER-A | methoxy-1,3-benzothiazol-2-yl)methyl]-2- | 10.53 (s, 1H), 9.08 (t, J = 6.0 Hz, |
| | methyl-1,1-dioxo-1,2,6-thiadiazinane-3,5- | 1H), 7.95 (dd, J = 6.8, 2.6 Hz, |
| | dicarboxamide | 1H), 7.81 (d, J = 8.9, |
| | | 2.7 Hz, 1H), 7.63 (d, J = 2.6 |
| | | Hz, 1H), 7.57-7.51 (m, 1H), 7.49- |
| | | 7.35 (m, 2H), 7.07 (dd, J = 8.9, |
| | | 2.7 Hz, 1H), 4.75-4.55 (m, 2H), |
| | | 4.29-4.15 (m, 2H), 3.80 (s, 3H), |
| | | 2.57 (s, 3H), 2.05-1.92 (m, 2H). |
| | | LCMS calcd for; |
| | | C₂₁H₂₁ClFN₅O₅S₂. Found: 542 (M + |
| | | 1). |
| 1011- | N3-(3-Chloro-4-fluoro-phenyl)-N5-[(6- | ¹H NMR (400 MHz, DMSO-d6): δ |
| ISOMER-B | methoxy-1,3-benzothiazol-2-yl)methyl]-2- | 10.53 (s, 1H), 9.08 (t, J = 6.0 Hz, |
| | methyl-1,1-dioxo-1,2,6-thiadiazinane-3,5- | 1H), 7.95 (dd, J = 6.9, |
| | dicarboxamide. | 2.5 Hz, 1H), 7.81 (d, J = |
| | | 8.9 Hz, 1H), 7.63 (d, J = 2.6 Hz, |
| | | 1H), 7.56-7.51 (m, 1H), 7.49-7.35 (m, |
| | | 2H), 7.07 (m, 1H), 4.75-4.55 (m, 2H), |
| | | 4.29-4.12 (m, 2H), 3.80 (s, 3H), 2.57 |
| | | (s, 3H), 2.05-1.92 (m, 2H). LCMS |
| | | calcd for; C₂₁H₂₁ClFN₅O₅S₂. Found; |
| | | 541.90 (M + 1). |
| 1012 | 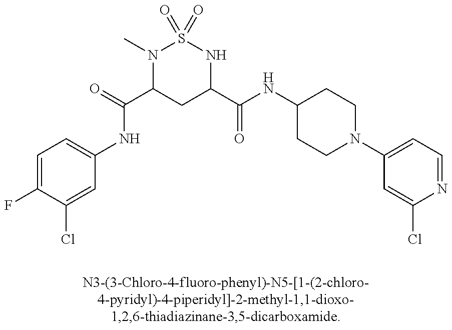 [SMILES: CN1C(C(=O)Nc2ccc(F)c(Cl)c2)CC(C(=O)NC2CCN(c3ccnc(Cl)c3)CC2)NS1(=O)=O] | ¹H NMR (400 MHz, DMSO-d6): δ 10.48 (s, 1H), 8.02 (d, J = 7.7 Hz, 1H), 7.97-7.85 (m, 2H), 7.53-7.48 (m, 1H), 7.36 (t, J = 9.1 Hz, 1H), 7.22 (d, J = 10.0 Hz, 1H), 6.82-6.80 (m, 2H), 4.20- 4.09 (m, 1H), 4.07-3.95 (m, 1H), 3.84- 3.83 (m, 2H), 3.41-3.32 (m, 1H), 3.03- 2.97 (m, 2H), 2.53 (s, 3H), 2.01-1.83 (m, 2H), 1.74-1.73 (m, 2H), 1.44-1.34 (m, 2H). LCMS calcd for; C₂₂H₂₅Cl₂FN₆O₄S. Found; 558.9 (M + |
| 1012- | N3-(3-Chloro-4-fluoro-phenyl)-N5-[1-(2-chloro- | ¹H NMR (400 MHz, DMSO-d6): δ |
| ISOMER-A | 4-pyridyl)-4-piperidyl]-2-methyl-1,1-dioxo- | 10.51 (s, 1H), 8.05 (d, J = |
| | 1,2,6-thiadiazinane-3,5-dicarboxamide. | 7.6 Hz, 1H), 8.00-7.89 (m, |
| | | 2H), 7.56-7.52 (m, 1H), |
| | | 7.39 (t, J = 9.2 Hz, 1H), 7.27-7.25 (m, |
| | | 1H), 6.85-6.83 (m, 2H), 4.23-4.14 (m, |
| | | 1H), 4.05-4.04 (m, 1H), 3.89-3.87 (m, |
| | | 3H), 3.06-3.00 (m, 2H), 2.57 (s, 3H), |
| | | 1.98-1.86 (m, 2H), 1.77-1.75 (m, 2H), |
| | | 1.48-1.31 (m, 2H). LCMS calcd for; |
| | | C₂₂H₂₅Cl₂FN₆O₄S. Found; 559.05 |
| | | (M + 1). |
| 1012- | N3-(3-Chloro-4-fluoro-phenyl)-N5-[1-(2-chloro- | ¹H NMR (400 MHz, DMSO-d6): δ |
| ISOMER-B | 4-pyridyl)-4-piperidyl]-2-methyl-1,1-dioxo- | 10.50 (s, 1H), 8.04 (d, J = |
| | 1,2,6-thiadiazinane-3,5-dicarboxamide. | 7.7 Hz, 1H), 7.98-7.89 (m, |
| | | 2H), 7.56-7.52 (m, 1H), |
| | | 7.39 (t, J = 9.1 Hz, 1H), 7.27-7.25 (m, |
| | | 1H), 6.88-6.80 (m, 2H), 4.17-4.15 (m, |
| | | 1H), 4.04-4.03 (m, 1H), 3.88-3.86 (m, |
| | | 3H), 3.09-2.97 (m, 2H), 2.56 (s, 3H), |
| | | 1.93-1.90 (m, 2H), 1.78-1.76 (m, 2H), |
| | | 1.51-1.33 (m, 2H). LCMS calcd for; |
| | | C₂₂H₂₅Cl₂FN₆O₄S. Found; 559.10 |
| | | (M + 1). |
| 1014 | 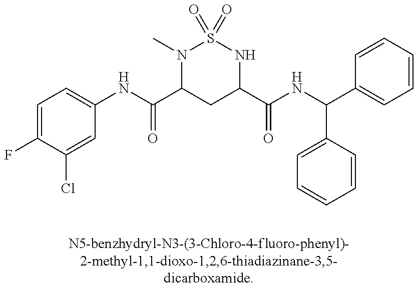 [SMILES: CN1C(C(=O)Nc2ccc(F)c(Cl)c2)CC(C(=O)NC(c2ccccc2)c2ccccc2)NS1(=O)=O] | ¹H NMR (400 MHz, DMSO-d6): δ 10.54 (s, 1H), 9.05 (d, J = 8.3 Hz, 1H), 7.96 (dd, J = 6.8, 2.6 Hz, 1H), 7.55-7.53 (m, 1H), 7.44-7.21 (m, 12H), 6.12 (d, J = 8.3 Hz, 1H), 4.31-4.19 (m, 2H), 2.57 (s, 3H), 2.06-1.88 (m, 2H). LCMS calcd for; C₂₅H₂₄ClFN₄O₄S. Found; 531.10 (M + 1). |
| 1014- | N5-benzhydryl-N3-(3-Chloro-4-fluoro-phenyl)- | ¹H NMR (400 MHz, DMSO-d6): δ |
| ISOMER-A | 2-methyl-1,1-dioxo-1,2,6-thiadiazinane-3,5- | 10.53 (s, 1H), 9.04 (d, J = 8.4 Hz, |
| | dicarboxamide. | 1H), 7.99-7.91 (m, 1H), |
| | | 7.55-7.53 (m, 1H), 7.44-7.21 (m, |
| | | 12H), 6.12 (d, J = 8.3 |
| | | Hz, 1H), 4.31-4.19 (m, 2H), 2.57 (s, |
| | | 3H), 2.05-1.88 (m, 2H). LCMS calcd |
| | | for; C₂₅H₂₄ClFN₄O₄S. Found; 531.20 |
| | | (M + 1). |
| 1014- | N5-benzhydryl-N3-(3-Chloro-4-fluoro-phenyl)- | ¹H NMR (400 MHz, DMSO-d6): δ |
| ISOMER-B | 2-methyl-1,1-dioxo-1,2,6-thiadiazinane-3,5- | 10.53 (s, 1H), 9.03 (d, J = 8.3 Hz, |
| | dicarboxamide. | 1H), 7.95 (dd, J = 6.8, |
| | | 2.6 Hz, 1H), 7.56-7.52 (m, 1H), |
| | | 7.44-7.20 (m, 12H), 6.11 (d, J = |
| | | 8.2 Hz, 1H), 4.29-4.21 (m, 2H), |
| | | 2.57 (s, 3H), 2.05-1.87 (m, 2H). |
| | | LCMS calcd for; C₂₅H₂₄ClFN₄O₄S. |
| | | Found; 531.20 (M + 1). |
| 1015 | 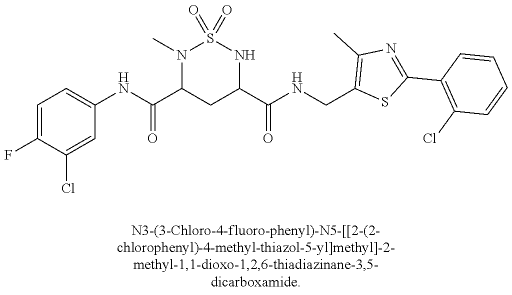 [SMILES: Cc1nc(-c2ccccc2Cl)sc1CNC(=O)C1CC(C(=O)Nc2ccc(F)c(Cl)c2)N(C)S(=O)(=O)N1] | ¹H NMR (400 MHz, DMSO-d6): δ 10.51 (s, 1H), 8.82 (t, J = 6.1 Hz, 1H), 8.16-8.10 (m, 1H), 7.94 (dd, J = 6.9, 2.6 Hz, 1H), 7.66-7.59 (m, 1H), 7.56-7.51 (m, 1H), 7.49-7.45 (m, 2H), 7.42- 7.35 (m, 2H), 4.55-4.40 (m, 2H), 4.20- 4.07 (m, 2H), 2.56 (s, 3H), 2.44 (s, 3H), 2.02-1.84 (m, 2H). LCMS calcd for; C₂₃H₂₂Cl₂FN₅O₄S₂. Found; 586.10 (M + 1). |
| 1015- | N3-(3-Chloro-4-fluoro-phenyl)-N5-[[2-(2- | ¹H NMR (400 MHz, DMSO-d6): δ |
| ISOMER-A | chlorophenyl)-4-methyl-thiazol-5-yl]methyl]-2- | 10.50 (s, 1H), 8.80-8.77 (m, 1H), 8.14 |
| | methyl-1,1-dioxo-1,2,6-thiadiazinane-3,5- | (dd, J = 6.1, 3.6 Hz, 1H), 7.95 (dd, |
| | dicarboxamide. | J =6 .9, 2.6 Hz, 1H), |
| | | 7.66-7.60 (m, 1H), 7.55-7.52 (m, |
| | | 1H), 7.48-7.45 (m, 3H), 7.39 (t, |
| | | J = 8.8 Hz, 1H), 4.56-4.40 (m, |
| | | 2H), 4.16-4.04 (m, 2H), 2.54 (s, 3H), |
| | | 2.43 (s, 3H), 2.02-1.85 (m, |
| | | 2H). LCMS calcd for; |
| | | C₂₃H₂₂Cl₂FN₅O₄S₂. Found; |
| | | 586.05 (M + 1). |
| 1015- | N3-(3-Chloro-4-fluoro-phenyl)-N5-[[2-(2- | ¹H NMR (400 MHz, DMSO-d6): δ |
| ISOMER-B | chlorophenyl)-4-methyl-thiazol-5-yl]methyl]-2- | 10.52 (s, 1H), 8.82-8.80 (m, 1H), 8.14 |
| | methyl-1,1-dioxo-1,2,6-thiadiazinane-3,5- | (dd, J = 6.1, 3.7 Hz, 1H), 7.94 (dd, |
| | dicarboxamide. | J = 7.0, 2.7 Hz, 1H), |
| | | 7.65-7.59 (m, 1H), 7.55-7.52 (m, |
| | | 1H), 7.48-7.45 (m, 2H), |
| | | 7.41-7.30 (m, 2H), 4.49-4.47 (m, 2H), |
| | | 4.22-4.05 (m, 2H), 2.55 (s, 3H), 2.43 |
| | | (s, 3H), 2.02-1.87 (m, 2H). LCMS |
| | | calcd for; C₂₃H₂₂Cl₂FN₅O₄S₂. Found; |
| | | 586.00 (M + 1). |
| 1016 | 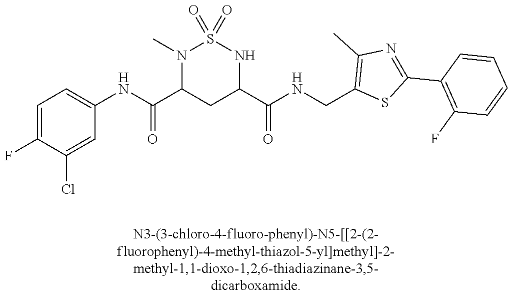 [SMILES: Cc1nc(-c2ccccc2F)sc1CNC(=O)C1CC(C(=O)Nc2ccc(F)c(Cl)c2)N(C)S(=O)(=O)N1] | ¹H NMR (400 MHz, DMSO-d6): δ 10.51 (s, 1H), 8.80 (t, J = 6.0 Hz, 1H), 8.19-8.14 (m, 1H), 7.94 (dd, J = 6.9, 2.5 Hz, 1H), 7.58-7.45 (m, 2H), 7.45-7.29 (m, 4H), 4.50-4.44 (m, 2H), 4.22- 4.07 (m, 2H), 2.56 (s, 3H), 2.44 (s, 3H), 2.01-1.88 (m, 2H). LCMS calcd for; C₂₃H₂₂ClF₂N₅O₄S₂. Found; 569.9 (M + 1). |
| 1016- | N3-(3-chloro-4-fluoro-phenyl)-N-[[2-(2- | ¹H NMR (400 MHz, DMSO-d6): δ |
| ISOMER-A | fluorophenyl)-4-methyl-thiazol-5-yl]methyl]-2- | 10.51 (s, 1H), 8.82-8.78 (m, 1H), |
| | methyl-1,1-dioxo-1,2,6-thiadiazinane-3,5- | 8.18-8.14 (m, 1H), |
| | dicarboxamide. | 7.96-7.93 (m, 1H), 7.55-7.50 (m, |
| | | 2H), 7.41-7.30 (m, 4H), 4.49- |
| | | 4.45 (m, 2H), 4.21-4.18 (m, 1H), 4.10- |
| | | 4.02 (m, 1H), 2.56 (s, 3H), 2.44 (s, |
| | | 3H), 2.00-1.90 (m, 2H). LCMS calcd |
| | | for; C₂₃H₂₂ClF₂N₅O₄S₂. Found; |
| | | 570.05 (M + 1). |
| 1016- | N3-(3-chloro-4-fluoro-phenyl)-N5-[[2-(2- | ¹H NMR (400 MHz, DMSO-d6): δ |
| ISOMER-B | fluorophenyl)-4-methyl-thiazol-5-yl]methyl]-2- | 10.52 (s, 1H), 8.82-8.78 (m, 1H), 8.18- |
| | methyl-1,1-dioxo-1,2,6-thiadiazinane-3,5- | 8.14 (m, 1H), 7.96-7.93 (m, |
| | dicarboxamide. | 1H), 7.55-7.48 (m, 2H), |
| | | 7.42-7.30 (m, 4H), 4.49- |
| | | 4.45 (m, 2H), 4.21-4.18 (m, 1H), |
| | | 4.10-4.05 (m, 1H), 2.56 (s, 3H), 2.44 |
| | | (s, 3H), 1.96-1.91 (m, 2H). LCMS |
| | | calcd for; C₂₃H₂₂ClF₂N₅O₄S₂. Found; |
| | | 570.05 (M + 1). |
| 1017 | 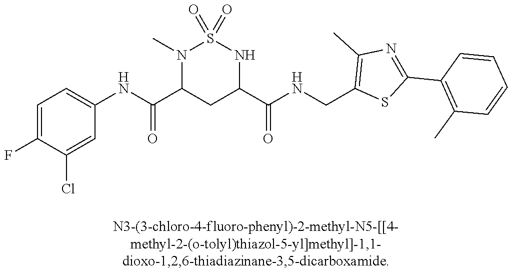 [SMILES: Cc1ccccc1-c1nc(C)c(CNC(=O)C2CC(C(=O)Nc3ccc(F)c(Cl)c3)N(C)S(=O)(=O)N2)s1] | ¹H NMR (400 MHz, DMSO-d6): δ 10.52 (s, 1H), 8.80-8.75 (m, 1H), 7.95 (dd, J = 6.8, 2.6 Hz, 1H), 7.67 (d, J = 7.4 Hz, 1H), 7.56-7.52 (m, 1H), 7.44- 7.25 (m, 5H), 4.54-4.38 (m, 2H), 4.21- 4.08 (m, 2H), 2.56 (s, 3H), 2.52 (s, 3H), 2.42 (s, 3H), 2.02-1.85 (m, 2H). LCMS calcd for; C₂₄H₂₅ClFN₅O₄S₂. Found; 566 (M + 1). |
| 1017- | N3-(3-chloro-4-fluoro-phenyl)-2-methyl-N5-[[4- | ¹H NMR (400 MHz, DMSO-d6): δ |
| ISOMER-A | methyl -2-(o-tolyl)thiazol-5-yl]methyl]-1,1- | 10.50 (s, 1H), 8.80-8.72 (m, 1H), 7.94 |
| | dioxo-1,2,6-thiadiazinane-3,5-dicarboxamide. | (dd, J = 6.8, 2.4 Hz, 1H), 7.67 (d, J = |
| | | 7.2 Hz, 1H), 7.55-7.53 (m, 1H), 7.42- |
| | | 7.26 (m, 5H), 4.50-4.42 (m, 2H), 4.19- |
| | | 4.15 (m, 1H), 4.09-4.05 (m, 1H), 2.56 |
| | | (s, 3H), 2.42 (s, 3H), 1.99-1.90 (m, |
| | | 2H). LCMS calcd for; |
| | | C₂₄H₂₅ClFN₅O₄S₂. Found; |
| | | 566.10 (M + 1). |
| 1017- | N3-(3-chloro-4-fluoro-phenyl)-2-methyl-N5-[[4- | ¹H NMR (400 MHz, DMSO-d6): δ |
| ISOMER-B | methyl-2-(o-tolyl)thiazol-5-yl]methyl]-1,1- | 10.51 (s, 1H), 8.80-8.72 (m, 1H), 7.94 |
| | dioxo-1,2,6-thiadiazinane-3,5-dicarboxamide. | (dd, J = 6.8, 2.4 Hz, 1H), 7.67 (d, J = |
| | | 7.2 Hz, 1H), 7.56-7.52 (m, 1H), 7.42- |
| | | 7.24 (m, 5H), 4.50-4.40 (m, 2H), 4.20- |
| | | 4.15 (m, 1H), 4.10-4.05 (m, 1H), 2.56 |
| | | (s, 3H), 2.42 (s, 3H), 2.00-1.87 (m, |
| | | 2H). LCMS calcd for; |
| | | C₂₄H₂₅ClFN₅O₄S₂. Found; |
| | | 566.10 (M + 1). |
| 1018 | 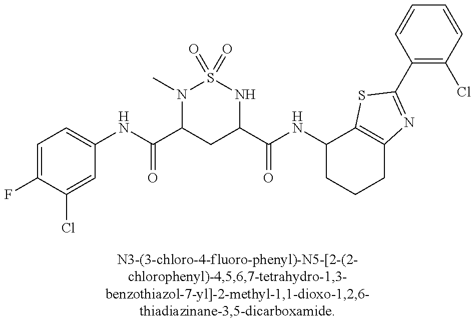 [SMILES: CN1C(C(=O)Nc2ccc(F)c(Cl)c2)CC(C(=O)NC2CCCc3nc(-c4ccccc4Cl)sc32)NS1(=O)=O] | ¹H NMR (400 MHz, DMSO-d6): δ 10.53 (s, 1H), 8.77-8.75 (m, 1H), 8.13-8.08 (m, 1H), 7.96-7.92 (m, 1H), 7.64-7.62 (m, 1H), 7.59-7.52 (m, 1H), 7.49-7.47 (m, 2H), 7.42-7.32 (m, 2H), 5.17- 5.14 (m, 1H), 4.21-4.10 (m, 2H), 2.80- 2.78 (m, 2H), 2.57 (s, 3H), 2.03-1.82 (m, 6H). LCMS calcd for; C₂₅H₂₄Cl₂FN₅O₄S₂. Found; 612.05 (M + 1). |
| 1019 | 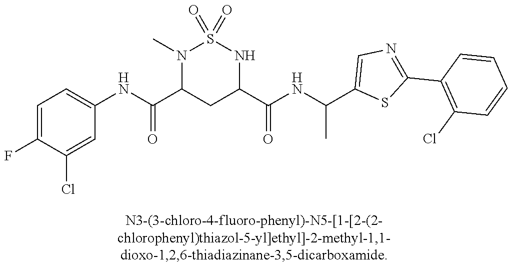 [SMILES: CC(NC(=O)C1CC(C(=O)Nc2ccc(F)c(Cl)c2)N(C)S(=O)(=O)N1)c1cnc(-c2ccccc2Cl)s1] | ¹H NMR (400 MHz, DMSO-d6): δ 10.54 (s, 1H), 8.82-8.78 (m, 1H), 8.17-8.12 (m, 1H), 7.97-7.83 (m, 1H), 7.90 (s, 1H), 7.66-7.60 (m, 1H), 7.57-7.53 (m, 1H), 7.52-7.47 (m, 2H), 7.42-7.37 (m, 2H), 5.34-5.28 (m, 1H), 4.24-4.09 (m, 2H), 2.57 (s, 3H), 2.08-1.97 (m, 2H), 1.55 (d, J = 7.2 Hz, 3H). LCMS calcd for; C₂₃H₂₂Cl₂FN₅O₄S₂. Found; 585.9 (M + 1). |
| 1025 | 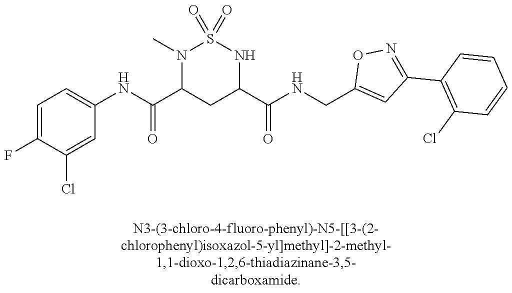 [SMILES: CN1C(C(=O)Nc2ccc(F)c(Cl)c2)CC(C(=O)NCc2cc(-c3ccccc3Cl)no2)NS1(=O)=O] | ¹H NMR (400 MHz, DMSO-d6): δ 10.53 (s, 1H), 8.85 (t, J = 6.0 Hz, 1H), 7.95 (dd, J = 6.8, 2.6 Hz, 1H), 7.67- 7.63 (m, 2H), 7.59-7.34 (m, 4H), 7.39 (t, J = 8.8 Hz, 1H), 6.80 (s, 1H), 4.60- 4.44 (m, 2H), 4.20-4.17 (m, 2H), 2.58 (s, 3H), 2.09-1.88 (m, 2H). LCMS calcd for; C₂₂H₂₀Cl₂FN₅O₅S. Found; 556. (M + 1). |
| 1025- | N3-(3-chloro-4-fluro-phenyl)-N5-[[3-(2- | .¹H NMR (400 MHz, DMSO-d6): δ |
| ISOMER-A | chlorophenyl)isoxazol-5-yl]methyl]-2-methyl- | 10.52 (s, 1H), 8.84 (t, J = 5.9 Hz, |
| | 1,1-dioxo-1,2,6-thiadiazinane-3,5- | 1H), 7.94 (dd, J = 6.8, |
| | dicarboxamide | 2.7 Hz, 1H), 7.69-7.59 (m, |
| | | 2H), 7.57-7.33 (m, 5H), 6.79 |
| | | (s, 1H), 4.59-4.42 (m, 2H), 4.22-4.13 |
| | | (m, 2H), 2.57 (s, 3H), 2.07-1.87 (m, |
| | | 2H). LCMS calcd for; |
| | | C₂₂H₂₀Cl₂FN₅O₅S. Found; 555.95 |
| | | (M + 1). |
| 1025- | N3-(3-chloro-4-fluoro-phenyl)-N5-[[3-(2- | ¹H NMR (400 MHz, DMSO-d6): δ |
| ISOMER-B | chlorophenyl)isoxazol-5-yl]methyl]-2-methyl- | 10.54 (s, 1H), 8.90-8.82 (m, 1H), |
| | 1,1-dioxo-1,2,6-thiadicarboxamidediazinane-3,5- | 7.98-7.91 (m, 1H), |
| | | 7.70-7.60 (m, 2H), 7.58-7.35 (m, |
| | | 5H), 6.80 (s, 1H), 4.57-4.50 |
| | | (m, 2H), 5.23-4.13 (m, 2H), 2.58 (s, |
| | | 3H), 2.04-1.95 (m, 2H). LCMS calcd |
| | | for; C₂₂H₂₀Cl₂FN₅O₅S. Found; 555.90 |
| | | (M + 1). |
| 1028 | 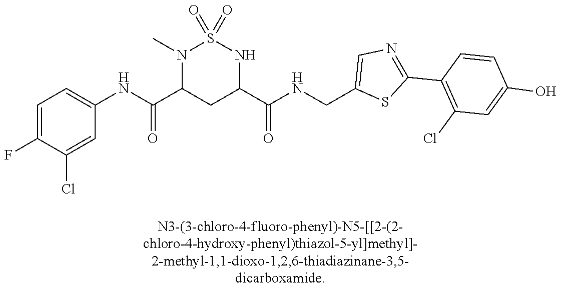 [SMILES: CN1C(C(=O)Nc2ccc(F)c(Cl)c2)CC(C(=O)NCc2cnc(-c3ccc(O)cc3Cl)s2)NS1(=O)=O] | ¹H NMR (400 MHz, DMSO-d6): δ 10.51 (s, 1H), 10.41 (s, 1H), 8.81 (t, J = 5.9 Hz, 1H), 8.01-7.91 (m, 2H), 7.76 (s, 1H), 7.56-7.52 (m, 1H), 7.45-7.34 (m, 2H), 6.95 (d, J = 2.5 Hz, 1H), 6.87 (dd, J = 8.7, 2.5 Hz, 1H), 4.60-4.45 (m, 2H), 4.20-4.19 (m, 1H), 4.15-4.03 (m, 1H), 2.57 (s, 3H), 2.03-1.86 (m, 2H). LCMS calcd for; C₂₂H₂₀Cl₂FN₅O₅S₂. Found; 588.10 (M + 1). |
| 1029 | 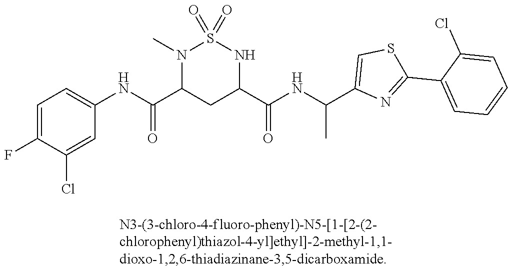 [SMILES: CC(NC(=O)C1CC(C(=O)Nc2ccc(F)c(Cl)c2)N(C)S(=O)(=O)N1)c1csc(-c2ccccc2Cl)n1] | ¹H NMR (400 MHz, DMSO-d6): δ 10.53 (bs, 1H), 8.60 (d, J = 8.0 Hz, 1H), 8.20-8.15 (m, 1H), 7.96-7.94 (m, 1H), 7.63-7.61 (m, 1H), 7.58-7.47 (m, 3H), 7.42-7.25 (m, 3H), 5.18-5.13 (m, 1H), 4.22-4.18 (m, 2H), 2.58 (s, 3H), 2.04-1.93 (m, 2H), 1.53-1.50 (m, 3H). LCMS calcd for; C₂₃H₂₂Cl₂FN₅O₄S₂. Found; 586.10 (M + 1). |
| 1030 | 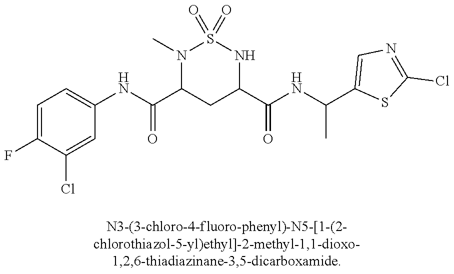 [SMILES: CC(NC(=O)C1CC(C(=O)Nc2ccc(F)c(Cl)c2)N(C)S(=O)(=O)N1)c1cnc(Cl)s1] | ¹H NMR (400 MHz, DMSO-d6): δ 10.53 (s, 1H), 8.80-8.75 (m, 1H), 7.96-7.94 (m, 1H), 7.59-7.51 (m, 2H), 7.43-7.33 (m, 2H), 5.20-5.10 (m, 1H), 4.22- 4.05 (m, 2H), 2.57 (s, 3H), 1.98-1.90 (m, 2H), 1.52-1.45 (m, 3H). LCMS calcd for; C₁₇H₁₈Cl₂FN₅O₄S₂. Found; 510 (M + 1). |
| 1031 | 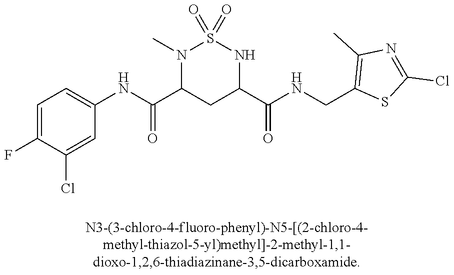 [SMILES: Cc1nc(Cl)sc1CNC(=O)C1CC(C(=O)Nc2ccc(F)c(Cl)c2)N(C)S(=O)(=O)N1] | ¹H NMR (400 MHz, DMSO-d6): δ 10.52 (s, 1H), 8.79 (t, J = 6.0 Hz, 1H), 7.95 (dd, J = 6.8, 2.4 Hz, 1H), 7.56-7.52 (m, 1H), 7.45-7.35 (m, 2H), 4.40-4.27 (m, 2H), 4.22-4.15 (m, 1H), 4.10- 4.02 (m, 1H), 2.57 (s, 3H), 2.31 (s, 3H), 2.00-1.80 (m, 2H). LCMS calcd for; C₁₇H₁₈Cl₂FN₅O₄S₂. Found; 509.95 (M + 1). |
| 1033 | 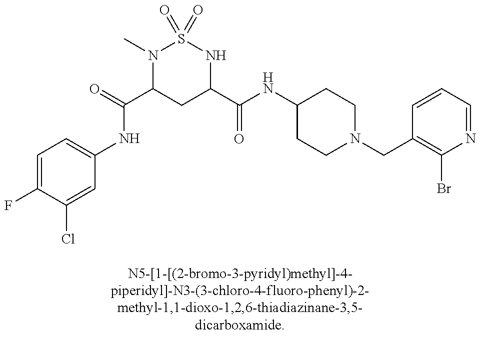 [SMILES: CN1C(C(=O)Nc2ccc(F)c(Cl)c2)CC(C(=O)NC2CCN(Cc3cccnc3Br)CC2)NS1(=O)=O] | ¹H NMR (400 MHz, DMSO-d6): δ 10.50 (s, 1H), 8.28-8.27 (m, 1H), 8.03-8.00 (m, 1H), 7.96-7.93 (m, 1H), 7.84-7.81 (m, 1H), 7.55-7.52 (m, 1H), 7.47-7.43 (m, 1H), 7.39 (t, J = 8.8 Hz, 1H), 7.22-7.20 (m, 1H), 4.21-4.17 (m, 1H), 4.07-4.05 (m, 1H), 3.61-3.57 (m, 1H), 3.51 (s, 2H), 2.77-2.75 (m, 2H), 2.56 (s, 3H), 2.20-2.14 (m, 2H), 1.95-1.89 (m, 2H), 1.74-1.72 (m, 2H), 1.53-1.42 (m, 2H). LCMS calcd for; C₂₃H₂₇BrClFN₆O₄S. Found; 617.05 (M + 1). |
| 1036 | 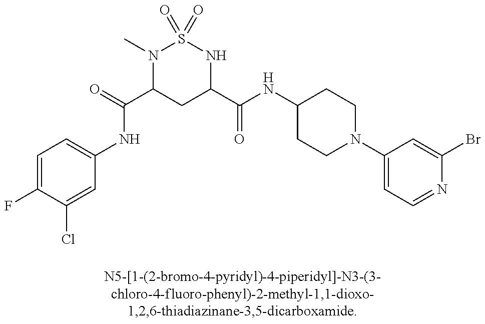 [SMILES: CN1C(C(=O)Nc2ccc(F)c(Cl)c2)CC(C(=O)NC2CCN(c3ccnc(Br)c3)CC2)NS1(=O)=O] | ¹H NMR (400 MHz, DMSO-d6): δ 10.50 (s, 1H), 8.06-8.04 (m, 1H), 7.95 (dd, J = 6.8, 2.4 Hz, 1H), 7.90 (d, J = 6.4 Hz, 1H), 7.56-7.52 (m, 1H), 7.39 (t, J = 9.2 Hz, 1H), 7.27-7.24 (m, 1H), 7.03-7.00 (m, 1H), 6.90-6.88 (m, 1H), 4.20-4.16 (m, 1H), 4.07-4.00 (m, 1H), 3.89-3.86 (m, 3H), 3.08-3.02 (m, 2H), 2.56 (s, 3H), 1.95-1.90 (m, 2H), 1.78- 1.76 (m, 2H), 1.45-1.40 (m, 2H). LCMS calcd for; C₂₂H₂₅BrClFN₆O₄S. Found; 605.10 (M + 2). |
| 1036- | N5-[1-(2-bromo-4-pyridyl)-4-piperidyl]-N3-(3- | ¹H NMR (400 MHz, DMSO-d6): δ |
| ISOMER-A | chloro-4-fluoro-phenyl)-2-methyl-1,1-dioxo- | 10.50 (s, 1H), 8.03-8.01 (m, 1H), |
| | 1,2,6-thiadiazinane-3,5-dicarboxamide. | 7.93-7.90 (m, 1H), 7.86-7.84 (m, 1H), |
| | | 7.54-7.48 (m, 1H), 7.36 (t, J = 9.2 Hz, |
| | | 1H), 7.26-7.18 (m, 1H), 6.96-6.92 (m, |
| | | 1H), 6.85-6.83 (m, 1H), 4.20-4.10 (m, |
| | | 1H), 4.05-3.95 (m, 1H), 3.90-3.80 (m, |
| | | 3H), 3.03-2.89 (m, 2H), 2.53 (s, 3H), |
| | | 1.90-1.85 (m, 2H), 1.76-1.72 (m, 2H), |
| | | 1.40-1.35 (m, 2H). LCMS calcd for; |
| | | C₂₂H₂₅BrClFN₆O₄S. Found; 605.05 |
| | | (M + 2). |
| 1036- | N5-[1-(2-bromo-4-pyridyl)-4-piperidyl]-N3-(3- | ¹H NMR (400 MHz, DMSO-d6): δ |
| ISOMER-B | chloro-4-fluoro-phenyl)-2-methyl-1,1-dioxo- | 10.50 (s, 1H), 8.03-8.01 (m, 1H), |
| | 1,2,6-thiadiazinane-3,5-dicarboxamide | 7.93-7.90 (m, 1H), 7.86-7.84 (m, 1H), |
| | | 7.53-7.49 (m, 1H), 7.36 (t, J = 9 .2 Hz, |
| | | 1H), 7.25-7.20 (m, 1H), 6.96-6.94 (m, |
| | | 1H), 6.85-6.83 (m, 1H), 4.17-4.12 (m, |
| | | 1H), 4.05-3.95 (m, 1H), 3.85-3.75 (m, |
| | | 3H), 3.03-2.95 (m, 2H), 2.53 (s, 3H), |
| | | 1.95-1.87 (m, 2H), 1.75-1.70 (m, 2H), |
| | | 1.42-1.34 (m, 2H). LCMS calcd for; |
| | | C₂₂H₂₅BrClFN₆O₄S. Found; 605.05 |
| | | (M + 2). |
| 1040 | 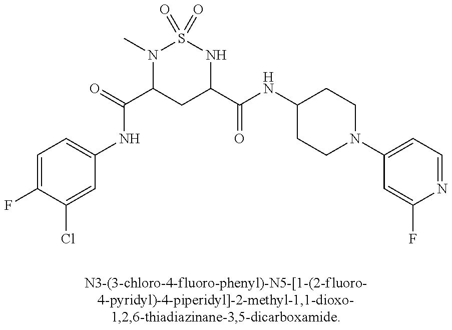 [SMILES: CN1C(C(=O)Nc2ccc(F)c(Cl)c2)CC(C(=O)NC2CCN(c3ccnc(F)c3)CC2)NS1(=O)=O] | ¹H NMR (400 MHz, DMSO-d6): δ 10.49 (s, 1H), 8.03 (d, J = 7.2 Hz, 1H), 7.93-7.90 (m, 1H), 7.74 (d, J = 6.0 Hz, 1H), 7.53-7.47 (m, 1H), 7.36 (t, J = 8.8 Hz, 1H), 7.24-7.22 (m, 1H), 6.76- 6.74 (m, 1H), 6.45 (s, 1H), 4.20-3.80 (m, 5H), 3.00 (t, J = 12 Hz, 2H), 2.53 (s, 3H), 1.98-1.70 (m, 4H), 1.47-1.35 (m, 2H). LCMS calcd for: C₂₂H₂₅ClF₂N₆O₄S. Found; 543.45 (M + 1). |
| 1040- | N3-(3-chloro-4-fluoro-phenyl)-N5-[1-(2-fluoro- | ¹H NMR (400 MHz, DMSO-d6): δ |
| ISOMER-A | 4-pyridyl)-4-piperidyl]-2-methyl-1,1-dioxo- | 10.50 (s, 1H), 8.05 (d, J = 7.2 Hz, |
| | 1,2,6-thiadiazinane-3,5-dicarboxamide. | 1H), 7.95-7.91 (m, |
| | | 1H), 7.77 (d, J = 6.0 Hz, 1H), |
| | | 7.54-7.47 (m, 1H), 7.36 (t, J = |
| | | 8.8 Hz, 1H), 7.24-7.22 (m, 1H), |
| | | 6.76-6.74 (m, 1H), 6.46 (s, 1H), 4.20- |
| | | 4.15 (m, 1H), 4.06-4.00 (m, 1H), 3.88- |
| | | 3.85 (m, 3H), 3.03 (t, J = 12.8 Hz, |
| | | 2H), 2.55 (s, 3H), 1.95-1.88 (m, 2H), |
| | | 1.80-1.75 (m, 1H), 1.47-1.38 (m, 2H). |
| | | LCMS calcd for; C₂₂H₂₅ClF₂N₆O₄S. |
| | | Found: 543.10 (M + 1). |
| 1040- | N3-(3-chloro-4-fluoro-phenyl)-N5-[1-(2-fluoro- | ¹H NMR (400 MHz, DMSO-d6): δ |
| ISOMER-B | 4-pyridyl)-4-piperidyl]-2-methyl-1,1-dioxo- | 10.50 (s, 1H), 8.05 (d, J = 7.6 Hz, |
| | | 1H), 7.94-7.92 (m, 1H), |
| | 1,2,6-thiadiazinane-3,5-dicarboxamide. | 7.77 (d, J = 6.0 Hz, 1H), |
| | | 7.55-7.50 (m, 1H), 7.36 (t, |
| | | J = 9.2 Hz, 1H), 7.30-7.07 (m, 1H), |
| | | 6.78-6.76 (m, 1H), 6.46 (s, 1H), 4.19- |
| | | 4.15 (m, 1H), 4.05-4.00 (m, 1H), 3.88- |
| | | 3.85 (m, 3H), 3.03 (t, J = 12 Hz, 2H), |
| | | 2.55 (s, 3H), 1.95-1.88 (m, 2H), 1.80- |
| | | 1.70 (m, 2H), 1.50-1.38 (m, 2H). |
| | | LCMS calcd for; C₂₂H₂₅ClF₂N₆O₄S. |
| | | Found; 543.10 (M + 1). |
| 1050 | 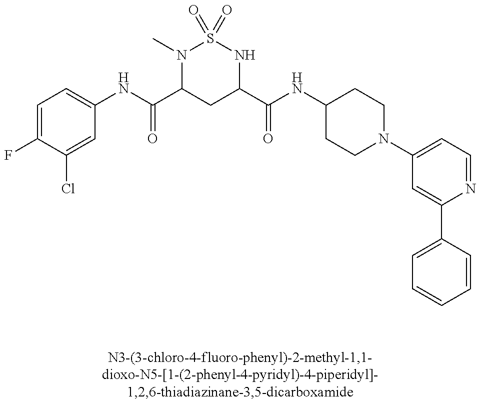 [SMILES: CN1C(C(=O)Nc2ccc(F)c(Cl)c2)CC(C(=O)NC2CCN(c3ccnc(-c4ccccc4)c3)CC2)NS1(=O)=O] | ¹H NMR (400 MHz, DMSO-d6): δ 10.51 (s, 1H), 8.25 (d, J = 6.0 Hz, 1H), 8.10-8.04 (m, 3H), 7.96-7.92 (m, 1H), 7.57-7.50 (m, 1H), 7.47-7.20 (m, 6H), 6.83-6.80 (m, 1H), 4.22-4.17 (m, 1H), 4.10-4.00 (m, 3H), 3.90-3.85 (m, 1H), 3.10-3.00 (m, 2H), 2.57 (s, 3H), 1.97- 1.80 (m, 4H), 1.55-1.45 (m, 2H). LCMS calcd for; C₂₈H₃₀ClFN₆O₄S. Found; 601.15 (M + 1). |
| 1050- | N3-(3-chloro-4-fluoro-phenyl)-2-methyl-1,1- | ¹H NMR (400 MHz, DMSO-d6): δ |
| ISOMER-A | dioxo-N5-[1-(2-phenyl-4-pyridyl)-4-piperidyl]- | 10.45-10.35 (m, 1H), 8.23 (d, J = 6.0 |
| | 1,2,6-thiadiazinane-3,5-dicarboxamide | Hz, 1H), 8.05 (d, J = 7.2 Hz, 2H), |
| | | 7.97-7.94 (m, 1H), 7.60-7.54 (m, 1H), |
| | | 7.45-7.30 (m, 7H), 6.82-6.79 (m, 1H), |
| | | 4.02-3.96 (m, 2H), 3.90-3.80 (m, 2H), |
| | | 3.05-2.95 (m, 2H), 2.45-2.35 (m, 3H), |
| | | 1.98-1.75 (m, 4H), 1.55-1.45 (m, 3H). |
| | | LCMS calcd for; C₂₈H₃₀ClFN₆O₄S. |
| | | Found; 601.10 (M + 1). |
| 1050- | N3-(3-chloro-4-fluoro-phenyl)-2-methyl-1,1- | ¹H NMR (400 MHz, DMSO-d6): δ |
| ISOMER-B | dioxo-N5-[1-(2-phenyl-4-pyridyl)-4-piperidyl]- | 10.40-10.30 (m, 1H), 8.23 (d, J = 6.0 |
| | 1,2,6-thiadiazinane-3,5-dicarboxamide. | Hz, 1H), 8.05 (d, J = 8.4 Hz, 2H), |
| | | 7.98-7.94 (m, 1H), 7.60-7.54 (m, 1H), |
| | | 7.45-7.30 (m, 7H), 6.83-6.79 (m, 1H), |
| | | 4.05-3.95 (m, 2H), 3.90-3.80 (m, 2H), |
| | | 3.05-2.97 (m, 2H), 2.49-2.40 (m, 3H), |
| | | 1.98-1.70 (m, 4H), 1.55-1.43 (m, |
| | | 3H). LCMS calcd for; |
| | | C₂₈H₃₀ClFN₆O₄S. Found; |
| | | 601.15 (M + 1). |
| 1051 | 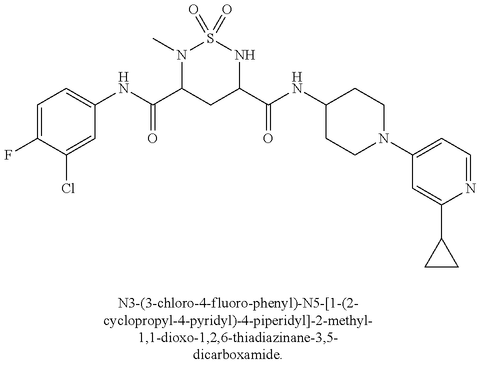 [SMILES: CN1C(C(=O)Nc2ccc(F)c(Cl)c2)CC(C(=O)NC2CCN(c3ccnc(C4CC4)c3)CC2)NS1(=O)=O] | ¹H NMR (400 MHz, DMSO-d6): δ 10.52 (s, 1H), 8.06 (d, J = 8.0 Hz, 1H), 8.00-7.91 (m, 2H), 7.56-7.52 (m, 1H), 7.39 (t, J = 8.8 Hz, 1H), 7.25 (d, J = 9.6 Hz, 1H), 6.75 (s, 1H), 6.63-6.61 (m, 1H), 4.21-4.16 (m, 1H), 4.06-4.03 (m, 1H), 3.88-3.85 (m, 3H), 3.00-.295 (m, 2H), 2.56 (s, 3H), 1.95-1.90 (m, 3H), 1.80-1.75 (m, 2H), 1.46-1.40 (m, 2H), 0.87-0.82 (m, 4H). LCMS calcd for; C₂₅H₃₀ClFN₆O₄S. Found; 565.10 (M + 1). |
| 1052 | 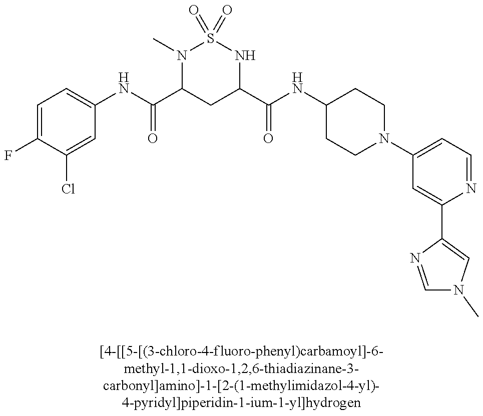 [SMILES: CN1C(C(=O)Nc2ccc(F)c(Cl)c2)CC(C(=O)NC2CCN(c3ccnc(-c4cn(C)cn4)c3)CC2)NS1(=O)=O] | ¹H NMR (400 MHz, DMSO-d6): δ 10.60 (s, 1H), 8.16 (s, 1H), 8.10-8.05 (m, 2H), 7.96-7.92 (m, 1H), 7.63-7.59 (m, 2H), 7.56-7.50 (m, 1H), 7.39 (t, J = 9.2 Hz, 1H), 7.35-7.20 (m, 2H), 6.70-6.65 (m, 1H), 4.21-4.15 (m, 1H), 4.05-4.00 (m, 1H), 3.90-3.85 (m, 3H), 3.69 (s, 3H), 3.06-2.98 (m, 2H), 2.56 (s, 3H), 1.96-1.80 (m, 4H), 1.55-1.40 (m, 2H). LCMS calcd for; C₂₆H₃₀ClFN₈O₄S. Found: 605 (M + 1). |
| 1054 | 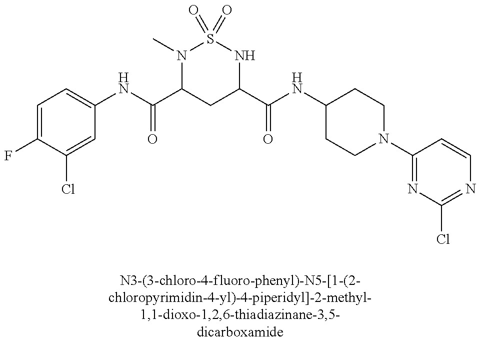 [SMILES: CN1C(C(=O)Nc2ccc(F)c(Cl)c2)CC(C(=O)NC2CCN(c3ccnc(Cl)n3)CC2)NS1(=O)=O] | ¹H NMR (400 MHz, DMSO-d6): δ 10.50 (s, 1H), 8.10-8.03 (m, 2H), 7.96-7.90 (m, 1H), 7.55-7.51 (m, 1H), 7.37 (t, J = 8.2 Hz, 1H), 7.25-7.20 (m, 1H), 6.86-6.84 (m, 1H), 4.30-3.80 (m, 4H), 3.15-3.07 (m, 2H), 1.95-1.79 (m, 4H), 1.41-1.35 (m, 3H). LCMS calcd for; C₂₁H₂₄Cl₂FN₇O₄S. Found; 560.05 (M + 1). |
| 1055 | 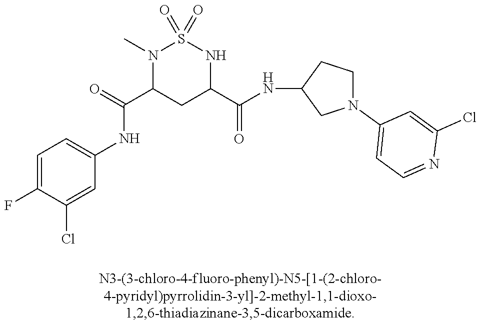 [SMILES: CN1C(C(=O)Nc2ccc(F)c(Cl)c2)CC(C(=O)NC2CCN(c3ccnc(Cl)c3)C2)NS1(=O)=O] | ¹H NMR (400 MHz, DMSO-d6): δ 10.51 (s, 1H), 8.45-8.38 (m, 1H), 7.98-7.85 (m, 2H), 7.58-7.50 (m, 1H), 7.39 (t, J = 9.0 Hz, 1H), 7.33-7.27 (m, 1H), 6.49-6.45 (m, 2H), 4.42-4.40 (m, 1H), 4.20-4.00 (m, 2H), 3.55-3.51 (m, 1H), 3.40-3.30 (m, 2H), 3.22-3.19 (m, 1H), 2.56 (s, 3H), 2.20-2.13 (m, 1H), 1.99-1.88 (m, 3H). LCMS calcd for; C₂₁H₂₃Cl₂FN₆O₄S. Found 545.10 (M + 1). |
| 1055-R | 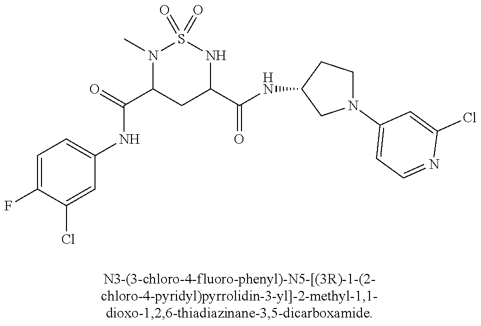 [SMILES: CN1C(C(=O)Nc2ccc(F)c(Cl)c2)CC(C(=O)N[C@@H]2CCN(c3ccnc(Cl)c3)C2)NS1(=O)=O] | ¹H NMR (400 MHz, DMSO-d6): δ 10.48 (s, 1H), 8.41-8.37 (m, 1H), 7.95-7.82 (m, 2H), 7.53-7.48 (m, 1H), 7.36 (t, J = 9.2 Hz, 1H), 7.27 (t, J = 10.4 Hz, 1H), 6.50-6.45 (m, 2H), 4.40-4.35 (m, 1H), 4.14-4.00 (m, 2H), 3.52-3.47 (m, 1H), 3.36-3.27 (m, 2H), 3.19-3.15 (m, 1H), 2.53 (s, 3H), 2.17-2.09 (m, 1H), 1.95-1.85 (m, 3H). LCMS calcd for; C₂₁H₂₃Cl₂FN₆O₄S. Found; 545.10 (M + 1). |
| 1055-R- | N3-(3-chloro-4-fluoro-phenyl)-N5-[(3R)-1-(2- | ¹H NMR (400 MHz, DMSO-d6): δ |
| ISOMER-A | chloro-4-pyridyl)pyrrolidin-3-yl]-2-methyl-1,1- | 10.51 (s, 1H), 8.41 (d, J = 6.8 Hz, |
| | dioxo-1,2,6-thiadiazinane-3,5-dicarboxamide | 1H), 7.98-7.85 (m, 2H), |
| | | 7.55-7.53 (m, 1H), 7.41-7.20 (m, |
| | | 2H), 6.48-6.46 (m, 2H), |
| | | 4.42-4.39 (m, 1H), 4.18-4.14 (m, 1H), |
| | | 4.07-4.02 (m, 1H), 3.54-3.50 (m, 1H), |
| | | 3.44-3.37 (m, 2H), 3.20-3.16 (m, 1H), |
| | | 2.56 (s, 3H), 2.19-2.14 (m, 1H), 2.00- |
| | | 1.87 (m, 3H). LCMS calcd for; |
| | | C₂₁H₂₃Cl₂FN₆O₄S. Found: |
| | | 545.10 (M + 1) |
| 1055-R- | N3-(3-chloro-4-fluoro-phenyl)-N5-[(3R)-1-(2- | ¹H NMR (400 MHz, DMSO-d6): δ |
| ISOMER-B | chloro-4-pyridyl)pyrrolidin-3-yl]-2-methyl-1,1- | 10.51 (s, 1H), 8.39 (d, J = 6.0 Hz, |
| | dioxo-1,2,6-thiadiazinane-3,5-dicarboxamide. | 1H), 7.99-7.86 (m, 2H), |
| | | 7.55-7.51 (m, 1H), 7.41-7.35 (m, |
| | | 2H), 6.49-6.47 (m, 2H), |
| | | 4.42-4.39 (m, 1H), 4.16-4.14 (m, |
| | | 1H), 4.06-4.01 (m, 1H), 3.55-3.51 (m, |
| | | 1H), 3.41-3.36 (m, 2H), 3.20-3.17 (m, |
| | | 1H), 2.54 (s, 3H), 2.18-2.14 (m, 1H), |
| | | 1.93-1.89 (m, 3H). LCMS calcd for; |
| | | C₂₁H₂₃Cl₂FN₆O₄S. FOund; |
| | | 545.10 (M + 1). |
| 1055-S | 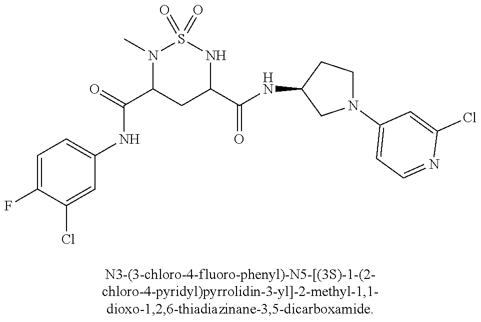 [SMILES: CN1C(C(=O)Nc2ccc(F)c(Cl)c2)CC(C(=O)N[C@H]2CCN(c3ccnc(Cl)c3)C2)NS1(=O)=O] | ¹H NMR (400 MHz, DMSO-d6): δ 10.52 (s, 1H), 8.44-8.40 (m, 1H), 7.96- 7.92 (m, 1H), 7.90-7.85 (m, 1H), 7.55- 7.52 (m, 1H), 7.39 (t, J = 8.8 Hz, 1H), 7.30 (t, J = 10 Hz, 1H), 6.50-6.46 (m, 2H), 4.40-4.37 (m, 1H), 4.20-4.17 (m, 1H), 4.07-4.03 (m, 1H), 3.54-3.50 (m, 1H), 3.39-3.32 (m, 2H), 3.21-3.16 (m, 1H), 2.56 (s, 3H), 2.19-2.14 (m, 1H), 1.97-1.91 (m, 3H). LCMS calcd for; C₂₁H₂₃Cl₂FN₆O₄S. Found; 545.1 (M + 1). |
| 1055-S- | N3-(3-chloro-4-fluoro-phenyl)-N5-[(3S)-1-(2- | ¹H NMR (400 MHz, DMSO-d6): δ |
| ISOMER-A | chloro-4-pyridyl)pyrrolidin-3-yl]-2-methyl-1,1- | 10.52 (s, 1H), 8.41 (d, J = 7.0 Hz, |
| | dioxo-1,2,6-thiadiazinane-3,5-dicarboxamide. | 1H), 7.99-7.86 (m, 2H), |
| | | 7.57-7.52 (m, 1H), 7.44-7.29 (m, |
| | | 2H), 6.49-6.46 (m, 2H), |
| | | 4.42-4.39 (m, 1H), 4.17-4.15 (m, 1H), |
| | | 4.09-4.00 (m, 1H), 3.56-3.50 (m, 1H), |
| | | 3.45-3.32 (m, 2H), 3.21-3.17 (m, 1H), |
| | | 2.55 (s, 3H), 2.23-2.04 (m, 1H), 2.01- |
| | | 1.84 (m, 3H). LCMS calcd for; |
| | | C₂₁H₂₃Cl₂FN₆O₄S. Found; |
| | | 545.05 (M + 1). |
| 1055-S- | N3-(3-chloro-4-fluoro-phenyl)-N5-[(3S)-1-(2- | ¹H NMR (400 MHz, DMSO-d6): δ |
| ISOMER-B | chloro-4-pyridyl)pyrrolidin-3-yl]-2-methyl-1,1- | 10.52 (s, 1H), 8.41 (d, J = 6.9 Hz, |
| | dioxo-1,2,6-thiadiazinane-3,5-dicarboxamde. | 1H), 7.98-7.85 (m, 2H), |
| | | 7.59-7.45 (m, 1H), |
| | | 7.42-7.37 (m, 2H), 6.49-6.46 (m, 2H), |
| | | 4.45-4.36 (m, 1H), 4.18-4.09 (m, 1H), |
| | | 4.03-4.00 (m, 1H), 3.53-3.49 (m, 1H), |
| | | 3.46-3.36 (m, 2H), 3.21-3.15 (m, 1H), |
| | | 2.54 (s, 3H), 2.23-2.10 (m, 1H), 2.03- |
| | | 1.81 (m, 3H). LCMS calcd for; |
| | | C₂₁H₂₃Cl₂FN₆O₄S. Found; |
| | | 545.10 (M + 1). |
| 1056 | 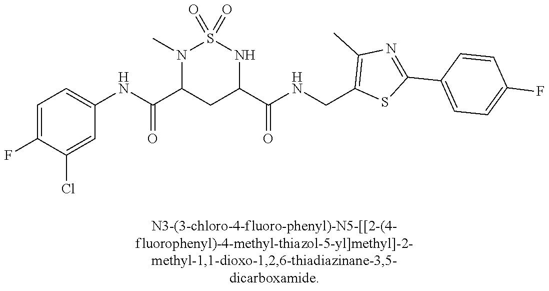 [SMILES: Cc1nc(-c2ccc(F)cc2)sc1CNC(=O)C1CC(C(=O)Nc2ccc(F)c(Cl)c2)N(C)S(=O)(=O)N1] | ¹H NMR (400 MHz, DMSO-d6): δ 10.52 (s, 1H), 8.80 (t, J = 5.8 Hz, 1H), 7.98-7.86 (m, 3H), 7.56-7.52 (m, 1H), 7.44-7.35 (m, 2H), 7.34-7.27 (m, 2H), 4.51-4.35 (m, 2H), 4.23-4.16 (m, 1H), 4.10-4.05 (m, 1H), 2.56 (s, 3H), 2.40 (s, 3H), 2.01-1.88 (m, 2H). LCMS calcd for; C₂₃H₂₂ClF₂N₅O₄S₂. Found; 570 (M + 1). |
| 1056- | N3-(3-chloro-4-fluoro-phenyl)-N5-[[2-(4- | ¹H NMR (400 MHz, DMSO-d6): δ |
| ISOMER-A | fluorophenyl)-4-methyl-thiazol-5-yl]methyl]-2- | 10.51 (s, 1H), 8.79-8.77 (m, 1H), |
| | methyl-1,1-dioxo-1,2,6-thiadiazinane-3,5- | 7.95-7.88 (m, 3H), |
| | dicarboxamide. | 7.55-7.53 (m, 1H), 7.41- |
| | | 7.37 (m, 2H), 7.32-7.28 (m, 2H), 4.44- |
| | | 4.40 (m, 2H), 4.21-4.06 (m, 2H), 2.56 |
| | | (s, 3H), 2.40 (s, 3H), 1.96-1.90 (m, |
| | | 2H). LCMS calcd for; |
| | | C₂₃H₂₂ClF₂N₅O₄S₂. Found; |
| | | 570.05 (M + 1). |
| 1056- | N3-(3-chloro-4-fluoro-phenyl)-N5-[[2-(4- | ¹H NMR (400 MHz, DMSO-d6): δ |
| ISOMER-B | fluorophenyl)-4-methyl-thiazol-5-yl]methyl]-2- | 10.51 (s, 1H), 8.79-8.77 (m, 1H), |
| | methyl-1,1-dioxo-1,2,6-thiadiazinane-3,5- | 7.95-7.88 (m, 3H), 7.55-7.53 (m, |
| | dicarboxamide. | 1H), 7.41-7.37 (m, 2H), |
| | | 7.32-7.28 (m, 2H), 4.44-4.40 (m, |
| | | 2H), 4.20-4.05 (m, 2H), 2.56 |
| | | (s, 3H), 2.40 (s, 3H), 1.95-1.90 (m, |
| | | 2H). LCMS calcd for; |
| | | C₂₃H₂₂ClF₂N₅O₄S₂. Found; |
| | | 570.05 (M + 1). |
| 1057 | 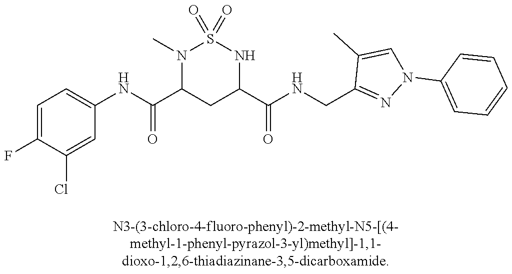 [SMILES: Cc1cn(-c2ccccc2)nc1CNC(=O)C1CC(C(=O)Nc2ccc(F)c(Cl)c2)N(C)S(=O)(=O)N1] | ¹H NMR (400 MHz, DMSO-d6): δ 10.50 (s, 1H), 8.56-8.48 (m, 1H), 8.24 (s, 1H), 7.98-7.92 (m, 1H), 7.74 (d, J = 8.0 Hz, 2H), 7.58-7.51 (m, 1H), 7.47-7.36 (m, 3H), 7.30-7.20 (m, 2H), 4.40-4.28 (m, 2H), 4.24-4.12 (m, 2H), 2.56 (s, 3H), 2.05 (s, 3H), 1.96-1.92 (m, 2H). LCMS calcd for; C₂₃H₂₄ClFN₆O₄S. Found; 535 (M + 1). |
| 1057- | N3-(3-chloro-4-fluoro-phenyl)-2-methyl-N5-[(4- | ¹H NMR (400 MHz, DMSO-d6): δ |
| ISOMER-A | methyl-1-phenyl-pyrazol-3-yl)methyl]-1,1- | 10.54 (s, 1H), 8.55-8.52 (m, 1H), 8.24 |
| | dioxo-1,2,6-thiadiazinane-3,5-dicarboxamide. | (s, 1H), 7.98-7.93 (m, 1H), 7.74 (d, |
| | | J = 7.6 Hz, 2H), 7.56-7.52 (m, |
| | | 1H), 7.47-7.36 (m, 3H), |
| | | 7.30-7.20 (m, 2H), 4.40-4.28 (m, |
| | | 2H), 4.24-4.12 (m, 2H), 2.56 |
| | | (s, 3H), 2.05 (s, 3H), 1.96-1.92 (m, |
| | | 2H). LCMS calcd for; |
| | | C₂₃H₂₄ClFN₆O₄S. Found: |
| | | 535 (M + 1). |
| 1057- | N3-(3-chloro-4-fluoro-phenyl)-2-methyl-N5-[(4- | ¹H NMR (400 MHz, DMSO-d6): δ |
| ISOMER-B | methyl-1-phenyl-pyrazol-3-yl)methyl]-1,1- | 10.53 (s, 1H), 8.55-8.52 (m, 1H), 8.24 |
| | dioxo-1,2,6-thiadiazinane-3,5-dicarboxamide. | (s, 1H), 7.98-7.93 (m, 1H), 7.74 (d, |
| | | J = 7.6 Hz, 2H), |
| | | 7.56-7.52 (m, 1H), 7.47- |
| | | 7.36 (m, 3H), 7.30-7.20 (m, 2H), 4.40- |
| | | 4.28 (m, 2H), 4.24-4.12 (m, 2H), 2.56 |
| | | (s, 3H), 2.05 (s, 3H), 1.96-1.92 (m, |
| | | 2H). LCMS calcd for; |
| | | C₂₃H₂₄ClFN₆O₄S. FOund; |
| | | 535.05 (M + 1). |
| 1062 | 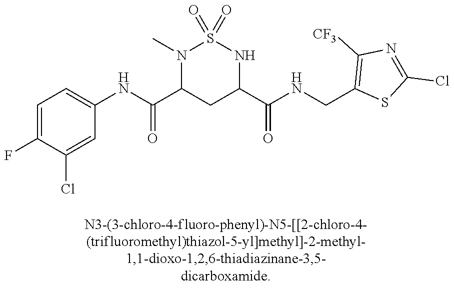 [SMILES: CN1C(C(=O)Nc2ccc(F)c(Cl)c2)CC(C(=O)NCc2sc(Cl)nc2C(F)(F)F)NS1(=O)=O] | ¹H NMR (400 MHz, DMSO-d6): δ 10.54 (s, 1H), 9.15-9.08 (m, 1H), 7.97-7.92 (m, 1H), 7.55-7.35 (m, 3H), 4.60-4.50 (m, 2H), 4.23-4.10 (m, 2H), 2.57 (s, 3H), 2.05-1.85 (m, 2H). LCMS calcd for; C₁₇H₁₅Cl₂F₄N₅O₄S₂. Found; 563.9 (M + 1). |
| 1063 | 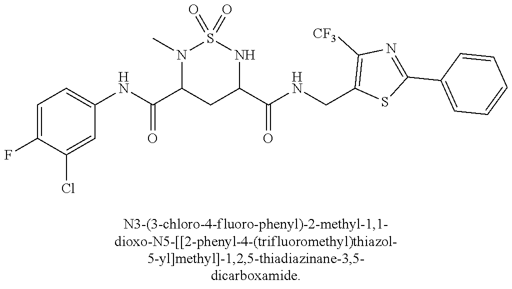 [SMILES: CN1C(C(=O)Nc2ccc(F)c(Cl)c2)CC(C(=O)NCc2sc(-c3ccccc3)nc2C(F)(F)F)NS1(=O)=O] | ¹H NMR (400 MHz, DMSO-d6): δ 10.51 (s, 1H), 9.08 (t, J = 5.2 Hz, 1H), 7.95-7.86 (m, 3H), 7.51-7.48 (m, 4H), 7.46-7.43 (m, 1H), 7.39 (t, J = 8.8 Hz, 1H), 4.63-4.60 (m, 2H), 4.21-4.16 (m, 2H), 2.54 (s, 3H), 1.97-1.93 (m, 2H). LCMS calcd for; C₂₃H₂₀ClF₄N₅O₄S₂. Found; 605.90 (M + 1). |
| 1064 | 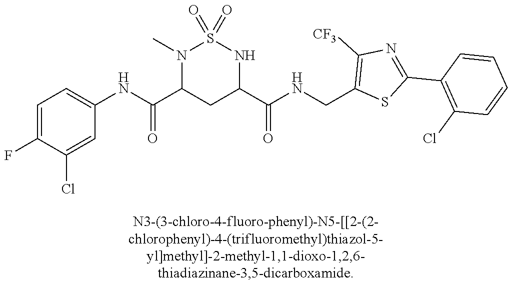 [SMILES: CN1C(C(=O)Nc2ccc(F)c(Cl)c2)CC(C(=O)NCc2sc(-c3ccccc3Cl)nc2C(F)(F)F)NS1(=O)=O] | ¹H NMR (400 MHz, DMSO-d6): δ 10.51 (s, 1H), 9.10-9.06 (m, 1H), 8.14-8.09 (m, 1H), 7.93-7.89 (m, 1H), 7.66-7.63 (m, 1H), 7.56-7.48 (m, 3H), 7.46-7.43 (m, 1H), 7.39-7.34 (m, 1H), 4.73- 4.61 (m, 2H), 4.21-4.11 (m, 2H), 2.54 (s, 3H), 1.99-1.85 (m, 2H). LCMS calcd for; C₂₃H₁₉Cl₂F₄N₅O₄S₂. Found; 640 (M + 1). |
| 1065 | 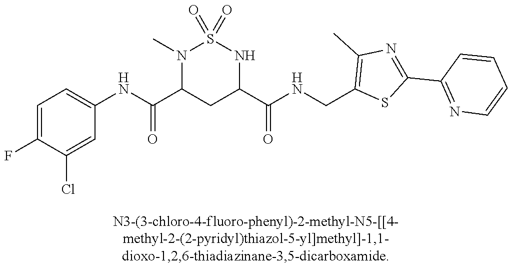 [SMILES: Cc1nc(-c2ccccn2)sc1CNC(=O)C1CC(C(=O)Nc2ccc(F)c(Cl)c2)N(C)S(=O)(=O)N1] | ¹H NMR (400 MHz, DMSO-d6): δ 10.53 (s, 1H), 8.83-8.80 (m, 1H), 8.59 (d, J = 4.4 Hz, 1H), 8.06-8.04 (m, 1H), 7.96-7.91 (m, 2H), 7.55-7.52 (m, 1H), 7.47-7.35 (m, 3H), 4.47-4.29 (m, 2H), 4.23- 4.18 (m, 1H), 4.10-4.05 (m, 1H), 2.56 (s, 3H), 2.42 (s, 3H), 1.97-1.93 (m, 2H). LCMS calcd for; C₂₂H₂₂ClFN₆O₄S₂. Found; 553.10 (M + 1). |
| 1065- | N3-(3-chloro-4-fluoro-phenyl)-2-methyl-N5-[[4- | .¹H NMR (400 MHz, DMSO-d6): δ |
| ISOMER-A | methyl-2-(2-pyridyl)thiazol-5-yl]methyl]-1,1- | 10.52 (s, 1H), 8.81 (t, J = 5.2 Hz, |
| | dioxo-1,2,6-thiadiazinane-3,5-dicarboxamide | 1H), 8.59 (d, J = 4.4 Hz, 1H), |
| | | 8.06-8.03 (m, 1H), 7.96-7.90 (m, |
| | | 2H), 7.56-7.52 (m, 1H), 7.48- |
| | | 7.34 (m, 3H), 4.52-4.40 (m, 2H), |
| | | 4.24-4.20 (m, 1H), 4.10-4.08 (m, |
| | | 1H), 2.57 (s, 3H), 2.42 (s, 3H), 1.97- |
| | | 1.86 (m, 2H). LCMS calcd for; |
| | | C₂₂H₂₂ClFN₆O₄S₂. Found; |
| | | 553.05 (M + 1). |
| 1065- | N3-(3-chloro-4-fluoro-phenyl)-2-methyl-N5-[[4- | ¹H NMR (400 MHz, DMSO-d6): δ |
| ISOMER-B | methyl-2-(2-pyridyl)thiazol-5-yl]methyl]-1,1- | 10.52 (s, 1H), 8.85-8.80 (m, 1H), 8.59 |
| | dioxo-1,2,6-thiadiazinane-3,5-dicarboxamide. | (d, J = 4.4 Hz, 1H), 8.06-8.03 (m, 1H), |
| | | 7.96-7.88 (m, 2H), |
| | | 7.56-7.52 (m, 1H), 7.48-7.35 (m, |
| | | 3H), 4.52-4.40 (m, 2H), 4.25- |
| | | 4.18 (m, 1H), 4.10-4.06 (m, 1H), |
| | | 2.56 (s, 3H), 2.42 (s, 3H), 1.97- |
| | | 1.86 (m, 2H). LCMS calcd for; |
| | | C₂₂H₂₂ClFN₆O₄S₂. Found; |
| | | 553.05 (M + 1). |
| 1066 | 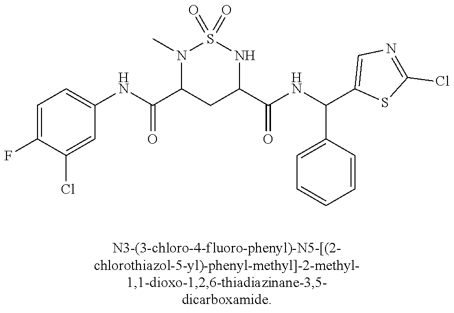 [SMILES: CN1C(C(=O)Nc2ccc(F)c(Cl)c2)CC(C(=O)NC(c2ccccc2)c2cnc(Cl)s2)NS1(=O)=O] | ¹H NMR (400 MHz, DMSO-d6): δ 10.55 (s, 1H), 9.35-9.30 (m, 1H), 7.98-7.92 (m, 1H), 7.50-7.30 (m, 9H), 6.40-6.36 (m, 1H), 4.25-4.20 (m, 2H), 2.57 (s, 3H), 1.98-1.94 (m, 2H). LCMS calcd for; C₂₂H₂₀Cl₂FN₅O₄S₂. Found: 572 (M + 1). |
| 1066- | N3-(3-chloro-4-fluoro-phenyl)-N5-[(R)-(2- | ¹H NMR (400 MHz, DMSO-d6): δ |
| ISOMER-A | chlorothiazol-5-yl)-phenyl-methyl]-2-methyl- | 10.53 (s, 1H), 9.30-9.25 (m, 1H), |
| | 1,1-dioxo-1,2,6-thiadiazinane-3,5- | 7.97-7.94 (m, 1H), |
| | dicarboxamide. | 7.57-7.52 (m, 1H), 7.46-7.34 (m, |
| | | 8H), 6.38 (d, J = 8.0 Hz, 1H), |
| | | 4.25-4.15 (m, 2H), 2.56 (s, 3H), 2.00- |
| | | 1.95 (m, 2H). LCMS calcd for; |
| | | C₂₂H₂₀Cl₂FN₅O₄S₂. Found; 572 (M + |
| | | 1). |
| 1066- | N3-(3-chloro-4-fluoro-phenyl)-N5-[(S)-(2- | ¹H NMR (400 MHz, DMSO-d6): δ |
| ISOMER-B | chlorothiazol-5-yl)-phenyl-methyl]-2-methyl- | 10.51 (s, 1H), 9.30 (d, J = 8.4 Hz, 1H), |
| | 1,1-dioxo-1,2,6-thiadiazinane-3,5- | 7.96-7.94 (m, 1H), 7.56-7.32 (m, |
| | | 9H), 6.38 (d, J = 8.0 Hz, 1H), |
| | | 4.24-4.20 (m, 2H), 2.57 (s, 3H), |
| | | 2.02-1.92 (m, 2H). |
| | | LCMS calcd for; |
| | | C₂₂H₂₀Cl₂FN₅O₄S₂. Found; 572 (M + |
| | | 1). |
| 1066- | N3-(3-chloro-4-fluoro-phenyl)-N5-[(R)-(2- | ¹H NMR (400 MHz, DMSO-d6): δ |
| ISOMER-C | chlorothiazol-5-yl)-phenyl-methyl]-2-methyl- | 10.53 (s, 1H), 9.32 (d, J = 8.4 Hz, |
| | 1,1-dioxo-1,2,6-thiadiazinane-3,5- | 1H), 7.94-7.92 (m, 1H), |
| | dicarboxamide. | 7.54-7.50 (m, 1H), 7.43-7.32 (m, |
| | | 8H), 6.38 (d, J = 8.0 Hz, 1H), |
| | | 4.22-4.15 (m, 2H), 2.54 (s, |
| | | 3H), 2.00-1.92 (m, 2H). LCMS |
| | | calcd for; C₂₂H₂₀Cl₂FN₅O₄S₂. Found; |
| | | 572 (M + 1). |
| 1066- | N3-(3-chloro-4-fluoro-phenyl)-N5-[(S)-(2- | ¹H NMR (400 MHz, DMSO-d6): δ |
| ISOMER-D | chlorothiazol-5-yl)-phenyl-methyl]-2-methyl- | 10.51 (s, 1H), 9.30-9.22 (m, 1H), |
| | 1,1-dioxo-1,2,6-thiadiazinane-3,5- | 7.96-7.94 (m, 1H), |
| | dicarboxamide. | 7.55-7.333 (m, 9H), 6.37 (d, J = |
| | | 7.2 Hz, 1H), 4.20-4.15 (m, 2H), |
| | | 2.55 (s, 3H), 1.98-1.90 (m, 2H). |
| | | LCMS calcd for; C₂₂H₂₀Cl₂FN₅O₄S₂. |
| | | Found; 572 (M + 1). |
| 1067 | 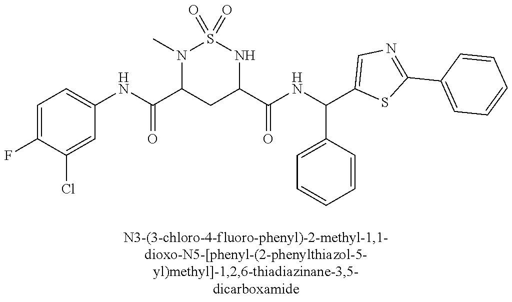 [SMILES: CN1C(C(=O)Nc2ccc(F)c(Cl)c2)CC(C(=O)NC(c2ccccc2)c2cnc(-c3ccccc3)s2)NS1(=O)=O] | .¹H NMR (400 MHz, DMSO-d6): δ 10.55 (s, 1H), 9.40-9.30 (m, 1H), 7.99-7.33 (m, 15H), 6.47-6.45 (m, 1H), 4.25-4.20 (m, 2H), 2.58 (s, 3H), 2.08- 1.87 (m, 2H). LCMS calcd for; C₂₈H₂₅ClFN₅O₄S₂. Found; 614.10 (M + 1). |
| 1067- | N3-(3-chloro-4-fluoro-phenyl)-2-methyl-1,1- | ¹H NMR (400 MHz, DMSO-d6): δ |
| ISOMER-A | dioxo-N5-[phenyl-(2-phenylthiazol-5- | 10.54 (s, 1H), 9.35 (d, J = 8.0 Hz, |
| | yl)methyl]-1,2,6-thiadiazinane-3,5- | 1H), 7.97-7.93 (m, 1H), |
| | dicarboxamide. | 7.89-7.86 (m, 2H), 7.68 (s, 1H), |
| | | 7.60-7.30 (m, 11H), 6.45 (d, J = |
| | | 8.0 Hz, 1H), 4.26-4.22 (m, 2H), |
| | | 2.58 (s, 3H), 2.02-1.92 (m, 2H). |
| | | LCMS calcd for; C₂₈H₂₅ClFN₅O₄S₂. |
| | | Found; |
| | | 614.05 (M + 1). |
| 1067- | N3-(3-chloro-4-fluoro-phenyl)-2-methyl-1,1- | ¹H NMR (400 MHz, DMSO-d6): δ |
| ISOMER-B | dioxo-N5-[(S)-phenyl-(2-phenylthiazol-5- | 10.55 (s, 1H), 9.33 (d, J = 8.0 Hz, |
| | yl)methyl]-1,2,6-thiadiazinane-3,5- | 1H), 7.97-7.94 (m, 1H), |
| | dicarboxamide. | 7.88-7.86 (m, 2H), 7.65 (s, |
| | | 1H), 7.55-7.52 (m, 1H), 7.46- |
| | | 7.33 (m, 10H), 6.46 (d, J = 7.6 Hz, |
| | | 1H), 4.30-4.20 (m, 2H), 2.57 (s, 3H), |
| | | 2.03-1.96 (m, 2H). LCMS calcd for; |
| | | C₂₈H₂₅ClFN₅O₄S₂. Found; |
| | | 614.05 (M + 1). |
| 1067- | (5S)-N3-(3-chloro-4-fluoro-phenyl)-2-methyl- | ¹H NMR (400 MHz, DMSO-d6): δ |
| ISOMER-C | 1,1-dioxo-N5-[(R)-phenyl-(2-phenylthiazol-5- | 10.54 (s, 1H), 9.32 (d, J = 7.6 Hz, |
| | yl)methyl]-1,2,6-thiadiazinane-3,5- | 1H), 7.97-7.94 (m, 1H), |
| | dicarboxamide. | 7.88-7.86 (m, 2H), 7.65 (s, 1H), |
| | | 7.55-7.52 (m, 1H), 7.46-7.33 (m, |
| | | 10H), 6.46 (d, J = 7.6 Hz, 1H), |
| | | 4.30-4.20 (m, 2H), 2.57 (s, 3H), |
| | | 2.03-1.96 (m, 2H). LCMS calcd for; |
| | | C₂₈H₂₅ClFN₅O₄S₂. Found; |
| | | 614.05 (M + 1). |
| 1067- | N3-(3-chloro-4-fluoro-phenyl)-2-methyl-1,1- | .¹H NMR (400 MHz, DMSO-d6): δ |
| ISOMER-D | dioxo-N5-[(S)-phenyl-(2-phenylthiazol-5- | 10.54 (s, 1H), 9.34 (d, J = 7.6 Hz, |
| | yl)methyl]-1,2,6-thiadiazinane-3,5- | 1H), 7.97-7.93 (m, 1H), |
| | dicarboxamide | 7.89-7.86 (m, 2H), 7.68 (s, 1H), |
| | | 7.60-7.50 (m, 1H), 7.49-7.32 (m, |
| | | 10H), 6.45 (d, J = 8.0 Hz, 1H), |
| | | 4.26-4.22 (m, 2H), 2.58 (s, 3H), |
| | | 2.02-1.92 (m, 2H). LCMS calcd for; |
| | | C₂₈H₂₅ClFN₅O₄S₂. Found; 614 (M + |
| | | 1). |
| 1068 | 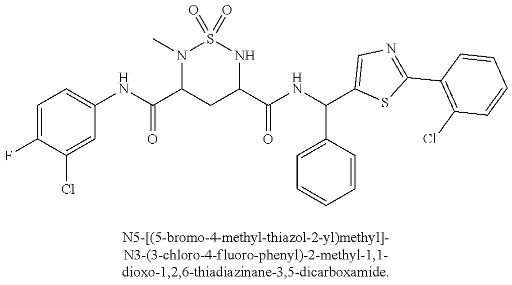 [SMILES: CN1C(C(=O)Nc2ccc(F)c(Cl)c2)CC(C(=O)NC(c2ccccc2)c2cnc(-c3ccccc3Cl)s2)NS1(=O)=O] | ¹H NMR (400 MHz, DMSO-d6): δ 10.55 (s, 1H), 9.46-9.36 (m, 1H), 8.17-7.33 (m, 14H), 6.53-6.50 (m, 1H), 4.25-4.20 (m, 2H), 2.58 (s, 3H), 2.03-1.93 (m, 2H). LCMS calcd for; C₂₈H₂₄Cl₂FN₅O₄S₂. Found; 648.10 (M + 1). |
| 1068- | N3-(3-chloro-4-fluoro-phenyl)-N5-[[2-(2- | ¹H NMR (400 MHz, DMSO-d6): δ |
| ISOMER-A | chlorophenyl)thiazol-5-yl]-phenyl-methyl]-2- | 10.54 (s, 1H), 9.38 (d, J = 7.6 Hz, |
| | methyl-1,1-dioxo-1,2,6-thiadiazinane-3,5- | 1H), 8.17-8.14 (m, 1H), |
| | dicarboxamide. | 7.97-7.93 (m, 1H), 7.79 (s, 1H), |
| | | 7.64-7.61 (m, 1H), 7.56-7.30 (m, |
| | | 10H), 6.51 (d, J = 8.0 Hz, 1H), |
| | | 4.26-4.20 (m, 2H), 2.58 (s, 3H), |
| | | 2.03-1.90 (m, 2H). LCMS calcd for; |
| | | C₂₈H₂₄Cl₂FN₅O₄S₂. Found; 648 (M + |
| | | 1). |
| 1068- | N3-(3-chloro-4-fluoro-phenyl)-N5-[(S)-[2-(2- | ¹H NMR (400 MHz, DMSO-d6): δ |
| ISOMER-B | chlorophenyl)thiazol-5-yl]-phenyl-methyl]-2- | 10.54 (s, 1H), 9.36 (d, J = 8.0 Hz, |
| | methyl-1,1-dioxo-1,2,6-thiadiazinane-3,5- | 1H), 8.16-8.12 (m, 1H), |
| | dicarboxamide. | 7.97-7.94 (m, 1H), 7.76 (s, 1H), |
| | | 7.64-7.34 (m, 11H), 6.51 (d, J = |
| | | 8.0 Hz, 1H), 4.30-4.20 (m, 2H), |
| | | 2.58 (s, 3H), 2.03-1.97 (m, 2H). |
| | | LCMS calcd for; |
| | | C₂₈H₂₄Cl₂FN₅O₄S₂. Found; |
| | | 648 (M + 1). |
| 1068- | 5S)-N3-(3-chloro-4-fluoro-phenyl)-N5-[(R)-[2- | (¹H NMR (400 MHz, DMSO-d6): δ |
| ISOMER-C | (2-chlorophenyl)thiazol-5-yl]-phenyl-methyl]-2- | 10.55 (s, 1H), 9.37 (d, J = 7.6 Hz, |
| | methyl-1,1-dioxo-1,2,6-thiadiazinane-3,5- | 1H), 8.16-8.12 (m, 1H), |
| | dicarboxamide | 7.97-7.94 (m, 1H), 7.76 (s, |
| | | 1H), 7.64-7.33 (m, 11H), 6.51 |
| | | (d, J = 8.0 Hz, 1H), 4.30-4.20 (m, |
| | | 2H), 2.57 (s, 3H), 2.03-1.97 (m, |
| | | 2H). LCMS |
| | | calcd for; C₂₈H₂₄Cl₂FN₅O₄S₂. |
| | | Found: 648 (M + 1). |
| 1068- | N3-(3-chloro-4-fluoro-phenyl)-N5-[(S)-[2-(4- | ¹H NMR (400 MHz, DMSO-d6): δ |
| ISOMER-D | chlorophenyl)thiazol-5-yl]-phenyl-methyl]-2- | 10.55 (s, 1H), 9.39 (d, J = 8.4 Hz, |
| | methyl-1,1-dioxo-1,2,6-thiadiazinane-3,5- | 1H), 8.17-8.13 (m, 1H), |
| | dicarboxamide. | 7.97-7.94 (m, 1H), 7.76 (s, 1H), |
| | | 7.64-7.62 (m, 1H), 7.56-7.30 (m, |
| | | 10H), 6.51 (d, J = 8.4 Hz, |
| | | 1H), 4.25-4.20 (m, 2H), 2.57 (s, 3H), |
| | | 2.07-1.93 (m, 2H). LCMS calcd for; |
| | | C₂₈H₂₄Cl₂FN₅O₄S₂. Found; |
| | | 648 (M + 1). |
| 1069 | 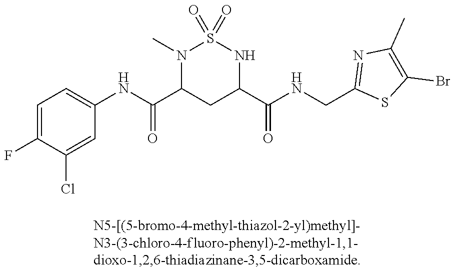 [SMILES: Cc1nc(CNC(=O)C2CC(C(=O)Nc3ccc(F)c(Cl)c3)N(C)S(=O)(=O)N2)sc1Br] | ¹H NMR (400 MHz, DMSO-d6): δ 10.54 (s, 1H), 9.00 (t, J = 5.6 Hz, 1H), 8.00-7.92 (m, 1H), 7.60-7.52 (m, 1H), 7.60-7.35 (m, 2H), 4.54-4.52 (m, 2H), 4.26-4.10 (m, 2H), 2.58 (s, 3H), 2.28 (s, 3H), 2.02-1.88 (m, 2H), LCMS calcd for: C₁₇H₁₈BrClFN₅O₄S₂. Found; 555.9 (M + 2). |
| 1070 | 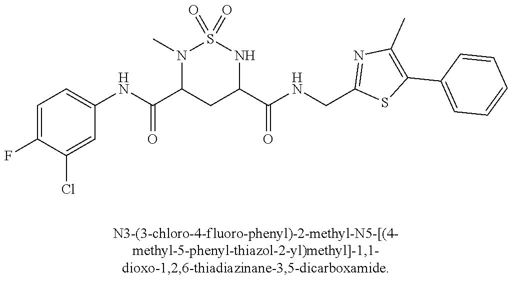 [SMILES: Cc1nc(CNC(=O)C2CC(C(=O)Nc3ccc(F)c(Cl)c3)N(C)S(=O)(=O)N2)sc1-c1ccccc1] | ¹H NMR (400 MHz, DMSO-d6): δ 10.53 (s, 1H), 9.05-9.00 (m, 1H), 7.96-7.92 (m, 1H), 7.58-7.52 (m, 1H), 7.48-7.36 (m, 7H), 4.60-4.50 (m, 2H), 4.26-4.16 (m, 2H), 2.58- (s, 3H), 2.39 (s, 3H), 2.00-1.92 (m, 2H). LCMS calcd for; C₂₃H₂₃ClFN₅O₄S₂. Found; 552.05 (M + 1). |
| 1071 | 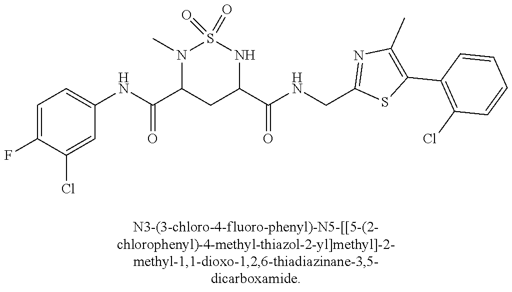 [SMILES: Cc1nc(CNC(=O)C2CC(C(=O)Nc3ccc(F)c(Cl)c3)N(C)S(=O)(=O)N2)sc1-c1ccccc1Cl] | ¹H NMR (400 MHz, DMSO-d6): δ 10.53 (s, 1H), 9.04 (t, 1H), 7.96-7.92 (m, 1H), 7.62 -7.60 (m, 1H), 7.56-7.52 (m, 1H), 7.50-7.35 (m, 5H),4.65- 4.48 (m, 2H), 4.26-4.14 (m, 2H), 2.57 (s, 3H), 2.17 (s, 3H), 2.07-1.90 (m, 2H). LCMS calcd for: C₂₃H₂₂Cl₂FN₅O₄S₂. Found; 586 (M + 1). |
| 1072 | 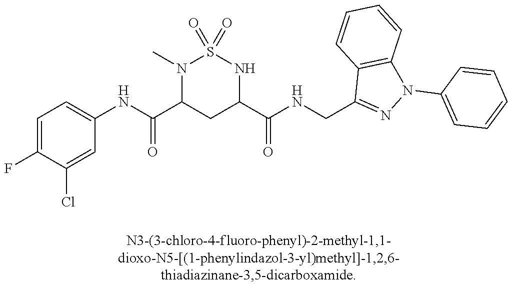 [SMILES: CN1C(C(=O)Nc2ccc(F)c(Cl)c2)CC(C(=O)NCc2nn(-c3ccccc3)c3ccccc23)NS1(=O)=O] | ¹H NMR (400 MHz, DMSO-d6): δ 10.52 (s, 1H), 8.83 (t, J = 5.6 Hz, 1H), 7.98-7.89 (m, 2H), 7.89-7.79 (m, 1H), 7.75 (d, J = 8.0 Hz, 2H), 7.63-7.45 (m, 4H), 7.44-7.33 (m, 3H), 7.27 (t, J = 8.0 Hz, 1H), 4.82-4.66 (m, 2H), 4.25- 4.12 (m, 2H), 2.56 (s, 3H), 2.02-1.92 (m, 2H). LCMS calcd for; C₂₆H₂₄ClFN₆O₄S. Found; 571.05 (M + 1). |
| 1072- | N3-(3-chloro-4-fluoro-phenyl)-2-methyl-1,1- | ¹H NMR (400 MHz, DMSO-d6): δ |
| ISOMER-A | dioxo-N5-[(1-phenylindazol-3-yl)methyl]-1,2,6- | 10.51 (bs, 1H), 8.85-8.80 (m, 1H), |
| | thiadiazinane-3,5-dicarboxamide. | 7.96-7.91 (m, 2H), 7.83 (d, J = 8.4 |
| | | Hz, 1H), 7.75 (d, J = 8.0 Hz, 2H), |
| | | 7.62-7.48 (m, 4H), 7.42-7.36 (m, |
| | | 3H), 7.30-7.24 (m, 1H), |
| | | 4.80-4.68 (m, 2H), 4.20- |
| | | 4.10 (m, 2H), 2.00-1.95 (m, |
| | | 2H). LCMS calcd for; |
| | | C₂₆H₂₄ClFN₆O₄S. Found; |
| | | 571.05 (M + 2). |
| 1072- | N3-(3-chloro-4-fluoro-phenyl)-2-methyl-1,1- | ¹H NMR (400 MHz, DMSO-d6): δ |
| ISOMER-B | dioxo-N5-[(1-phenylindazol-3-yl)methyl]-1,2,6- | 10.53 (bs, 1H), 8.85-8.80 (m, 1H), |
| | thiadiazinane-3,5-dicarboxamide. | 7.96-7.91 (m, 2H), 7.83 (d, J = 8.4 |
| | | Hz, 1H), 7.75 (d, J = 7.6 Hz, |
| | | 2H), 7.63-7.47 (m, 4H), |
| | | 7.42-7.36 (m, 3H), 7.30-7.24 (m, |
| | | 1H), 4.80-4.68 (m, 2H), 4.22- |
| | | 4.12 (m, 2H), 1.99-1.95 (m, |
| | | 2H). LCMS calcd for; |
| | | C₂₆H₂₄ClFN₆O₄S. Found; |
| | | 571.00 (M + 2). |
| 1073 | 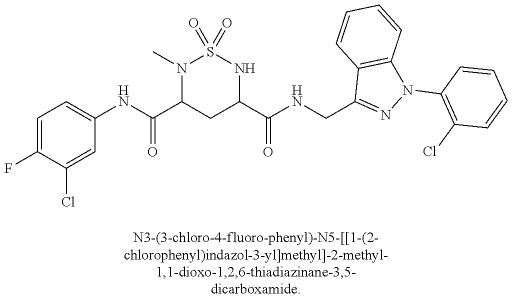 [SMILES: CN1C(C(=O)Nc2ccc(F)c(Cl)c2)CC(C(=O)NCc2nn(-c3ccccc3Cl)c3ccccc23)NS1(=O)=O] | ¹H NMR (400 MHz, DMSO-d6): δ 10.54 (s, 1H), 8.85 (t, J = 5.2 Hz, 1H), 7.96-7.91 (m, 2H), 7.77-7.74 (m, 1H), 7.62-7.52 (m, 4H), 7.47-7.37 (m, 3H), 7.27-7.20 (m, 2H), 4.79-4.67 (m, 2H), 4.22-4.17 (m, 2H), 2.57 (s, 3H), 2.04- 1.93 (m, 2H). LCMS calcd for; C₂₆H₂₃Cl₂FN₆O₄S. Found; 605.10 (M + 1). |
| 1074 | 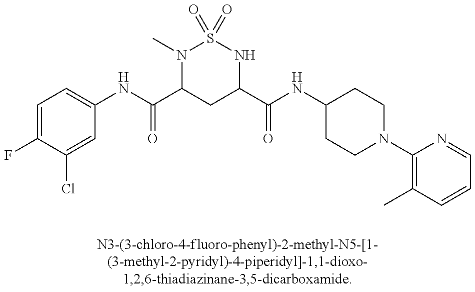 [SMILES: Cc1cccnc1N1CCC(NC(=O)C2CC(C(=O)Nc3ccc(F)c(Cl)c3)N(C)S(=O)(=O)N2)CC1] | ¹H NMR (400 MHz, DMSO-d6): δ 10.53 (s, 1H), 8.14-8.04 (m, 2H), 7.97-7.94 (m, 1H), 7.59-7.53 (m, 1H), 7.48-7.47 (m, 1H), 7.39 (t, J = 9.2 Hz, 1H), 7.25 (d, J = 9.6 Hz, 1H), 6.91-6.88 (m, 1H), 4.22-4.18 (m, 1H), 4.11-4.05 (m, 1H), 3.81-3.69 (m, 1H), 2.85-2.72 (m, 2H), 2.57 (s, 3H), 2.22 (s, 3H), 2.00-1.77 (m, 4H), 1.64-1.55 (m, 2H). LCMS calcd for; C₂₃H₂₈ClFN₆O₄S. Found; 539.15 (M + 1). |
| 1081 | 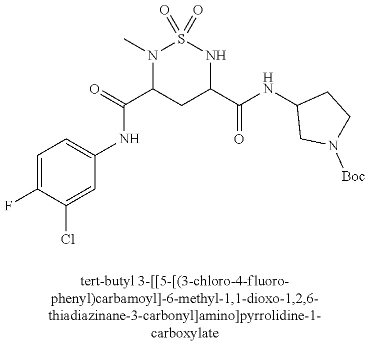 [SMILES: CN1C(C(=O)Nc2ccc(F)c(Cl)c2)CC(C(=O)NC2CCN(C(=O)OC(C)(C)C)C2)NS1(=O)=O] | .¹H NMR (400 MHz, DMSO-d6): δ 10.49 (s, 1H), 8.36-8.24 (m, 1H), 7.96-7.88 (m, 1H), 7.58-7.46 (m, 1H), 7.36 (t, J = 9.2 Hz, 1H), 7.25-7.20 (m, 1H), 4.20-4.10 (m, 2H), 4.05-4.00 (m, 1H), 3.45-3.38 (m, 1H), 3.10-3.00 (m, 1H), 2.53 (s, 3H), 1.97-1.70 (m, 4H), 1.36 (s, 9H), 0.95-0.77 (m, 2H). LCMS calcd for; C₂₁H₂₉ClFN₅O₆S. Found; 532.20 (M − 1). |
| 1082 | 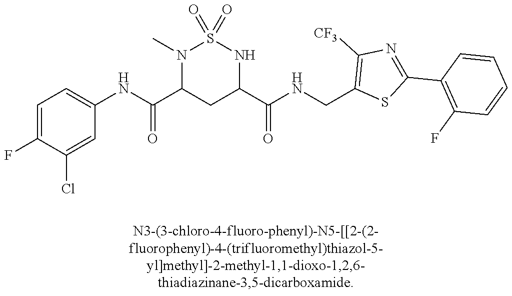 [SMILES: CN1C(C(=O)Nc2ccc(F)c(Cl)c2)CC(C(=O)NCc2sc(-c3ccccc3F)nc2C(F)(F)F)NS1(=O)=O] | ¹H NMR (400 MHz, DMSO-d6): δ 10.52 (s, 1H), 9.11-9.08 (m, 1H), 8.19-8.14 (m, 1H), 7.96-7.92 (m, 1H), 7.64-7.31 (m, 6H), 4.70-4.65 (m, 2H), 4.24-4.15 (m, 2H), 2.57 (s, 3H), 2.08-1.90 (m, 2H). LCMS calcd for; C₂₃H₁₉ClF₅N₅O₄S₂. Found; 623.90 (M + 1). |
| 1082- | N3-(3-chloro-4-fluoro-phenyl)-N5-[[2-(2- | ¹H NMR (400 MHz, DMSO-d6): δ |
| ISOMER-A | fluorophenyl)-4-(trifluoromethyl)thiazol-5- | 10.53 (s ,1H), 9.12-9.07 (m, 1H), |
| | yl]methyl]-2-methyl-1,1-dioxo-1,2,6- | 8.19-8.14 (m, 1H), 7.96-7.92 (m, |
| | thiadiazinane-3,5-dicarboxamide | 1H), 7.62-7.57 (m, 1H), |
| | | 7.56-7.52 (m, 1H), 7.50-7.43 (m, |
| | | 2H), 7.42-7.36 (m, 2H), 4.75- |
| | | 4.60 (m, 2H), 4.25-4.10 (m, 2H), 2.57 |
| | | (s, 3H), 2.02-1.90 (m, 2H). LCMS |
| | | calcd for; C₂₃H₁₉ClF₅N₅O₄S₂. Found; |
| | | 645.95 (M + 23).⁺ . |
| 1082- | N3-(3-chloro-4-fluoro-phenyl)-N5-[[2-(2- | ¹H NMR (400 MHz, DMSO-d6): δ |
| ISOMER-B | fluorophenyl)-4-(trifluoromethyl)thiazol-5- | 10.53 (s, 1H), 9.09 (d, J = 9.0 Hz, |
| | yl]methyl]-2-methyl-1,1-dioxo-1,2,6- | 1H), 8.20-8.14 (m, 1H), |
| | thiadiazinane-3,5-dicarboxamide. | 7.98-7.92 (m, 1H), 7.64-7.57 (m, |
| | | 1H), 7.56-7.52 (m, 1H), |
| | | 7.50-7.43 (m, 2H), 7.42-7.35 (m, 2H), |
| | | 4.75-4.65 (m, 2H), 4.25-4.10 (m, 2H), |
| | | 2.57 (s, 3H), 2.02-1.91 (m, 2H). |
| | | LCMS calcd for; C₂₃H₁₉ClF₅N₅O₄S₂. |
| | | Found; 624 (M + 1). |
| 1083 | 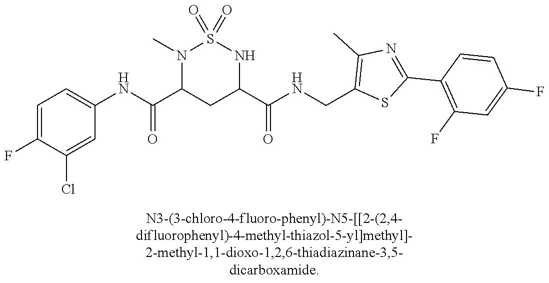 [SMILES: Cc1nc(-c2ccc(F)cc2F)sc1CNC(=O)C1CC(C(=O)Nc2ccc(F)c(Cl)c2)N(C)S(=O)(=O)N1] | ¹H NMR (400 MHz, DMSO-d6): δ 10.52 (s, 1H), 8.81 (t, J = 5.6 Hz, 1H), 8.23-8.17 (m, 1H), 7.95 (dd, J = 6.9, 2.6 Hz, 1H), 7.58-7.34 (m, 4H), 7.26-7.22 (m, 1H), 4.50-4.40 (m, 2H), 4.22- 4.18 (m, 1H), 4.08-4.06 (m, 1H), 2.56 (s, 3H), 2.43 (s, 3H), 2.02-1.84 (m, 2H). LCMS calcd for; C₂₃H₂₁ClF₃N₅O₄S₂. Found; 588 (M +1). |
| 1083- | N3-(3-chloro-4-fluoro-phenyl)-N5-[[2-(2,4- | ¹H NMR (400 MHz, DMSO-d6): δ |
| ISOMER-A | difluorophenyl)-4-methyl-thiazol-5-yl]methyl]- | 10.52 (s, 1H), 8.85-8.80 (m, 1H), |
| | 2-methyl-1,1-dioxo-1,2,6-thiadiazinane-3,5- | 8.23-8.16 (m, 1H), |
| | dicarboxamide. | 7.96-7.92 (m, 1H), 7.53- |
| | | 7.35 (m, 4H), 7.27-7.22 (m, 1H), 4.48- |
| | | 4.40 (m, 2H), 4.22-4.06 (m, 2H), 2.56 |
| | | (s, 3H), 2.43 (s, 3H), 2.00-1.84 (m, |
| | | 2H). LCMS calcd for; |
| | | C₂₃H₂₃ClF₃N₅O₄S₂. Found; |
| | | 588.10 (M + 1). |
| 1083- | N3-(3-chloro-4-fluoro-phenyl)-N5-[[2-(2,4- | ¹H NMR (400 MHz, DMSO-d6): δ |
| ISOMER-B | difluroophenyl)-4-methyl-thiazol-5-yl]methyl]- | 10.49 (s, 1H), 8.80-8.74 (m, 1H), 8.23- |
| | 2-methyl-1,1-dioxo-1,2,6-thiadiazinane-3,5- | 8.16 (m, 1H), 7.96-7.92 (m, 1H), 7.56- |
| | dicarboxamide. | 7.44 (m, 2H), 7.41-7.38 (m, 2H), 7.28- |
| | | 7.20 (m, 1H), 4.52-4.40 (m, 2H), 4.16- |
| | | 4.00 (m, 2H), 2.53 (s, 3H), 2.43 (s, |
| | | 3H), 2.00-1.82 (m, 2H). LCMS calcd |
| | | for: C₂₃H₂₁ClF₃N₅O₄S₂. Found; |
| | | 588.05 (M + 1). |
| 1084 | 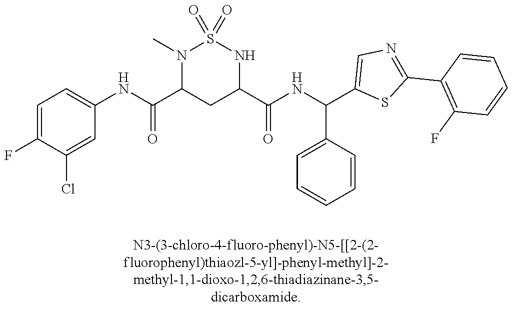 [SMILES: CN1C(C(=O)Nc2ccc(F)c(Cl)c2)CC(C(=O)NC(c2ccccc2)c2cnc(-c3ccccc3F)s2)NS1(=O)=O] | ¹H NMR (400 MHz, DMSO-d6): δ 10.55 (s, 1H), 9.40-9.35 (m, 1H), 8.21-7.33 (m, 14H), 6.52-6.50 (m, 1H), 4.27-4.20 (m, 2H), 2.58 (s, 3H), 2.03-1.90 (m, 2H). LCMS calcd for; C₂₈H₂₄ClF₂N₅O₄S₂. Found; 632 (M + 1). |
| 1085 | 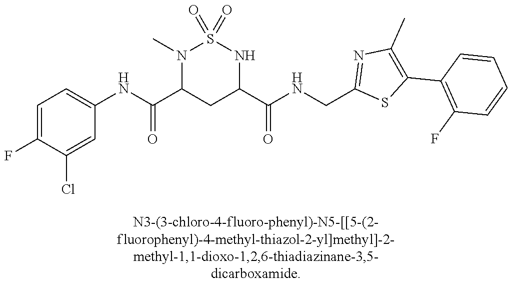 [SMILES: Cc1nc(CNC(=O)C2CC(C(=O)Nc3ccc(F)c(Cl)c3)N(C)S(=O)(=O)N2)sc1-c1ccccc1F] | ¹H NMR (400 MHz, DMSO-d6): δ 10.53 (s, 1H), 9.04 (t, J = 6.0 Hz, 1H), 7.96-7.94 (m, 1H), 7.56-7.27 (m, 7H), 4.64-4.50 (m, 2H), 4.26-4.16 (m, 2H), 2.57 (s, 3H), 2.27 (s, 3H), 2.04-1.92 (m, 2H). LCMS calcd for; C₂₃H₂₂ClF₂N₅O₄S₂. Found; 570.05 (M + 1). |
| 1086 | 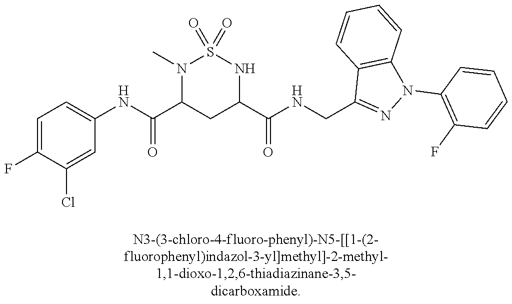 [SMILES: CN1C(C(=O)Nc2ccc(F)c(Cl)c2)CC(C(=O)NCc2nn(-c3ccccc3F)c3ccccc23)NS1(=O)=O] | ¹H NMR (400 MHz, DMSO-d6): δ 10.51 (s, 1H), 8.84 (t, J = 6.0 Hz, 1H), 7.97-7.88 (m, 2H), 7.66-7.63 (m, 1H), 7.55-7.35 (m, 8H), 7.27-7.23 (m, 1H), 4.74-4.70 (m, 2H), 4.23-4.11 (m, 2H), 2.55 (s, 3H), 2.01-1.89 (m, 2H). LCMS calcd for; C₂₆H₂₃ClF₂N₆O₄S. Found; 589.10 (M + 1). |
| 1086- | N3-(3-chloro-4-fluoro-phenyl)-N5-[[1-(2- | ¹H NMR (400 MHz, DMSO-d6): δ |
| ISOMER-A | fluorophenyl)indazol-3-yl]methyl]-2-methyl- | 10.52 (s, 1H), 8.84 (t, J = 5.2 Hz, |
| | 1,1-dioxo-1,2,6-thiadiazinane-3,5- | 1H), 7.96-7.90 (m, 2H), |
| | dicarboxamide. | 7.68-7.64 (m, 1H), 7.56-7.38 (m, |
| | | 8H), 7.29-7.24 (m, 1H), |
| | | 4.78-4.72 (m, 2H), 4.22-4.16 (m, 2H), |
| | | 2.57 (s, 3H), 2.03-1.94 (m, 2H). |
| | | LCMS calcd for: |
| | | C₂₆H₂₃ClF₂N₆O₄S. Found; |
| | | 589.05 (M + 1). |
| 1086- | N3-(3-chloro-4-fluoro-phenyl)-N5-[[1-(2- | ¹H NMR (400 MHz, DMSO-d6): δ |
| ISOMER-B | fluorophenyl)indazol-3-yl]methyl]-2-methyl- | 10.51 (s, 1H), 8.85-8.83 (m, 1H), |
| | 1,1-dioxo-1,2,6-thiadiazinane-3,5- | 7.96-7.90 (m, 2H), |
| | dicarboxamide. | 7.70-7.60 (m, 1H), 7.56- |
| | | 7.35 (m, 9H), 7.29-7.24 (m, 1H), 4.80- |
| | | 4.70 (m, 2H), 4.25-4.10 (m, 1H), 2.57 |
| | | (s, 3H), 2.00-1.92 (m, 2H). LCMS |
| | | calcd for; C₂₆H₂₃ClF₂N₆O₄S. Found; |
| | | 589.05 (M + 1). |
| 1087 | 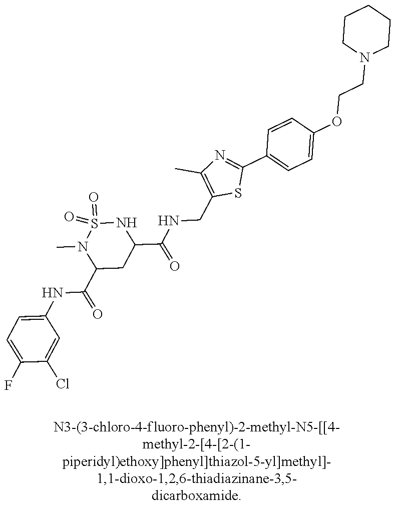 [SMILES: Cc1nc(-c2ccc(OCCN3CCCCC3)cc2)sc1CNC(=O)C1CC(C(=O)Nc2ccc(F)c(Cl)c2)N(C)S(=O)(=O)N1] | ¹H NMR (400 MHz, DMSO-d6): δ 10.52 (s, 1H), 8.77-8.75 (m, 1H), 7.95 (dd, J = 6.8, 2.7 Hz, 1H), 7.78-7.74 (m, 2H), 7.58-7.49 (m, 1H), 7.41-7.37 (m, 2H), 7.01 (d, J = 8.5 Hz, 2H), 4.42-4.40 (m, 2H), 4.23-4.15 (m, 1H), 4.12-4.09 (m, 3H), 2.65 (t, J = 5.8 Hz, 2H), 2.56 (s, 3H), 2.44-2.40 (m, 4H), 2.37 (s, 3H), 2.00-1.87 (m, 2H), 1.49- 1.47 (m, 4H), 1.38-1.35 (m, 2H). LCMS calcd for; C₃₀H₃₆ClFN₆O₅S₂. Found: 679.10 (M + 1). |
| 1087- | N3-(3-chloro-4-fluoro-phenyl)-2-methyl-N5-[[4- | ¹H NMR (400 MHz, DMSO-d6): δ |
| ISOMER-A | methyl-2-[4-[2-(1- | 10.39 (s, 1H), 8.60-8.50 (m, 1H), |
| | piperidyl)ethoxy]phenyl]thiazol-5-yl]methyl]- | 7.96-7.94 (m, 1H), 7.76 (d, J = |
| | 1,1-dioxo-1,2,6-thiadiazinane-3,5- | 8.8 Hz, 2H), 7.56-7.52 (m, 1H), |
| | dicarboxamide. | 7.37 (t, J = 9.2 Hz, 2H), 7.00 (d, |
| | | J = 8.4 Hz, 2H), 4.41 (d, |
| | | J = 5.2 Hz, 2H), 4.10 (t, J = 5.6 Hz, |
| | | 2H), 4.00-3.94 (m, 2H), 2.65 (t, J = |
| | | 6.0 Hz, 2H), 2.48-2.40 (m, 7H), 2.37 |
| | | (s, 3H), 2.00-1.94 (m, 1H), 1.90-1.72 |
| | | (m, 1H), 1.55-1.45 (m, 4H), 1.40-1.35 |
| | | (m, 2H). LCMS calcd for; |
| | | C₃₀H₃₆ClFN₆O₅S₂. Found; |
| | | 679.10 (M + 1). |
| 1087- | N3-(3-chloro-4-fluoro-phenyl)-2-methyl-N5-[[4- | ¹H NMR (400 MHz, DMSO-d6): δ |
| ISOMER-B | methyl-2-[4-[2-(1- | 10.50 (s, 1H), 8.80-8.70 (m, 1H), |
| | piperidyl)ethoxy]phenyl]thiazol-5-yl]methyl]- | 7.96-7.94 (m, 1H), 7.76 (d, J = |
| | 1,1-dioxo-1,2,6-thiadiazinane-3,5- | 8.0 Hz, 2H), 7.56-7.52 (m, 1H), |
| | dicarboxamide | 7.41-7.36 (m, 2H), 7.01 (d, J = |
| | | 8.8 Hz, 2H), 4.46-4.38 (m, |
| | | 2H), 4.22-4.08 (m, 4H), 2.65 (t, J = |
| | | 5.6 Hz, 2H), 2.55 (s, 3H), 2.45-2.40 |
| | | (m, 4H), 2.37 (s, 3H), 2.00-1.95 (m, |
| | | 2H), 1.55-1.45 (m, 4H), 1.42-1.36 (m, |
| | | 2H). LCMS calcd for; |
| | | C₃₀H₃₆ClFN₆O₅S₂. Found; |
| | | 679.10 (M + 1). |
| 1089 | 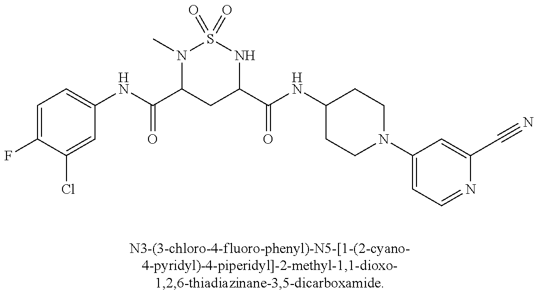 [SMILES: CN1C(C(=O)Nc2ccc(F)c(Cl)c2)CC(C(=O)NC2CCN(c3ccnc(C#N)c3)CC2)NS1(=O)=O] | ¹H NMR (400 MHz, DMSO-d6): δ 10.52 (s, 1H), 8.21 (d, J = 6.4 Hz, 1H), 8.07-8.04 (m, 1H), 7.96-7.93 (m, 1H), 7.59-7.46 (m, 2H), 7.39 (t, J = 9.2 Hz, 1H), 7.28-7.25 (m, 1H), 7.08-7.06 (m, 1H), 4.21-4.15 (m, 1H), 4.05-4.03 (m, 1H), 3.99-3.92 (m, 3H), 3.11-3.05 (m, 2H), 2.56 (s, 3H), 1.95-1.90 (m, 2H), 1.85-1.75 (m, 2H), 1.49-1.39 (m, 2H). LCMS calcd for; C₂₃H₂₅ClFN₇O₄S. Found; 550.15 (M + 1). |
| 1090 | 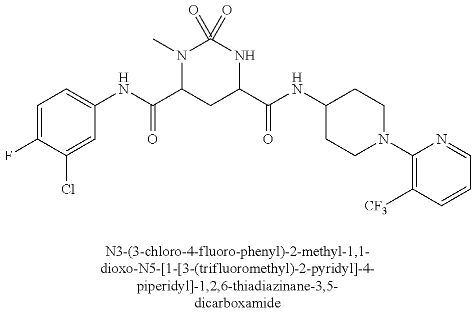 | ¹H NMR (400 MHz, DMSO-d6): δ 10.54 (s, 1H), 8.51-8.49 (m, 1H), 8.13 (d, J = 7.6 Hz, 1H), 8.04 (d, J = 8.0 Hz, 1H), 7.96-7.94 (m, 1H), 7.55-7.53 (m, 1H), 7.40 (t, J = 9.2 Hz, 1H), 7.27- 7.24 (m, 1H), 7.18-7.15 (m, 1H), 4.20-4.17 (m, 1H), 4.10-4.00 (m, 1H), 3.85-3.75 (m, 1H), 3.50-3.45 (m, 2H), 2.96 (t, J = 11.8 Hz, 2H), 2.57 (s, 3H), 1.93-1.80 (m, 4H), 1.62-1.52 (m, 2H). LCMS calcd for; C₂₃H₂₅ClF₄N₆O₄S. Found; 593.05 (M + 1). |
| 1093 | 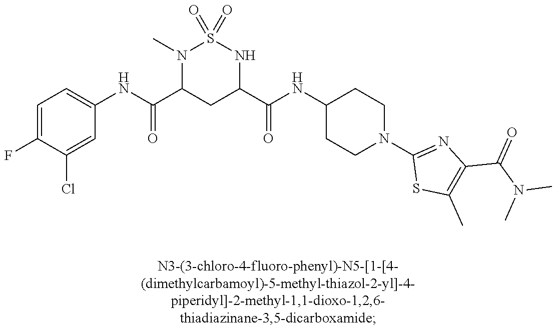 [SMILES: Cc1sc(N2CCC(NC(=O)C3CC(C(=O)Nc4ccc(F)c(Cl)c4)N(C)S(=O)(=O)N3)CC2)nc1C(=O)N(C)C] | ¹H NMR (400 MHz, DMSO-d6): δ 10.49 (s, 1H), 8.06 (d, J = 6.8 Hz, 1H), 7.92-7.89 (m, 1H), 7.51-7.48 (m, 1H), 7.36 (t, J = 8.8 Hz, 1H), 7.25-7.22 (m, 1H), 4.17-4.12 (m, 1H), 4.05-4.00 (m, 1H), 3.85-3.65 (m, 2H), 3.09-3.01 (m, 3H), 2.89 (d, J = 4.8 Hz, 6H), 2.53 (s, 3H), 2.20 (s, 3H), 1.92-1.84 (m, 2H), 1.80-1.70 (m, 2H), 1.52-1.42 (m, 2H). LCMS calcd for; C₂₄H₃₁ClFN₇O₅S₂. Found; 616.05 (M + 1). |
| 1098 | 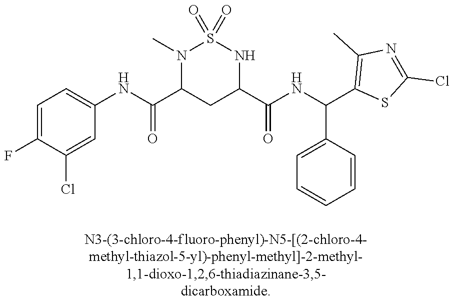 [SMILES: Cc1nc(Cl)sc1C(NC(=O)C1CC(C(=O)Nc2ccc(F)c(Cl)c2)N(C)S(=O)(=O)N1)c1ccccc1] | ¹H NMR (400 MHz, DMSO-d6): δ 10.55 (s, 1H), 9.29-9.22 (m, 1H), 7.97-7.95 (m, 1H), 7.58-7.48 (m, 1H), 7.43-7.26 (m, 7H), 6.32 (d, J = 8.0 Hz, 1H), 4.24-4.20 (m, 2H), 2.57 (s, 3H), 2.24 (s, 3H), 1.99-1.92 (m, 2H). Mixture of diastereomers. LCMS calcd for; C₂₃H₂₂Cl₂FN₅O₄S₂. Found; 586.05 (M + 1). |
| 1099 | 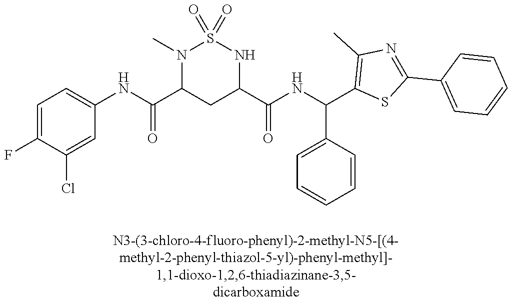 [SMILES: Cc1nc(-c2ccccc2)sc1C(NC(=O)C1CC(C(=O)Nc2ccc(F)c(Cl)c2)N(C)S(=O)(=O)N1)c1ccccc1] | ¹H NMR (400 MHz, DMSO-d6): δ 10.54-10.53 (m, 1H), 9.27 (t, J = 7.6 Hz, 1H), 7.97-7.94 (m, 1H), 7.84-7.80 (m, 2H), 7.56-7.53 (m, 1H), 7.46-7.30 (m, 10 H), 6.41-6.38 (m, 1H), 4.26- 4.21 (m, 2H), 2.58 (s, 3H), 2.34 (s, 3H), 2.00-1.92 (m, 2H). Mixture of diastereomers. LCMS calcd for; C₂₉H₂₇ClFN₅O₄S₂. Found; 628.10 (M + 1). |
| 1100 | 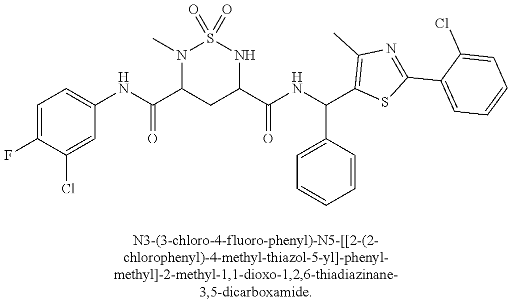 [SMILES: Cc1nc(-c2ccccc2Cl)sc1C(NC(=O)C1CC(C(=O)Nc2ccc(F)c(Cl)c2)N(C)S(=O)(=O)N1)c1ccccc1] | ¹H NMR (400 MHz, DMSO-d6): δ 10.54-10.53 (m, 1H), 9.31 (t, J = 7.2 Hz, 1H), 8.17-8.14 (m, 1H), 7.96-7.94 (m, 1H), 7.61-7.53 (m, 2H), 7.48-7.28 (m, 9 H), 6.46-6.41 (m, 1H), 4.26-4.21 (m, 2H), 2.57 (s, 3H), 2.37 (s, 3H), 2.00-1.92 (m, 2H). Mixture of diastereomers LCMS calcd for; C₂₉H₂₆Cl₂FN₅O₄S₂. Found: 662.10 (M + 1). |
| 1100- | N3-(3-chloro-4-fluoro-phenyl)-N5-[(R)-[2-(2- | ¹H NMR (400 MHz, DMSO-d6): δ |
| ISOMER-A | chlorophenyl)-4-methyl-thiazol-5-yl]-phenyl- | 10.54 (s, 1H), 9.33-9.30 (m, 1H), |
| | methyl]-2-methyl-1,1-dioxo-1,2,6-thiadiazinane- | 8.17-8.14 (m, 1H), 7.97-7.94 (m, |
| | 3,5-dicarboxamide. | 1H), 7.62-7.52 (m, 2H), |
| | | 7.48-7.44 (m, 2H), 7.44-7.30 (m, |
| | | 7H), 6.44 (d, J = 7.6 Hz, 1H), |
| | | 4.28-4.22 (m, 2H), 2.57 (s, 3H), 2.43 |
| | | (s, 3H), 2.02-1.95 (m, 2H). LCMS |
| | | calcd for; C₂₉H₂₆Cl₂FN₅O₄S₂. Found; |
| | | 661.95 (M + 1). |
| 1100- | N3-(3-chloro-4-fluoro-phenyl)-N5-[(S)-[2-(2- | ¹H NMR (400 MHz, DMSO-d6): δ |
| ISOMER-B | chlorophenyl)-4-methyl-thiazol-5-yl]-phenyl- | 10.54 (s, 1H), 9.31 (d, J = 7.6 Hz, |
| | methyl]-2-methyl-1,1-dioxo-1,2,6-thiadiazinane- | 1H), 8.17-8.14 (m, 1H), |
| | 3,5-dicarboxamide | 7.96-7.94 (m, 1H), 7.60-7.52 (m, |
| | | 2H), 7.50-7.30 (m, 9H), 6.42 (d, |
| | | J = 8.0 Hz, 1H), 4.26-4.22 (m, |
| | | 2H), 2.57 (s, 3H), 2.37 (s, 3H), 2.00- |
| | | 1.94 (m, 2H). LCMS calcd for, |
| | | C₂₉H₂₆Cl₂FN₅O₄S₂. Found; |
| | | 661.95 (M + 1). |
| 1100- | N3-(3-chloro-4-fluoro-phenyl)-N5-[(R)-[2-(2- | ¹H NMR (400 MHz, DMSO-d6): δ |
| ISOMER-C | chlorophenyl)-4-methyl-thiazol-5-yl]-phenyl- | 10.53 (s, 1H), 9.29-9.27 (m, 1H), |
| | methyl]-2-methyl-1,1-dioxo-1,2,6-thiadiazinane- | 8.20-8.10 (m, 1H), 7.97-7.93 (m, |
| | 3,5-dicarboxamide. | 1H), 7.57-7.52 (m, 2H), |
| | | 7.46-7.30 (m, 9H), 6.42 (d, J = |
| | | 7.2 Hz, 1H), 4.30-4.20 (m, 2H), |
| | | 2.57 (s, 3H), 2.37 (s, 3H), 2.02-1.96 |
| | | (m, 2H). LCMS calcd for; |
| | | C₂₉H₂₆Cl₂FN₅O₄S₂. Found; |
| | | 662.05 (M + 1). |
| 1100- | N3-(3-chloro-4-fluoro-phenyl)-N5-[(S)-[2-(2- | ¹H NMR (400 MHz, DMSO-d6): δ |
| ISOMER-D | chlorophenyl)-4-methyl-thiazol-5-yl]-phenyl- | 10.54 (s, 1H), 9.29-9.27 (m, 1H), |
| | methyl]-2-methyl-1,1-dioxo-1,2,6-thiadiazinane- | 8.20-8.10 (m, 1H), 7.97-7.93 (m, |
| | 3,5-dicarboxamide. | 1H), 7.57-7.52 (m, 2H), |
| | | 7.46-7.30 (m, 9H), 6.42 (d, J = |
| | | 7.2 Hz, 1H), 4.30-4.20 (m, 2H), |
| | | 2.57 (s, 3H), 2.37 (s, 3H), 2.02-1.96 |
| | | (m, 2H). LCMS calcd for; |
| | | C₂₉H₂₆Cl₂FN₅O₄S₂. Found; 662 (M + |
| | | 1). |
| 1101 | 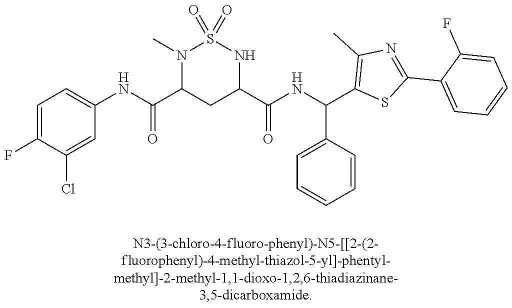 [SMILES: Cc1nc(-c2ccccc2F)sc1C(NC(=O)C1CC(C(=O)Nc2ccc(F)c(Cl)c2)N(C)S(=O)(=O)N1)c1ccccc1] | ¹H NMR (400 MHz, DMSO-d6): δ 10.55-10.53 (m, 1H), 9.32-9.28 (m, 1H), 8.18-8.14 (m, 1H), 7.96-7.94 (m, 1H), 7.55-7.32 (m, 11H), 6.45-6.42 (m, 1H), 4.26-4.22 (m, 2H), 2.57 (s, 3H), 2.38 (s, 3H), 2.07-1.90 (m, 2H). Mixture of diasteromers LCMS calcd for; C₂₉H₂₆ClF₂N₅O₄S₂. Found; 646.10 (M + 1). |
| 1101- | N3-(3-chloro-4-fluoro-phenyl)-N5-[(R)-[2-(2- | ¹H NMR (400 MHz, DMSO-d6): δ |
| ISOMER-A | fluorophenyl)-4-methyl-thiazol-5-yl]-phenyl- | 10.53 (s, 1H), 9.28 (d, J = 7.6 Hz, |
| | methyl]-2-methyl-1,1-dioxo-1,2,6-thiadiazinane- | 1H), 8.14 (t, J = 7.6 Hz, 1H), |
| | 3,5-dicarboxamide | 7.94-7.91 (m, 1H), 7.54-7.45 (m, |
| | | 2H), 7.42-7.28 (m, 9H), 6.41 (d, |
| | | J = 7.6 Hz, 1H), 4.30- |
| | | 4.20 (m, 2H), 2.56 (s, 3H), 2.37 (s, |
| | | 3H), 2.00-1.91 (m, 2H). LCMS calcd |
| | | for; C₂₉H₂₆ClF₂N₅O₄S₂. Found; |
| | | 646 (M + 1). |
| 1101- | N3-(3-chloro-4-fluoro-phenyl)-N5-[(S)-[2-(2- | ¹H NMR (400 MHz, DMSO-d6): δ |
| ISOMER-B | fluorophenyl)-4-methyl-thiazol-5-yl]-phenyl- | 10.55 (s, 1H), 9.31-9.28 (m, 1H), |
| | methyl]-2-methyl-1,1-dioxo-1,2,6-thiadiazinane- | 8.19-8.14 (m, 1H), 7.97-7.94 (m, |
| | 3,5-dicarboxamide. | 1H), 7.56-7.46 (m, 2H), |
| | | 7.41-7.30 (m, 9H), 6.44 (d, J = |
| | | 7.6 Hz, 1H), 4.27-4.21 (m, 2H), |
| | | 2.57 (s, 3H), 2.43 (s, 3H), 2.08-1.95 |
| | | (m, 2H). LCMS calcd for; |
| | | C₂₉H₂₆ClF₂N₅O₄S₂. Found; 646 (M + |
| | | 1). |
| 1101- | N3-(3-chloro-4-fluoro-phenyl)-N5-[(R)-[2-(2- | ¹H NMR (400 MHz, DMSO-d6): δ |
| ISOMER-C | fluorophenyl)-4-methyl-thiazol-5-yl]-phenyl- | 9.18-9.04 (m, 1H), 8.60-8.22 (m, 1H), |
| | methyl]-2-methyl-1,1-dioxo-1,2,6-thiadiazinane- | 8.20-8.12 (m, 1H), 7.98-7.94 (m, 1H), |
| | 3,5-dicarboxamide. | 7.58-7.28 (m, 10H), 6.45-6.40 (m, |
| | | 1H), 4.20-4.05 (m, 2H), 2.38 (s, 3H), |
| | | 2.09 (s, 3H), 2.00-1.85 (m, 2H). |
| | | LCMS calcd |
| | | for; C₂₉H₂₆ClF₂N₅O₄S₂. Found; |
| | | 645.95 (M + 1). |
| 1101- | N3-(3-chloro-4-fluoro-phenyl)-N5-[(S)-[2-(2- | ¹H NMR (400 MHz, DMSO-d6): δ |
| ISOMER-D | fluorophenyl)-4-methyl-thiazol-5-yl]-phenyl- | 9.25-9.15 (m, 1H), 8.50-8.40 (m, 1H), |
| | methyl]-2-methyl-1,1-dioxo-1,2,6-thiadiazinane- | 8.19-8.14 (m, 1H), 7.97-7.94 (m, 1H), |
| | 3,5-dicarboxamde. | 7.57-7.31 (m, 10H), 6.45-6.40 (m, |
| | | 1H), 4.20-4.05 (m, 2H), 2.41 (s, 3H), |
| | | 2.09 (s, 3H), 2.00-1.87 (m, 2H). |
| | | LCMS calcd |
| | | for; C₂₉H₂₆ClF₂N₅O₄S₂. Found; |
| | | 646 (M + 1). |
| 1104 | 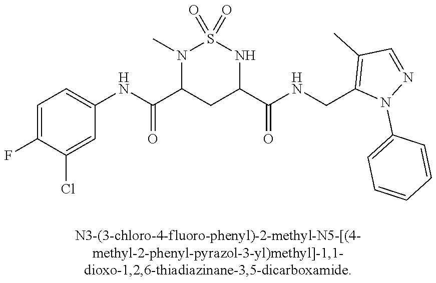 [SMILES: Cc1cnn(-c2ccccc2)c1CNC(=O)C1CC(C(=O)Nc2ccc(F)c(Cl)c2)N(C)S(=O)(=O)N1] | ¹H NMR (400 MHz, DMSO-d6): δ 10.49 (s, 1H), 8.48 (t, J = 4.4 Hz, 1H), 7.95-7.92 (m, 1H), 7.56-7.50 (m, 1H), 7.48-7.44 (m, 5H), 7.41-7.35 (m, 2H), 7.25-7.21 (m, 1H), 4.40-4.24 (m, 2H), 4.16-4.00 (m, 2H), 2.53 (s, 3H), 2.05 (s, 3H), 1.90-1.70 (m, 2H). LCMS calcd for; C₂₃H₂₄ClFN₆O₄S. Found; 535 (M + 1). |
| 1116 | 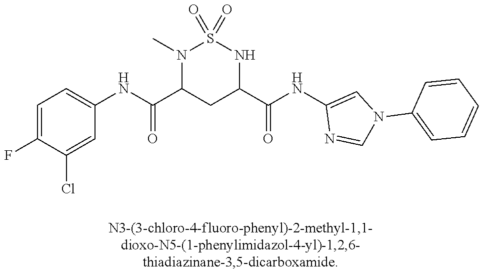 [SMILES: CN1C(C(=O)Nc2ccc(F)c(Cl)c2)CC(C(=O)Nc2cn(-c3ccccc3)cn2)NS1(=O)=O] | ¹H NMR (400 MHz, DMSO-d6): δ 10.72 (bs, 1H), 10.56 (bs, 1H), 8.14 (s, 1H), 7.97-7.95 (m, 1H), 7.72 (s, 1H), 7.64-7.62 (m, 2H), 7.65-7.49 (m, 3H), 7.42-7.36 (m, 3H), 4.39-4.36 (m, 1H), 4.27-4.24 (m, 1H), 2.59 (s, 3H), 2.06- 1.99 (m, 2H). LCMS calcd for; C₂₁H₂₀ClFN₆O₄S. Found; 507 (M + 1). |
| 1132 | 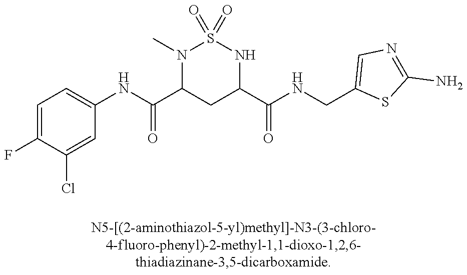 [SMILES: CN1C(C(=O)Nc2ccc(F)c(Cl)c2)CC(C(=O)NCc2cnc(N)s2)NS1(=O)=O] | ¹H NMR (400 MHz, DMSO-d6): δ 10.51 (s, 1H), 8.55 (t, J = 6.0 Hz, 1H), 7.96-7.92 (m, 1H), 7.56-7.52 (m, 1H), 7.39 (t, J = 8.8 Hz, 1H), 7.32-7.29 (m, 1H), 6.79-6.76 (m, 3H), 4.30-4.15 (m, 3H), 4.08-4.00 (m, 1H), 2.56 (s, 3H), 1.96-1.88 (m, 2H). LCMS calcd for; C₁₆H₁₈ClFN₆O₄S₂. Found; 477 (M + 1). |
| 1132- | N5-[(2-aminothiazol-5-yl)methyl]-N3-(3-chloro- | ¹H NMR (400 MHz, DMSO-d6): δ |
| ISOMER-A | 4-fluoro-phenyl)-2-methyl-1,1-dioxo-1,2,6- | 10.52 (s, 1H), 8.58 (t, J = 5.6 Hz, |
| | thiadiazinane-3,5-dicarboxamide. | 1H), 7.96-7.94 (m, 1H), |
| | | 7.56-7.52 (m, 1H), 7.42-7.32 (m, |
| | | 2H), 7.10-7.05 (m, 2H), 6.82 (s, |
| | | 1H), 4.30-4.18 (m, 3H), 4.10- |
| | | 4.05 (m, 1H), 2.56 (s, 3H), 1.94-1.90 |
| | | (m, 2H). LCMS calcd for; |
| | | C₁₆H₁₈ClFN₆O₄S₂. Found; |
| | | 476.95 (M + 1). |
| 1132- | N5-[(2-aminothiazol-5-yl)methyl]-N3-(3-chloro- | ¹H NMR (400 MHz, DMSO-d6): δ |
| ISOMER-B | 4-fluoro-phenyl)-2-methyl-1,1-dioxo-1,2,6- | 10.52 (s, 1H), 8.60-8.55 (m, 1H), |
| | thiadiazinane-3,5-dicarboxamide. | 7.96-7.94 (m, 1H), |
| | | 7.56-7.52 (m, 1H), 7.42-7.30 (m, |
| | | 2H), 7.05-6.90 (m, 2H), 6.80 |
| | | (s, 1H), 4.30-4.16 (m, 3H), 4.10-4.02 |
| | | (m, 1H), 2.56 (s, 3H), 1.94-1.88 (m, |
| | | 2H). LCMS calcd for; |
| | | C₁₆H₁₈ClFN₆O₄S₂. Found; 477 (M + |
| | | 1). |
| 1134 | 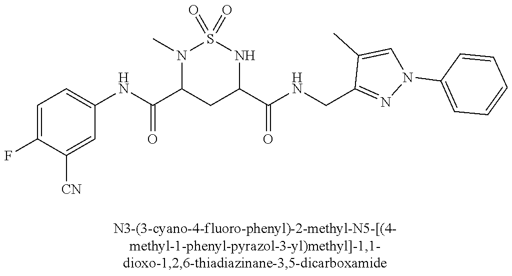 [SMILES: Cc1cn(-c2ccccc2)nc1CNC(=O)C1CC(C(=O)Nc2ccc(F)c(C#N)c2)N(C)S(=O)(=O)N1] | ¹H NMR (400 MHz, DMSO-d6): δ 10.70 (s, 1H), 8.54-8.48 (m, 1H), 8.23 (s, 1H), 8.16-8.14 (m, 1H), 7.94-7.86 (m, 1H), 7.74 (d, J = 8.4 Hz, 2H), 7.52 (t, J = 8.8 Hz, 1H), 7.47-7.41 (m, 2H), 7.30-7.20 (m, 2H), 4.40-4.13 (m, 4H), 2.58 (s, 3H), 2.05 (s, 3H), 1.99-1.92 (m, 2H). LCMS calcd for; C₂₄H₂₄FN₇O₄S. Found; 526 (M + 1). |
| 1134- | N3-(3-cyano-4-fluoro-phenyl)-2-methyl-N5-[(4- | ¹H NMR (400 MHz, DMSO-d6): δ |
| ISOMER-A | methyl-1-phenyl-pyrazol-3-yl)methyl]-1,1- | 10.68 (bs, 1H), 8.54-8.50 (m, 1H), |
| | dioxo-1,2,6-thiadiazinane-3,5-dicarboxamide. | 8.24 (s, 1H), 8.16-8.14 (m, 1H), 7.94- |
| | | 7.88 (m, 1H), 7.74 (d, J = 8.4 Hz, |
| | | 2H), 7.52 (t, J = 8.8 Hz, 1H), |
| | | 7.47-7.42 (m, 2H), 7.30-7.20 (m, |
| | | 2H), 4.40-4.14 (m, 4H), 2.57 (s, |
| | | 3H), 2.05 (s, 3H), 1.98-1.93 (m, |
| | | 2H). LCMS calcd for; |
| | | C₂₄H₂₄FN₇O₄S. Found; 526 (M + 1). |
| 1134- | N3-(3-cyano-4-fluoro-phenyl)-2-methyl-N5-[(4- | ¹H NMR (400 MHz, DMSO-d6): δ |
| ISOMER-B | methyl-1-phenyl-pyrazol-3-yl)methyl]-1,1- | 10.68 (bs, 1H), 8.54-8.50 (m, 1H), |
| | dioxo-1,2,6-thiadiazinane-3,5-dicarboxamide. | 8.24 (s, 1H), 8.17-8.14 (m, 1H), 7.93- |
| | | 7.89 (m, 1H), 7.72 (d, J = 8.0 Hz, 2H), |
| | | 7.52 (t, J = 8.8 Hz, 1H), 7.47-7.42 (m, |
| | | 2H), 7.29-7.22 (m, 2H), 4.40-4.14 (m, |
| | | 4H), 2.57 (s, 3H), 2.05 (s, 3H), 1.98- |
| | | 1.93 (m, 2H). LCMS calcd for; |
| | | C₂₄H₂₄FN₇O₄S. Found; 526 (M + 1). |
| 1135 | 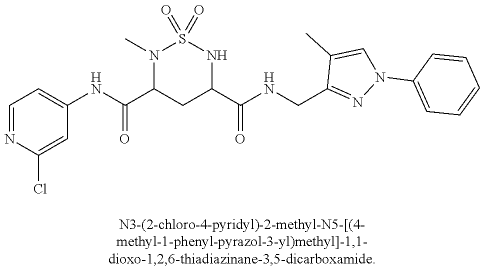 [SMILES: Cc1cn(-c2ccccc2)nc1CNC(=O)C1CC(C(=O)Nc2ccnc(Cl)c2)N(C)S(=O)(=O)N1] | ¹H NMR (400 MHz, DMSO-d6): δ 10.90 (br.s, 1H), 8.58-8.48 (m, 1H), 8.32-8.23 (m, 2H), 7.84-7.70 (m, 3H), 7.60-7.18 (m, 5H), 4.40-4.20 (m, 3H), 4.18-4.10 (m, 1H), 2.56 (s, 3H), 2.05 (s, 3H), 2.00-1.92 (m, 2H). LCMS calcd for; C2₂H₂₄ClN₇O₄S. Found; 518 (M + 1). |
| 1136 | 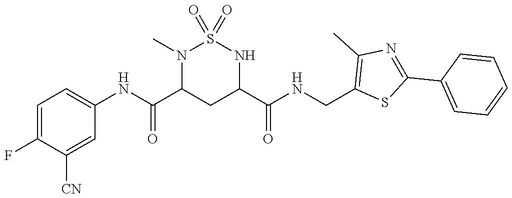 [SMILES: Cc1nc(-c2ccccc2)sc1CNC(=O)C1CC(C(=O)Nc2ccc(F)c(C#N)c2)N(C)S(=O)(=O)N1] | ¹H NMR (400 MHz, DMSO-d6): δ 10.65 (s, 1H), 8.80 (t, J = 5.6 Hz, 1H), 8.16-8.12 (m, 1H), 7.9-27.84 (m, 3H), 7.56-7.38 (m, 5H), 4.46-4.38 (m, 2H), 4.26-4.22 (m, 1H), 412-4.04 (m, 1H), 2.57 (s, 3H), 2.41 (s, 3H), 2.00-1.90 (m, 2H). LCMS calcd for; C₂₄H₂₃FN₆O₄S₂. Found; 543 (M + 1). |
| 1136- | N3-(3-cyano-4-fluoro-phenyl)-2-methyl-N5-[(4- | .¹H NMR (400 MHz, DMSO-d6): δ |
| ISOMER-A | methyl-2-phenyl-thiazol-5-yl)methyl]-1,1- | 10.67 (s, 1H), 8.85-8.80 (m, 1H), |
| | dioxo-1,2,6-thiadiazinane-3,5-dicarboxamide | 8.16-8.12 (m, 1H), |
| | | 7.92-7.84 (m, 3H), 7.55-7.45 (m, |
| | | 5H), 4.46-4.40 (m, 2H), 4.25- |
| | | 4.20 (m, 1H), 4.12-4.05 (m, 1H), 2.57 |
| | | (s, 3H), 2.40 (s, 3H), 1.98-1.92 (m, |
| | | 2H). LCMS calcd for; |
| | | C₂₄H₂₃FN₆O₄S₂. |
| | | Found; 543 (M + 1). |
| 1136- | N3-(3-cyano-4-fluoro-phenyl)-2-methyl-N5-[(4- | ¹H NMR (400 MHz, DMSO-d6): δ |
| ISOMER-B | methyl-2-phenyl-thiazol-5-yl)methyl]-1,1- | 10.67 (s, 1H), 8.85-8.78 (m, 1H), |
| | dioxo-1,2,6-thiadiazinane-3,5-dicarboxamide. | 8.16-8.14 (m, 1H), |
| | | 7.92-7.84 (m, 3H), 7.56- |
| | | 7.44 (m, 5H), 4.50-4.38 (m, 2H), 4.26- |
| | | 4.22 (m, 1H), 4.10-4.06 (m, 1H), 2.57 |
| | | (s, 3H), 2.40 (s, 3H), 2.00-1.89 (m, |
| | | 2H). LCMS calcd for; |
| | | C₂₄H₂₃FN₆O₄S₂. |
| | | Found; 542.95 (M + 1). |
| 1137 | 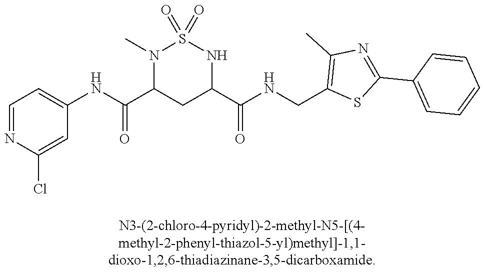 [SMILES: Cc1nc(-c2ccccc2)sc1CNC(=O)C1CC(C(=O)Nc2ccnc(Cl)c2)N(C)S(=O)(=O)N1] | ¹H NMR (400 MHz, DMSO-d6): δ 10.88 (s, 1H), 8.80-8.75 (m, 1H), 8.29 (d, J = 5.2 Hz, 1H), 7.88-7.80 (m, 3H), 7.58-7.44 (m, 5H), 4.46-4.04 (m, 4H), 2.55 (s, 3H), 2.41 (s, 3H), 2.00-1.86 (m, 2H). LCMS calcd for; C₂₂H₂₃ClN₆O₄S₂. Found; 534.90 (M + 1). |
| 1141 | 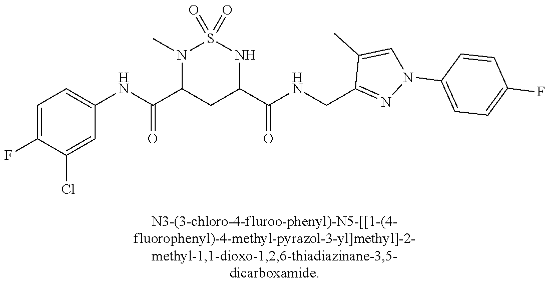 [SMILES: Cc1cn(-c2ccc(F)cc2)nc1CNC(=O)C1CC(C(=O)Nc2ccc(F)c(Cl)c2)N(C)S(=O)(=O)N1] | ¹H NMR (400 MHz, DMSO-d6): δ 10.53 (s, 1H), 8.55 (t, J = 5.6 Hz, 1H), 8.20 (s, 1H), 7.96-7.94 (m, 1H), 7.78-7.74 (m, 2H), 7.56-7.51 (m, 1H), 7.38 (t, J = 8.8 Hz, 1H), 7.31-7.24 (m, 3H), 4.39-4.28 (m, 2H), 4.22-4.12 (m, 2H), 2.56 (s, 3H), 2.04 (s, 3H), 1.98-1.92 (m, 2H). LCMS calcd for; C₂₃H₂₃ClF₂N₆O₄S. Found; 553.15 (M + 1). |
| 1141- | N3-(3-chloro-4-fluoro-phenyl)-N5-[[1-(4- | ¹H NMR (400 MHz, DMSO-d6): δ |
| ISOMER-A | fluorophenyl)-4-methyl-pyrazol-3-yl]methyl]-2- | 10.53 (s, 1H), 8.56-8.50 (m, 1H), 8.21 |
| | methyl-1,1-dioxo-1,2,6-thiadiazinane-3,5- | (s, 1H), 7.98-7.92 (m, 1H), 7.80-7.72 |
| | dicarboxamide. | (m, 2H), 7.56-7.50 (m, 1H), 7.39 (t, |
| | | J = 8.8 Hz, 1H), 7.31-7.24 (m, 3H), |
| | | 4.40-4.28 (m, 2H), 4.22-4.12 (m, 2H), |
| | | 2.56 (s, 3H), 2.04 (s, 3H), 1.98-1.92 |
| | | (m, 2H). LCMS calcd for; |
| | | C₂₃H₂₃ClF₂N₆O₄S. Found; 553.05 |
| | | (M + 1). |
| 1141- | N3-(3-chloro-4-fluoro-phenyl)-N5-[[1-(4- | ¹H NMR (400 MHz, DMSO-d6): δ |
| ISOMER-B | fluorophenyl)-4-methyl-pyrazol-3-yl]methyl]-2- | 10.53 (s, 1H), 8.55-8.48 (m, 1H), 8.21 |
| | methyl-1,1-dioxo-1,2,6-thiadiazinane-3,5- | (s, 1H), 7.98-7.92 (m, 1H), 7.80-7.74 |
| | dicarboxamide | (m, 2H), 7.56-7.50 (m, 1H), 7.39 (t, |
| | | J = 8.8 Hz, 1H), 7.31-7.24 (m, 3H), |
| | | 4.40-4.26 (m, 2H), 4.22-4.10 (m, 2H), |
| | | 2.56 (s, 3H), 2.04 (s, 3H), 1.98-1.92 |
| | | (m, 2H). LCMS calcd for; |
| | | C₂₃H₂₃ClF₂N₆O₄S. Found; 553.05 |
| | | (M + 1). |
| 1142 | 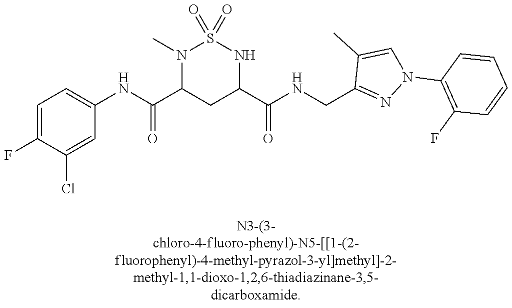 [SMILES: Cc1cn(-c2ccccc2F)nc1CNC(=O)C1CC(C(=O)Nc2ccc(F)c(Cl)c2)N(C)S(=O)(=O)N1] | ¹H NMR (400 MHz, DMSO-d6): δ 10.52 (s, 1H), 8.54-8.50 (m, 1H), 7.98-7.92 (m, 2H), 7.78-7.72 (m, 1H), 7.56-7.52 (m, 1H), 7.46-7.25 (m, 5H), 4.40- 4.30 (m, 2H), 4.24-4.14 (m, 2H), 2.57 (s, 3H), 2.06 (s, 3H), 1.99-1.92 (m, 2H). LCMS calcd for; C₂₃H₂₃ClF₂N₆O₄S. Found; 553.10 (M + 1). |
| 1142- | N3-(3-chloro-4-fluoro-phenyl)-N5-[[1-(2- | ¹H NMR (400 MHz, DMSO-d6): δ |
| ISOMER-A | fluorophenyl)-4-methyl-pyrazol-3-yl]methyl]-2- | 10.53 (s, 1H), 8.52-8.50 (m, 1H), |
| | methyl-1,1-dioxo-1,2,6-thiadiazinane-3,5- | 7.98-7.92 (m, 2H), |
| | dicarboxamide | 7.80-7.74 (m, 1H), 7.56-7.50 (m, |
| | | 1H), 7.45-7.25 (m, 5H), 4.40- |
| | | 4.30 (m, 2H), 4.24-4.14 (m, 2H), 2.57 |
| | | (s, 3H), 2.06 (s, 3H), 1.99-1.92 (m, |
| | | 2H). LCMS calcd for; |
| | | C₂₃H₂₃ClF₂N₆O₄S. Found; 553.05 |
| | | (M + 1). |
| 1142- | N3-(3-chloro-4-fluoro-phenyl)-N5-[[1-(2- | ¹H NMR (400 MHz, DMSO-d6): δ |
| ISOMER-B | fluorophenyl)-4-methyl-pyrazol-3-yl]methyl]-2- | 10.53 (s, 1H), 8.52 (t, J = 5.2 Hz, |
| | methyl-1,1-dioxo-1,2,6-thiadiazinane-3,5- | 1H), 7.96-7.92 (m, 2H), |
| | dicarboxamide. | 7.78-7.72 (m, 1H), 7.56-7.52 (m, |
| | | 1H), 7.46-7.26 (m, 5H), |
| | | 4.40-4.30 (m, 2H), 4.24-4.12 (m, 2H), |
| | | 2.56 (s, 3H), 2.05 (s, 3H), 1.99-1.90 |
| | | (m, 2H). LCMS calcd for; |
| | | C₂₃H₂₃ClF₂N₆O₄S. Found; 553.05 |
| | | (M + 1). |
| 1143 | 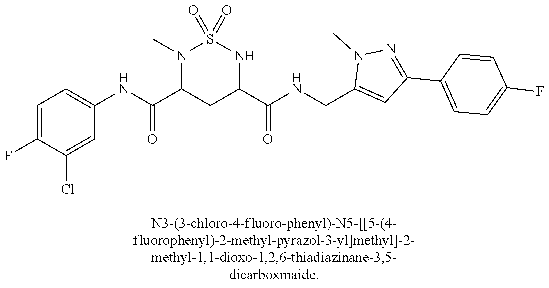 [SMILES: CN1C(C(=O)Nc2ccc(F)c(Cl)c2)CC(C(=O)NCc2cc(-c3ccc(F)cc3)nn2C)NS1(=O)=O] | ¹H NMR (400 MHz, DMSO-d6): δ 10.53 (s, 1H), 8.68-8.64 (m, 1H), 7.96-7.94 (m, 1H), 7.78-7.74 (m, 2H), 7.55-7.52 (m, 1H), 7.46-7.36 (m, 2H), 7.24-7.18 (m, 2H), 6.59 (s, 1H), 4.42- 4.30 (m, 2H), 4.22-4.14 (m, 2H), 3.81 (s, 3H), 2.58 (s, 3H), 2.00-1.94 (m, 2H). LCMS calcd for; C₂₃H₂₃ClF₂N₆O₄S. Found; 553.05 (M + 1). |
| 1143- | N3-(3-chloro-4-fluoro-phenyl)-N5-[[5-(4- | ¹H NMR (400 MHz, DMSO-d6): δ |
| ISOMER-A | fluorophenyl)-2-methyl-pyrazol-3-yl]methyl]-2- | 10.53 (s, 1H), 8.68 (t, J = 4.8 Hz, |
| | methyl-1,1-dioxo-1,2,6-thiadiazinane-3,5- | 1H), 7.96-7.92 (m, 1H), |
| | dicarboxamide. | 7.80-7.72 (m, 2H), 7.58-7.52 (m, |
| | | 1H), 7.46-7.36 (m, 2H), |
| | | 7.24-7.18 (m, 2H), 6.59 (s, 1H), 4.44- |
| | | 4.30 (m, 2H), 4.22-4.10 (m, 2H), 3.81 |
| | | (s, 3H), 2.58 (s, 3H), 2.02-1.92 (m, |
| | | 2H). LCMS calcd for; |
| | | C₂₃H₂₃ClF₂N₆O₄S. Found; |
| | | 553.05 (M + 1). |
| 1143- | N3-(3-chloro-4-fluoro-phenyl)-N5-[[5-(4- | ¹H NMR (400 MHz, DMSO-d6): δ |
| ISOMER-B | fluorophenyl)-2-methyl-pyrazol-3-yl]methyl]-2- | 10.53 (s, 1H), 8.70-8.64 (m, 1H), |
| | methyl-1,1-dioxo-1,2,6-thiadiazinane-3,5- | 7.98-7.92 (m, 1H), 7.80-7.72 (m, |
| | dicarboxamide. | 2H), 7.58-7.52 (m, 1H), |
| | | 7.46-7.36 (m, 2H), 7.24- |
| | | 7.16 (m, 2H), 6.59 (s, 1H), 4.44-4.34 |
| | | (m, 2H), 4.22-4.10 (m, 2H), 3.81 (s, |
| | | 3H), 2.58 (s, 3H), 2.02-1.96 (m, 2H). |
| | | LCMS calcd for; C₂₃H₂₃ClF₂N₆O₄S. |
| | | Found; 553.10 (M + 1). |
| 1144 | 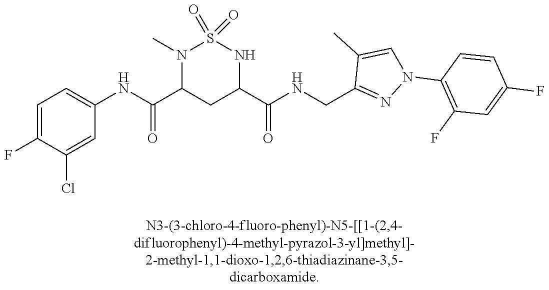 [SMILES: Cc1cn(-c2ccc(F)cc2F)nc1CNC(=O)C1CC(C(=O)Nc2ccc(F)c(Cl)c2)N(C)S(=O)(=O)N1] | ¹H NMR (400 MHz, DMSO-d6): δ 10.50 (s, 1H), 8.50 (t, J = 5.2 Hz, 1H), 7.97-7.90 (m, 2H), 7.80-7.72 (m, 1H), 7.57-7.48 (m, 2H), 7.39 (t, J = 8.8 Hz, 1H), 7.26-7.19 (m, 2H), 4.40-4.28 (m, 2H), 4.23-4.14 (m, 2H), 2.57 (s, 3H), 2.05 (s, 3H), 2.02-1.92 (m, 2H). LCMS calcd for; C₂₃H₂₂ClF₃N₆O₄S. Found; 571.05 (M + 1). |
| 1144- | N3-(3-chloro-4-fluoro-phenyl)-N5-[[1-(2,4- | .¹H NMR (400 MHz, DMSO-d6): δ |
| ISOMER-A | difluorophenyl)-4-methyl-pyrazol-3-yl]methyl]- | 10.50 (s, 1H), 8.52-8.48 (m, 1H), |
| | 2-methyl-1,1-dioxo-1,2,6-thiadiazinane-3,5- | 7.96-7.90 (m, 2H), |
| | dicarboxamide | 7.78-7.73 (m, 1H), 7.58- |
| | | 7.46 (m, 2H), 7.38 (t, J = 8.8 Hz, 1H), |
| | | 7.27-7.19 (m, 2H), 4.35-4.25 (m, 2H), |
| | | 4.21-4.12 (m, 2H), 2.55 (s, 3H), 2.04 |
| | | (s, 3H), 1.95-1.91 (m, 2H). LCMS |
| | | calcd for; C₂₃H₂₂ClF₃N₆O₄S. Found; |
| | | 571 (M + 1). |
| 1144- | N3-(3-chloro-4-fluoro-phenyl)-N5-[[1-(2,4- | ¹H NMR (400 MHz, DMSO-d6): δ |
| ISOMER-B | difluorophenyl)-4-methyl-pyrazol-3-yl]methyl]- | 10.50 (s, 1H), 8.49 (t, J = 5.6 Hz, |
| | 2-methyl-1,1-dioxo-1,2,6-thiadiazinane-3,5- | 1H), 7.95-7.90 (m, 2H), |
| | dicarboxamide | 7.80-7.71 (m, 1H), 7.54-7.47 (m, |
| | | 2H), 7.38 (t, J = 8.8 Hz, |
| | | 1H), 7.27-7.17 (m, 2H), 4.37-4.28 (m, |
| | | 2H), 4.21-4.12 (m, 2H), 2.55 (s, 3H), |
| | | 2.04 (s, 3H), 2.00-1.92 (m, 2H). |
| | | LCMS calcd for; C₂₃H₂₂ClF₃N₆O₄S. |
| | | Found; 571 (M + 1). |
| 1145 | 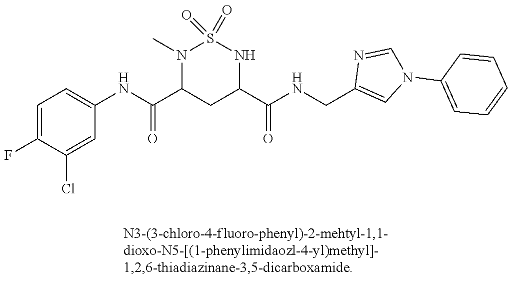 [SMILES: CN1C(C(=O)Nc2ccc(F)c(Cl)c2)CC(C(=O)NCc2cn(-c3ccccc3)cn2)NS1(=O)=O] | ¹H NMR (400 MHz, DMSO-d6): δ 10.56 (s, 1H), 8.56-8.53 (m, 1H), 8.45-8.43 (m, 1H), 7.97-7.94 (m, 1H), 7.69- 7.62 (m, 3H), 7.57-7.49 (m, 3H), 7.42- 7.37 (m, 3H), 4.30-4.15 (m, 4H), 2.58 (s, 3H), 2.04-1.94 (m, 2H). LCMS calcd for; C₂₂H₂₂ClFN₆O₄S. Found; 521 (M + 1). |
| 1145- | N3-(3-chloro-4-fluoro-phenyl)-2-methyl-1,1- | ¹H NMR (400 MHz, DMSO-d6): δ |
| ISOMER-A | dioxo-N5-[(1-phenylimidazol-4-yl)methyl]- | 10.54 (s, 1H), 8.46 (t, J = 5.6 Hz, |
| | 1,2,6-thiadiazinane-3,5-dicarboxamide. | 1H), 8.23 (s, 1H), |
| | | 7.96-7.93 (m, 1H), 7.65- |
| | | 7.60 (m, 3H), 7.57-7.48 (m, 3H), 7.42- |
| | | 7.31 (m, 3H), 4.31-4.12 (m, 4H), 2.58 |
| | | (s, 3H), 2.00-1.94 (m, 2H). LCMS |
| | | calcd for; C₂₂H₂₂ClFN₆O₄S. Found; |
| | | 521.05 (M + 1). |
| 1145- | N3-(3-chloro-4-fluoro-phenyl)-2-methyl-1,1- | ¹H NMR (400 MHz, DMSO-d6): δ |
| ISOMER-B | dioxo-N5-[(1-phenylimidazol-4-yl)methyl]- | 10.54 (s, 1H), 8.54-8.46 (m, 2H), |
| | 1,2,6-thiadiazinane-3,5-dicarboxamide. | 7.96-7.94 (m, 1H), |
| | | 7.68 (s, 1H), 7.65-7.62 |
| | | (m, 2H), 7.56-7.52 (m, 3H), 7.42-7.34 |
| | | (m, 3H), 4.34-4.13 (m, 4H), 2.58 (s, |
| | | 3H), 2.04-1.95 (m, 2H). LCMS calcd |
| | | for; C₂₂H₂₂ClFN₆O₄S. Found; |
| | | 521.05 (M + 1). |
| 1147 | 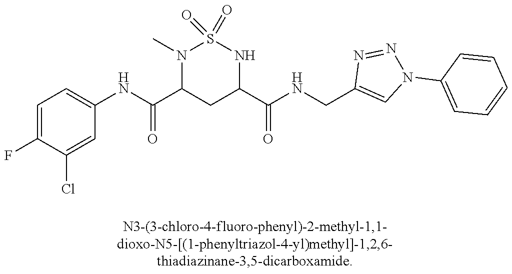 [SMILES: CN1C(C(=O)Nc2ccc(F)c(Cl)c2)CC(C(=O)NCc2cn(-c3ccccc3)nn2)NS1(=O)=O] | ¹H NMR (400 MHz, DMSO-d6): δ 10.54 (s, 1H), 8.71 (t, J = 5.2 Hz, 1H), 8.61 (s, 1H), 7.96-7.93 (m, 1H), 7.86 (d, J = 8.0 Hz, 2H), 7.62-7.58 (m, 2H), 7.56-7.47 (m, 2H), 7.42-7.36 (m, 2H), 4.50-4.38 (m, 2H), 4.20-4.13 (m, 2H), 2.58 (s, 3H), 2.07-1.92 (m, 2H). LCMS calcd for; C₂₁H₂₁ClFN₇O₄S. Found; 522.05 (M + 1). |
| 1148 | 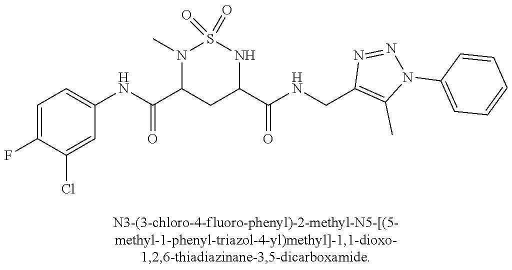 [SMILES: Cc1c(CNC(=O)C2CC(C(=O)Nc3ccc(F)c(Cl)c3)N(C)S(=O)(=O)N2)nnn1-c1ccccc1] | ¹H NMR (400 MHz, DMSO-d6): δ 10.54 bs, 1H), 8.69-8.65 (m, 1H), 7.96-7.92 (m, 1H), 7.65-7.50 (m, 6H), 7.42-7.30 (m, 2H), 4.39 (d, J = 4.8 Hz, 2H), 4.22-4.09 (m, 2H), 2.56 (s, 3H), 2.31 (s, 3H), 1.98-1.92 (m, 2H). LCMS calcd for; C₂₂H₂₃ClFN₇O₄S. Found; 536.05 (M + 1). |
| 1148- | N3-(3-chloro-4-fluoro-phenyl)-2-methyl-N5-[(5- | ¹H NMR (400 MHz, DMSO-d6): δ |
| ISOMER-A | methyl-1-phenyl-triazol-4-yl)methyl]-1,1-dioxo- | 10.53 (s, 1H), 8.66 (t, J = 5.6 Hz, |
| | 1,2,6-thiadiazinane-3,5-dicarboxamide. | 1H), 7.96-7.94 (m, 1H), |
| | | 7.65-7.54 (m, 6H), 7.42-7.29 (m, |
| | | 2H), 4.39 (d, J = 5.2 Hz, 2H), |
| | | 4.22-4.08 (m, 2H), 2.56 (s, 3H), |
| | | 2.31 (s, 3H), 1.97-1.90 (m, 2H). |
| | | LCMS calcd for; C₂₂H₂₃ClFN₇O₄S. |
| | | Found; 536.10 (M + 1). |
| 1148- | N3-(3-chloro-4-fluoro-phenyl)-2-methyl-N5-[(5- | ¹H NMR (400 MHz, DMSO-d6): δ |
| ISOMER-B | methyl-1-phenyl-triazol-4-yl)methyl]-1,1-dioxo- | 10.53 (s, 1H), 8.66 (t, J = 5.6 Hz, |
| | 1,2,6-thiadiazinane-3,5-dicarboxamide. | 1H), 7.96-7.93 (m, 1H), |
| | | 7.64-7.51 (m, 6H), 7.42-7.30 (m, |
| | | 2H), 4.39 (d, J = 5.2 Hz, |
| | | 2H), 4.22-4.10 (m, 2H), 2.56 (s, 3H), |
| | | 2.31 (s, 3H), 1.97-1.92 (m, 2H). |
| | | LCMS calcd for; |
| | | C₂₂H₂₃ClFN₇O₄S. Found; |
| | | 536 (M + 1). |
| 1149 | 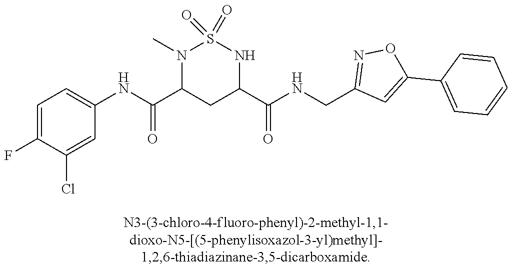 [SMILES: CN1C(C(=O)Nc2ccc(F)c(Cl)c2)CC(C(=O)NCc2cc(-c3ccccc3)on2)NS1(=O)=O] | ¹H NMR (400 MHz, DMSO-d6): δ 10.56 (s, 1H), 8.85-8.75 (m, 1H), 7.97-7.94 (m, 1H), 7.85-7.83 (m, 2H), 7.55-7.35 (m, 6H), 6.92 (s, 1H), 4.41-4.37 (m, 2H), 4.22-4.16 (m, 2H), 2.58 (s, 3H), 2.03-1.95 (m, 2H). LCMS calcd for; C₂₂H₂₁ClFN₅O₅S. Found; 522.10 (M + 1). |
| 1149- | N3-(3-chloro-4-fluoro-phenyl)-2-methyl-1,1- | ¹H NMR (400 MHz, DMSO-d6): δ |
| ISOMER-A | dioxo-N5-[(5-phenylisoxazol-3-yl)methyl]- | 10.60 (s, 1H), 8.79 (t, J = 6.0 Hz, 1H), |
| | 1,2,6-thiadiazinane-3,5-dicarboxamide. | 7.97-7.94 (m, 1H), 7.85-7.81 (m, 2H), |
| | | 7.56-7.48 (m, 4H), 7.42-7.37 (m, 2H), |
| | | 6.91 (s, 1H), 4.39 (d, J = 6.0 Hz, 2H), |
| | | 4.22-4.18 (m, 2H), 2.59 (s, 3H), 2.06- |
| | | 1.93 (m, 2H). LCMS calcd for; |
| | | C₂₂H₂₁ClFN₅O₅S. Found; |
| | | 522.05 (M + 1). |
| 1149- | N3-(3-chloro-4-fluoro-phenyl)-2-methyl-1,1- | ¹H NMR (400 MHz, DMSO-d6): δ |
| ISOMER-B | dioxo-N5-[(5-phenylisoxazol-3-yl)methyl]- | 10.50 (s, 1H), 8.79 (t, J = 6.0 Hz, |
| | 1,2,6-thiadiazinane-3,5-dicarboxamide. | 1H), 7.97-7.94 (m, 1H), |
| | | 7.85-7.82 (m, 2H), 7.56-7.48 (m, |
| | | 4H), 7.42-7.37 (m, 2H), |
| | | 6.91 (s, 1H), 4.39 (d, J = 6.0 Hz, 2H), |
| | | 4.22-4.15 (m, 2H), 2.59 (s, 3H), 2.08- |
| | | 1.93 (m, 2H). LCMS calcd for; |
| | | C₂₂H₂₁ClFN₅O₅S. Found; |
| | | 522.05 (M + 1). |
| 1150 | 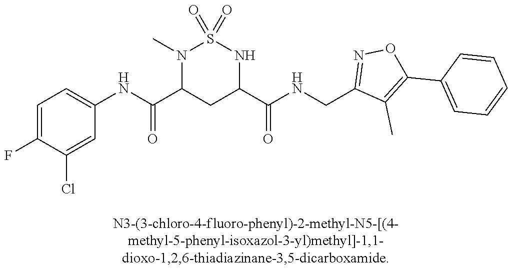 [SMILES: Cc1c(CNC(=O)C2CC(C(=O)Nc3ccc(F)c(Cl)c3)N(C)S(=O)(=O)N2)noc1-c1ccccc1] | ¹H NMR (400 MHz, DMSO-d6): δ 10.55 (s, 1H), 8.75-8.70 (m, 1H), 7.97-7.94 (m, 1H), 7.73-7.69 (m, 2H), 7.59- 7.49 (m, 4H), 7.42-7.37 (m, 2H), 4.45- 4.35 (m, 2H), 4.22-4.10 (m, 2H), 2.57 (s, 3H), 2.17 (s, 3H), 1.97-1.93 (m, 2H). LCMS calcd for; C₂₃H₂₃ClFN₅O₅S. Found; 536.10 (M + 1). |
| 1150- | N3-(3-chloro-4-fluoro-phenyl)-2-methyl-N5-[(4- | ¹H NMR (400 MHz, DMSO-d6): δ |
| ISOMER-A | methyl-5-phenyl-isoxazol-3-yl)methyl]-1,1- | 10.53 (s, 1H), 8.71 (d, J = 6.0 Hz, |
| | dioxo-1,2,6-thiadiazinane-3,5-dicarboxamide. | 1H), 7.97-7.94 (m, 1H), |
| | | 7.73-7.69 (m, 2H), 7.58-7.47 (m, |
| | | 4H), 7.42-7.35 (m, 2H), |
| | | 4.48-4.35 (m, 2H), 4.24-4.10 (m, 2H), |
| | | 2.57 (s, 3H), 2.17 (s, 3H), 1.99-1.94 |
| | | (m, 2H). LCMS calcd for; |
| | | C₂₃H₂₃ClFN₅O₅S. Found; |
| | | 536.05 (M + 1). |
| 1150- | N3-(3-chloro-4-fluoro-phenyl)-2-methyl-N5-[(4- | ¹H NMR (400 MHz, DMSO-d6): δ |
| ISOMER-B | methyl-5-phenyl-isoxazol-3-yl)methyl]-1,1- | 10.53 (s, 1H), 8.72 (d, J = 5.6 Hz, |
| | dioxo-1,2,6-thiadiazinane-3,5-dicarboxamide. | 1H), 7.97-7.94 (m, 1H), |
| | | 7.73-7.69 (m, 2H), |
| | | 7.58-7.48 (m, 4H), 7.42-7.35 (m, 2H), |
| | | 4.48-4.35 (m, 2H), 4.24-4.13 (m, 2H), |
| | | 2.57 (s, 3H), 2.17 (s, 3H), 1.99-1.94 |
| | | (m, 2H). LCMS calcd for; |
| | | C₂₃H₂₃ClFN₅O₅S. Found; |
| | | 536.05 (M + 1). |
| 1151 | 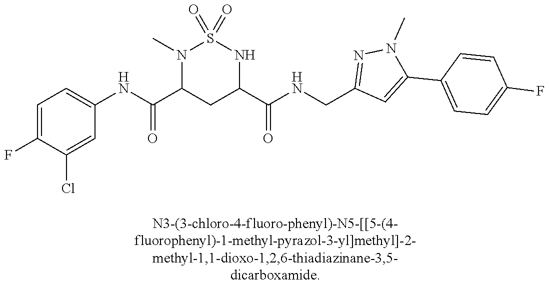 [SMILES: CN1C(C(=O)Nc2ccc(F)c(Cl)c2)CC(C(=O)NCc2cc(-c3ccc(F)cc3)n(C)n2)NS1(=O)=O] | ¹H NMR (400 MHz, DMSO-d6): δ 10.52 (s, 1H), 8.54-8.50 (m, 1H), 7.96-7.94 (m, 1H), 7.56-7.48 (m, 3H), 7.44- 7.32 (m, 4H), 6.31 (s, 1H), 4.26-4.12 (m, 4H), 3.78 (s, 3H), 2.57 (s, 3H), 1.96-1.92 (m, 2H). LCMS calcd for; C₂₃H₂₃ClF₂N₆O₄S. Found; 553.05 (M + 1). |
| 1151- | N3-(3-chloro-4-fluoro-phenyl)-N5-[[5-(4- | ¹H NMR (400 MHz, DMSO-d6): δ |
| ISOMER-A | fluorophenyl)-1-methyl-pyrazol-3-yl]methyl]-2- | 10.53 (s, 1H), 8.53 (d, J = 5.6 Hz, |
| | methyl-1,1-dioxo-1,2,6-thiadiazinane-3,5- | 1H), 7.96-7.92 (m, |
| | dicarboxamide | 1H), .58-7.52 (m, 3H), |
| | | 7.42-7.30 (m, 4H), 6.31 (s, 1H), 4.26- |
| | | 4.24 (m, 2H), 4.22-4.08 (m, 2H), 3.78 |
| | | (s, 3H), 2.57 (s, 3H), 2.00-1.92 (m, |
| | | 2H). LCMS calcd for; |
| | | C₂₃H₂₃ClF₂N₆O₄S. Found; 553 (M + |
| | | 1). |
| 1151- | N3-(3-chloro-4-fluoro-phenyl)-N5-[[5-(4- | ¹H NMR (400 MHz, DMSO-d6): δ |
| ISOMER-B | fluorophenyl)-1-methyl-pyrazol-3-yl]methyl]-2- | 10.53 (s, 1H), 8.53 (d, J = 5.6 Hz, 1H), |
| | methyl-1,1-dioxo-1,2,6-thiadiazinane-3,5- | 7.96-7.92 (m, 1H), 7.60-7.52 (m, 3H), |
| | dicarboxamide. | 7.42-7.28 (m, 4H), 6.31 (s, 1H), 4.26- |
| | | 4.23 (m, 2H), 4.22-4.10 (m, 2H), 3.78 |
| | | (s, 3H), 2.57 (s, 3H), 1.98-1.92 (m, |
| | | 2H). LCMS calcd for; |
| | | C₂₃H₂₃ClF₂N₆O₄S. Found; 553. (M + |
| | | 1). |
| 1153 | 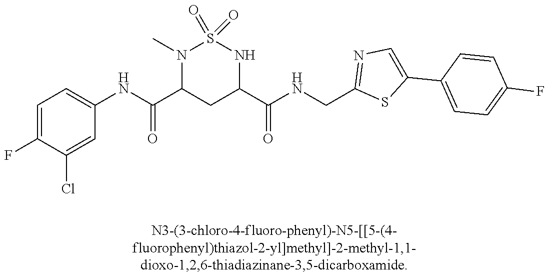 [SMILES: CN1C(C(=O)Nc2ccc(F)c(Cl)c2)CC(C(=O)NCc2ncc(-c3ccc(F)cc3)s2)NS1(=O)=O] | ¹H NMR (400 MHz, DMSO-d6): δ 10.53 (s, 1H), 9.06-9.00 (m, 1H), 8.06 (s, 1H), 7.97-7.94 (m, 1H), 7.70-7.65 (m, 2H), 7.57-7.53 (m, 1H), 7.46-7.35 (m, 2H), 7.31-7.24 (m, 2H), 4.65-4.40 (m, 2H), 4.25-4.14 (m, 2H), 2.58 (s, 3H), 2.04-1.95 (m, 2H). LCMS calcd for; C₂₂H₂₀ClF₂N₅O₄S₂. Found: 556 (M + 1). |
| 1153- | N3-(3-chloro-4-fluoro-phenyl)-N5-[[5-(4- | ¹H NMR (400 MHz, DMSO-d6): δ |
| ISOMER-A | fluorophenyl)thiazol-2-yl]methyl]-2-methyl-1,1- | 10.55 (s, 1H), 9.06 (t, J = 6.0 Hz, |
| | dioxo-1,2,6-thiadiazinane-3,5-dicarboxamide. | 1H), 8.07 (s, 1H), 7.97-7.94 (m, |
| | | 1H), 7.71-7.65 (m, 2H), |
| | | 7.57-7.53 (m, 1H), 7.48- |
| | | 7.45 (m, 1H), 7.40 (t, J = 8.8 Hz, 1H), |
| | | 7.31-7.24 (m, 2H), 4.65-4.51 (m, 2H), |
| | | 4.26-4.16 (m, 2H), 2.58 (s, 3H), 2.04- |
| | | 1.91 (m, 2H). LCMS calcd for; |
| | | C₂₂H₂₀ClF₂N₅O₄S₂. Found; 556 (M + |
| | | 1). |
| 1153- | N3-(3-chloro-4-fluoro-phenyl)-N5-[[5-(4- | ¹H NMR (400 MHz, DMSO-d6): δ |
| ISOMER-B | fluorophenyl)thiazol-2-yl]methyl]-2-methyl-1,1- | 10.55 (s, 1H), 9.07 (t, J = 5.6 Hz, |
| | dioxo-1,2,6-thiadiazinane-3,5-dicarboxamide. | 1H), 8.07 (s, 1H), 7.97-7.94 (m, |
| | | 1H), 7.70-7.66 (m, 2H), |
| | | 7.57-7.52 (m, 1H), 7.46 (d, |
| | | J = 10 Hz, 1H), 7.40 (t, J = 8.8 Hz, |
| | | 1H), 7.31-7.24 (m, 2H), 4.65-4.51 (m, |
| | | 2H), 4.26-4.14 (m, 2H), 2.58 (s, 3H), |
| | | 2.04-1.92 (m, 2H). LCMS calcd for; |
| | | C₂₂H₂₀ClF₂N₅O₄S₂. Found; |
| | | 556.10 (M + 1). |
| 1157 | 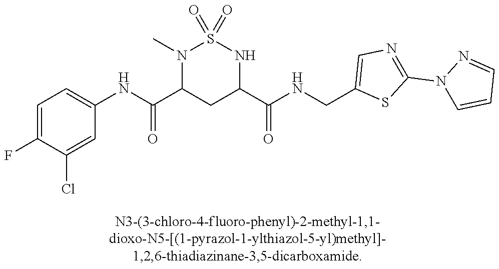 [SMILES: CN1C(C(=O)Nc2ccc(F)c(Cl)c2)CC(C(=O)NCc2cnc(-n3cccn3)s2)NS1(=O)=O] | ¹H NMR (400 MHz, DMSO-d6): δ 10.53 (s, 1H), 8.86-8.82 (m, 1H), 8.47-8.45 (m, 1H), 7.97-.73 (m, 1H), 7.84 (s, 1H), 7.58-7.49 (m, 2H), 7.42-7.35 (m, 2H), 6.61 (s, 1H), 4.45-4.36 (m, 2H), 4.20-4.10 (m, 2H), 2.57 (s, 3H), 2.09-1.98 (m, 2H). LCMS calcd for; C₁₉H₁₉ClFN₇O₄S₂. Found; 528. (M + 1). |
| 1158 | 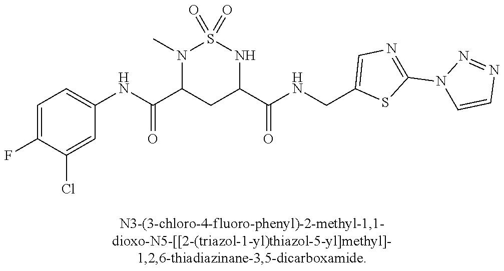 [SMILES: CN1C(C(=O)Nc2ccc(F)c(Cl)c2)CC(C(=O)NCc2cnc(-n3ccnn3)s2)NS1(=O)=O] | ¹H NMR (400 MHz, DMSO-d6): δ 10.51 (s, 1H), 8.86-8.82 (m, 1H), 8.47-8.45 (m, 1H), 8.22 (s, 1H), 8.03 (s, 1H), 7.95-7.92 (m, 1H), 7.55-7.51 (m, 1H), 7.46-7.34 (m, 2H), 4.51-4.46 (m, 2H), 4.22-4.06 (m, 2H), 2.56 (s, 3H), 2.02-1.90 (m, 2H). LCMS calcd for; C₁₈H₁₈ClFN₈O₄S₂. Found; 528.95 (M + 1). |
| 1159 | 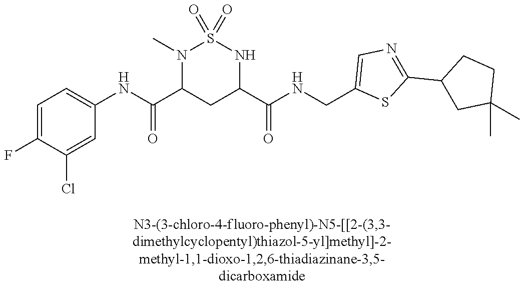 [SMILES: CN1C(C(=O)Nc2ccc(F)c(Cl)c2)CC(C(=O)NCc2cnc(C3CCC(C)(C)C3)s2)NS1(=O)=O] | ¹H NMR (400 MHz, DMSO-d6): δ 10.52 (s, 1H), 8.73 (t, J = 5.6 Hz, 1H), 7.97-7.92 (m, 1H), 7.55-7.53 (m, 1H), 7.48 (s, 1H), 7.42-7.37 (m, 2H), 4.49- 4.40 (m, 2H), 4.21-4.17 (m, 1H), 4.10- 4.05 (m, 1H), 3.58-3.52 (m, 1H), 2.57 (s, 3H), 2.18-2.13 (m, 1H), 1.96-1.81 (m, 4H), 1.63-1.45 (m, 3H), 1.05-1.02 (m, 6H). LCMS calcd for, C₂₃H₂₉ClFN₅O₄S₂. Found; 558.20 (M + 1). |
| 1160 | 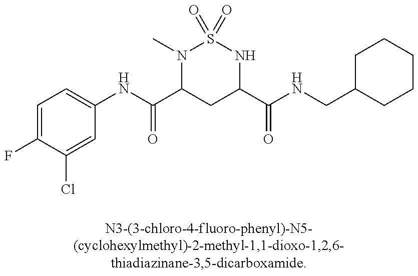 [SMILES: CN1C(C(=O)Nc2ccc(F)c(Cl)c2)CC(C(=O)NCC2CCCCC2)NS1(=O)=O] | ¹H NMR (400 MHz, DMSO-d6): δ 10.50 (s, 1H), 8.04 (t, J = 5.6 Hz, 1H), 7.96-7.94 (m, 1H), 7.58-7.52 (m, 1H), 7.39 (t, J = 8.8 Hz, 1H), 7.24 (d, J = 10 Hz, 1H), 4.21-4.16 (m, 1H), 4.10- 4.03 (m, 1H), 3.00-2.91 (m, 2H), 2.85 (s, 3H), 1.95-1.89 (m, 2H), 1.70-1.55 (m, 5H), 1.45-1.35 (m, 1H), 1.22-1.08 (m, 3H), 0.89-0.80 (m, 2H). LCMS Calcd for; C₁₉H₂₆ClFN₄O₄S. Found; 461.05 (M + 1). |
| 1160- | N3-(3-chloro-4-fluoro-phenyl)-N5- | ¹H NMR (400 MHz, DMSO-d6): δ |
| ISOMER-A | (cyclohexylmethyl)-2-methyl-1,1-dioxo-1,2,6- | 10.50 (s, 1H), 8.06-8.03 (m, 1H), |
| | thiadiazinane-3,5-dicarboxamide. | 7.95-7.92 (m, 1H), 7.55-7.51 (m, |
| | | 1H), 7.38 (t, J = 8.8 Hz, 1H), |
| | | 7.25-7.20 (m, 1H), |
| | | 4.20-4.15 (m, 1H), 4.10-4.04 (m, 1H), |
| | | 2.95-2.90 (m, 2H), 2.55 (s, 3H), 1395- |
| | | 1.86 (m, 2H), 1.70-1.55 (m, 5H), 1.42- |
| | | 1.35 (m, 1H), 1.25-1.10 (m, 3H), 0.86- |
| | | 0.82 (m, 2H). LCMS calcd for; |
| | | C₁₉H₂₆ClFN₄O₄S. Found; |
| | | 461.10 (M + 1). |
| 1160- | N3-(3-chloro-4-fluoro-phenyl)-N5- | ¹H NMR (400 MHz, DMSO-d6): δ |
| ISOMER-B | (cyclohexylmethyl)-2-methyl-1,1-dioxo-1,2,6- | 10.53 (s, 1H), 8.06 (t, J = 5.6 Hz, |
| | thiadiazinane-3,5-dicarboxamide. | 1H), 7.96-7.94 (m, 1H), |
| | | 7.56-7.52 (m, 1H), 7.40 (t, J = |
| | | 8.8 Hz, 1H), 7.27-7.24 (m, |
| | | 1H), 4.21-4.16 (m, 1H), 4.10-4.02 (m, |
| | | 1H), 2.95-2.90 (m, 2H), 2.56 (s, 3H), |
| | | 1.95-1.88 (m, 2H), 1.70-1.54 (m, 5H), |
| | | 1.45-1.35 (m, 1H), 1.25-1.09 (m, 3H), |
| | | 0.90-0.87 (m, 2H). LCMS calcd for; |
| | | C₁₉H₂₆ClFN₄O₄S. FOund; |
| | | 461.05 (M + 1). |
| 1161 | 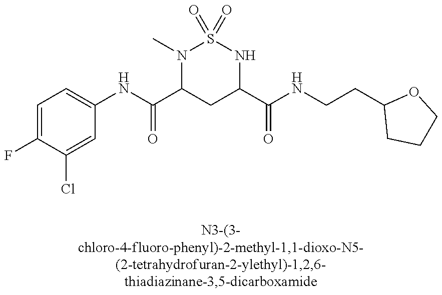 [SMILES: CN1C(C(=O)Nc2ccc(F)c(Cl)c2)CC(C(=O)NCCC2CCCO2)NS1(=O)=O] | ¹H NMR (400 MHz, DMSO-d6): δ 10.51 (s, 1H), 8.09-8.06 (m, 1H), 7.96-7.93 (m, 1H), 7.56-7.52 (m, 1H), 7.39 (t, J = 8.8 Hz, 1H), 7.27-7.25 (m, 1H), 4.22-4.15 (m, 1H), 4.10-4.00 (m, 1H), 3.78-3.69 (m, 2H), 3.60-3.54 (m, 1H), 3.21-3.07 (m, 2H), 1.99-1.89 (m, 3H), 1.86-1.73 (m, 2H), 1.62-1.55 (m, 2H), 1.42-1.33 (m, 2H). LCMS calcd for; C₁₈H₂₄ClFN₄O₅S. Found; 463.15 (M + 1). |
| 1162 | 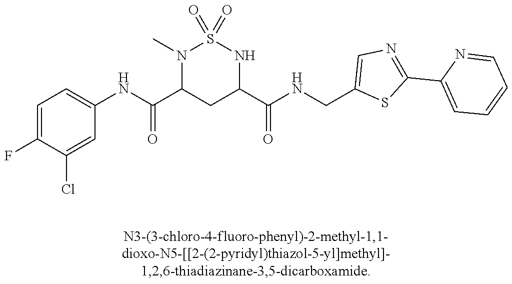 [SMILES: CN1C(C(=O)Nc2ccc(F)c(Cl)c2)CC(C(=O)NCc2cnc(-c3ccccn3)s2)NS1(=O)=O] | ¹H NMR (400 MHz, DMSO-d6): δ 10.51 (s, 1H), 8.85-8.83 (m, 1H), 8.59 (d, J = 4.4 Hz, 1H), 8.06 (d, J = 7.6 Hz, 1H), 7.95-7.90 (m, 2H), 7.81 (s, 1H), 7.53-7.35 (m, 4H), 4.52-4.51 (m, 2H), 4.21-4.17 (m, 1 H), 4.12-4.08 (m, 1H), 2.56 (s, 3H), 1.99-1.92 (m, 2H). LCMS calcd for; C₂₁H₂₀ClFN₆O₄S₂. Found; 539.05 (M + 1). |
| 1197 | 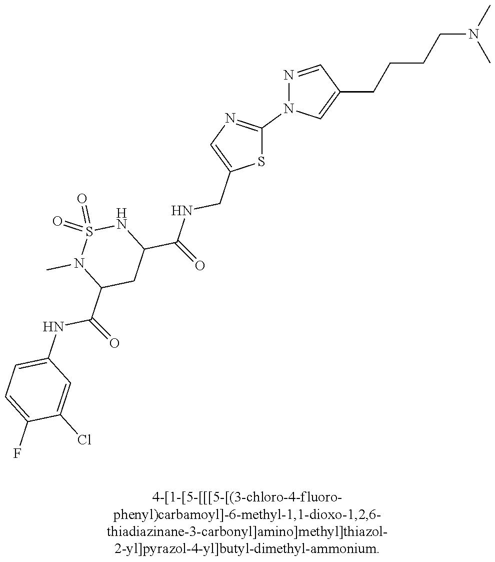 [SMILES: CN(C)CCCCc1cnn(-c2ncc(CNC(=O)C3CC(C(=O)Nc4ccc(F)c(Cl)c4)N(C)S(=O)(=O)N3)s2)c1] | ¹H NMR (400 MHz, DMSO-d6): δ 10.52 (s, 1H), 8.82 (t, J = 6.0 Hz, 1H), 8.28 (s, 1H), 7.95-7.92 (m, 1H), 7.71 (s, 1H), 7.54-7.50 (m, 1H), 7.45-7.35 (m, 3H), 4.43-4.40 (m, 2H), 4.22-4.17 (m, 1H), 4.10-4.05 (m, 1H), 3.10-2.96 (m, 2H), 2.72 (s, 6H), 2.56 (s, 3H), 1.98-1.86 (m, 2H), 1.65-1.55 (m, 4H). LCMS calcd for; C₂₅H₃₂ClFN₈O₄S₂. Found; 627.05 (M + 1). |
| 1197- | N3-(3-chloro-4-fluoro-phenyl)-N5-[[2-[4-[4- | ¹H NMR (400 MHz, DMSO-d6): δ |
| ISOMER-A | (dimethylamino)butyl]pyrazol-1-yl]thiazol-5- | 10.52 (s, 1H), 8.82-8.78 (m, 1H), 8.24 |
| | yl]methyl]-2-methyl-1,1-dioxo-1,2,6- | (s, 1H), 7.96-7.93 (m, 1H), 7.70 (m, |
| | thiadiazinane-3,5-dicarboxamide. | 1H), 7.55-7.51 (m, 1H), 7.46-7.38 (m, |
| | | 3H), 4.44-4.40 (m, 2H), 4.20-4.05 (m, |
| | | 2H), 2.57 (s, 3H), 2.25-2.18 (m, 6H), |
| | | 1.97-1.94 (m, 2H), 1.57-1.43 (m, |
| | | 4H). LCMS calcd for; |
| | | C₂₅H₃₂ClFN₈O₄S₂. Found; |
| | | 627.05 (M + 1). |
| 1197- | N3-(3-chloro-4-fluoro-phenyl)-N5-[[2-[4-[4- | ¹H NMR (400 MHz, DMSO-d6): δ |
| ISOMER-B | (dimethylamino)butyl]pyrazol-1-yl]thiazol-5- | 10.52 (s, 1H), 8.84-8.78 (m, 1H), 8.24 |
| | yl]methyl]-2-methyl-1,1-dioxo-1,2,6- | (s, 1H), 7.96-7.93 (m, 1H), 7.69 (m, |
| | thiadiazinane-3,5-dicarboxamide. | 1H), 7.58-7.36 (m, 4H), 4.46-4.40 (m, |
| | | 2H), 4.22-4.18 (m, 2H), 2.57 (s, 3H), |
| | | 2.33-2.26 (m, 2H), 2.15 (s, 6H), 1.98- |
| | | 1.94 (m, 2H), 1.56-1.42 (m, |
| | | 4H). LCMS calcd for; |
| | | C₂₅H₃₂ClFN₈O₄S₂. Found; |
| | | 627.05 (M + 1). |
| 1199 | 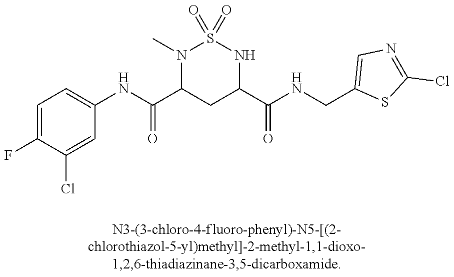 [SMILES: CN1C(C(=O)Nc2ccc(F)c(Cl)c2)CC(C(=O)NCc2cnc(Cl)s2)NS1(=O)=O] | ¹H NMR (400 MHz, DMSO-d6) : δ 10.53 (s, 1H), 8.84 (t, J = 5.6 Hz, 1H), 7.96-7.94 (m, 1H), 7.56-7.52 (m, 2H), 7.46-7.37 (m, 2H), 4.41 (d, J = 6.0 Hz, 2H), 4.21-4.17 (m, 1H), 4.12-4.05 (m, 1H), 2.57 (s, 3H), 2.00-1.86 (m, 2H). LCMS calcd for; C₁₆H₁₆Cl₂FN₅O₄S₂. Found; 495.95 (M + 1). |
| 1199- | N3-(3-chloro-4-fluoro-phenyl)-N5-[(2- | ¹H NMR (400 MHz, DMSO-d6): δ |
| ISOMER-A | chlorothiazol-5-yl)methyl]-2-methyl-1,1-dioxo- | 10.52 (s, 1H), 8.84-8.80 (m, 1H), |
| | 1,2,6-thiadiazinane-3,5-dicarboxamide. | 7.96-7.92 (m, 1H), 7.58-7.52 (m, |
| | | 2H), 7.46-7.36 (m, 2H), 4.42 (d, |
| | | J = 5.2 Hz, 2H), |
| | | 4.21-4.17 (m, 1H), 4.12-4.08 (m, 1H), |
| | | 2.57 (s, 3H), 2.00-1.86 (m, 2H). |
| | | LCMS calcd for; C₁₆H₁₆Cl₂FN₅O₄S₂. |
| | | Found; 496.05 (M + 1). |
| 1199- | N3-(3-chloro-4-fluoro-phenyl)-N5-[(2- | ¹H NMR (400 MHz, DMSO-d6): δ |
| ISOMER-B | chlorothiazol-5-yl)methyl]-2-methyl-1,1-dioxo- | 10.53 (s, 1H), 8.84-8.80 (m, 1H), |
| | 1,2,6-thiadiazinane-3,5-dicarboxamide. | 7.96-7.92 (m, 1H), 7.58-7.50 (m, |
| | | 2H), 7.42-7.36 (m, 2H), 4.42 (d, J = |
| | | 5.2 Hz, 2H), 4.21-4.17 (m, 1H), |
| | | 4.12-4.06 (m, 1H), |
| | | 2.57 (s, 3H), 2.00-1.86 (m, 2H). |
| | | LCMS calcd for; |
| | | C₁₆H₁₆Cl₂FN₅O₄S₂. |
| | | Found; 496.05 (M + 1). |
| 1202 | 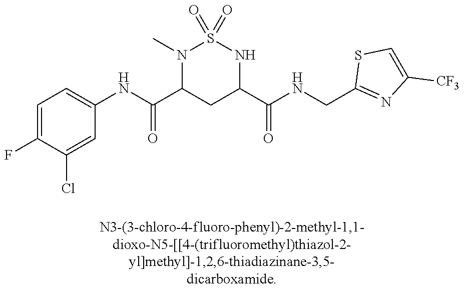 [SMILES: CN1C(C(=O)Nc2ccc(F)c(Cl)c2)CC(C(=O)NCc2nc(C(F)(F)F)cs2)NS1(=O)=O] | ¹H NMR (400 MHz, DMSO-d6): δ 10.55 (s, 1H), 9.12 (t, J = 6.0 Hz, 1H), 8.42 (s, 1H), 7.97-7.94 (m, 1H), 7.56-7.48 (m, 2H), 7.42 (t, J = 8.8 Hz, 1H), 7.65-7.58 (m, 2H), 4.25-4.14 (m, 2H), 2.58 (s, 3H), 2.04-1.93 (m, 2H). LCMS calcd for; C₁₇H₁₆ClF₄N₅O₄S₂. Found; 529.95 (M + 1). |
| 1202- | N3-(3-chloro-4-fluoro-phenyl)-2-methyl-1,1- | ¹H NMR (400 MHz, DMSO-d6): δ |
| ISOMER-A | dioxo-N5-[[4-(trifluoromethyl)thiazol-2- | 10.54 (s, 1H), 9.15-9.10 (m, 1H), 8.42 |
| | yl]methyl]-1,2,6-thiadiazinane-3,5- | (s, 1H), 7.96-7.93 (m, 1H), 7.55-7.48 |
| | dicarboxamide. | (m, 2H), 7.39 (t, J = 8.8 Hz, 1H), |
| | | 4.68-4.56 (m, 2H), 4.26-4.17 (m, |
| | | 2H), 2.58 (s, 3H), 2.04-1.94 (m, |
| | | 1H). LCMS calcd for; |
| | | C₁₇H₁₆ClF₄N₅O₄S₂. Found; |
| | | 529.95 (M + 1). |
| 1202- | N3-(3-chloro-4-fluoro-phenyl)-2-methyl-1,1- | ¹H NMR (400 MHz, DMSO-d6): δ |
| ISOMER-B | dioxo-N5-[[4-(trifluoromethyl)thiazol-2- | 10.55 (s, 1H), 9.15-9.10 (m, 1H), 8.42 |
| | yl]methyl]-1,2,6-thiadiazinane-3,5- | (s, 1H), 7.96-7.94 (m, 1H), 7.55-7.49 |
| | dicarboxamide. | (m, 2H), 7.39 (t, J = 8.8 Hz, 1H), |
| | | 4.68-4.55 (m, 2H), 4.26-4.17 (m, |
| | | 2H), 2.58 (s, 3H), 2.04-1.90 (m, |
| | | 2H). LCMS calcd for; |
| | | C₁₇H₁₆ClF₄N₅O₄S₂. Found; |
| | | 530.10 (M + 1). |
| 1205 | 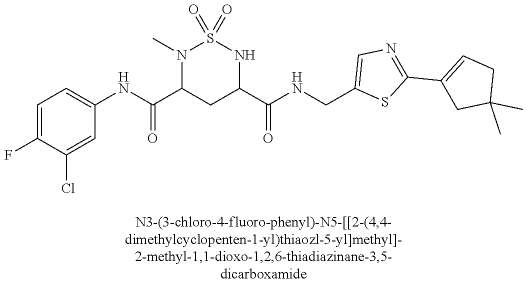 [SMILES: CN1C(C(=O)Nc2ccc(F)c(Cl)c2)CC(C(=O)NCc2cnc(C3=CCC(C)(C)C3)s2)NS1(=O)=O] | .¹H NMR (400 MHz, DMSO-d6): δ 10.52 (bs, 1H), 8.76 (t, J = 5.6 Hz, 1H), 7.96-7.93 (m, 1H), 7.60-7.51 (m, 2H), 7.42-7.36 (m, 2H), 6.32 (s, 1H), 4.46-4.45 (m, 2H), 4.22-4.17 (m, 1H), 4.10-4.06 (m, 1H), 2.56 (s, 3H), 2.33 (s, 2H), 1.98-1.89 (m, 2H), 1.11 (s, 6H). LCMS calcd for; C₂₃H₂₇ClFN₅O₄S₂. Found; 556.20 (M + 1). |
| 476 | 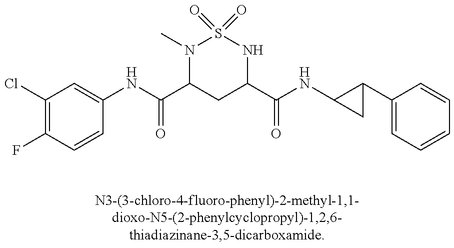 [SMILES: CN1C(C(=O)Nc2ccc(F)c(Cl)c2)CC(C(=O)NC2CC2c2ccccc2)NS1(=O)=O] | ¹H NMR (400 MHz, DMSO-d6): δ 10.52 (s, 1H), 8.41-8.38 (m, 1H), 7.96-7.94 (m, 1H), 7.54-7.52 (m, 1H), 7.39 (t, J = 8.8 Hz, 1H), 7.27-7.23 (m, 3H), 7.17-7.09 (m, 3H), 4.18-4.01 (m, 2H), 2.85-2.83 (m, 1H), 2.56 (s, 3H), 1.98- 1.93 (m, 3H), 1.26-1.15 (m, 2H). LCMS calcd for; C₂₁H₂₂ClFN₄O₄S. Found; 481.1 (M + 1). |
| 477 | 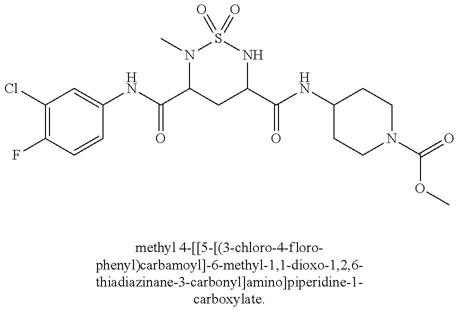 [SMILES: COC(=O)N1CCC(NC(=O)C2CC(C(=O)Nc3ccc(F)c(Cl)c3)N(C)S(=O)(=O)N2)CC1] | ¹H NMR (400 MHz, DMSO-d6): δ 10.50 (s, 1H), 8.06-8.04 (m, 1H), 7.95-7.93 (m, 1H), 7.56-7.53 (m, 1H), 7.39 (t, J = 9.2 Hz, 1H), 7.27-7.24 (m, 1H), 4.21-4.17 (m, 1H), 4.06-4.04 (m, 1H), 3.87-3.75 (m, 3H), 3.58 (s, 3H), 2.93- 2.90 (m, 2H), 2.56 (s, 3H), 1.95-1.89 (m, 2H), 1.72-1.70 (m, 2H), 1.37-1.25 (m, 2H). LCMS calcd for; C₁₉H₂₅ClFN₅O₆S. Found; 506.25 (M + 1). |
| 477- | methyl 4-[[5-[(3-chloro-4-fluoro- | ¹H NMR (400 MHz, DMSO-d6): δ |
| ISOMER-A | phenyl)carbamoyl]-6-methyl-1,1-dioxo-1,2,6- | 10.52 (s, 1H), 8.05 (d, J = 7.6 Hz, |
| | thiadiazinane-3-carbonyl]amino]piperidine-1- | 1H), 7.96-7.93 (m, 1H), |
| | carboxylate. | 7.56-7.53 (m, 1H), 7.39 (t, J = |
| | | 8.8 Hz, 1H), 7.26 (s, 1H), |
| | | 4.21-4.17 (m, 1H), 4.05-4.02 (m, 1H), |
| | | 3.86-3.80 (m, 2H), 3.79-3.74 (m, 1H), |
| | | 3.72 (s, 3H), 2.93-2.90 (m, 2H), 2.56 |
| | | (s, 3H), 1.92-1.87 (m, 2H), 1.74-1.70 |
| | | (m, 2H), 1.38-1.26 (m, 2H). LCMS |
| | | calcd for.; C₁₉H₂₅ClFN₅O₆S. Found; |
| | | 506.1 (M + 1). |
| 477- | methyl 4-[[5-[(3-chloro-4-fluoro- | ¹H NMR (400 MHz, DMSO-d6): δ |
| ISOMER-B | phenyl)carbamoyl]-6-methyl-1,1-dioxo-1,2,6- | 10.50 (s, 1H), 8.06-7.92 (m, 2H), |
| | thiadiazinane-3-carbonyl]amino]piperidine-1- | 7.56-7.53 (m, 1H), 7.39 (t, |
| | carboxylate. | J = 8.8 Hz, 1H), 7.27-7.25 (m, |
| | | 1H), 4.20-4.17 (m, 1H), |
| | | 4.05-4.02 (m, 1H), 3.87-3.75 (m, 3H), |
| | | 3.58 (s, 3H), 2.93-2.90 (m, 2H), 2.56 |
| | | (s, 3H), 1.97-1.89 (m, 2H), 1.72-1.70 |
| | | (m, 2H), 1.37-1.25 (m, 2H). |
| | | LCMS calcd for; |
| | | C₁₉H₂₅ClFN₅O₆S. |
| | | Found; 506.30 (M + 1). |
| 478 | 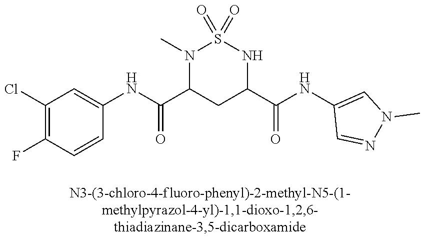 [SMILES: CN1C(C(=O)Nc2ccc(F)c(Cl)c2)CC(C(=O)Nc2cnn(C)c2)NS1(=O)=O] | ¹H NMR (400 MHz, DMSO-d6): δ 10.55 (s, 1H), 10.20 (s, 1H), 7.97-7.95 (m, 1H), 7.89 (s, 1H), 7.57-7.54 (m, 2H), 7.48 (s, 1H), 7.40 (t, J = 9.2 Hz, 1H), 4.25-4.20 (m, 2H), 3.79 (s, 3H), 2.58 (s, 3H), 2.03-1.98 (m, 2H). LCMS calcd for; C₁₆H₁₈ClFN₆O₄S. Found; 444.95 (M + 1). |
| 481 | 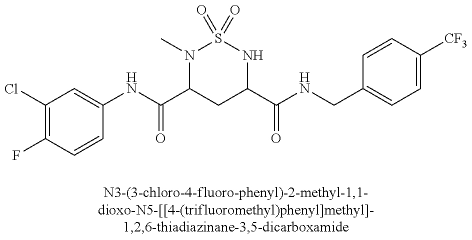 [SMILES: CN1C(C(=O)Nc2ccc(F)c(Cl)c2)CC(C(=O)NCc2ccc(C(F)(F)F)cc2)NS1(=O)=O] | ¹H NMR (400 MHz, DMSO-d6): δ 10.54 (s, 1H), 8.74 (t, J = 5.6 Hz, 1H), 7.95 (dd, J = 6.8, 2.4 Hz, 1H), 7.68 (d, J = 8.4 Hz, 2H), 7.57-7.52 (m, 1H), 7.49 (d, J = 8.4 Hz, 2H), 7.45-7.43 (m, 1H) 7.39 (t, J = 9.2 Hz, 1H), 4.39-4.37 (m, 2H), 4.22-4.13 (m, 2H), 2.58 (s, 3H), 2.04-1.94 (m, 2H). LCMS calcd for; C₂₀H₁₉ClF₄N₄O₄S. Found; 523.1 (M + 1). |
| 482 | 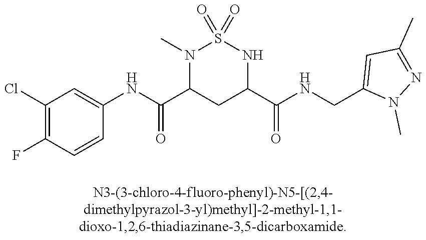 [SMILES: Cc1cc(CNC(=O)C2CC(C(=O)Nc3ccc(F)c(Cl)c3)N(C)S(=O)(=O)N2)n(C)n1] | ¹H NMR (400 MHz, DMSO-d6): δ 10.54 (s, 1H), 8.58 (t, J = 5.2 Hz, 1H), 7.95 (dd, J = 6.8, 2.4 Hz, 1H), 7.56- 7.52 (m, 1H), 7.42-7.37 (m, 2H), 5.92 (s, 1H), 4.28-4.26 (m, 2H), 4.21-4.17 (m, 1H), 4.10-4.08 (m, 1H), 3.67 (s, 3H), 2.57 (s, 3H), 2.07 (s, 3H), 1.96- 1.92 (m, 2H). LCMS calcd for; C₁₈H₂₂ClFN₆O₄S. Found; 473.1 (M + 1). |
| 482- | N3-(3-chloro-4-fluoro-phenyl)-N5-[(2,5- | ¹H NMR (400 MHz, DMSO-d6): δ |
| ISOMER-A | dimethylpyrazol-3-yl)methyl]-2-methyl-1,1- | 10.52 (s, 1H), 8.55-8.54 (m, 1H), 7.96 |
| | dioxo-1,2,6-thiadiazinane-3,5-dicarboxamide. | (dd, J = 6.9, 2.7 Hz, 1H), 7.55-7.53 |
| | | (m, 1H), 7.41-7.39 (m, 2H), 5.92 (s, |
| | | 1H), 4.28-4.26 (m, 2H), 4.23-4.05 (m, |
| | | 2H), 3.67 (s, 3H), 2.55 (s, 3H), 2.08 |
| | | (s, 3H), 1.95-1.90 (m, 2H). LCMS |
| | | calcd for; C₁₈H₂₂ClFN₆O₄S. Found; |
| | | 473.20 (M + 1). |
| 482- | N3-(3-chloro-4-fluoro-phenyl)-N5-[(2,5- | ¹H NMR (400 MHz, DMSO-d6): δ |
| ISOMER-B | dimethylpyrazol-3-yl)methyl]-2-methyl-1,1- | 10.52 (s, 1H), 8.55-8.54 (m, 1H), 7.96 |
| | dioxo-1,2,6-thiadiazinane-3,5-dicarboxamide. | (dd, J = 6.9, 2.6 Hz, 1H), 7.57-7.53 |
| | | (m, 1H), 7.42-7.40 (m, 2H), 5.92 (s, |
| | | 1H), 4.28-4.27 (m, 2H), 4.24-4.07 (m, |
| | | 2H), 3.67 (s, 3H), 2.55 (s, 3H), 2.08 |
| | | (s, 3H), 1.95-1.89 (m, 2H). LCMS |
| | | calcd for; C₁₈H₂₂ClFN₆O₄S. |
| | | Found; 473.25 (M + 1). |
| 485 | 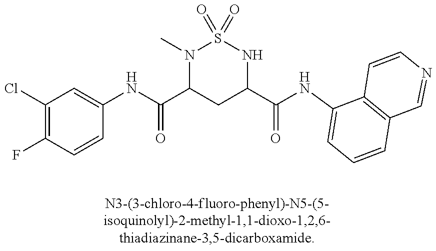 [SMILES: CN1C(C(=O)Nc2ccc(F)c(Cl)c2)CC(C(=O)Nc2cccc3cnccc23)NS1(=O)=O] | ¹H NMR (400 MHz, DMSO-d6): δ 10.60 (s, 1H), 10.29 (s, 1H), 9.34 (s, 1H), 8.55 (d, J = 6.0 Hz, 1H), 8.02- 7.97 (m, 2H), 7.93-7.88 (m, 2H), 7.72- 7.63 (m, 2H), 7.59-7.56 (m, 1H), 7.43- 7.38 (m, 1H), 4.57-4.52 (m, 1H), 4.33- 4.30 (m, 1H), 2.64 (s, 3H), 2.16-2.07 (m, 2H). LCMS calcd for; C₂₁H₁₉ClFN₅O₄S. Found; 492.1 (M + 1). |
| 489 | 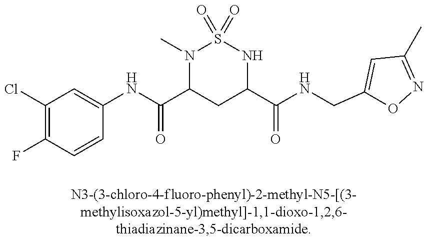 [SMILES: Cc1cc(CNC(=O)C2CC(C(=O)Nc3ccc(F)c(Cl)c3)N(C)S(=O)(=O)N2)on1] | ¹H NMR (400 MHz, DMSO-d6): δ 10.54 (s, 1H), 8.76 (t, J = 5.6 Hz, 1H), 7.95 (dd, J = 6.8, 2.4 Hz, 1H), 7.56-7.53 (m, 1H), 7.44-7.37 (m, 2H), 6.22 (s, 1H), 4.38-4.36 (m, 2H), 4.21-4.12 (m, 2H), 2.58 (s, 3H), 2.19 (s, 3H). 2.02-1.92 (m, 2H). LCMS calcd for; C₁₇H₁₉ClFN₅O₅S. Found; 460 (M + 1). |
| 493 | 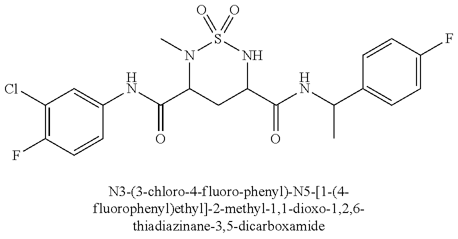 [SMILES: CC(NC(=O)C1CC(C(=O)Nc2ccc(F)c(Cl)c2)N(C)S(=O)(=O)N1)c1ccc(F)cc1] | ¹H NMR (400 MHz, DMSO-d6): δ 10.49 (s, 1H), 8.53-8.48 (m, 1H), 7.92-7.90 (m, 1H), 7.51-7.49 (m, 1H), 7.38-7.32 (m, 3H), 7.20-7.10 (m, 3H), 4.89-4.86 (m, 1H), 4.17-4.07 (m, 2H), 2.53 (s, 3H), 1.92-1.85 (m, 2H), 1.35-1.33 (m, 3H). LCMS calcd for; C₂₀H₂₁ClF₂N₄O₄S. Found; 487.1 (M + 1). |
| 493-R- ISOMER-A | 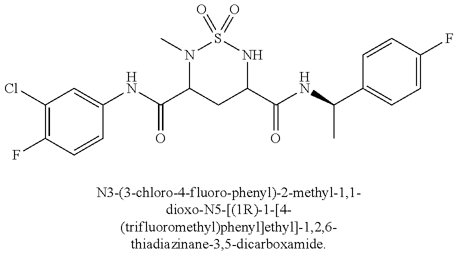 [SMILES: C[C@@H](NC(=O)C1CC(C(=O)Nc2ccc(F)c(Cl)c2)N(C)S(=O)(=O)N1)c1ccc(F)cc1] | ¹H NMR (400 MHz, DMSO-d6): δ 10.54 (d, J = 3.7 Hz, 1H), 8.56 (dd, J = 12.5, 7.9 Hz, 1H), 7.96-7.94 (m, 1H), 7.57-7.52 (m, 1H), 7.45-7.26 (m, 4H), 7.17-7.11 (m, 2H), 4.95-4.90 (m, 1H), 4.26-4.16 (m, 1H), 4.12-4.10 (m, 1H), 2.57 (s, 3H), 2.01-1.84 (m, 2H), 1.37- 1.35 (m, 3H). LCMS calcd for; C₂₀H₂₁ClF₂N₄O₄S. Found; 487 (M + 1). |
| 493-R- | N3-(3-chloro-4-fluoro-phenyl)-2-methyl-1,1- | ¹H NMR (400 MHz, DMSO-d6): δ |
| ISOMER-A | ioxo-N5-[(1R)-1-[4- | 10.52 (s, 1H), 8.56 (d, J = 7.8 Hz, |
| | (trifluoromethyl)phenyl]ethyl]-1,2,6- | 1H), 7.99-7.91 (m, 1H), |
| | thiadiazinane-3,5-dicarboxamide. | 7.58-7.49 (m, 1H), 7.45-7.27 (m, |
| | | 4H), 7.19-7.09 (m, 2H), 4.92 (p, |
| | | J = 7.0 Hz, 1H), 4.25-4.14 (m, |
| | | 1H), 4.11-4.09 (m, 1H), 2.56 (s, |
| | | 3H), 1.91-1.87 (m, 2H), 1.37 (d, J = |
| | | 6.9 Hz, 3H). LCMS calcd for; |
| | | C₂₀H₂₁ClF₂N₄O₄S. Found; 487.25 |
| | | (M + 1). |
| 493-R- | N3-(3-chloro-4-fluoro-phenyl)-2-methyl-1,1- | ¹H NMR (400 MHz, DMSO-d6): δ |
| ISOMER-B | dioxo-N5-[(1R)-1-[4- | 10.52 (s, 1H), 8.56 (d, J = 7.8 Hz, |
| | (trifluoromethyl)phenyl]ethyl]-1,2,6- | 1H), 7.99-7.91 (m, 1H), |
| | thiadiazinane-3,5-dicarboxamide. | 7.58-7.49 (m, 1H), 7.45-7.27 (m, |
| | | 4H), 7.19-7.09 (m, 2H), 4.92 (p, |
| | | J = 7.0 Hz, 1H), 4.25-4.14 (m, |
| | | 1H), 4.11 (t, J = 7.7 Hz, 1H), 2.56 (s, |
| | | 3H), 1.92-1.86 (m, 2H), 1.37 (d, J = |
| | | 6.9 Hz, 3H). LCMS calcd for; |
| | | C₂₀H₂₁ClF₂N₄O₄S. Found; 487.25 |
| | | (M + 1). |
| 493-S | 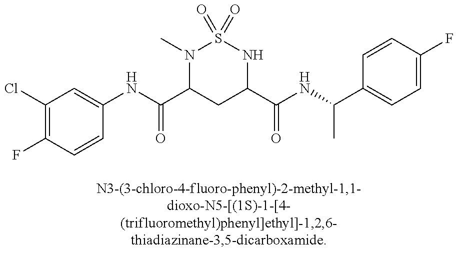 [SMILES: C[C@H](NC(=O)C1CC(C(=O)Nc2ccc(F)c(Cl)c2)N(C)S(=O)(=O)N1)c1ccc(F)cc1] | ¹H NMR (400 MHz, DMSO-d6): δ 10.54 (d, J = 3.7 Hz, 1H), 8.56 (dd, J = 12.5, 7.8 Hz, 1H), 7.96-7.94 (m, 1H), 7.59-7.49 (m, 1H), 7.45-7.26 (m, 4H), 7.17-7.11 (m, 2H), 4.95-4.90 (m, 1H), 4.27-4.06 (m, 2H), 2.57-2.56 (m, 3H), 2.01-1.84 (m, 2H), 1.37-1.35 (m, 3H). LCMS calcd for; C₂₀H₂₁ClF₂N₄O₄S. Found; 487.1 (M + 1). |
| 493-S- | N3-(3-chloro-4-fluoro-phenyl)-2-methyl-1,1- | ¹H NMR (400 MHz, DMSO-d6): δ |
| ISOMER-A | dioxo-N5-[(1S)-1-[4- | 10.52 (s, 1H), 8.56 (d, J = 7.8 Hz, |
| | (trifluoromethyl)phenyl]ethyl]-1,2,6- | 1H), 7.96-7.93 (m, 1H), |
| | thiadiazinane-3,5-dicarboxamide. | 7.55-7.52 (m, 1H), 7.44-7.25 (m, |
| | | 4H), 7.19-7.09 (m, 2H), 4.92 (p, |
| | | J = 7.2 Hz, 1H), 4.21-4.18 (m, |
| | | 1H), 4.11-4.09 (m, 1H), 2.56 (s, 3H), |
| | | 1.90-1.86 (m, 2H), 1.37 (d, J = 7.0 |
| | | Hz, 3H). LCMS calcd for; |
| | | C₂₀H₂₁ClF₂N₄O₄S. Found; 487.25 |
| | | (M + 1). |
| 493-S- | N3-(3-chloro-4-fluoro-phenyl)-2-methyl-1,1- | ¹H NMR (400 MHz, DMSO-d6): δ |
| ISOMER-B | (trifluoromethyl)phenyl]ethyl]-1,2,6- | 10.54 (s, 1H), 8.54 (s, J = 7.9 Hz, |
| | thiadiazinane-3,5-dicarboxamide. | 1H), 7.95 (dd, J = 6.9, 2.5 Hz, |
| | | 1H), 7.55-7.52 (m, 1H), |
| | | 7.46-7.34 (m, 3H), 7.32- |
| | | 7.21 (m, 2H), 7.15 (t, J = 8.8 Hz, 1H), |
| | | 4.97-4.93 (m, 1H), 4.25-4.07 (m, 2H), |
| | | 2.57 (s, 3H), 2.06-1.89 (m, 2H), 1.36 |
| | | (d, J = 6.9 Hz, 3H). LCMS calcd for; |
| | | C₂₀H₂₁ClF₂N₄O₄S. Found; 487.20 |
| | | (M + 1). |
| 494 | 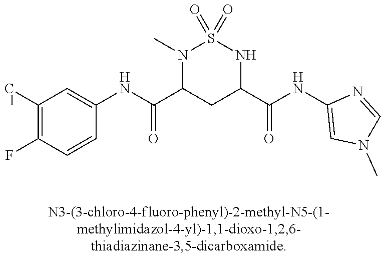 [SMILES: CN1C(C(=O)Nc2ccc(F)c(Cl)c2)CC(C(=O)Nc2cn(C)cn2)NS1(=O)=O] | ¹H NMR (400 MHz, DMSO-d6): δ 10.56 (s, 1H), 10.48 (s, 1H), 7.95 (dd, J = 6.4, 2.4 Hz, 1H), 7.57-7.53 (m, 1H), 7.42-7.37 (m, 2H), 7.32-7.29 (m, 1H), 7.21 (s, 1H), 4.35-4.30 (m, 1H), 4.26-4.22 (m, 1H), 3.62 (s, 3H), 2.58 (s, 3H), 2.05-1.91 (m, 2H). LCMS calcd for; C₁₆H₁₈ClFN₆O₄S. Found; 445.00 (M + 1). |
| 495 | 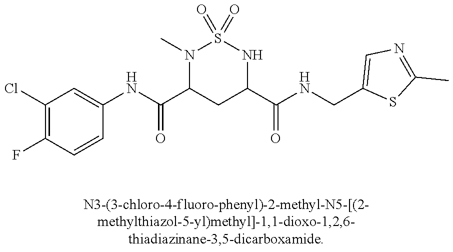 [SMILES: Cc1ncc(CNC(=O)C2CC(C(=O)Nc3ccc(F)c(Cl)c3)N(C)S(=O)(=O)N2)s1] | ¹H NMR (400 MHz, DMSO-d6): δ 10.53 (s, 1H), 8.75 (t, J = 5.2 Hz, 1H), 7.96-7.94 (m, 1H), 7.55-7.53 (m, 1H), 7.47 (s, 1H), 7.42-7.37 (m, 2H), 4.43- 4.37 (m, 2H), 4.22-4.17 (m, 1H), 4.07- 4.04 (m, 1H), 2.59 (s, 3H), 2.56 (s, 3H), 1.95-1.89 (m, 2H). LCMS calcd for; C₁₇H₁₉ClFN₅O₄S₂. Found; 476.1 (M + 1). |
| 495- | N3-(3-chloro-4-fluroo-phenyl)-2-methyl-N5-[(2- | ¹H NMR (400 MHz, DMSO-d6): δ |
| ISOMER-A | methylthiazol-5-yl)methyl]-1,1-dioxo-1,2,6- | 10.49 (s, 1H), 8.72-8.69 (m, 1H), |
| | thiadiazinane-3,5-dicarboxamide. | 7.92-7.90 (m, 1H), 7.52-7.49 (m, |
| | | 1H), 7.43 (s, 1H), 7.38-7.33 (m, |
| | | 2H), 4.43-4.33 (m, 2H), |
| | | 4.18-4.14 (m, 1H), 4.04-4.02 |
| | | (m, 1H), 2.55-2.52 (m, 6H), 1.92-1.85 |
| | | (m, 2H). LCMS calcd for; |
| | | C₁₇H₁₉ClFN₅O₄S₂. Found; 476.1 |
| | | (M + 1). |
| 495- | N3-(3-chloro-4-fluoro-phenyl)-2-methyl-N5-[(2- | ¹H NMR (400 MHz, DMSO-d6): δ |
| ISOMER-B | methylthiazol-5-yl)methyl]-1,1-dioxo-1,2,6- | 10.49 (s, 1H), 8.70 (t, J = 6.0 Hz, |
| | thiadiazinane-3,5-dicarboxamide. | 1H), 7.93-7.90 (m, 1H), |
| | | 7.53-7.49 (m, 1H), 7.43 (s, |
| | | 1H), 7.38-7.33 (m, 2H), 4.44- |
| | | 4.34 (m, 2H), 4.18-4.14 (m, 1H), 4.04- |
| | | 4.02 (m, 1H), 2.55-2.53 (m, 6H), |
| | | 1.94-1.83 (m, 2H). LCMS calcd for; |
| | | C₁₇H₁₉ClFN₅O₄S₂. Found; 476.1 (M + 1). |
| 498 | 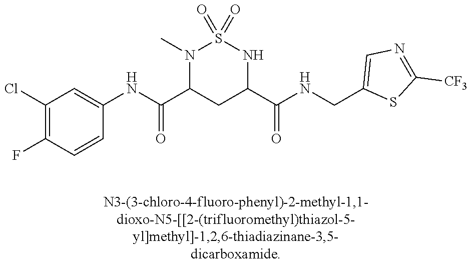 [SMILES: CN1C(C(=O)Nc2ccc(F)c(Cl)c2)CC(C(=O)NCc2cnc(C(F)(F)F)s2)NS1(=O)=O] | ¹H NMR (400 MHz, DMSO-d6): δ 10.53 (s, 1H), 8.88 (t, J = 5.6 Hz, 1H), 8.01 (s, 1H), 7.96-7.93 (m, 1H), 7.56-7.52 (m, 1H), 7.48-7.45 (m, 1H), 7.39 (t, J = 8.8 Hz, 1H), 4.57 (d, J = 5.6 Hz, 2H), 4.21-4.08 (m, 2H), 2.57 (s, 3H), 2.03-1.88 (m, 2H). LCMS calcd for; C₁₇H₁₆ClF₄N₅O₄S₂. Found; 530 (M + 1). |
| 498- | N3-(3-chloro-4-fluoro-phenyl)-2-methyl-1,1- | ¹H NMR (400 MHz, DMSO-d6): δ |
| ISOMER-A | dioxo-N5-[[2-(trifluoromethyl)thiazol-5- | 10.49 (s, 1H), 8.88 (t, J = 6.0 Hz, |
| | yl]methyl]-1,2,6-thiadiazinane-3,5- | 1H), 7.98 (s, 1H), 7.93-7.90 (m, |
| | dicarboxamide. | 1H), 7.53-7.49 (m, 1H), |
| | | 7.43-7.42 (m, 1H), 7.36 |
| | | (t, J = 8.8 Hz, 1H), 4.54 (d, J = 6.0 |
| | | Hz, 2H), 4.17-4.07 (m, 2H), 2.543 (s, |
| | | 3H), 2.00-1.84 (m, 2H). LCMS calcd |
| | | for; C₁₇H₁₆ClF₄N₅O₄S₂. Found; 530 |
| | | (M + 1). |
| 498- | N3-(3-chloro-4-fluoro-phenyl)-2-methyl-1,1- | ¹H NMR (400 MHz, DMSO-d6): δ |
| ISOMER-B | dioxo-N5-[[2-(trifluoromethyl)thiazol-5- | 10.49 (s, 1H), 8.88 (t, J = 5.2 Hz, |
| | yl]methyl]-1,2,6-thiadiazinane-3,5- | 1H), 7.98 (s, 1H), 7.93-7.90 (m, |
| | dicarboxamide. | 1H), 7.53-7.49 (m, 1H), |
| | | 7.43-7.42 (m, 1H), 7.36 |
| | | (t, J = 8.8 Hz, 1H), 4.54 (d, J = 6.0 |
| | | Hz, 2H), 4.18-4.07 (m, 2H), 2.54 (s, |
| | | 3H), 2.00-1.84 (m, 2H). LCMS calcd |
| | | for; C₁₇H₁₆ClF₄N₅O₄S₂. Found; 530 |
| | | (M + 1). |
| 682 | 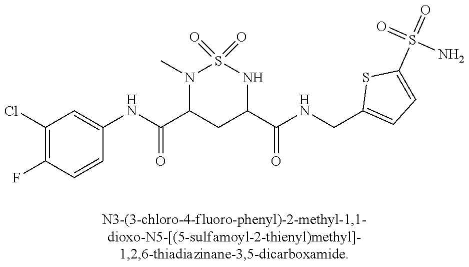 [SMILES: CN1C(C(=O)Nc2ccc(F)c(Cl)c2)CC(C(=O)NCc2ccc(S(N)(=O)=O)s2)NS1(=O)=O] | ¹H NMR (400 MHz, DMSO-d6): δ 10.54 (s, 1H), 8.88-8.86 (m, 1H), 7.99-7.92 (m, 1H), 7.61-7.53 (m, 3H), 7.49-7.31 (m, 3H), 6.99-6.98 (m, 1H), 4.47- 4.45 (m, 2H), 4.23-4.20 (m, 1H), 4.11- 4.09 (m, 1H), 2.57 (s, 3H), 2.00-1.89 (m, 2H). LCMS calcd for; C₁₇H₁₉ClFN₅O₆S₃. Found; 540.20 (M + 1). |
| 684 | 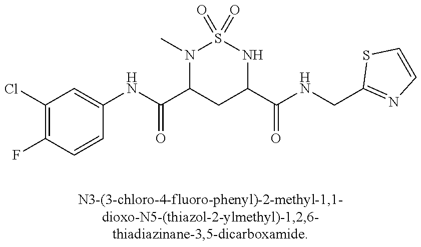 [SMILES: CN1C(C(=O)Nc2ccc(F)c(Cl)c2)CC(C(=O)NCc2nccs2)NS1(=O)=O] | ¹H NMR (400 MHz, DMSO-d6): δ 10.54 (s, 1H), 9.02 (t, J = 6.1 Hz, 1H), 7.96 (dd, J = 6.9, 2.6 Hz, 1H), 7.72 (d, J = 3.3 Hz, 1H), 7.64 (d, J = 3.3 Hz, 1H), 7.57-7.53 (m, 1H), 7.44-7.37 (m, 2H), 4.69-4.49 (m, 2H), 4.31-4.10 (m, 2H), 2.58 (s, 3H), 2.04-1.88 (m, 2H). LCMS calcd for; C₁₆H₁₇ClFN₅O₄S₂. Found; 462.15 (M + 1). |
| 685 | 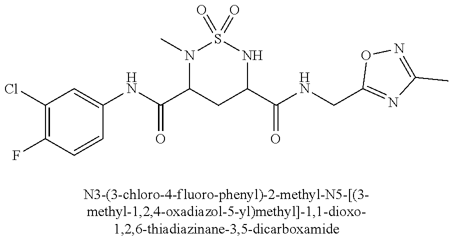 [SMILES: Cc1noc(CNC(=O)C2CC(C(=O)Nc3ccc(F)c(Cl)c3)N(C)S(=O)(=O)N2)n1] | .¹H NMR (400 MHz, DMSO-d6): δ 10.54 (s, 1H), 8.94 (t, J = 5.8 Hz, 1H), 7.95 (dd, J = 6.8, 2.6 Hz, 1H), 7.57-7.53 (m, 1H), 7.48-7.35 (m, 2H), 4.62-4.48 (m, 2H), 4.28-4.11 (m, 2H), 2.57 (s, 3H), 2.32 (s, 3H), 2.06-1.88 (m, 2H). LCMS calcd for; C₁₆H₁₈ClFN₆O₅S. Found; 460.07 (M + 1). |
| 686 | 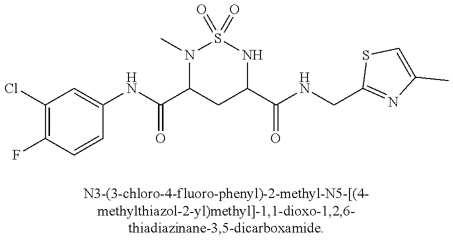 [SMILES: Cc1csc(CNC(=O)C2CC(C(=O)Nc3ccc(F)c(Cl)c3)N(C)S(=O)(=O)N2)n1] | ¹H NMR (400 MHz, DMSO-d6): δ 10.53 (s, 1H), 8.98 (t, J = 6.0 Hz, 1H), 7.96 (dd, J = 6.8, 2.6 Hz, 1H), 7.57- 7.55 (m, 1H), 7.42-7.37 (m, 2H), 7.15 (s, 1H), 4.63-4.43 (m, 2H), 4.26-4.21 (m, 1H), 4.17-4.15 (m, 1H), 2.58 (s, 3H), 2.32 (s, 3H), 2.03-1.87 (m, 2H). LCMS calcd for; C₁₇H₁₉ClFN₅O₄S₂. Found; 476.20 (M + 1). |
| 686- | N3-(3-chloro-4-fluoro-phenyl)-2-methyl-N5-[(4- | ¹H NMR (400 MHz, DMSO-d6): δ |
| ISOMER-A | methylthiazol-2-yl)methyl]-1,1-dioxo-1,2,6- | 10.54 (s, 1H), 8.98 (t, J = |
| | thiadiazinane-3,5-dicarboxamide. | 5.9 Hz, 1H), 7.95 (dd, |
| | | J = 6.9, 2.5 Hz, 1H), 7.56- |
| | | 7.53 (m, 1H), 7.42-7.37 (m, 2H), 7.15 |
| | | (s, 1H), 4.63-4.43 (m, 2H), 4.25-4.15 |
| | | (m, 2H), 2.57 (s, 3H), 2.32 (s, 3H), |
| | | 2.03-1.90 (m, 2H). LCMS calcd for; |
| | | C₁₇H₁₉ClFN₅O₄S₂. Found; 476.20 |
| | | (M + 1). |
| 686- | N3-(3-chloro-4-fluoro-phenyl)-2-methyl-N5-[(4- | ¹H NMR (400 MHz, DMSO-d6): δ |
| ISOMER-B | methylthiazol-2-yl)methyl]-1,1-dioxo-1,2,6- | 10.54 (s, 1H), 8.98 (t, J = 5.9 Hz, |
| | thiadiazinane-3,5-dicarboxamide. | 1H), 7.95 (dd, J = 6.9, |
| | | 2.6 Hz, 1H), 7.57-7.52 (m, |
| | | 1H), 7.42-7.37 (m, 2H), 7.15 |
| | | (s, 1H), 4.63-4.43 (m, 2H), 4.30-4.09 |
| | | (m, 2H), 2.57 (s, 3H), 2.32 (s, 3H), |
| | | 2.03-1.91 (m, 2H). LCMS calcd for; |
| | | C₁₇H₁₉ClFN₅O₄S₂. Found; 476.20 |
| | | (M + 1). |
| 687 | 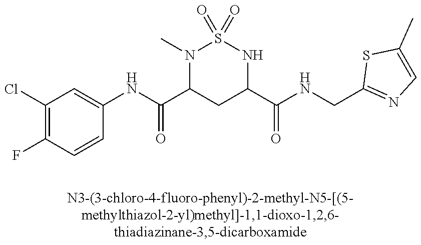 [SMILES: Cc1cnc(CNC(=O)C2CC(C(=O)Nc3ccc(F)c(Cl)c3)N(C)S(=O)(=O)N2)s1] | .¹H NMR (400 MHz, DMSO-d6): δ 10.54 (s, 1H), 8.96 (t, J = 6.0 Hz, 1H), 7.96 (dd, J = 6.8, 2.6 Hz, 1H), 7.56-7.54 (m, 1H), 7.44-7.34 (m, 3H), 4.59-4.39 (m, 2H), 4.30-4.19 (m, 1H), 4.14- 4.12 (m, 1H), 2.58 (s, 3H), 2.40 (s, 3H), 2.02-1.89 (m, 2H). LCMS calcd for; C₁₇H₁₉ClFN₅O₄S₂. Found; 476.20 (M + 1). |
| 687- | N3-(3-chloro-4-fluoro-phenyl)-2-methyl-N5-[(5- | ¹H NMR (400 Mhz, DMSO-d6): δ |
| ISOMER-A | methylthiazol-2-yl)methyl]-1,1-dioxo-1,2,6- | 10.58 (s, 1H), 8.98-8.96 (m, 1H), 7.95 |
| | thiadiazinane-3,5-dicarboxamide. | (dd, J = 6.8, 2.4 Hz, 1H), 7.57-7.53 |
| | | (m, 1H), 7.42-7.36 (m, 3H), 4.63-4.43 |
| | | (m, 2H), 4.30-4.09 (m, 2H), 2.57 (s, |
| | | 3H), 2.32 (s, 3H), 2.03-1.91 (m, |
| | | 2H). LCMS calcd for; |
| | | C₁₇H₁₉ClFN₅O₄S₂. Found; 476.20 |
| | | (M + 1). |
| 687- | N3-(3-chloro-4-fluoro-phenyl)-2-methyl-N5-[(5- | ¹H NMR (400 MHz, DMSO-d6): δ |
| ISOMER-B | methylthiazol-2-yl)methyl]-1,1-dioxo-1,2,6- | 10.53 (s, 1H), 8.95 (t, J = 6.0 Hz, |
| | thiadiazinane-3,5-dicarboxamide. | 1H), 7.96 (dd, J = 6.9, |
| | | 2.5 Hz, 1H), 7.60-7.51 (m, |
| | | 1H), 7.44-7.34 (m, 3H), 4.59- |
| | | 4.39 (m, 2H), 4.20-4.01 (m, 2H), 2.57 |
| | | (s, 3H), 2.39 (s, 3H), 2.02-1.87 (m, |
| | | 2H). LCMS calcd for; |
| | | C₁₇H₁₉ClFN₅O₄S₂. Found; 476.20 |
| | | (M + 1). |
| 689 | 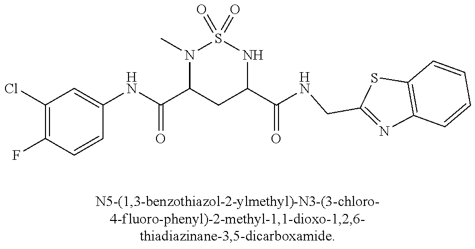 [SMILES: CN1C(C(=O)Nc2ccc(F)c(Cl)c2)CC(C(=O)NCc2nc3ccccc3s2)NS1(=O)=O] | ¹H NMR (400 MHz, DMSO-d6): δ 10.55 (s, 1H), 9.15 (t, J = 6.0 Hz, 1H), 8.08 (d, J = 7.9 Hz, 1H), 8.00-7.91 (m, 2H), 7.60-7.35 (m, 5H), 4.82-4.62 (m, 2H), 4.31-4.16 (m, 2H), 2.59 (s, 3H), 2.10-1.91 (m, 1H). LCMS calcd for; C₂₀H₁₉ClFN₅O₄S₂. Found; 512.1 (M + 1). |
| 689- | N5-(1,3-benzothiazol-2-ylmethyl)-N3-(3-chloro- | ¹H NMR (400 MHz, DMSO-d6): δ |
| ISOMER-A | 4-fluoro-phenyl)-2-methyl-1,1-dioxo-1,2,6- | 10.56 (s, 1H), 9.15 (t, J = 6.1 Hz, |
| | thiadiazinane-3,5-dicarboxamide. | 1H), 8.08 (d, J = 7.9 Hz, 1H), |
| | | 8.01-7.92 (m, 2H), 7.61-7.36 (m, |
| | | 5H), 4.83-4.62 (m, 2H), |
| | | 4.31-4.17 (m, 2H), 2.59 (s, 3H), |
| | | 2.09-1.92 (m, 2H). LCMS calcd for; |
| | | C₂₀H₁₉ClFN₅O₄S₂. Found; |
| | | 512.25 (M + 1). |
| 689- | N5-(1,3-benzothiazol-2-ylmethyl)-N3-(3-chloro- | ¹H NMR (400 MHz, DMSO-d6): δ |
| ISOMER-B | 4-fluoro-phenyl)-2-methyl-1,1-dioxo-1,2,6- | 10.57 (s, 1H), 9.16 (t, J = 6.0 Hz, |
| | thiadiazinane-3,5-dicarboxamide. | 1H), 8.09 (d, J = 7.9 Hz, 1H), |
| | | 7.98-7.94 (m, 2H), 7.61-7.49 (m, |
| | | 3H), 7.45-7.38 (m, 2H), |
| | | 4.82-4.62 (m, 2H), 4.29-4.20 (m, |
| | | 2H), 2.59 (s, 3H), 2.08-1.92 (m, |
| | | 2H). LCMS calcd for; |
| | | C₂₀H₁₉ClFN₅O₄S₂. Found; |
| | | 512.25 (M + 1). |
| 691 | 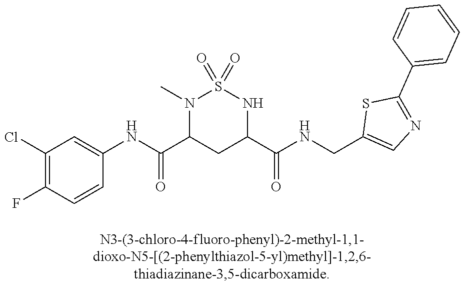 [SMILES: CN1C(C(=O)Nc2ccc(F)c(Cl)c2)CC(C(=O)NCc2cnc(-c3ccccc3)s2)NS1(=O)=O] | ¹H NMR (400 MHz, DMSO-d6): δ 10.53 (s, 1H), 8.85-8.83 (m, 1H), 7.96-7.88 (m, 3H), 7.76 (s, 1H), 7.49-7.36 (m, 6H), 4.52 (d, J = 6.0 Hz, 2H), 4.20-4.10 (m, 2H), 2.57 (s, 3H), 1.99- 1.96 (m, 2H). LCMS calcd for; C₂₂H₂₁ClFN₅O₄S₂. Found; 538 (M + 1). |
| 691- | N3-(3-chloro-4-fluoro-phenyl)-2-methyl-1,1- | ¹H NMR (400 MHz, DMSO-d6): δ |
| ISOMER-A | dioxo-N5-[(2-phenylthiazol-5-yl)methyl]-1,2,6- | 10.55 (s, 1H), 8.85 (t, J = 5.8 Hz, 1H), |
| | thiadiazinane-3,5-dicarboxamide. | 7.99-7.85 (m, 3H), 7.76 (s, 1H), 7.59- |
| | | 7.34 (m, 6H), 4.52 (d, J = 5.8 Hz, 2H), |
| | | 4.25-4.16 (m, 1H), 4.14-4.06 (m, 1H), |
| | | 2.57 (s, 3H), 2.05-1.87 (m, 2H). LCMS |
| | | calcd for; C₂₂H₂₁ClFN₅O₄S₂. Found; |
| | | 538.25 (M + 1). |
| 691- | N3-(3-chloro-4-fluoro-phenyl)-2-methyl-1,1- | ¹H NMR (400 MHz, DMSO-d6): δ |
| ISOMER-B | dioxo-N5-[(2-phenylthiazol-5-yl)methyl]-1,2,6- | 10.55 (s, 1H), 8.85 (t, J = 5.9 Hz, 1H), |
| | thiadiazinane-3,5-dicarboxamide. | 7.96-7.88 (m, 3H), 7.76 (s, 1H), 7.59- |
| | | 7.42 (m, 5H), 7.39 (t, J = 9.1 Hz, 1H), |
| | | 4.52 (d, J = 5.8 Hz, 2H), 4.20-4.17 (m, |
| | | 1H), 4.12-4.08 (m, 1H), 2.57 (s, 3H), |
| | | 2.05-1.86 (m, 2H). LCMS calcd for; |
| | | C₂₂H₂₁ClFN₅O₄S₂. Found; 538.25 |
| | | (M + 1). |
| 704 | 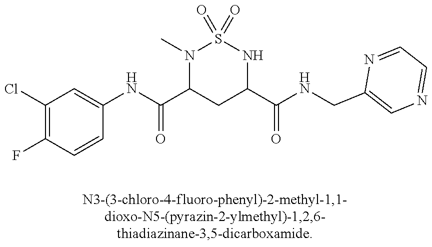 [SMILES: CN1C(C(=O)Nc2ccc(F)c(Cl)c2)CC(C(=O)NCc2cnccn2)NS1(=O)=O] | ¹H NMR (400 MHz, DMSO-d6): δ 10.54 (s, 1H), 8.79 (t, J = 5.9 Hz, 1H), 8.64 (s, 1H), 8.58-8.51 (m, 2H), 7.96 (dd, J = 6.8, 2.5 Hz, 1H), 7.59-7.46 (m, 2H), 7.40 (t, J = 8.8 Hz, 1H), 4.54- 4.37 (m, 2H), 4.22-4.19 (m, 2H), 2.58 (s, 3H), 2.09-1.88 (m, 2H). LCMS calcd for; C₁₇H₁₈ClFN₆O₄S. Found; 457.1 (M + 1) |
| 706 | 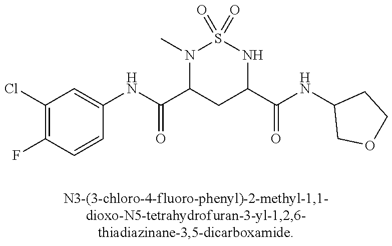 [SMILES: CN1C(C(=O)Nc2ccc(F)c(Cl)c2)CC(C(=O)NC2CCOC2)NS1(=O)=O] | ¹H NMR (400 MHz, DMSO-d6): δ 10.52 (s, 1H), 8.33-8.32 (m, 1H), 7.95 (dd, J = 6.8, 2.6 Hz, 1H), 7.59-7.50 (m, 1H), 7.40 (t, J = 9.1 Hz, 1H), 7.30- 7.23 (m, 1H), 4.31-4.15 (m, 2H), 4.08- 4.07 (m, 1H), 3.84-3.61 (m, 3H), 3.51- 3.46 (m, 1H), 2.56 (s, 3H), 2.12-2.05 (m, 1H), 2.04-1.90 (m, 2H), 1.79-1.75 (m, 1H). LCMS calcd for; C₁₆H₂₀ClFN₄O₅S. Found; 435.20 (M + 1). |
| 708 | 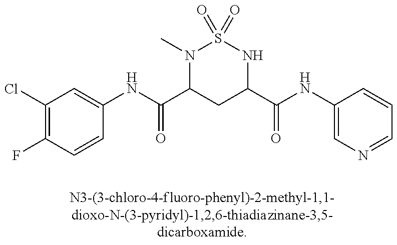 [SMILES: CN1C(C(=O)Nc2ccc(F)c(Cl)c2)CC(C(=O)Nc2cccnc2)NS1(=O)=O] | ¹H NMR (400 MHz, DMSO-d6): δ 10.57 (s, 1H), 10.36 (s, 1H), 8.80 (d, J = 2.6 Hz, 1H), 8.30 (d, J = 4.8 Hz, 1H), 8.08-8.06 (m, 1H), 7.96 (dd, J = 6.9, 2.7 Hz, 1H), 7.62-7.52 (m, 2H), 7.45-7.33 (m, 2H), 4.39-4.24 (m, 2H), 2.60 (s, 3H), 2.07-2.03 (m, 1H). LCMS calcd for; C₁₇H₁₇ClFN₅O₄S. Found; 442.40 (M + 1). |
| 710 | 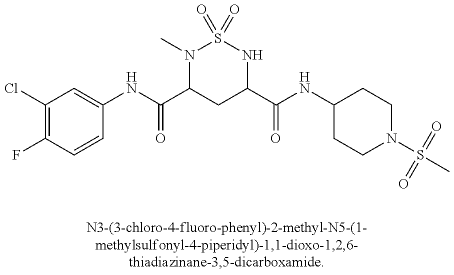 [SMILES: CN1C(C(=O)Nc2ccc(F)c(Cl)c2)CC(C(=O)NC2CCN(S(C)(=O)=O)CC2)NS1(=O)=O] | ¹H NMR (400 MHz, DMSO-d6): δ 10.54 (s, 1H), 8.12 (d, J = 7.6 Hz, 1H), 7.96-7.94 (m, 1H), 7.57-7.52 (m, 1H), 7.39 (t, J = 9.2 Hz, 1H), 7.30- 7.28 (m, 1H), 4.21-4.17 (m, 1H), 4.06-4.05 (m, 1H), 3.72-3.67 (m, 1H), 3.51-3.48 (m, 2H), 2.91-2.80 (m, 5H), 2.57 (s, 3H), 1.95-1.90 (m, 2H), 1.81- 1.80 (m, 2H), 1.54-1.43 (m, 2H). LCMS calcd for; C₁₈H₂₅ClFN₅O₆S₂. Found; 426.30 (M + 1). |
| 711 | 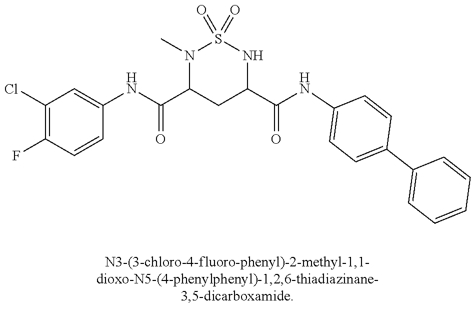 [SMILES: CN1C(C(=O)Nc2ccc(F)c(Cl)c2)CC(C(=O)Nc2ccc(-c3ccccc3)cc2)NS1(=O)=O] | ¹H NMR (400 MHz, DMSO-d6): δ 10.57 (s, 1H), 10.26 (s, 1H), 7.97 (dd, J = 6.9, 2.6 Hz, 1H), 7.74 (d, J = 8.6 Hz, 2H), 7.69-7.50 (m, 6H), 7.49-7.29 (m, 4H), 4.35-4.27 (m, 2H), 2.61 (s, 3H), 2.10-1.98 (m, 2H). LCMS calcd for; C₂₄H₂₂ClFN₄O₄S. Found; 517.1 (M + 1). |
| 715 | 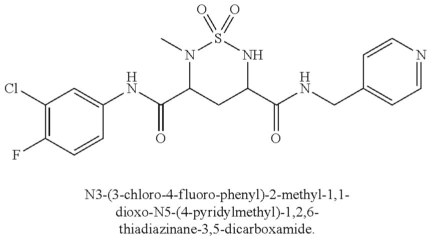 [SMILES: CN1C(C(=O)Nc2ccc(F)c(Cl)c2)CC(C(=O)NCc2ccncc2)NS1(=O)=O] | ¹H NMR (400 MHz, DMSO-d6): δ 10.57 (s, 1H), 8.76 (t, J = 6.2 Hz, 1H), 8.51 (d, J = 5.0 Hz, 2H), 7.98-7.97 (m, 1H), 7.61-7.48 (m, 2H), 7.42 (t, J = 9.1 Hz, 1H), 7.30 (d, J = 5.0 Hz, 2H), 4.35-4.30 (m, 2H), 4.23-4.18 (m, 2H), 2.60 (s, 3H), 2.10-1.91 (m, 2H). LCMS calcd for; C₁₈H₁₉ClFN₅O₄S. Found; 456 (M + 1). |
| 716 | 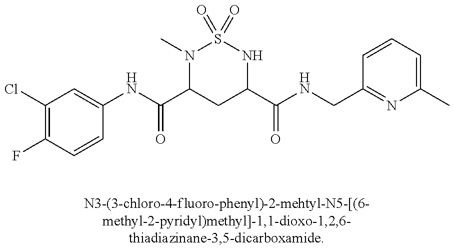 [SMILES: Cc1cccc(CNC(=O)C2CC(C(=O)Nc3ccc(F)c(Cl)c3)N(C)S(=O)(=O)N2)n1] | ¹H NMR (400 MHz, DMSO-d6): δ 10.54 (s, 1H), 8.68 (t, J = 5.9 Hz, 1H), 7.96 (dd, J = 6.9, 2.6 Hz, 1H), 7.64 (t, J = 7.7 Hz, 1H), 7.57-7.53 (m, 1H), 7.40 (t, J = 8.7 Hz, 2H), 7.11 (d, J = 7.7 Hz, 2H), 4.34 (d, J = 5.9 Hz, 2H), 4.22-4.18 (m, 2H), 2.58 (s, 3H), 2.44 (s, 3H), 2.08-1.89 (m, 2H). LCMS calcd for; C₁₉H₂₁ClFN₅O₄S. Found; 470.25 (M + 1). |
| 716- | N3-(3-chloro-4-fluoro-phenyl)-2-methyl-N5-[(6- | ¹H NMR (400 MHz, DMSO-d6): δ |
| ISOMER-A | methyl-2-pyridyl)methyl]-1,1-dioxo-1,2,6- | 10.54 (s ,1H), 8.67 (t, J = 6.0 Hz, 1H), |
| | thiadiazinane-3,5-dicarboxamide. | 7.96 (dd, J = 6.9, 2.5 Hz, 1H), 7.64 (t, |
| | | J = 7.7 Hz, 1H), 7.59-7.50 (m, 1H), |
| | | 7.42-7.37 (m, 2H), 7.12-7.10 (m, 2H), |
| | | 4.34 (d, J = 5.8 Hz, 2H), 4.22-4.20 (m, |
| | | 2H), 2.58 (s, 3H), 2.44 (s, 3H), 2.07- |
| | | 1.93 (m, 2H). LCMS calcd for; |
| | | C₁₉H₂₁ClFN₅O₄S. Found; |
| | | 470.20 (M + 1). |
| 716- | N3-(3-chloro-4-fluoro-phenyl)-2-methyl-N5-[(6- | ¹H NMR (400 MHz, DMSO-d6): δ |
| ISOMER-B | methyl-2-pyridyl)methyl]-1,1-dioxo-1,2,6- | 10.54 (s, 1H), 8.67 (t, J = 5.9 Hz, 1H), |
| | thiadiazinane-3,5-dicarboxamde. | 7.96 (dd, J = 6.8, 2.6 Hz, 1H), 7.64 (t, |
| | | J = 7.7 Hz, 1H), 7.57-7.52 (m, 1H), |
| | | 7.40 (t, J = 9.1 Hz, 2H), 7.15-7.08 (m, |
| | | 2H), 4.34 (d, J = 5.9 Hz, 2H), 4.22- |
| | | 4.20 (m, 2H), 2.58 (s, 3H), 2.44 (s, |
| | | 3H), 2.07-1.89 (m, 2H). LCMS calcd |
| | | for; C₁₉H₂₁ClFN₅O₄S. Found; |
| | | 470.25 (M + 1). |
| 717 | 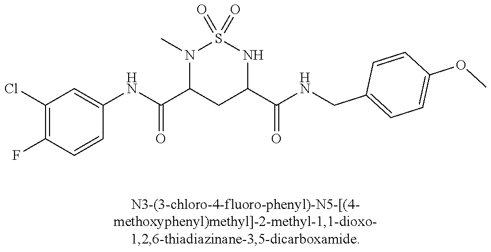 [SMILES: COc1ccc(CNC(=O)C2CC(C(=O)Nc3ccc(F)c(Cl)c3)N(C)S(=O)(=O)N2)cc1] | N3-(3-chloro-4-fluoro-phenyl)-N5-[(4- methoxyphenyl)methyl]-2-methyl-1,1- dioxo-1,2,6-thiadiazinane-3,5- dicarboxamide. ¹H NMR (400 MHz, DMSO-d6): δ 10.53 (s, 1H), 8.56 (t, J = 6.0 Hz, 1H), 7.95 (dd, J = 6.8, 2.6 Hz, 1H), 7.55-7.53 (m, 1H), 7.42-7.36 (m, 2H), 7.19 (d, J = 8.2 Hz, 2H), 6.88 (d, J = 8.1 Hz, 2H), 4.23-4.17 (m, 3H), 4.11 (t, J = 7.8 Hz, 1H), 3.72 (s, 3H), 2.57 (s, 3H), 1.97-1.89 (m, 2H). LCMS calcd for; C₂₀H₂₂ClFN₄O₅S. Found; 485.25 (M + 1). |
| 717- | N3-(3-chloro-4-fluoro-phenyl)-N5-[(4- | ¹H NMR (400 MHz, DMSO-d6): δ |
| ISOMER-A | methoxyphenyl)methyl]-2-methyl-1,1-dioxo- | 10.53 (s, 1H), 8.56 (t, J = 5.6 Hz, 1H), |
| | 1,2,6-thiadiazinane-3,5-dicarboxamide. | 7.96 (dd, J = 6.9, 2.6 Hz, 1H), 7.56- |
| | | 7.54 (m, 1H), 7.42-7.36 (m, 2H), 7.20 |
| | | (d, J = 8.2 Hz, 2H), 6.88 (d, J = 8.4 |
| | | Hz, 2H), 4.24-4.18 (m, 2H), 4.12-4.08 |
| | | (m, 2H), 3.73 (s, 3H), 2.57 (s, 3H), |
| | | 1.98-1.92 (m, 2H). LCMS calcd for; |
| | | C₂₀H₂₂ClFN₄O₅S. Found; 485.25 |
| | | (M + 1). |
| 717- | N3-(3-chloro-4-fluoro-phenyl)-N5-[(4- | ¹H NMR (400 MHz, DMSO-d6): δ |
| ISOMER-B | methoxyphenyl)methyl]-2-methyl-1,1-dioxo- | 10.29 (s, 1H), 8.25-8.15 (m, 1H), 7.97 |
| | 1,2,6-thiadiazinane-3,5-dicarboxamide. | (dd, J = 6.9, 2.6 Hz, 1H), 7.58-7.56 |
| | | (m, 1H), 7.42-7.37 (m, 1H), 7.18 (d, J = |
| | | 8.1 Hz, 2H), 6.87 (d, J = 8.4 Hz, |
| | | 2H), 4.21 (t, J = 5.9 Hz, 2H), 3.85- |
| | | 3.54 (m, 2H), 3.73 (s, 3H), 2.40 (s, |
| | | 3H), 1.99-1.95 (m, 2H). NH protons |
| | | not observed. LCMS calcd for; |
| | | C₂₀H₂₂ClFN₄O₅S. Found; 485.25 |
| | | (M + 1). |
| 718 | 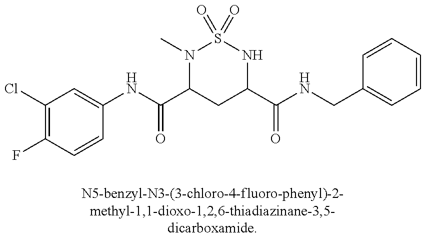 [SMILES: CN1C(C(=O)Nc2ccc(F)c(Cl)c2)CC(C(=O)NCc2ccccc2)NS1(=O)=O] | .¹H NMR (400 MHz, DMSO-d6): δ 10.53 (s, 1H), 8.63-8.60 (m, 1H), 7.96- 7.93 (m, 1H), 7.54-7.52 (m, 1H), 7.38 (t, J = 9.2 Hz, 1H), 7.33-7.22 (m, 6H), 4.29 (d, J = 6.0 Hz, 2H), 4.21-4.10 (m, 2H), 2.56 (s, 3H), 1.98-1.96 (m, 2H); LCMS calcd for; C₁₉H₂₀ClFN₄O₄S. Found; 455.20 (M + 1). |
| 718- | N5-benzyl-N3-(3-chloro-4-fluoro-phenyl)-2- | ¹H NMR (400 MHz, DMSO-d6): δ |
| ISOMER-A | methyl-1,1-dioxo-1,2,6-thiadiazinane-3,5- | 10.53 (bs, 1H), 8.65-8.63 (m, 1H), |
| | dicarboxamide. | 7.95 (dd, J = 7.0, 2.6 Hz, 1H), 7.57- |
| | | 7.53 (m, 1H), 7.42-7.37 (m, 2H), 7.34- |
| | | 7.23 (m, 5H), 4.30 (d, J = 5.9 Hz, 2H), |
| | | 4.20-4.13 (m, 2H), 12.57 (s, 3H), 1.99- |
| | | 1.96 (m, 2H). LCMS calcd for; |
| | | C₁₉H₂₀ClFN₄O₄S. Found; 455.20 |
| | | (M + 1). |
| 718- | N5-benzyl-N3-(3-chloro-4-fluorophenyl)-2- | ¹H NMR (400 MHz, DMSO-d6): δ |
| ISOMER-B | methyl-1,1-dioxo-1,2,6-thiadiazinane-3,5- | 10.49 (s, 1H), 8.57-8.55 (m, 1H), 7.96 |
| | dicarboxamide. | (dd, J = 6.9, 2.6 Hz, 1H), 7.57-7.53 |
| | | (m, 1H), 7.43-7.419 (m, 7H), 4.30 (d, |
| | | J = 6.0 Hz, 2H), 4.10-4.06 (m, 2H), 2.54 |
| | | (s, 3H), 2.03-1.81 (m, 1H). LCMS |
| | | calcd for; C₁₉H₂₀ClFN₄O₄S. Found; |
| | | 455.20 (M + 1). |
| 719 | 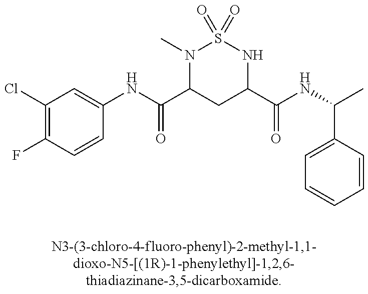 [SMILES: C[C@@H](NC(=O)C1CC(C(=O)Nc2ccc(F)c(Cl)c2)N(C)S(=O)(=O)N1)c1ccccc1] | ¹H NMR (400 MHz, DMSO-d6): δ 10.53 (br.s, 1H), 8.57-8.53 (m, 1H), 7.97-7.94 (m, 1H), 7.54-7.52 (m, 1H), 7.41-7.22 (m, 7H), 4.95-4.90 (m, 1H), 4.21-4.12 (m, 2H), 2.56 (s, 3H), 1.96- 1.87 (m, 2H), 1.38-1.36 (m, 3H). LCMS calcd for; C₂₀H₂₂ClFN₄O₄S. Found; 469.25 (M + 1). |
| 719- | N3-(3-chloro-4-fluoro-phenyl)-2-methyl-1,1- | ¹H NMR (400 MHz, DMSO-d6): δ |
| ISOMER-A | dioxo-N5-[(1R)-1-phenylethyl]-1,2,6- | 10.53 (s, 1H), 8.54-8.51 (m, 1H), 7.95 |
| | thiadiazinane-3,5-dicarboxamide. | (dd, J = 6.9, 2.6 Hz, 1H), 7.59-7.50 |
| | | (m, 1H), 7.42-7.37 (m, 1H), 7.35-73.0 |
| | | (m, 4H), 7.26-7.23 (m, 2H), 4.92 (t, J = |
| | | 7.2 Hz, 1H), 4.20-4.12 (m, 2H), |
| | | 2.56 (s, 3H), 2.01-1.88 (m, 2H), 1.37 |
| | | (d, J = 7.0 Hz, 3H). LCMS calcd for; |
| | | C₂₀H₂₂ClFN₄O₄S. Found; 469.25 |
| | | (M + 1). |
| 719- | N3-(3-chloro-4-fluoro-phenyl)-2-methyl-1,1- | ¹H NMR (400 MHz, DMSO-d6):: δ |
| ISOMER-B | dioxo-N5-[(1R)-1-phenylethyl]-1,2,6- | 10.51 (s, 1H), 8.55-8.53 (m, 1H), 7.95 |
| | thiadiazinane-3,5-dicarboxamide. | (dd, J = 6.8, 2.6 Hz, 1H), 7.56-7.52 |
| | | (m, 1H), 7.38 (t, J = 9.2 Hz, 1H), |
| | | 7.32-7.30 (m, 4H), 7.24-7.21 (m, 2H), |
| | | 4.92 (t, J = 7.3 Hz, 1H), 4.20-4.11 (m, |
| | | 2H), 2.55 (s, 3H), 1.89-1.87 (m, 2H), |
| | | 1.38 (d, J = 7.0 Hz, 3H). LCMS calcd |
| | | for; C₂₀H₂₂ClFN₄O₄S. Found; 469.25 |
| | | (M + 1). |
| 721 | 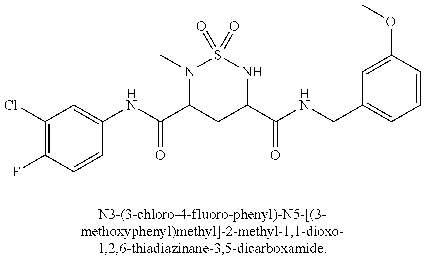 [SMILES: COc1cccc(CNC(=O)C2CC(C(=O)Nc3ccc(F)c(Cl)c3)N(C)S(=O)(=O)N2)c1] | ¹H NMR (400 MHz, DMSO-d6): δ 10.53 (s, 1H), 8.62 (t, J = 6.0 Hz, 1H), 7.95 (dd, J = 6.9, 2.6 Hz, 1H), 7.55- 7.53 (m, 1H), 7.44-7.30 (m, 2H), 7.23 (t, J = 8.0 Hz, 1H), 6.88-6.77 (m, 3H), 4.27 (d, J = 6.0 Hz, 2H), 4.23-4.09 (m, 2H), 3.73 (s, 3H), 2.58 (s, 3H), 2.04-1.90 (m, 2H). LCMS calcd for; C₂₀H₂₂ClFN₄O₅S. Found; 485 (M + 1). |
| 721- | N3-(3-chloro-4-fluoro-phenyl)-N5-[(3- | ¹H NMR (400 MHz, DMSO-d6): δ |
| ISOMER-A | methoxyphenyl)methyl]-2-methyl-1,1-dioxo- | 10.33 (s, 1H), 8.32 (s, 1H), 7.97 (dd, J = |
| | 1,2,6-thiadiazinane-3,5-dicarboxamide. | 6.9, 2.6 Hz, 1H), 7.59-7.55 (m, 1H), |
| | | 7.38 (t, J = 8.8 Hz, 2H), 7.23 (t, J = |
| | | 7.9 Hz, 1H), 6.8-6.77 (m, 3H), 4.34- |
| | | 4.19 (m, 2H), 3.95-3.87 (m, 2H), 3.74 |
| | | (s, 3H), 2.42 (s, 3H), 2.00-1.97 (m, |
| | | 2H). LCMS calcd for; |
| | | C₂₀H₂₂ClFN₄O₅S. Found; |
| | | 485.25 (M + 1). |
| 721- | N3-(3-chloro-4-fluoro-phenyl)-N5-[(3- | ¹H NMR (400 MHz, DMSO-d6): δ |
| ISOMER-B | methoxyphenyl)methyl]-2-methyl-1,1-dioxo- | 10.52 (s, 1H), 8.61 (t, J = 6.1 Hz, 1H), |
| | 1,2,6-thiadiazinane-3,5-dicarboxamide. | 7.95 (dd, J = 6.9, 2.6 Hz, 1H), 7.56- |
| | | 7.52 (m, 1H), 7.39 (t, J = 9.1 Hz, 2H), |
| | | 7.23 (t, J = 8.0 Hz, 1H), 6.88-6.77 (m, |
| | | 3H), 4.24 (d, J = 6.0 Hz, 2H), 4.22- |
| | | 4.18 (m, 1H), 4.15-4.13 (m, 1H), 3.73 |
| | | (s, 3H), 2.57 (s, 3H), 1.99-1.90 (m, |
| | | 2H). LCMS calcd for; |
| | | C₂₀H₂₂ClFN₄O₅S. Found; 485.25 |
| | | (M + 1). |
| 724 | 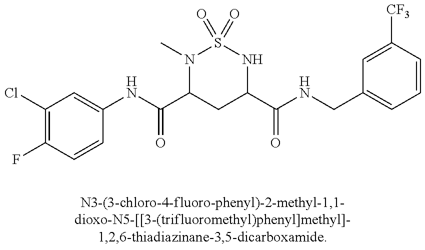 [SMILES: CN1C(C(=O)Nc2ccc(F)c(Cl)c2)CC(C(=O)NCc2cccc(C(F)(F)F)c2)NS1(=O)=O] | ¹H NMR (400 MHz, DMSO-d6): δ 10.54 (s, 1H), 8.73 (t, J = 6.0 Hz, 1H), 7.96-7.94 (m, 1H), 7.64-7.52 (m, 5H), 7.42-7.39 (m, 2H), 4.42-4.35 (m, 2H), 4.21-4.13 (m, 2H), 2.58 (s, 3H), 2.04- 1.91 (m, 2H). LCMS calcd for; C₂₀H₁₉ClF₄N₄O₄S. Found; 523.30 (M + 1). |
| 725 | 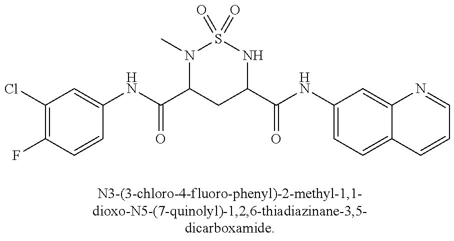 [SMILES: CN1C(C(=O)Nc2ccc(F)c(Cl)c2)CC(C(=O)Nc2ccc3cccnc3c2)NS1(=O)=O] | ¹H NMR (400 MHz, DMSO-d6): δ 10.58 (s, 1H), 10.51 (s, 1H), 8.85 (dd, J = 4.2, 1.7 Hz, 1H), 8.46 (d, J = 2.0 Hz, 1H), 8.32-8.25 (m, 1H), 8.01-7.90 (m, 2H), 7.78 (dd, J = 8.9, 2.1 Hz, 1H), 7.63-7.51 (m, 2H), 7.48-7.36 (m, 2H), 4.39-4.37 (m, 1H), 4.31 (dd, J = 10.9, 4.1 Hz, 1H), 2.62 (s, 3H), 2.19- 2.02 (m, 2H). LCMS calcd for; C₂₁H₁₉ClFN₅O₄S. Found; 492.1 (M + 1). |
| 726 | 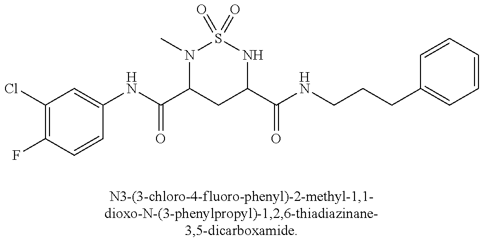 [SMILES: CN1C(C(=O)Nc2ccc(F)c(Cl)c2)CC(C(=O)NCCCc2ccccc2)NS1(=O)=O] | ¹H NMR (400 MHz, DMSO-d6): δ 10.52 (s, 1H), 8.13 (t, J = 5.,7 Hz, 1H), 7.95 (dd, J = 6.8, 2.5 Hz, 1H), 7.56- 7.52 (m, 1H), 7.39 (t, J = 9.1 Hz, 1H), 7.33-7.12 (m, 6H), 4.23-4.13 (m, 1H), 4.07-4.05 (m, 1H), 3.10 (q, J = 6.6 Hz, 2H), 2.59-2.55 (m, 5H), 1.99-1.89 (m, 2H), 1.74-1.70 (m, 2H). LCMS calcd for; C₂₁H₂₄ClFN₄O₄S. Found; 483.1 (M + 1). |
| 728 | 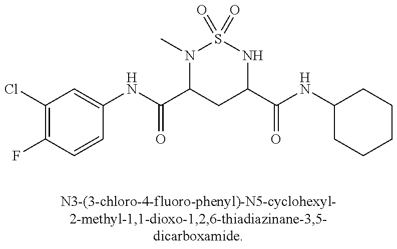 [SMILES: CN1C(C(=O)Nc2ccc(F)c(Cl)c2)CC(C(=O)NC2CCCCC2)NS1(=O)=O] | ¹H NMR (400 MHz, DMSO-d6): δ 10.52 (s, 1H), 7.99-7.90 (m, 2H), 7.56- 7.52 (m, 1H), 7.40 (t, J = 9.1 Hz, 1H), 7.20-71.8 (m, 1H), 4.18 (dd, J = 11.1, 3.9 Hz, 1H), 4.05-4.03 (m, 1H), 3.55- 3.52 (m, 1H), 2.56 (s, 3H), 2.00-1.82 (m, 2H), 1.72-1.65 (m, 4H), 1.56-1.53 (m, 1H), 1.34-1.05 (m, 5H). LCMS calcd for; C₁₈H₂₄ClFN₄O₄S. Found; 447.1 (M + 1). |
| 729 | 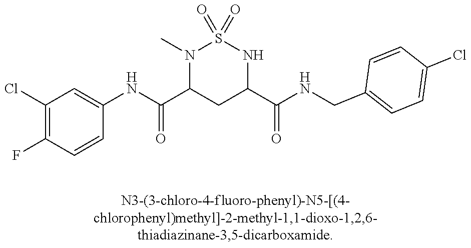 [SMILES: CN1C(C(=O)Nc2ccc(F)c(Cl)c2)CC(C(=O)NCc2ccc(Cl)cc2)NS1(=O)=O] | ¹H NMR (400 MHz, DMSO-d6): δ 10.53 (s, 1H), 8.67 (t, J = 6.1 Hz, 1H), 7.95 (dd, J = 6.9, 2.5 Hz, 1H), 7.55- 7.52 (m, 1H), 7.45-7.32 (m, 4H), 7.33- 7.26 (m, 2H), 4.28 (d, J = 6.0 Hz, 2H), 4.21-4.09 (m, 2H), 2.58 (s, 3H), 2.04-1.88 (m, 2H). LCMS calcd for; C₁₉H₁₉Cl₂FN₄O₄S. Found; 489 (M + 1). |
| 729- | N3-(3-chloro-4-fluoro-phenyl)-N5-[(4- | .¹H NMR (400 MHz, DMSO-d6): δ |
| ISOMER-A | chlorophenyl)methyl]-2-methyl-1,1-dioxo-1,2,6- | 10.50 (s, 1H), 8.63 (t, J = 5.4 Hz, 1H), |
| | thiadiazinane-3,5-dicarboxamide | 7.94-7.92 (m, 1H), 7.57-7.48 (m, 1H), |
| | | 7.42-7.32 (m, 4H), 7.28 (d, J = 8.1 Hz, |
| | | 2H), 4.30-4.23 (m, 2H), 4.18-4.08 (m, |
| | | 2H), 2.55 (s, 3H), 2.02-1.88 (m, |
| | | 2H). LCMS calcd for; |
| | | C₁₉H₁₉Cl₂FN₄O₄S. FOund; 489.20 |
| | | (M + 1). |
| 729- | N3-(3-chloro-4-fluoro-phenyl)-N5-[(4- | ¹H NMR (400 MHz, DMSO-d6): δ |
| ISOMER-B | chlorophenyl)methyl]-2-methyl-1,1-dioxo-1,2,6- | 10.52 (s, 1H), 8.68-8.60 (m, 1H), 7.97- |
| | thiadiazinane-3,5-dicarboxamide. | 7.90 (m, 1H), 7.52-7.50 (m, 1H), |
| | | 7.43-7.32 (m, 4H), 7.29-7.26 (m, 2H), |
| | | 4.27-4.26 (m, 2H), 4.21-4.07 (m, 2HH), |
| | | 2.48 (s, 3H), 1.97-1.91 (m, 2H). LCMS |
| | | calcd for; C₁₉H₁₉Cl₂FN₄O₄S. FOund; |
| | | 489.20 (M + 1). |
| 730 | 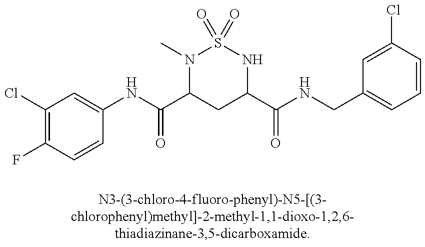 [SMILES: CN1C(C(=O)Nc2ccc(F)c(Cl)c2)CC(C(=O)NCc2cccc(Cl)c2)NS1(=O)=O] | ¹H NMR (400 MHz, DMSO-d6): δ 10.54 (s, 1H), 8.67 (t, J = 6.1 Hz, 1H), 7.96 (dd, J = 6.9, 2.6 Hz, 1H), 7.57- 7.52 (m, 1H), 7.49-7.21 (m, 6H), 4.36- 4.24 (m, 2H), 4.21-4.13 (m, 2H), 2.58 (s, 3H), 2.07-1.88 (m, 2H). LCMS calcd for; C₁₉H₁₉Cl₂FN₄O₄S. Found; 489 (M + 1). |
| 730- | N3-(3-chloro-4-fluoro-phenyl)-N5-[(3- | ¹H NMR (400 MHz, DMSO-d6): δ |
| ISOMER-A | chlorophenyl)methyl]-2-methyl-1,1-dioxo-1,2,6- | 10.54 (s, 1H), 8.67 (t, J = 6.0 Hz, 1H), |
| | thiadiazinane-3,5-dicarboxamide. | 7.97-7.93 (m, 1H), 7.59-7.44 (m, 2H), |
| | | 7.44-7.20 (m, 5H), 4.36-4.24 (m, 2H), |
| | | 4.20-4.12 (m, 2H), 2.58 (s, 3H), 2.03- |
| | | 1.98 (m, 2H) LCMS calcd for; |
| | | C₁₉H₁₉Cl₂FN₄O₄S. Found; 489.20 |
| | | (M + 1). |
| 730- | N3-(3-chloro-4-fluorophenyl)-N5-[(3- | ¹H NMR (400 MHz, DMSO-d6): δ |
| ISOMER-B | chlorophenyl)methyl]-2-methyl-1,1-dioxo-1,2,6- | 10.53 (s, 1H), 8.67 (t, J = 6.1 Hz, 1H), |
| | thiadiazinane-3,5-dicarboxamide. | 7.96-7.94 (m, 1H), 7.59-7.45 (m, 2H), |
| | | 7.46-7.20 (m, 5H), 4.38-4.10 (m, 4H), |
| | | 2.58 (s, 3H), 2.02-1.90 (m, 2H). LCMS |
| | | calcd for; C₁₉H₁₉Cl₂FN₄OS. Found; |
| | | 489.20 (M + 1). |
| 731 | 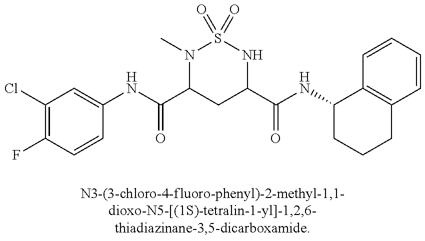 [SMILES: CN1C(C(=O)Nc2ccc(F)c(Cl)c2)CC(C(=O)N[C@H]2CCCc3ccccc32)NS1(=O)=O] | ¹H NMR (400 MHz, DMSO-d6): δ 10.54 (s, 1H), 8.50-8.48 (m, 1H), 7.97- 7.95 (m, 1H), 7.56-7.53 (m, 1H), 7.40 (t, J = 9.0 Hz, 1H), 7.29-7.06 (m, 5H), 5.02-4.92 (m, 1H), 4.25-4.09 (m, 2H), 2.74-2.72 (m, 2H), 2.58 (s, 3H), 2.09- 1.80 (m, 4H), 1.78-1.66 (m, 2H). LCMS calcd for; C₂₂H₂₄ClFN₄O₄S. Found; 495.30 (M + 1). |
| 732 | 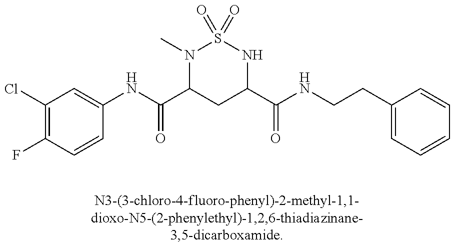 [SMILES: CN1C(C(=O)Nc2ccc(F)c(Cl)c2)CC(C(=O)NCCc2ccccc2)NS1(=O)=O] | ¹H NMR (400 MHz, DMSO-d6): δ 10.51 (s, 1H), 8.19 (t, J = 5.7 Hz, 1H), 7.95 (dd, J = 6.9, 2.6 Hz, 1H), 7.57- 7.52 (m, 1H), 7.40 (t, J = 9.1 Hz, 1H), 7.30-7.28 (m, 3H), 7.27-7.17 (m, 3H), 4.20-4.16 (m, 1H), 4.04-4.02 (m, 1H), 3.42-3.21 (m, 2H), 2.74 (t, J = 7.4 Hz, 2H), 2.56-(s, 3H), 1.94-1.85 (m, 2H). LCMS calcd for; C₂₀H₂₂ClFN₄O₄S. Found; 469.25 (M + 1). |
| 734 | 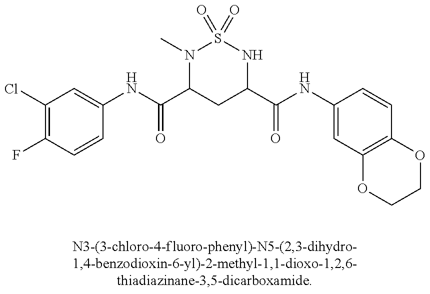 [SMILES: CN1C(C(=O)Nc2ccc(F)c(Cl)c2)CC(C(=O)Nc2ccc3c(c2)OCCO3)NS1(=O)=O] | ¹H NMR (400 MHz, DMSO-d6): δ 10.55 (s, 1H), 9.98 (s, 1H), 7.97-7.94 (m, 1H), 7.58-7.54 (m, 1H), 7.47-7.38 (m, 2H), 7.25 (s, 1H), 7.04-7.01 (m, 1H), 6.79 (d, J = 8.8 Hz, 1H), 4.27- 4.20 (m, 6H), 2.59 (s, 3H), 2.05-1.95 (m, 2H). LCMS calcd for; C₂H₂₀ClFN₄O₆S. Found; 499.25 (M + 1). |
| 736 | 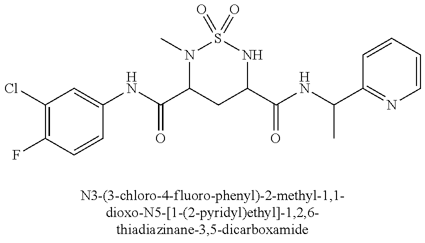 [SMILES: CC(NC(=O)C1CC(C(=O)Nc2ccc(F)c(Cl)c2)N(C)S(=O)(=O)N1)c1ccccn1] | ¹H NMR (400 MHz, DMSO-d6): δ 10.53 (s, 1H), 8.60-8.48 (m, 2H), 7.95 (dd, J = 6.9, 2.6 Hz, 1H), 7.78-7.73 (m, 1H), 7.56-7.52 (m, 1H), 7.45-7.22 (m, 4H), 4.97 (p, J = 7.2 Hz, 1H), 4.21-4.18 (m, 2H), 2.56 (s, 3H), 2.01- 1.81 (m, 2H), 1.41-1.36 (m, 3H). LCMS calcd for; C₁₉H₂₁ClFN₅O₄S. Found; 470 (M + 1). |
| 738 | 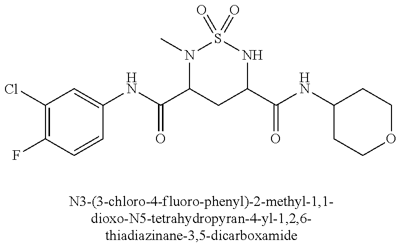 [SMILES: CN1C(C(=O)Nc2ccc(F)c(Cl)c2)CC(C(=O)NC2CCOCC2)NS1(=O)=O] | ¹H NMR (400 MHz, DMSO-d6): δ 10.53 (s, 1H), 8.07 (d, J = 7.6 Hz, 1H), 7.95 (dd, J = 6.8 Hz, 2.4 Hz, 1H), 7.57-7.52 (m, 1H), 7.40 (t, J = 8.8 Hz, 1H), 7.28-7.25 (m, 1H), 4.21-4.17 (m, 1H), 4.07-4.03 (m, 1H), 3.83-3.75 (m, 3H), 3.37-3.33 (m, 2H), 2.57 (s, 3H), 1.95-1.90 (m, 2H), 1.68-1.65 (m, 2H), 1.47-1.40 (m, 2H). LCMS calcd for; C₁₇H₂₂ClFN₄O₅S. Found; 449.25 (M + 1). |
| 739 | 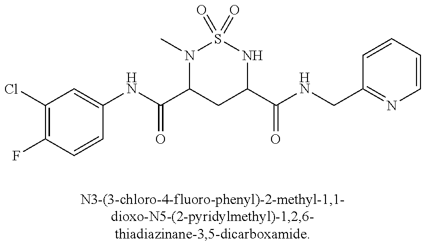 [SMILES: CN1C(C(=O)Nc2ccc(F)c(Cl)c2)CC(C(=O)NCc2ccccn2)NS1(=O)=O] | ¹H NMR (400 MHz, DMSO-d6): δ 10.54 (s, 1H), 8.69 (t, J = 6.0 Hz, 1H), 8.53-8.47 (m, 1H), 7.96 (dd, J = 6.9, 2.6 Hz, 1H), 7.78-7.74 (m, 1H), 7.57- 7.53 (m, 1H), 7.44-7.37 (m, 2H), 7.34- 7.31 (m, 1H), 7.28-7.24 (m, 1H), 4.40 (d, J = 5.9 Hz, 2H), 4.23-4.19 (m, 2H), 2.58 (s, 3H), 2.08-1.89 (m, 2H). LCMS calcd for; C₁₈H₁₉ClFN₅O₄S. Found; 456.0 (M + 1). |
| 740 | 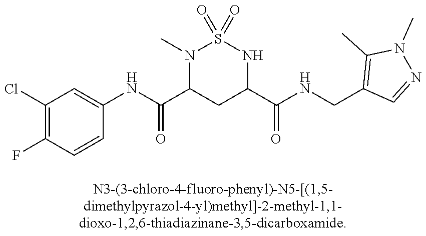 [SMILES: Cc1c(CNC(=O)C2CC(C(=O)Nc3ccc(F)c(Cl)c3)N(C)S(=O)(=O)N2)cnn1C] | ¹H NMR (400 MHz, DMSO-d6): δ 10.51 (s, 1H), 8.31-7.28 (m, 1H), 7.96- 7.94 (m, 1H), 7.55-7.52 (m, 1H), 7.39 (t, J = 9.2 Hz, 1H), 7.29-7.23 (m, 2H), 4.20-4.15 (m, 1H), 4.06-4.05 (m, 3H), 3.67 (s, 3H), 2.56 (s, 3H), 2.19 (s, 3H), 1.94-1.89 (m, 2H). LCMS calcd for; C₁₈H₂₂ClFN₆O₄S. Found; 473.1 (M + 1). |
| 740- | N3-(3-chloro-4-fluoro-phenyl)-N5-[(1,5- | ¹H NMR (400 MHz, DMSO-d6): δ |
| ISOMER-A | dimethylpyrazol-4-yl)methyl]-2-methyl-1,1- | 10.51 (s, 1H), 8.30 (t, J = 5.5 Hz, 1H), |
| | dioxo-1,2,6-thiadiazinane-3,5-dicarboxamide. | 7.94 (dd, J = 6.8, 2.6 Hz, 1H), 7.56- |
| | | 7.51 (m, 1H), 7.39 (t, J = 9.1 Hz, 1H), |
| | | 7.26-7.23 (m, 2H), 4.22-4.13 (m, 1H), |
| | | 4.11-3.99 (m, 3H), 3.67 (s, 3H), 2.56 |
| | | (s, 3H), 2.19 (s, 3H), 1.96-1.86 (m, |
| | | 2H). LCMS calcd for; |
| | | C₁₈H₂₂ClFN₆O₄S. Found; |
| | | 473.20 (M + 1). |
| 740- | N3-(3-chloro-4-fluoro-phenyl)-N5-[(1,5- | ¹H NMR (400 MHz, DMSO-d6): δ |
| ISOMER-B | dimethylpyrazol-4-yl)methyl]-2-methyl-1,1- | 10.51 (s, 1H), 8.31 (t, J = 5.5 Hz, 1H), |
| | dioxo-1,2,6-thiadiazinane-3,5-dicarboxamide. | 7.94 (dd, J = 6.9, 2.5 Hz, 1H), 7.56- |
| | | 7.51 (m, 1H), 7.39 (t, J = 9.1 Hz, 1H), |
| | | 7.26-7.23 (m, 2H), 4.22-4.13 (m, 1H), |
| | | 4.11-3.99 (m, 3H), 3.67 (s, 3H), 2.56 |
| | | (s, 3H), 2.19 (s, 3H), 1.96-1.86 (m, |
| | | 2H). LCMS calcd for; |
| | | C₁₈H₂₂ClFN₆O₄S. Found; |
| | | 473.25 (M + 1). |
| 741 | 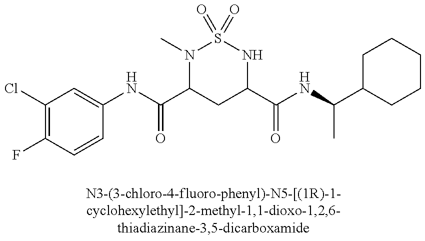 [SMILES: C[C@@H](NC(=O)C1CC(C(=O)Nc2ccc(F)c(Cl)c2)N(C)S(=O)(=O)N1)C1CCCCC1] | ¹H NMR (400 MHz, DMSO-d6): δ 10.53 (s, 1H), 7.96-7.94 (m, 1H), 7.88-7.80 (m, 1H), 7.55-7.53 (m, 1H), 7.40 (t, J = 8.8 Hz, 1H), 7.25-7.20 (m, 1H), 4.45-4.05 (m, 2H), 3.61-3.59 (m, 1H), 2.57 (s, 3H), 1.92-1.87 (m, 2H), 1.68-1.60 (m, 5H), 1.40-1.11 (m, 4H), 1.03-0.98 (m, 3H), 0.93-0.88 (m, 2H). LCMS calcd for; C₂₀H₂₈ClFN₄O₄S. Found; 475.30 (M + 1). |
| 742 | 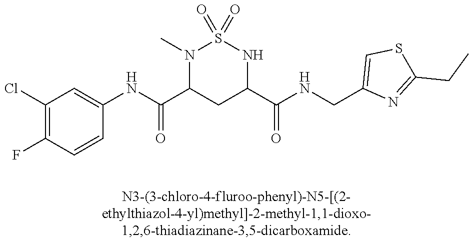 [SMILES: CCc1nc(CNC(=O)C2CC(C(=O)Nc3ccc(F)c(Cl)c3)N(C)S(=O)(=O)N2)cs1] | ¹H NMR (400 MHz, DMSO-d6): δ 10.54 (s, 1H), 8.62 (t, J = 4.8 Hz, 1H), 7.95 (dd, J = 6.8, 2.6 Hz, 1H), 7.57- 7.54 (m, 1H), 7.45-7.33 (m, 2H), 7.26 (s, 1H), 4.42-4.26 (m, 2H), 4.20-4.15 (m, 2H), 2.95 (q, J = 7.6, 2H), 2.58 (s, 3H), 2.05-1.88 (m, 2H), 1.28 (t, J = 7.6 Hz, 3H). LCMS calcd for; C₁₈H₂₁ClFN₅O₄S₂. Found; 490.1 (M + 1). |
| 742- | N3-(3-chloro-4-fluoro-phenyl)-N5-[(2- | ¹H NMR (400 MHz, DMSO-d6): δ |
| ISOMER-A | ethylthiazol-4-yl)methyl]-2-methyl-1,1-dioxo- | 10.53 (s, 1H), 8.62-8.60 (m, 1H), 7.95 |
| | 1,2,6-thiadiazinane-3,5-dicarboxamide. | (dd, J = 6.9, 2.6 Hz, 1H), 7.57-7.52 |
| | | (m, 1H), 7.40 (t, J = 9.1 Hz, 2H), 7.25 |
| | | (s, 1H), 4.42-4.29 (m, 2H), 4.20-4.16 |
| | | (m, 2H), 2.95 (q, J = 7.5 Hz, 2H), 2.57 |
| | | (s, 3H), 2.05-1.87 (m, 2H), 1.28 (t, J = |
| | | 7.5 Hz, 3H). LCMS calcd for; |
| | | C₁₈H₂₁ClFN₅O₄S₂. Found; |
| | | 490.25 (M + 1). |
| 742- | N3-(3-chloro-4-fluoro-phenyl)-N5-[(2- | ¹H NMR (400 MHz, DMSO-d6): δ |
| ISOMER-B | ethylthiazol-4-yl)methyl]-2-methyl-1,1-dioxo- | 10.53 (s, 1H), 8.62-8.60 (m, 1H), 7.97- |
| | 1,2,6-thiadiazinane-3,5-dicarboxamide. | 7.94 (m, 1H), 7.57-7.52 (m, 1H), 7.42- |
| | | 7.37 (m, 2H), 7.25 (s, 1H), 4.39-4.29 |
| | | (m, 2H), 4.20-4.16 (m, 2H), 2.95 (q, J = |
| | | 7.7 Hz, 2H), 2.57 (s, 3H), 2.05-1.87 |
| | | (m, 2H), 1.28 (t, J = 7.2 Hz, |
| | | 3H). LCMS calcd for; |
| | | C₁₈H₂₁ClFN₅O₄S₂, Found; |
| | | 490.25 (M + 1). |
| 744 | 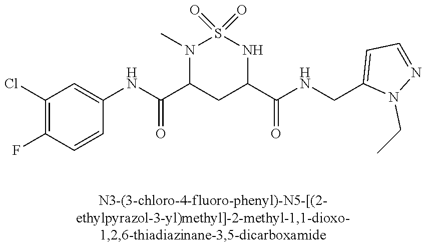 [SMILES: CCn1nccc1CNC(=O)C1CC(C(=O)Nc2ccc(F)c(Cl)c2)N(C)S(=O)(=O)N1] | .¹H NMR (400 MHz, DMSO-d6): δ 10.53 (s, 1H), 8.63 (t, J = 5.7 Hz, 1H), 7.95 (dd, J = 6.8, 2.6 Hz, 1H), 7.56- 7.52 (m, 1H), 7.44-7.30 (m, 3H), 6.14 (d, J = 1.8 Hz, 1H), 4.35 (d, J = 5.7 Hz, 2H), 4.24-4.17 (m, 1H), 4.12-4.05 (m, 3H), 2.57 (s, 3H), 1.97-1.88 (m, 2H), 1.29 (t, J = 7.2 Hz, 3H). LCMS calcd for; C₁₈H₂₂ClFN₆O₄S. Found; 473.1 (M + 1). |
| 745 | 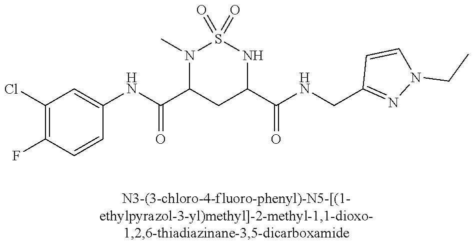 [SMILES: CCn1ccc(CNC(=O)C2CC(C(=O)Nc3ccc(F)c(Cl)c3)N(C)S(=O)(=O)N2)n1] | .¹H NMR (400 MHz, DMSO-d6): δ 10.53 (s, 1H), 8.49 (t, J = 5.7 Hz, 1H), 7.95 (dd, J = 6.8, 2.6 Hz, 1H), 7.63 (d, J = 2.0 Hz, 1H), 7.56-7.52 (m, 1H), 7.40 (t, J = 9.1 Hz, 1H), 7.30 (s, 1H), 6.11 (d, J = 2.2 Hz, 1H), 4.24-4.17 (m, 3H), 4.11-4.03 (m, 3H), 2.56 (s, 3H), 1.96-1.91 (m, 2H), 1.33 (t, J = 7.3 Hz, 3H). LCMS calcd for; C₁₈H₂₂ClFN₆O₄S. Found; 473.1 (M + 1). |
| 755 | 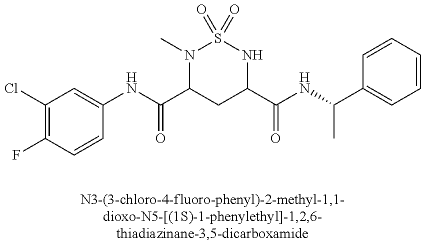 [SMILES: C[C@H](NC(=O)C1CC(C(=O)Nc2ccc(F)c(Cl)c2)N(C)S(=O)(=O)N1)c1ccccc1] | ¹H NMR (400 MHz, DMSO-d6) δ 10.53 (d, J = 4.0 Hz, 1H), 8.57-8.52 (m, 1H), 7.96-7.94 (m, 1H), 7.56-7.53 (m, 1H), 7.42-7.37 (m, 1H), 7.34-7.30 (m, 4H), 7.25-7.23 (m, 2H), 4.94-4.90 (m, 1H), 4.23-4.11 (m, 2H), 2.56 (d, J = 2.8 HZ, 3H), 1.96-1.87 (m, 2H), 1.37 (t, J = 6.4 Hz, 3H). LCMS calcd for; C₂₀H₂₂ClFN₄O₄S. Found; 469.2 (M + 1). |
| 766 | 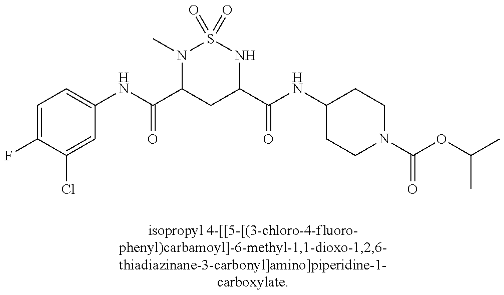 [SMILES: CC(C)OC(=O)N1CCC(NC(=O)C2CC(C(=O)Nc3ccc(F)c(Cl)c3)N(C)S(=O)(=O)N2)CC1] | ¹H NMR (400 MHz, DMSO-d6): δ 10.53 (s, 1H), 8.05 (d, J = 7.7 Hz, 1H), 7.95 (dd, J = 6.8, 2.6 Hz, 1H), 7.56- 7.52 (m, 1H), 7.40 (t, J = 9.1 Hz, 1H), 7.29-7.26 (m, 1H), 4.78-4.73 (m, 1H), 4.23-4.14 (m, 1H), 4.05-4.02 (m, 1H), 3.83-3.72 (m, 3H), 2.90-2.84 (m, 2H), 2.56 (s, 3H), 1.97-1.85 (m, 2h), 1.70-1.68 (m, 2H), 1.33-1.24 (m, 2H), 1.18 (d, J = 6.4 Hz, 6H). LCMS calcd for; C₂₁H₂₉ClFN₅O₆S. Found; 534.35 (M + 1). |
| 766- | isopropyl 4-[[5-[(3-chloro-4-fluoro- | ¹H NMR (400 MHz, DMSO-d6): δ |
| ISOMER-A | phenyl)carbamoyl]-6-methyl-1,1-dioxo-1,2,6- | 10.52 (s, 1H), 8.04 (d, J = 7.2 Hz, 1H), |
| | thiadiazinane-3-carbonyl]amino]piperidine-1- | 7.96-7.93 (m, 1H), 7.56-7.53 (m, 1H), |
| | carboxylate. | 7.40 (t, J = 8.8 Hz, 1H), 7.26-7.24 (m, |
| | | 1H), 4.79-4.72 (m, 1H), 4.21-4.16 (m, |
| | | 1H), 4.05-4.03 (m, 1H), 3.87-3.73 (m, |
| | | 3H), 2.89-2.87 (m, 2H), 2.56 (s, 3H), |
| | | 1.95-1.89 (m, 2H), 1.71-1.69 (m, 2H), |
| | | 1.33-1.27 (m, 2H), 1.17 (d, J = 6.0 |
| | | Hz, 6H). LCMS calcd for; |
| | | C₂₁H₂₉ClFN₅O₆S. Found; |
| | | 534.35 (M + 1). |
| 766- | isopropyl 4-[[5-[(3-chloro-4-fluoro- | ¹H NMR (400 MHz, DMSO-d6): δ |
| ISOMER-B | phenyl)carbamoyl]-6-methyl-1,1-dioxo-1,2,6- | 10.52 (s, 1H), 8.04 (d, J = 8.0 Hz, 1H), |
| | thiadiazinane-3-carbonyl]amino]piperidine-1- | 7.96-7.93 (m, 1H), 7.56-7.52 (m, 1H), |
| | carboxylate. | 7.40 (t, J = 8.8 Hz, 1H), 7.26-7.24 (m, |
| | | 1H), 4.79-4.72 (m, 1H), 4.20-4.16 (m, |
| | | 1H), 4.05-4.03 (m, 1H), 3.87-3.73 (m, |
| | | 3H), 2.89-2.87 (m, 2H), 2.56 (s, 3H), |
| | | 1.95-1.89 (m, 2H), 1.71-1.69 (m, 2H), |
| | | 1.36-1.28 (m, 2H), 1.17 (d, J = 6.4 |
| | | Hz, 6H). LCMS calcd for; |
| | | C₂₁H₂₉ClFN₅O₆S. Found; |
| | | 534.35 (M + 1). |
| 767 | 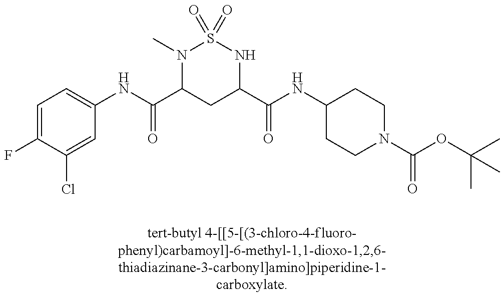 [SMILES: CN1C(C(=O)Nc2ccc(F)c(Cl)c2)CC(C(=O)NC2CCN(C(=O)OC(C)(C)C)CC2)NS1(=O)=O] | ¹H NMR (400 MHz, DMSO-d6): δ 10.53 (s, 1H), 8.05 (d, J = 7.7 Hz, 1H), 7.95 (dd, J = 6.8, 2.6 Hz, 1H), 7.56- 7.52 (m, 1H), 7.40 (t, J = 9.1 Hz, 1H), 7.29-7.26 (m, 1H), 4.23-4.14 (m, 1H), 4.05-4.02 (m, 1H), 3.83-3.72 (m, 3H), 2.90-2.84 (m, 2H), 2.56 (s, 3H), 1.97- 1.85 (m, 2H), 1.70-1.68 (m, 2H), 1.39 (s, 9H), 1.37-1.21 (m, 2H). LCMS cacld for; C₂₂H₃₁ClFN₅O₆S. Found; 448.0 (M − 100). |
| 767- | tert-butyl 4-[[5-[(3-chloro-4-fluoro- | ¹H NMR (400 MHz, DMSO-d6): δ |
| ISOMER-A | phenyl)carbamoyl]-6-methyl-1,1-dioxo-1,2,6- | 10.42 (s, 1H), 7.95 (dd, J = 6.9, 2.6 |
| | thiadiazinane-3-carbonyl]amino]piperidine-1- | Hz, 1H), 7.90-7.82 (m, 1H), 7.56-7.53 |
| | carboxylate. | (m, 1H), 7.38 (t, J = 9.1 Hz, 1H), 7.30- |
| | | 7.28 (m, 1H), 3.85-3.82 (m, 3H), 3.79- |
| | | 3.66 (m, 2H), 2.85-2.83 (m, 2H), 2.45 |
| | | (s, 3H), 1.95-1.90 (m, 1H), 1.85-1.78 |
| | | (m, 1H), 1.71-1.66 (m, 2H), 1.39 (s, |
| | | 9H), 1.34-1.23 (m, 2H. LCMS calcd |
| | | for; C₂₂H₃₁ClFN₅O₆S. Found; |
| | | 448.25 (M − 100). |
| 767- | tert-butyl 4-[[5-[(3-chloro-4-fluoro- | ¹H NMR (400 MHz, DMSO-d6): δ |
| ISOMER-B | phenyl)carbamoyl]-6-methyl-1,1-dioxo-1,2,6- | 10.28 (s, 1H), 7.96 (dd, J = 6.9, 2.5 |
| | thiadiazinane-3-carbonyl]amino]piperidine-1- | Hz, 1H), 7.76-7.72 (m, 1H), 7.58-7.54 |
| | carboxylate. | (m, 1H), 7.36 (t, J = 9.1 Hz, 1H), 7.27- |
| | | 7.25 (m, 1H), 3.86-3.82 (m, 3H), 3.77- |
| | | 3.67 (m, 2H), 2.83-2.78 (m, 2H), 2.40 |
| | | (s, 3H), 1.95-1.91 (m, 1H), 1.70-1.67 |
| | | (m, 3H), 1.39 (s, 9H), 1.32-1.23 (m, |
| | | 2H). LCMS calcd for; |
| | | C₂₂H₃₁ClFN₅O₆S. Found; 448.25 (M − |
| | | 100). |
| 769 | 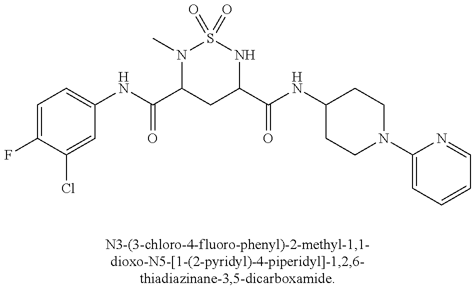 [SMILES: CN1C(C(=O)Nc2ccc(F)c(Cl)c2)CC(C(=O)NC2CCN(c3ccccn3)CC2)NS1(=O)=O] | ¹H NMR (400 MHz, DMSO-d6): δ 10.50 (s, 1H), 8.10-8.04 (m, 2H), 7.96- 7.93 (m, 1H), 7.55-7.48 (m, 2H), 7.39 (t, J = 8.8 Hz, 1H), 7.25 (d, J = 10.4 Hz, 1H), 6.83 (d, J = 8.4 Hz, 2H), 6.59 (t, J = 6.0 Hz, 1H), 4.20-4.16 (m, 3H), 4.08-4.01 (m, 1H), 3.85-3.82 (m, 1H), 2.94 (t, J = 12 Hz, 2H), 2.56 (s, 3H), 1.99-1.90 (m, 2H), 1.79-1.75 (m, 2H), 1.46-1.35 (m, 2H). LCMS calcd for; C₂₂H₂₆ClFN₆O₄S. Found; 525.50 (M + 1). |
| 769- | N3-(3-chloro-4-fluoro-phenyl)-2-methyl-1,1- | ¹H NMR (400 MHz, DMSO-d6): δ |
| ISOMER-A | dioxo-N5-[1-(2-pyridyl)-4-piperidyl]-1,2,6- | 10.34 (bs ,1H), 8.08-8.06 (m, 1H), |
| | thiadiazinane-3,5-dicarboxamide. | 7.94 (dd, J = 6.8, 2.6 Hz, 1H), 7.84- |
| | | 7.82 (m, 1H), 7.59-7.44 (m, 2H), 7.36 |
| | | (t, J = 9.1 Hz, 1H), 7.26 (s, 1H), 6.82 |
| | | (d, J = 8.6 Hz, 1H), 6.59-6.56 (m, |
| | | 1H), 4.21-4.12 (m, 2H), 3.87-3.73 (m, |
| | | 3H), 2.97-2.85 (m, 2H), 2.43 (s, 3H), |
| | | 1.94-1.75 (m, 4H), 1.43-1.31 (m, |
| | | 2H). LCMS calcd for; |
| | | C₂₂H₂₆ClFN₆O₄S. Found; 525.1 (M + |
| | | 1). |
| 769- | N3-(3-chloro-4-fluoro-phenyl)-2-methyl-1,1- | ¹H NMR (400 MHz, DMSO-d6): δ |
| ISOMER-B | dioxo-N5-[1-(2-pyridyl)-4-piperidyl]-1,2,6- | 10.51 (s, 1H), 8.12-8.01 (m, 2H), 7.95 |
| | thiadiazinane-3,5-dicarboxamide | (dd, J = 6.8, 2.5 Hz, 1H), 7.59-7.45 |
| | | (m, 2H), 7.39 (t, J = 9.0 Hz, 1H), 7.25- |
| | | 7.22 (m, 1H), 6.83 (d, J = 8.6 Hz, 1H), |
| | | 6.61-6.57 (m, 1H), 4.20-4.18 (m, 3H), |
| | | 4.04-4.02 (m, 1H), 3.83-3.80 (m, 1H), |
| | | 2.94 (t, J = 12.6 Hz, 2H), 2.56 (s, 3H), |
| | | 2.01-1.85 (m, 2H), 1.77-1.75 (m, 2H), |
| | | 1.47-1.36 (m, 2H). LCMS calcd for; |
| | | C₂₂H₂₆ClFN₆O₄S. Found; 525.1 (M + |
| | | 1). |
| 770 | 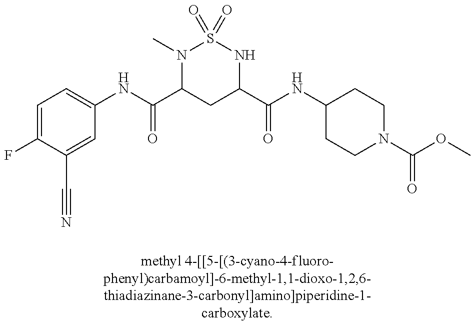 [SMILES: COC(=O)N1CCC(NC(=O)C2CC(C(=O)Nc3ccc(F)c(C#N)c3)N(C)S(=O)(=O)N2)CC1] | ¹H NMR (400 MHz, DMSO-d6): δ 10.68 (s, 1H), 8.17-8.15 (m, 1H), 8.07 (d, J = 7.2 Hz, 1H), 7.95-7.90 (m, 1H), 7.53 (t, J = 9.2 Hz, 1H), 7.29 (d, J = 9.6 Hz, 1H), 4.25-4.21 (m, 1H), 4.07- 4.04 (m, 1H), 3.87-3.75 (m, 3H), 3.59 (s, 3H), 2.95-2.92 (m, 2H), 2.58 (s, 3H), 1.95-1.90 (m, 2H), 1.75-1.72 (m, 2H), 1.35-1.29 (m, 2H). LCMS calcd for; C₂₀H₂₅FN₆O₆S. Found; 497.30 (M + 1). |
| 772 | 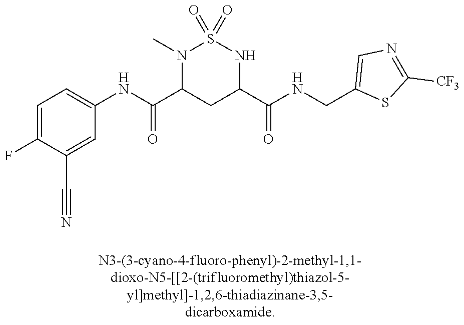 [SMILES: CN1C(C(=O)Nc2ccc(F)c(C#N)c2)CC(C(=O)NCc2cnc(C(F)(F)F)s2)NS1(=O)=O] | ¹H NMR (400 MHz, DMSO-d6): δ 10.69 (s, 1H), 8.94-8.93 (m, 1H), 8.16- 7.91 (m, 3H), 7.56-7.51 (m, 2H), 4.59-4.57 (m, 2H), 4.25-4.11 (m, 2H), 2.58 (s, 3H), 2.08-1.87 (m, 2H). LCMS calcd for; C₁₈H₁₆F₄N₆O₄S₂. Found; 521.20 (M + 1). |
| 773 | 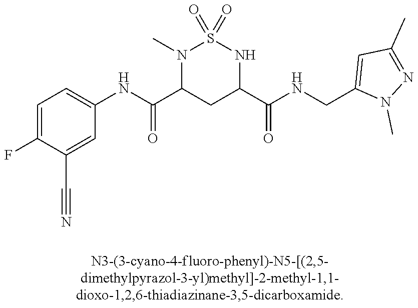 [SMILES: Cc1cc(CNC(=O)C2CC(C(=O)Nc3ccc(F)c(C#N)c3)N(C)S(=O)(=O)N2)n(C)n1] | ¹H NMR (400 MHz, DMSO-d6): δ 10.68 (s, 1H), 8.58 (t, J = 5.8 Hz, 1H), 8.15 (dd, J = 5.8, 2.7 Hz, H), 7.93- 7.88 (m, 1H), 7.52 (t, J = 9.1 Hz, 1H), 7.43-7.35 (m, 1H), 5.92 (s, 1H), 4.31- 4.17 (m, 3H), 4.16-4.05 (m, 1H), 3.67 (s, 3H), 2.58 (s, 3H), 2.07 (s, 3H), 2.01-1.89 (m, 2H). LCMS calcd for; C₁₉H₂₂FN₇O₄S. Found; 464.25 (M + 1). |
| 774 | 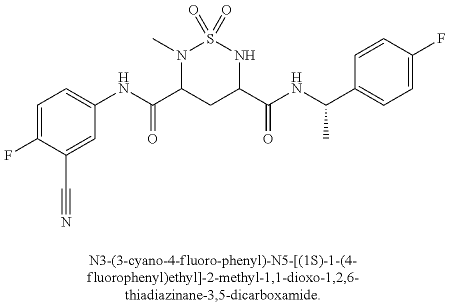 [SMILES: C[C@H](NC(=O)C1CC(C(=O)Nc2ccc(F)c(C#N)c2)N(C)S(=O)(=O)N1)c1ccc(F)cc1] | ¹H NMR (400 MHz, DMSO-d6): δ 10.68 (s, 1H), 8.58 (d, J = 7.9 Hz, 1H), 8.15 (dd, J = 5.6, 2.7 Hz, 1H), 7.93- 7.92 (m, 1H), 7.53 (t, J = 9.2 Hz, 1H), 7.43-7.27 (m, 3H), 7.25-7.09 (m, 2H), 4.94-4.90 (m, 1H), 4.24 (dd, J = 8.9, 5.9 Hz, 1H), 4.13-4.11 (m, 1H), 2.57 (s, 3H), 1.98-1.84 (m, 2H), 1.38 (d, J = 6.9 Hz, 3H). LCMS calcd for; C₂₁H₂₁F₂N₅O₄S. Found; 478.1 (M + 1). |
| 776 | 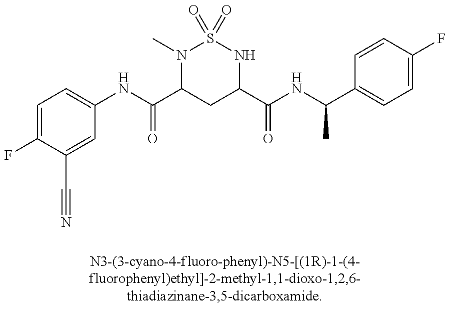 [SMILES: C[C@@H](NC(=O)C1CC(C(=O)Nc2ccc(F)c(C#N)c2)N(C)S(=O)(=O)N1)c1ccc(F)cc1] | ¹H NMR (400 MHz, DMSO-d6): δ 10.68 (s, 1H), 8.58 (d, J = 8.0 Hz, 1H), 8.16-8.15 (m, 1H), 7.92-7.90 (m, 1H), 7.53 (t, J = 9.0 Hz, 1H), 7.43-7.27 (m, 3H), 7.16-7.12 (m, 2H), 4.94-4.91 (m, 1H), 4.26-4.22 (m, 1H), 4.13-4.11 (m, 1H), 2.57 (s, 3H), 1.98-1.84 (m, 2H), 1.38 (d, J = 6.9 Hz, 3H). LCMS calcd for; C₂₁H₂₁F₂N₅O₄S. Found; 478.0 (M + 1). |
| 817 | 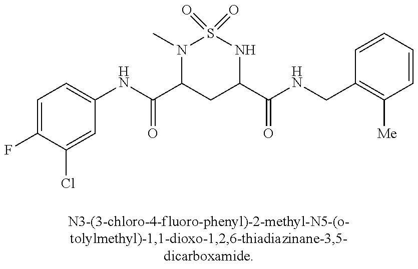 [SMILES: Cc1ccccc1CNC(=O)C1CC(C(=O)Nc2ccc(F)c(Cl)c2)N(C)S(=O)(=O)N1] | ¹H NMR (400 MHz, DMSO-d6): δ 10.54 (s, 1H), 8.52 (t, J = 5.7 Hz, 1H), 7.96 (dd, J = 6.9, 2.6 Hz, 1H), 7.57- 7.53 (m, 1H), 7.45-7.35 (m, 2H), 7.23- 7.16 (m, 4H), 4.31-4.07 (m, 4H), 2.58 (s, 3H), 2.27 (s, 3H), 2.02-1.93 (m, 2H). LCMS calcd for; C₂₀H₂₂ClFN₄O₄S. Found; 469.25 (M + 1). |
| 818 | 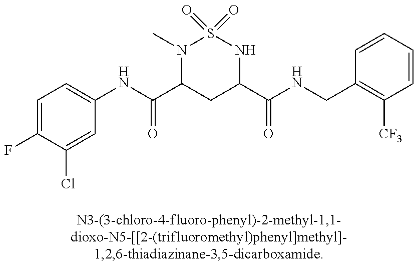 [SMILES: CN1C(C(=O)Nc2ccc(F)c(Cl)c2)CC(C(=O)NCc2ccccc2C(F)(F)F)NS1(=O)=O] | ¹H NMR (400 MHz, DMSO-d6): δ 10.56 (s, 1H), 8.73 (t, J = 5.8 Hz, 1H), 7.97-7.95 (m, 1H), 7.73-7.65 (m, 2H), 7.57-7.45 (m, 4H), 7.43-7.38 (m, 1H), 4.57-4.40 (m, 2H), 4.24-4.19 (m, 2H), 2.60 (s, 3H), 2.09-1.91 (m, 2H). LCMS calcd for; C₂₀H₁₉ClF₄N₄O₄S. Found; 523.08 (M + 1). |
| 819 | 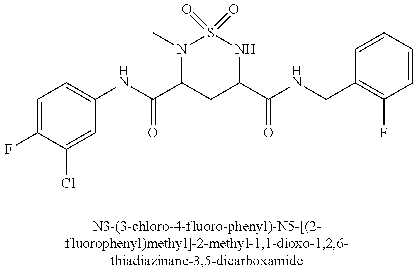 [SMILES: CN1C(C(=O)Nc2ccc(F)c(Cl)c2)CC(C(=O)NCc2ccccc2F)NS1(=O)=O] | .¹H NMR (400 MHz, DMSO-d6): δ 10.55 (s, 1H), 8.65 (t, J = 5.9 Hz, 1H), 7.96 (dd, J = 6.9, 2.6 Hz, 1H), 7.57- 7.55 (m, 1H), 7.45-7.27 (m, 4H), 7.22- 7.13 (m, 2H), 4.35 (d, J = 5.7 Hz, 2H), 4.25-4.14 (m, 2H), 2.58 (m, 3H), 2.03- 1.88 (m, 2H). LCMS calcd for; C₁₉H₃₉ClF₂N₄O₄S. Found; 473.20 (M + 1). |
| 820 | 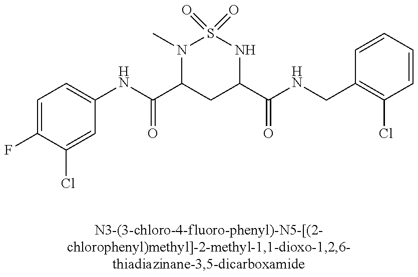 [SMILES: CN1C(C(=O)Nc2ccc(F)c(Cl)c2)CC(C(=O)NCc2ccccc2Cl)NS1(=O)=O] | ¹H NMR (400 MHz, DMSO-d6): δ 10.56 (s, 1H), 8.66 (t, J = 5.9 Hz, 1H), 7.96 (dd, J = 6.9, 2.6 Hz, 1H), 7.57- 7.53 (m, 1H), 7.46-7.25 (m, 6H), 4.44- 4.31 (m, 2H), 4.23-4.19 (m, 2H), 2.59 (s, 3H), 2.07-1.90 (m, 2H). LCMS calcd for; C₁₉H₁₉Cl₂FN₄O₄S. Found; 489.15 (M + 1). |
| 821 | 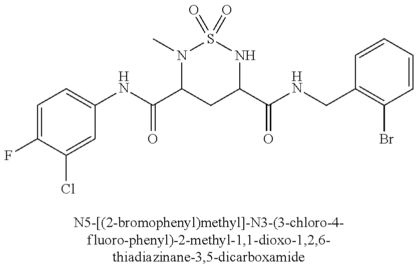 [SMILES: CN1C(C(=O)Nc2ccc(F)c(Cl)c2)CC(C(=O)NCc2ccccc2Br)NS1(=O)=O] | ¹H NMR (400 MHz, DMSO-d6): δ 10.54 (s, 1H), 8.66 (t, J = 5.9 Hz, 1H), 7.96 (dd, J = 7.1, 2.5 Hz, 1H), 7.61- 7.59 (m, 1H), 7.57-7.52 (m, 1H), 7.47- 7.46 (m, 1H), 7.41-7.36 (m, 3H), 7.26- 7.17 (m, 1H), 4.37-4.28 (m, 2H), 4.23- 4.18 (m, 2H), 2.59 (s, 3H), 2.07-1.90 (m, 2H). LCMS calcd for; C₁₉H₁₉BrClFN₄O₄S. Found; 535.20 (M + 2). |
| 822 | 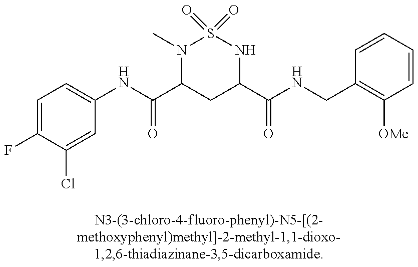 [SMILES: COc1ccccc1CNC(=O)C1CC(C(=O)Nc2ccc(F)c(Cl)c2)N(C)S(=O)(=O)N1] | ¹H NMR (400 MHz, DMSO-d6): δ 10.55 (s, 1H), 8.44 (t, J = 5.9 Hz, 1H), 7.96 (dd, J = 6.8, 2.6 Hz, 1H, 7.56- 7.54 (m, 1H), 7.4-57.36 (m, 2H), 7.29- 7.16 (m, 2H), 6.98 (d, J = 8.0 Hz, 1H), 6.91 (t, J = 7.2 Hz, 1H), 4.30-4.14 (m, 4H), 3.81 (s, 3H), 2.58 (s, 3H), 2.02- 1.92 (m, 2H). LCMS calcd for; C₂₀H₂₂ClFN₄O₅S. Found; 485.25 (M + 1). |
| 823 | 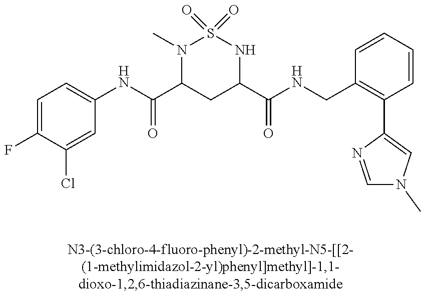 [SMILES: CN1C(C(=O)Nc2ccc(F)c(Cl)c2)CC(C(=O)NCc2ccccc2-c2cn(C)cn2)NS1(=O)=O] | ¹H NMR (400 MHz, DMSO-d6): δ 10.50 (s, 1H), 8.63 (t, J = 6.0 Hz, 1H), 8.10-8.08 (m, 1H), 7.94-7.91 (m, 1H), 7.56-7.50 (m, 3H), 7.40-7.32 (m, 3H), 7.30-7.28 (m, 2H), 4.44 (d, J = 5.6 Hz, 2H), 4.18-4.08 (m, 2H), 3.74 (s, 3H), 2.54 (s, 3H), 1.94-1.88 (m, 2H). LCMS calcd for; C₂₃H₂₄ClFN₆O₄S; Found; 535.35 (M + 1). |
| 823- | N3-(3-chloro-4-fluoro-phenyl)-2-methyl-N5-[[2- | ¹H NMR (400 MHz, DMSO-d6): δ |
| ISOMER-A | (1-methylimidazol-4-yl)phenyl]methyl]-1,2- | 10.49 (s, 1H), 8.63 (t, J = 6.0 Hz, 1H), |
| | dioxo-1,2,6-thiadiazinane-3,5-dicarboxamide. | 7.93-7.90 (m, 1H), 7.67 (s, 1H), 7.60- |
| | | 7.57 (m, 1H), 7.53-7.49 (m, 1H), 7.40- |
| | | 7.33 (m, 3H), 7.30-7.28 (m, 1H), 7.25- |
| | | 7.16 (m, 2H), 4.54-4.43 (m, 2H), 4.18- |
| | | 7.16 (m, 2H), 4.54-4.43 (m, 2H), 4.18- |
| | | 4.09 (m, 2H), 3.68 (s, 3H), 2.54 (s, |
| | | 3H), 1.98-1.85 (m, 2H). LCMS calcd |
| | | for; C₂₃H₂₄ClFN₆O₄S. FOund; |
| | | 535.15 (M + 1). |
| 823- | N3-(3-chloro-4-fluoro-phenyl)-2-methyl-N5-[[2- | ¹H NMR (400 MHz, DMSO-d6): δ |
| ISOMER-B | (1-methylimidazol-4-yl)phenyl]methyl]-1,1- | 10.49 (s, 1H), 8.62 (t, J = 5.6 Hz, 1H), |
| | dioxo-1,2,6-thiadiazinane-3,5-dicarboxamide. | 7.93-7.91 (m, 1H), 7.67 (s, 1H), 7.60- |
| | | 7.58 (m, 1H), 7.53-7.49 (m, 1H), 7.40- |
| | | 7.33 (m, 3H), 7.30-7.28 (m, 1H), 7.25- |
| | | 7.16 (m, 2H), 4.54-4.43 (m, 2H), |
| | | 4.18-4.08 (m, 2H), 3.68 (s, 3H), 2.54 |
| | | (s, 3H), 1.98-1.85 (m, 2H). LCMS |
| | | calcd for; C₂₃H₂₄ClFN₆O₄S. Found; |
| | | 535.10 (M + 1). |
| 826 | 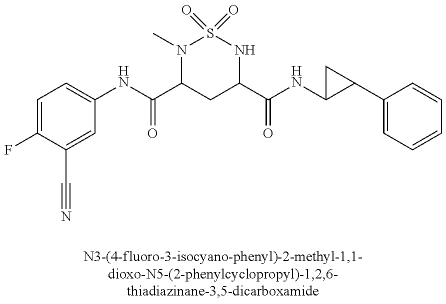 [SMILES: CN1C(C(=O)Nc2ccc(F)c(C#N)c2)CC(C(=O)NC2CC2c2ccccc2)NS1(=O)=O] | ¹H NMR (400 MHz, DMSO-d6): δ 10.67 (s, 1H), 8.41 (t, J = 5.5 Hz, 1H), 8.15 (dd, J = 5.8, 2.6 Hz, 1H), 7.92- 7.90 (m, 1H), 7.53 (t, J = 9.1 Hz, 1H), 7.37-7.21 (m, 3H), 7.20-7.07 (m, 3H), 4.26-4.22 (m, 1H), 4.05-4.03 (m, 1H), 2.85-2.83 (m, 1H), 2.57 (s, 3H), 2.04- 1.89 (m, 3H), 1.30-1.11 (m, 2H). LCMS calcd for; C₂₂H₂₂FN₅O₄S. Found; 472.25 (M + 1). |
| 828 | 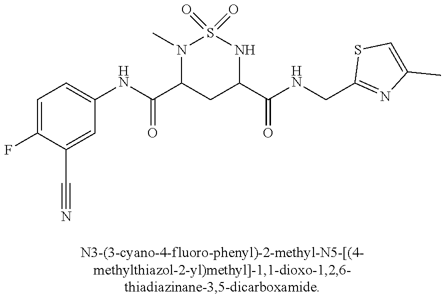 [SMILES: Cc1csc(CNC(=O)C2CC(C(=O)Nc3ccc(F)c(C#N)c3)N(C)S(=O)(=O)N2)n1] | ¹H NMR (400 MHz, DMSO-d6): δ 10.68 (s, 1H), 8.98 (t, J = 6.1 Hz, 1H), 8.16 (dd, J = 5.8, 2.7 Hz, 1H), 7.94- 7.89 (m, 1H), 7.53 (t, J = 9.1 Hz, 1H), 7.42 (d, J = 9.4 Hz, 1H), 7.16 (s, 1H), 4.63-4.43 (m, 2H), 4.30-4.26 (m, 1H), 4.16-4.14 (m, 1H), 2.58 (s, 3H), 2.32 (s, 3H), 2.03-1.90 (m, 2H). LCMS calcd for; C₁₈H₁₉FN₆O₄S₂. Found; 467.20 (M + 1). |
| 829 | 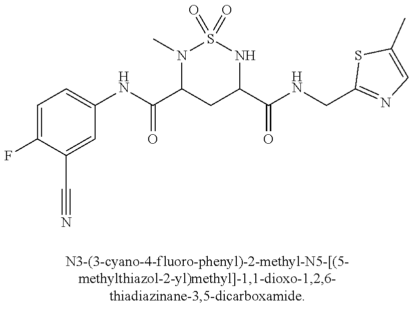 [SMILES: Cc1cnc(CNC(=O)C2CC(C(=O)Nc3ccc(F)c(C#N)c3)N(C)S(=O)(=O)N2)s1] | ¹H NMR (400 MHz, DMSO-d6): δ 10.70 (s, 1H), 8.98 (t, J = 6.0 Hz, 1H), 8.17 (dd, J = 5.8, 2.7 Hz, 1H), 7.94- 7.90 (m, 1H), 7.54 (t, J = 9.1 Hz, 1H), 7.46-7.35 (m, 2H), 4.60-4.40 (m, 2H), 4.30-4.27 (m, 1H), 4.15-4.13 (m, 1H), 2.59 (s, 3H), 2.40 (s, 3H), 2.03-1.87 (m, 2H). LCMS calcd for; C₁₈H₁₉FN₆O₄S₂. Found; 467 (M + 1). |
| 830 | 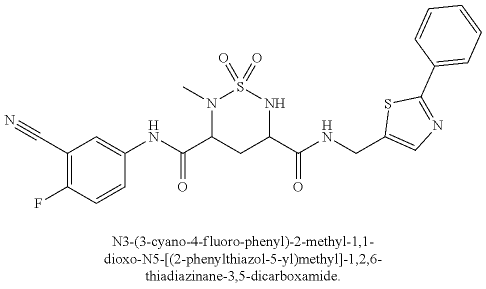 [SMILES: CN1C(C(=O)Nc2ccc(F)c(C#N)c2)CC(C(=O)NCc2cnc(-c3ccccc3)s2)NS1(=O)=O] | ¹H NMR (400 MHz, DMSO-d6): δ 10.67 (s, 1H), 8.85-8.83 (m, 1H), 8.16- 8.14 (m, 1H), 7.93-7.88 (m, 3H), 7.76 (s, 1H), 7.54-7.44 (m, 5H), 4.52 (d, J = 6.0 Hz, 2H), 4.25-4.22 (m, 1H), 4.12-4.09 (m, 1H), 2.58 (s, 3H), 2.02- 1.92 (m, 2H). LCMS calcd for; C₂₃H₂₁FN₆O₄S₂. Found; 529.45 (M + 1). |
| 831 | 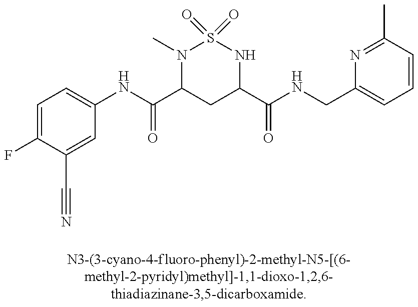 [SMILES: Cc1cccc(CNC(=O)C2CC(C(=O)Nc3ccc(F)c(C#N)c3)N(C)S(=O)(=O)N2)n1] | ¹H NMR (400 MHz, DMSO-d6): δ 10.70 (s, 1H), 8.69 (t, J = 6.0 Hz, 1H), 8.16 (dd, J = 5.7, 2.7 Hz, 1H), 7.94- 7.90 (m, 1H), 7.65 (t, J = 7.7 Hz, 1H), 7.54 (t, J = 9.1 Hz, 1H), 7.46-7.44 (m, 1H), 7.13-7.11 (m, 2H), 4.35 (d, J = 5.9 Hz, 2H), 4.33-4.13 (m, 2H), 2.60 (s, 3H), 2.45 (s, 3H), 2.09-1.90 (m, 2H). LCMS calcd for; C₂₀H₂₁FN₆O₄S. Found; 461.30 (M + 1). |
| 832 | 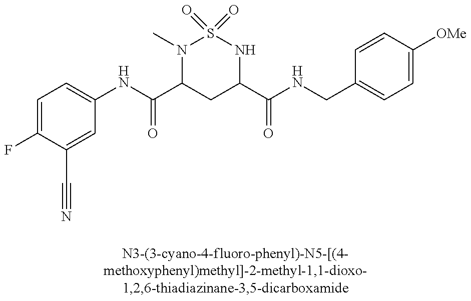 [SMILES: COc1ccc(CNC(=O)C2CC(C(=O)Nc3ccc(F)c(C#N)c3)N(C)S(=O)(=O)N2)cc1] | .¹H NMR (400 MHz, DMSO-d6): δ 10.67 (s, 1H), 8.56 (t, J = 5.9 Hz, 1H), 8.16-8.14 (m, 1H), 7.93-7.88 (m, 1H), 7.52 (t, J = 9.1 Hz, 1H), 7.36-7.35 (m, 1H), 7.20 (d, J = 8.4 Hz, 2H), 6.88 (d, J = 8.0 Hz, 2H), 4.25-4.21 (m, 3H), 4.11-4.09 (m, 1H), 3.72 (s, 3H), 2.58 (s, 3H), 2.00-1.90 (m, 2H). LCMS calcd for; C₂₁H₂₂FN₅O₅S. Found; 476.30 (M + 1). |
| 833 | 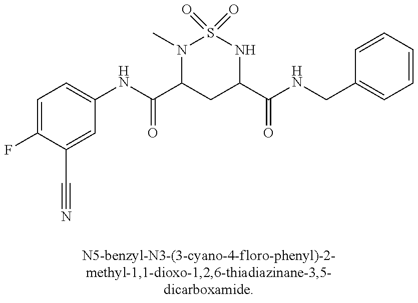 [SMILES: CN1C(C(=O)Nc2ccc(F)c(C#N)c2)CC(C(=O)NCc2ccccc2)NS1(=O)=O] | ¹H NMR (400 MHz, DMSO-d6): δ 10.68 (s, 1H), 8.65 (t, J = 5.9 Hz, 1H), 8.15 (dd, J = 5.7, 2.7 Hz, 1H), 7.93- 7.88 (m, 1H), 7.52 (t, J = 9.1 Hz, 1H), 7.41-7.39 (m, 1H), 7.31-7.21 (m, 5H), 4.33-4.20 (m, 3H), 4.15-4.13 (m, 1H), 2.58 (s, 3H), 2.03-1.88 (m, 2H). LCMS calcd for; C₂₀H₂₀FN₅O₄S. Found; 446.25 (M + 1). |
| 834 | 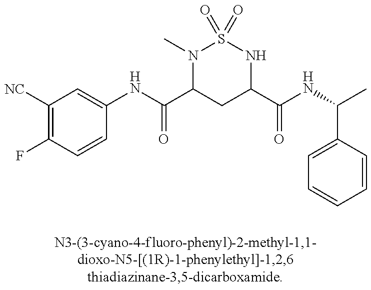 [SMILES: C[C@@H](NC(=O)C1CC(C(=O)Nc2ccc(F)c(C#N)c2)N(C)S(=O)(=O)N1)c1ccccc1] | ¹H NMR (400 MHz, DMSO-d6): δ 10.67 (d, J = 4.7 Hz, 1H), 8.55 (dd, J = 12.3, 7.9 Hz, 1H), 8.16-8.14 (m, 1H), 7.96-7.86 (m, 1H), 7.55-7.49 (m, 1H), 7.38-7.17 (m, 6H), 4.95-4.90 (m, 1H), 4.27-4.23 (m, 1H), 4.15-4.12 (m, 1H), 2.58-2.56 (m, 3H), 2.01-1.85 (m, 2H), 1.39-1.36 (m, 3H). LCMS calcd for; C₂₁H₂₂FN₅O₄S. Found; 460.30 (M + 1). |
| 835 | 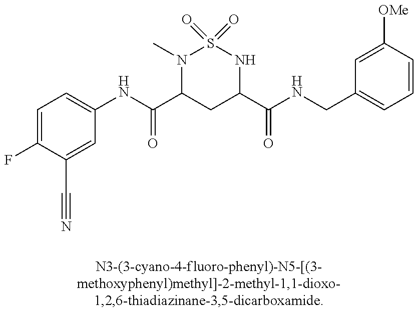 [SMILES: COc1cccc(CNC(=O)C2CC(C(=O)Nc3ccc(F)c(C#N)c3)N(C)S(=O)(=O)N2)c1] | ¹H NMR (400 MHz, DMSO-d6): δ 10.68 (s, 1H), 8.62 (t, J = 5.8 Hz, 1H), 8.17-8.13 (m, 1H), 7.93-7.88 (m, 1H), 7.52 (t, J = 9.2 Hz, 1H), 7.41-7.39 (m, 1H), 7.23 (t, J = 8.0 Hz, 1H), 6.88- 6.77 (m, 3H), 4.31-4.23 (m, 3H), 4.15- 4.12 (m, 1H), 3.73 (s, 3H), 2.58 (s, 3H), 2.04-1.88 (m, 2H). LCMS calcd for; C₂₁H₂₂FN₅O₅S. Found; 476.25 (M + 1). |
| 836 | 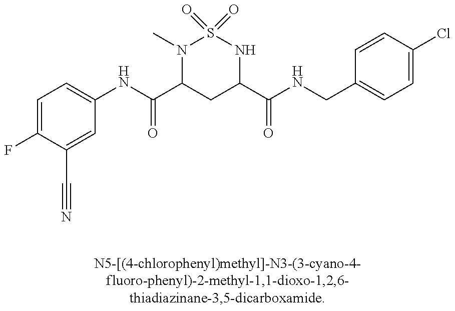 [SMILES: CN1C(C(=O)Nc2ccc(F)c(C#N)c2)CC(C(=O)NCc2ccc(Cl)cc2)NS1(=O)=O] | ¹H NMR (400 MHz, DMSO-d6): δ 10.69 (s, 1H), 8.67 (t, J = 5.2 Hz, 1H), 81.7-8.15 (m, 1H), 7.93-7.90 (m, 1H), 7.53 (t, J = 9.1 Hz, 1H), 7.48-7.45 (m, 1H), 7.38 (d, J = 8.0 Hz, 2H), 7.31 (d, J = 8.0 Hz, 2H), 4.32-4.20 (m, 3H), 4.17-4.11 (m, 1H), 2.59 (s, 3H), 2.05- 1.87 (m, 2H). LCMS calcd for; C₂₀H₁₉ClFN₅O₄S. Found; 480 (M + 1). |
| 837 | 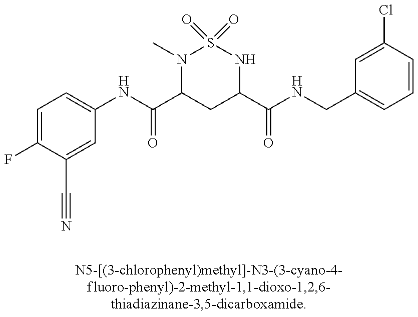 [SMILES: CN1C(C(=O)Nc2ccc(F)c(C#N)c2)CC(C(=O)NCc2cccc(Cl)c2)NS1(=O)=O] | ¹H NMR (400 MHz, DMSO-d6): δ 10.69 (s, 1H), 8.69 (t, J = 6.0 Hz, 1H), 8.17-8.15 (m, 1H), 7.94-7.90 (m, 1H), 7.55-7.47 (m, 2H), 7.40-7.21 (m, 4H), 4.39-4.20 (m, 3H), 4.17-4.14 (m, 1H), 2.59 (s, 3H), 2.08-1.88 (m, 2H). LCMS calcd for; C₂₀H₁₉ClFN₅O₄S. Found; 480 (M + 1). |
| 838 | 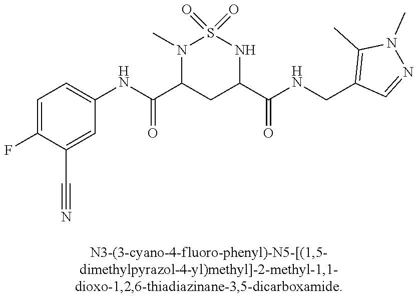 [SMILES: Cc1c(CNC(=O)C2CC(C(=O)Nc3ccc(F)c(C#N)c3)N(C)S(=O)(=O)N2)cnn1C] | ¹H NMR (400 MHz, DMSO-d6): δ 10.67 (s, 1H), 8.33 (t, J = 5.5 Hz, 1H), 8.17-8.14 (m, 1H), 7.93-7.88 (m, 1H), 7.53 (t, J = 9.0 Hz, 1H), 7.30-7.28 (m, 1H), 7.24 (s, 1H), 4.24-4.20 (m, 1H), 4.07-4.01 (m, 3H), 3.68 (s, 3H), 2.57 (s, 3H), 2.19 (s, 3H), 1.97-1.87 (m, 2H). LCMS calcd for; C₁₉H₂₃FN₇O₄S. Found; 464 (M + 1). |
| 851 | 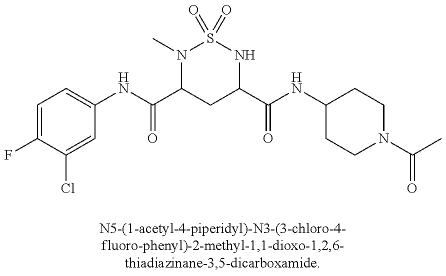 [SMILES: CC(=O)N1CCC(NC(=O)C2CC(C(=O)Nc3ccc(F)c(Cl)c3)N(C)S(=O)(=O)N2)CC1] | ¹H NMR (400 MHz, DMSO-d6): δ 10.66 (s, 1H), 8.16-8.13 (m, 1H), 7.97- 7.95 (m, 1H), 7.57-7.55 (m, 1H), 7.39 (t, J = 9.2 Hz, 1H), 7.25 (d, J = 9.6 Hz, 1H), 4.21-4.18 (m, 2H), 4.08-4.05 (m, 1H), 3.14-3.07 (m, 2H), 2.76-2.66 (m, 2H), 2.56 (s, 3H), 1.98 (s, 3H), 1.93-1.91 (m, 2H), 1.73-1.69 (m, 2H), 1.39-1.23 (m, 2H). LCMS calcd for; C₁₉H₂₅ClFN₅O₅S. Found; 490.50 (M + 1). |
| 852 | 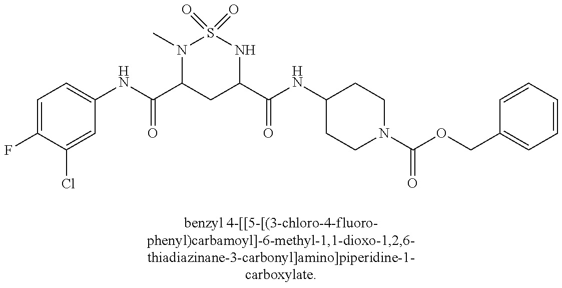 [SMILES: CN1C(C(=O)Nc2ccc(F)c(Cl)c2)CC(C(=O)NC2CCN(C(=O)OCc3ccccc3)CC2)NS1(=O)=O] | ¹H NMR (400 MHz, DMSO-d6): δ 10.51 (s, 1H), 8.05 (d, J = 8.0 Hz, 1H), 7.95 (dd, J = 6.4, 2.0 Hz, 1H), 7.55-7.52 (m, 1H), 7.42-7.31 (m, 6H), 7.26-7.24 (m, 1H), 5.07 (s, 2H), 4.21- 4.16 (m, 1H), 4.06-4.04 (m, 1H), 3.94-3.90 (m, 2H), 3.78-3.76 (m, 1H), 2.98-2.96 (m, 2H), 2.56 (s, 3H), 1.98- 1.89 (m, 2H), 1.74-1.71 (m, 2H), 1.39- 1.27 (m, 2H). LCMS calcd for; C₂₅H₂₉ClFN₅O₆S. Found; 582.35 (M + 1). |
| 854 | 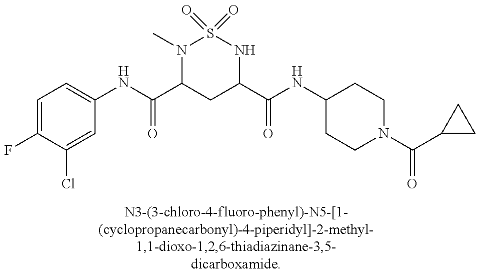 [SMILES: CN1C(C(=O)Nc2ccc(F)c(Cl)c2)CC(C(=O)NC2CCN(C(=O)C3CC3)CC2)NS1(=O)=O] | ¹H NMR (400 MHz, DMSO-d6): δ 10.52 (s, 1H), 8.06 (d, J = 8.0 Hz, 1H), 7.96-7.93 (m, 1H), 7.56-7.52 (m, 1H), 7.39 (t, J = 8.8 Hz, 1H), 7.26-7.24 (m, 1H), 4.20-4.17 (m, 3H), 4.07-4.04 (m, 1H), 3.85-3.82 (m, 1H), 3.28-3.21 (m, 1H), 2.76-2.65 (m, 1H), 2.57 (s, 3H), 1.99-1.90 (m, 3H), 1.80-1.70 (m, 2H), 1.40-1.25 (m, 2H), 0.70-0.65 (m, 4H). LCMS calcd for; C₂₁H₂₇ClFN₅O₅S. Found; 516.1 (M + 1). |
| 854- | N3-(3-chloro-4-fluoro-phenyl)-N5-[1- | ¹H NMR (400 MHz, DMSO-d6): δ |
| ISOMER-A | (cyclopropanecarbonyl)-4-piperidyl]-2-methyl- | 10.52 (s, 1H), 8.06 (d, J = 7.6 Hz, 1H), |
| | 1,1-dioxo-1,2,6-thiadiazinane-3,5- | 7.96-7.94 (m, 1H), 7.55-7.53 (m, 1H), |
| | dicarboxamide. | 7.39 (t, J = 8.8 Hz, 1H), 7.26-7.25 (m, |
| | | 1H), 4.19-4.17 (m, 3H), 4.05-4.03 (m, |
| | | 1H), 3.85-3.81 (m, 1H), 3.22-3.20 |
| | | (m, 1H), 2.76-2.67 (m, 1H), 2.57 (s, |
| | | 3H), 1.99-1.90 (m, 3H), 1.80-1.71 (m, |
| | | 2H), 1.38-1.24 (m, 2H), 0.70-0.66 (m, |
| | | 4H). LCMS calcd for; |
| | | C₂₁H₂₇ClFN₅O₅S. Found; |
| | | 516.30 (M + 1). |
| 854- | N3-(3-chloro-4-fluoro-phenyl)-N5-[1- | ¹H NMR (400 MHz, DMSO-d6): δ |
| ISOMER-B | (cyclopropanecarbonyl)-4-piperidyl]-2-methyl- | 10.53 (s, 1H), 8.06 (d, J = 7.2 Hz, |
| | 1,1-dioxo-1,2,6-thiadiazinane-3,5- | 1H), 7.96-7.93 (m, 1H), 7.57-7.54 (m, |
| | dicarboxamide. | 1H), 7.39 (t, J = 8.8 Hz, 1H), 7.27- |
| | | 7.25 (m, 1H), 4.21-4.17 (m, 3H), 4.07- |
| | | 4.03 (m, 1H), 3.85-3.83 (m, 1H), |
| | | 3.22-3.19 (m, 1H), 2.75-2.66 (m, 1H), |
| | | 2.57 (s, 3H), 1.99-1.91 (m, 3H), 1.80- |
| | | 1.70 (m, 2H), 1.39-1.24 (m, 2H), 0.70- |
| | | 0.66 (m, 4H). LCMS calcd for; |
| | | C₂₁H₂₇ClFN₅O₅S. FOund; |
| | | 516.30 (M + 1). |
| 855 | 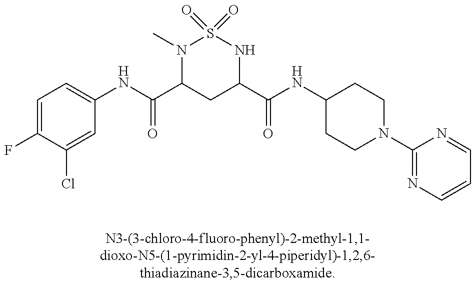 [SMILES: CN1C(C(=O)Nc2ccc(F)c(Cl)c2)CC(C(=O)NC2CCN(c3ncccn3)CC2)NS1(=O)=O] | ¹H NMR (400 MHz, DMSO-d6): δ 10.54 (s, 1H), 8.50-8.33 (m, 2H), 8.08- 7.94 (m, 2H), 7.54-7.52 (m, 1H), 7.39 (t, J = 8.8 Hz, 1H), 7.16-7.12 (m, 1H), 6.59 (t, J = 4.8 Hz, 1H), 4.50-4.48 (m, 2H), 4.21-4.17 (m, 1H), 4.07-4.03 (m, 1H), 3.89-3.87 (m, 1H), 3.06 (t, J = 12 Hz, 2H), 2.56 (s, 3H), 1.99-1.90 (m, 2H), 1.80-1.75 (m, 2H), 1.39-1.36 (m, 2H). LCMS calcd for; C₂₁H₂₅ClFN₇O₄S. Found; 526.35 (M + 1). |
| 855- | N3-(3-chloro-4-fluoro-phenyl)-2-methyl-1,1- | ¹H NMR (400 MHz, DMSO-d6): δ |
| ISOMER-A | dioxo-N5-(1-pyrimidin-2-yl-4-piperidyl)-1,2,6- | 10.48 (s, 1H), 8.33-8.30 (m, 2H), |
| | thiadiazinane-3,5-dicarboxamide. | 8.02-8.00 (m, 1H), 7.93-7.70 (m, 1H), |
| | | 7.53-7.50 (m, 1H), 7.36 (t, J = 9.2 Hz, |
| | | 1H), 7.25-7.20 (m, 1H), 6.56 (t, J = |
| | | 4.8 Hz, 1H), 4.48-4.45 (m, 2H), 4.13- |
| | | 4.00 (m, 2H), 3.85-3.82 (m, 1H), 3.03 |
| | | (t, J = 12.8 Hz, 2H), 2.52 (s, 3H), 1.90- |
| | | 1.87 (m, 2H), 1.75-1.73 (m, 2H), |
| | | 1.39-1.28 (m, 2H). LCMS calcd for; |
| | | C₂₁H₂₅ClFN₇O₄S. Found; 526.1 (M + |
| | | 1). |
| 855- | N3-(3-chloro-4-fluoro-phenyl)-2-methyl-1,1- | ¹H NMR (400 MHz, DMSO-d60: δ |
| ISOMER-B | dioxo-N5-(1-pyrimidin-2-yl-4-piperidyl)-1,2,6- | 10.50 (s, 1H), 8.31 (d, J = 4.8 Hz, |
| | thiadiazinane-3,5-dicarboxamide. | 2H), 8.03 (d, J = 7.2 Hz, 1H), 7.93- |
| | | 7.91 (m, 1H), 7.53-7.50 (m, 1H), 7.36 |
| | | (t, J = 8.8 Hz, 1H), 7.25-7.20 (m, |
| | | 1H), 6.56 (t, J = 4.8 Hz, 1H), 4.48- |
| | | 4.45 (m, 2H), 4.18-4.14 (m, 1H), 4.04- |
| | | 4.00 (m, 1H), 3.85-3.82 (m, 1H), |
| | | 3.03 (t, J = 11.6 Hz, 2H), 2.53 (s, 3H), |
| | | 1.93-1.87 (m, 2H), 1.75-1.73 (m, |
| | | 2H), 1.39-1.30 (m, 2H). LCMS calcd |
| | | for, C₂₁H₂₅ClFN₇O₄S. Found; |
| | | 526.1 (M + 1). |
| 856 | 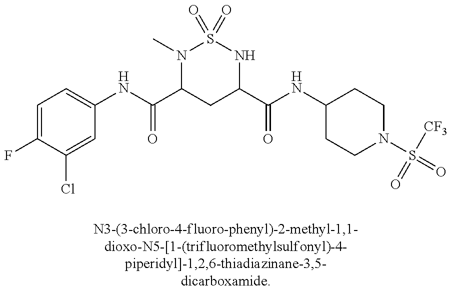 [SMILES: CN1C(C(=O)Nc2ccc(F)c(Cl)c2)CC(C(=O)NC2CCN(S(=O)(=O)C(F)(F)F)CC2)NS1(=O)=O] | ¹H NMR (400 MHz, DMSO-d6): δ 10.53 (s, 1H), 8.17 (d, J = 7.7 Hz, 1H), 7.95 (dd, J = 6.9, 2.5 Hz, 1H), 7.57- 7.53 (m, 1H), 7.40 (t, J = 9.2 Hz, 1H), 7.32 (d, J = 10.4 Hz, 1H), 4.21-4.17 (m, 1H), 4.07-4.04 (m, 1H), 3.89-3.87 (m, 1H), 3.81-3.71 (m, 2H), 2.57 (s, 3H), 1.98-1.81 (m, 4H), 1.60-1.42 (m, 2H). LCMS calcd for; C₁₈H₂₂ClF₄N₅O₆S₂. Found; 580 (M + 1). |
| 858 | 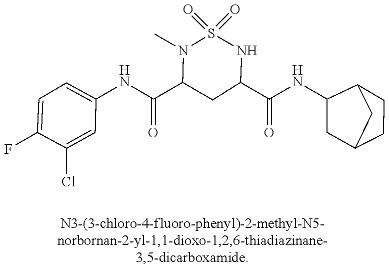 [SMILES: CN1C(C(=O)Nc2ccc(F)c(Cl)c2)CC(C(=O)NC2CC3CCC2C3)NS1(=O)=O] | ¹H NMR (400 MHz, DMSO-d6): δ 10.52 (d, J = 2.4 Hz, 1H), 8.03 (t, J = 7.2 Hz, 1H), 7.96-7.93 (m, 1H), 7.57- 7.52 (m, 1H), 7.39 (t, J = 9.2 Hz, 1H), 7.20-7.13 (m, 1H), 4.27-4.02 (m, 2H), 3.91-3.89 (m, 1H), 2.56 (s, 3H), 2.29- 2.28 (m, 1H), 2.15-2.14 (m, 1H), 2.02- 1.78 (m, 3H), 1.63-1.42 (m, 2H), 1.38- 1.36 (m, 1H), 1.35-1.19 (m, 3H), 0.95- 0.87 (m, 1H). LCMS calcd for; C₁₉H₂₄ClFN4O4S. Found; 459.30 (M + 1). |
| 859 | 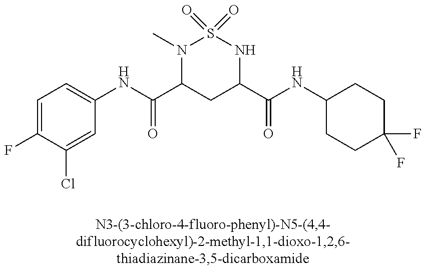 [SMILES: CN1C(C(=O)Nc2ccc(F)c(Cl)c2)CC(C(=O)NC2CCC(F)(F)CC2)NS1(=O)=O] | .¹H NMR (400 MHz, DMSO-d6): δ 10.52 (s, 1H), 8.08 (d, J = 7.7 Hz, 1H), 7.95 (dd, J = 7.0, 2.5 Hz, 1H), 7.55- 7.53 (m, 1H), 7.39 (t, J = 9.1 Hz, 1H), 7.26-7.25 (m, 1H), 4.20-4.17 (m, 1H), 4.06-4.04 (m, 1H), 3.78-3.75 (m, 1H), 2.56 (s, 3H), 2.06-1.84 (m, 6H), 1.78- 1.76 (m, 2H), 1.61-1.47 (m, 2H). LCMS calcd for; C₁₈H₂₂ClF₃N₄O₄S. Found; 483.25 (M + 1). |
| 862 | 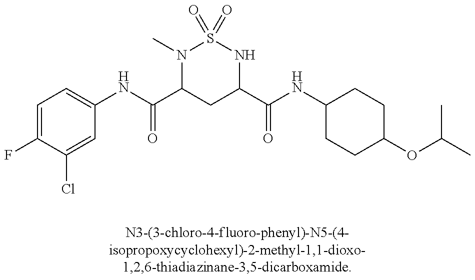 [SMILES: CC(C)OC1CCC(NC(=O)C2CC(C(=O)Nc3ccc(F)c(Cl)c3)N(C)S(=O)(=O)N2)CC1] | ¹H NMR (400 MHz, DMSO-d6): δ 10.52 (s, 1H), 7.98-7.93 (m, 2H), 7.57- 7.52 (m, 1H), 7.40 (t, J = 9.2 Hz, 1H), 7.15-7.12 (m, 1H), 4.21-4.08 (m, 2H), 3.64-3.59 (m, 2H), 3.48-3.46 (m, 1H), 2.57 (s, 3H), 1.98-1.85 (m, 2H), 1.68- 1.62 (m, 2H), 1.57-1.48 (m, 6H), 1.06 (d, J = 6.0 Hz, 6H). LCMS calcd for; C₂₁H₃₀ClFN₄O₅S. Found; 505.1 (M + 1). |
| 862- | N3-(3-chloro-4-fluoro-phenyl)-N5-(4- | ¹H NMR (400 MHz, DMSO-d6): δ |
| ISOMER-A | isopropoxycyclohexyl)-2-methyl-1,1-dioxo- | 10.51 (s, 1H), 7.96-7.93 (m, 2H), 7.56- |
| | 1,2,6-thiadiazinane-3,5-dicarboxamide. | 7.51 (m, 1H), 7.39 (t, J = 9.2 Hz, 1H), |
| | | 7.18-7.10 (m, 1H), 4.22-4.06 (m, 2H), |
| | | 3.66-3.60 (m, 2H), 3.50-3.45 (m, 1H), |
| | | 2.56 (s, 3H), 1.96-1.82 (m, 2H), 1.68- |
| | | 1.44 (m, 8H), 1.06 (d, J = 6.0 Hz, |
| | | 6H). LCMS calcd for; |
| | | C₂₁H₃₀ClFN₄O₅S. Found; |
| | | 505.20 (M + 1). |
| 862- | N3-(3-chloro-4-fluoro-phenyl)-N5-(4- | ¹H NMR (400 MHz, DMSO-d6): δ |
| ISOMER-B | isopropoxycyclohexyl)-2-methyl-1,1-dioxo- | 10.51 (s 1H), 7.99-7.91 (m, 2H), 7.57- |
| | 1,2,6-thiadiazinane-3,5-dicarboxamide. | 7.52 (m, 1H), 7.39 (t, J = 9.2 Hz, 1H), |
| | | 7.18-7.08 (m, 1H), 4.19-4.04 (m, 2H), |
| | | 3.65-3.60 (m, 2H), 3.49-3.45 (m, 1H), |
| | | 2.56 (s, 3H), 1.96-1.82 (m, 2H), 1.64- |
| | | 1.60 (m, 2H), 1.59-1.42 (m, 6H), 1.06 |
| | | (d, J = 6.0 Hz, 6H). LCMS calcd for, |
| | | C₂₁H₃₀ClFN₄O₅S. Found; |
| | | 505.15 (M + 1). |
| 866 | 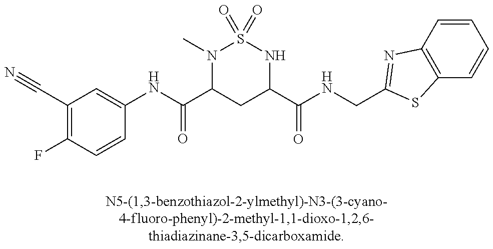 [SMILES: CN1C(C(=O)Nc2ccc(F)c(C#N)c2)CC(C(=O)NCc2nc3ccccc3s2)NS1(=O)=O] | ¹H NMR (400 MHz, DMSO-d6): δ 10.71 (s, 1H), 9.16 (t, J = 6.0 Hz, 1H), 81.8-8.16 (m, 1H), 8.12-8.04 (m, 1H), 7.99-7.88 (m, 2H), 7.59-7.39 (m, 4H), 4.83-4.62 (m, 2H), 4.31-4.21 (m, 2H), 2.60 (s, 3H), 2.10-1.91 (m, 2H). LCMS calcd for; C₂₁H₁₉FN₆O₄S₂. Found; 503 (M + 1). |
| 868 | 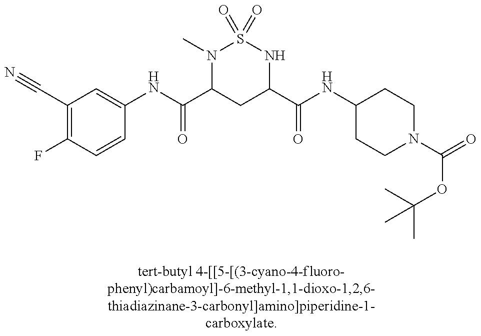 [SMILES: CN1C(C(=O)Nc2ccc(F)c(C#N)c2)CC(C(=O)NC2CCN(C(=O)OC(C)(C)C)CC2)NS1(=O)=O] | ¹H NMR (400 MHz, DMSO-d6): δ 10.68 (d, J = 7.5 Hz, 1H), 8.15-8.14 (m, 1H), 8.05 (d, J = 7.7 Hz, 1H), 7.91-7.89 (m, 1H), 7.53 (t, J = 9.1 Hz, 1H), 7.29 (d, J = 10.1 Hz, 1H), 4.24- 4.20 (m, 1H), 4.10-3.99 (m, 1H), 3.83- 3.73 (m, 2H), 3.26-3.21 (m, 1H), 2.89- 2.80 (m, 2H), 2.57 (s, 3H), 1.91-1.88 (m, 2H), 1.74-1.64 (m, 2H), 1.39 (s, 9H), 1.37-1.20 (m, 2H). LCMS calcd for; C₂₃H₃₁FN₆O₆S. Found; 561.40 (M + 23). |
| 888 | 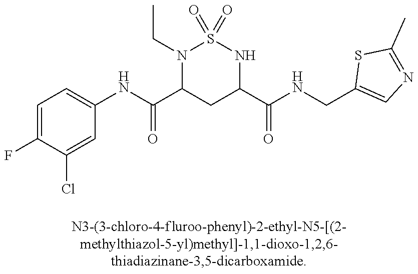 [SMILES: CCN1C(C(=O)Nc2ccc(F)c(Cl)c2)CC(C(=O)NCc2cnc(C)s2)NS1(=O)=O] | ¹H NMR (400 MHz, DMSO-d6): δ 10.57 (s, 1H), 8.72 (t, J = 5.6 Hz, 1H), 7.95 (dd, J = 6.8, 2.4 Hz, 1H), 7.58- 7.53 (m, 1H), 7.47 (s, 1H), 7.39 (t, J = 9.2 Hz, 1H), 7.29-7.26 (m, 1H), 4.46- 4.34 (m, 3H), 4.09-4.04 (m, 1H), 3.29- 3.22 (m, 1H), 2.92-2.82 (m, 1H), 2.59 (s, 3H), 1.98-1.94 (m, 1H), 1.86-1.76 (m, 1H), 1.08 (t, J = 7.6 Hz, 3H). LCMS calcd for; C₁₈H₂₁ClFN₅O₄S₂. Found; 490 (M + 1). |
| 888- | N3-(3-chloro-4-fluoro-phenyl)-2-ethyl-N5-[(2- | ¹H NMR (400 MHz, DMSO-d6): δ |
| ISOMER-A | methylthiazol-5-yl)methyl]-1,1-dioxo-1,2,6- | 10.58 (s, 1H), 8.72 (t, J = 5.6 Hz, 1H), |
| | thiadiazinane-3,5-dicarboxamide. | 7.95 (dd, J = 6.8, 2.4 Hz, 1H), 7.58- |
| | | 7.54 (m, 1H), 7.46 (s, 1H), 7.40 (t, J = |
| | | 9.2 Hz, 1H), 7.29-7.27 (m, 1H), 4.43- |
| | | 4.35 (m, 3H), 4.08-4.04 (m, 1H), 3.28- |
| | | 3.23 (m, 1H), 2.90-2.84 (m, 1H), 2.59 |
| | | (s, 3H), 1.98-1.94 (m, 1H), 1.83-1.76 |
| | | (m, 1H), 1.08 (t, J = 7.6 Hz, |
| | | 3H). LCMS calcd for; |
| | | C₁₈H₂₁ClFN₅O₄S₂. Found; 490 (M + 1). |
| 888- | N3-(3-chloro-4-fluoro-phenyl)-2-ethyl-N5-[(2- | ¹H NMR (400 MHz, DMSO-d6): δ |
| ISOMER-B | methylthiazol-5-yl)methyl]-1,1-dioxo-1,2,6- | 10.58 (s, 1H), 8.72 (t, J = 6.0 Hz, 1H), |
| | thiadiazinane-3,5-dicarboxamide. | 7.96-7.93 (m, 1H), 7.57-7.54 (m, 1H), |
| | | 7.46 (s, 1H), 7.40 (t, J = 9.2 Hz, 1H), |
| | | 7.28-7.25 (m, 1H), 4.43-4.35 (m, 3H), |
| | | 4.08-4.04 (m, 1H), 3.28-3.23 (m, 1H), |
| | | 2.90-2.84 (m, 1H), 2.59 (s, 3H), 1.98- |
| | | 1.94 (m, 1H), 1.86-1.76 (m, 1H), 1.08 |
| | | (t, J = 7.2 Hz, 3H). LCMS calcd for; |
| | | C₁₈H₂₁ClFN₅O₄S₂. Found; 490 (M + 1). |
| 890 | 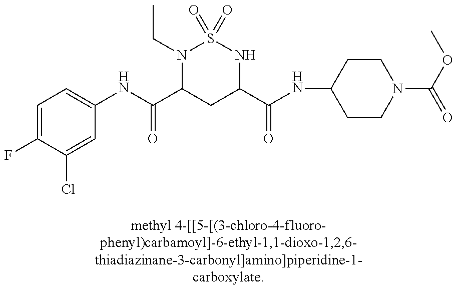 [SMILES: CCN1C(C(=O)Nc2ccc(F)c(Cl)c2)CC(C(=O)NC2CCN(C(=O)OC)CC2)NS1(=O)=O] | ¹H NMR (400 MHz, DMSO-d6): δ 10.58 (s, 1H), 8.03 (d, J = 7.7 Hz, 1H), 7.95 (dd, J = 6.8, 2.6 Hz, 1H), 7.58- 7.53 (m, 1H), 7.40 (t, J = 9.1 Hz, 1H), 7.17-7.15 (m, 1H), 4.38-4.34 (m, 1H), 4.05-4.03 (m, 1H), 3.86-3.76 (m, 3H), 3.58 (s, 3H), 3.31-3.21 (m, 1H), 2.95- 2.83 (m, 3H), 1.97-1.78 (m, 4H), 1.37- 1.26 (m, 2H), 1.08 (t, J = 7.1 Hz, 3h). LCMS calcd for; C₂₀H₂₇ClFN₅O₆S. Found; 520.35 (M + 1). |
| 890- | methyl 4-[[5-[(3-chloro-4-fluoro- | ¹H NMR (400 MHz, DMSO-d6): δ |
| ISOMER-A | phenyl)carbamoyl]-6-ethyl-1,1-dioxo-1,2,6- | 10.54 (bs, 1H), 7.96-7.93 (m, 2H), |
| | thiadiazinane-3-carbonyl]amino]piperidine-1- | 7.57-7.54 (m, 1H), 7.39 (t, J = 8.8 Hz, |
| | carboxylate. | 1H), 7.20-7.15 (m, 1H), 4.32-4.10 |
| | | (m, 1H), 4.00-3.98 (m, 1H), 3.87-3.85 |
| | | (m, 1H), 3.75-3.74 (m, 2H), 3.58 (s, |
| | | 3H), 2.92-2.85 (m, 4H), 1.94-1.90 (m, |
| | | 1H), 1.85-1.79 (m, 1H), 1.73-1.68 (m, |
| | | 2H), 1.34-1.23 (m, 2H), 1.08 (t, J = |
| | | 6.8 Hz, 3H). LCMS calcd for; |
| | | C₂₀H₂₇ClFN₅O₆S. Found; 520.30 |
| | | (M + 1). |
| 890- | methyl 4-[[5-[(3-chloro-4-fluoro- | ¹H NMR (400 MHz, DMSO-d6): δ |
| ISOMER-B | phenyl)carbamoyl]-6-ethyl-1,1-dioxo-1,2,6- | 10.50 (bs, 1H), 7.97-7.94 (m, 2H), |
| | thiadiazinane-3-carbonyl]amino]piperidine-1- | 7.58-7.54 (m, 1H), 7.39 (t, J = 8.8 Hz, |
| | carboxylate. | 1H), 7.20-7.15 (m, 1H), 4.25-4.20 (m, |
| | | 1H), 3.95-3.84 (m, 3H), 3.76-3.74 (m, |
| | | 1H), 3.58 (s, 3H), 2.92-2.86 (m, 4H), |
| | | 1.95-1.91 (m, 1H), 1.83-1.76 (m, 1H), |
| | | 1.73-1.66 (m, 2H), 1.32-1.24 (m, |
| | | 2H), 1.08 (t, J = 6.8 3H). LCMS calcd |
| | | for; C₂₀H₂₇ClFN₅O₆S. Found; 520.35 |
| | | (M + 1). |
| 891 | 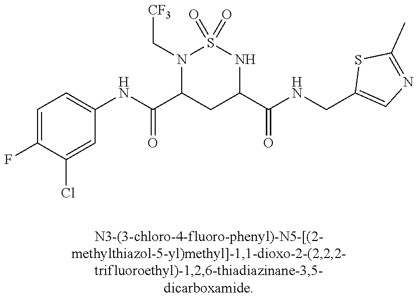 [SMILES: Cc1ncc(CNC(=O)C2CC(C(=O)Nc3ccc(F)c(Cl)c3)N(CC(F)(F)F)S(=O)(=O)N2)s1] | ¹H NMR (400 MHz, DMSO-d6): δ 10.80 (s, 1H), 8.79 (t, J = 5.2 Hz, 1H), 7.86-7.83 (m, 1H), 7.48-7.44 (m, 2H), 7.39-7.12 (m, 2H), 4.70-4.66 (m, 1H), 4.46-4.35 (m, 2H), 4.19-4.12 (m, 2H), 3.81-3.72 (m, 1H), 2.56 (s, 3H), 1.78- 1.64 (m, 2H). LCMS calcd for; C₁₈H₁₈ClF₄N₅O₄S₂. Found; 544.25 (M + 1). |
| 891- | N3-(3-chloro-4-fluoro-phenyl)-N5-[(2- | ¹H NMR (400 MHz, DMSO-d6): δ |
| ISOMER-A | methylthiazol-5-yl)methyl]-1,1-dioxo-2-(2,2,2- | 10.90 (s, 1H), 8.80 (t, J = 5.6 Hz, 1H), |
| | trifluoroethyl)-1,2,6-thiadiazinane-3,5- | 7.89 (dd, J = 7.2, 2.8 Hz, 1H), 7.52- |
| | dicarboxamide. | 7.48 (m, 2H), 7.42-7.38 (m, 2H), 4.73- |
| | | 4.69 (m, 1H), 4.48-4.38 (m, 2H), |
| | | 4.20-4.13 (m, 2H), 3.85-3.78 (m, |
| | | 1H), 2.59 (s, 3H), 1.81-1.71 (m, |
| | | 2H). LCMS calcd for; |
| | | C₁₈H₁₈ClF₄N₅O₄S₂. Found; |
| | | 544.05 (M + 1). |
| 891- | N3-(3-chloro-4-fluoro-phenyl)-N5-[(2- | ¹H NMR (400 MHz, DMSO-d6): δ |
| ISOMER-B | methylthiazol-5-yl)methyl]-1,1-dioxo-2-(2,2,2- | 10.90 (s, 1H), 8.80 (t, J = 6.0 Hz, 1H), |
| | trifluoroethyl)-1,2,6-thiadiazinane-3,5- | 7.89 (dd, J = 6.4, 2.8 Hz, 1H), 7.52- |
| | dicarboxamide. | 7.47 (m, 2H), 7.42-7.37 (m, 2H), 4.73- |
| | | 4.69 (m, 1H), 4.45-4.42 (m, 2H), |
| | | 4.20-4.13 (m, 2H), 3.85-3.74 (m, |
| | | 1H), 2.59 (s, 3H), 1.81-1.71 (m, |
| | | 2H). LCMS calcd for; |
| | | C₁₈H₁₈ClF₄N₅O₄S₂. Found; |
| | | 544.05 (M + 1). |
| 893 | 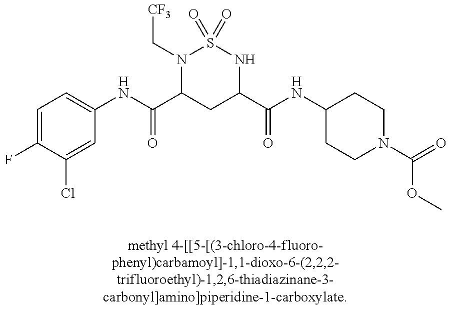 [SMILES: COC(=O)N1CCC(NC(=O)C2CC(C(=O)Nc3ccc(F)c(Cl)c3)N(CC(F)(F)F)S(=O)(=O)N2)CC1] | ¹H NMR (400 MHz, DMSO-d6): δ 10.90 (s, 1H), 8.10 (d, J = 7.6 Hz, 1H), 7.86-7.84 (m, 1H), 7.48-7.45 (m, 1H), 7.37 (t, J = 8.8 Hz, 1H), 73.2-7.20 (m, 1H), 4.70-4.66 (m, 1H), 4.18-4.10 (m, 2H), 3.82-3.73 (m, 4H), 3.55 (s, 3H), 2.95-2.90 (m, 2H), 1.73-1.64 (m, 4H), 1.32-1.20 (m, 2H). LCMS calcd for; C₂₀H₂₄ClF₄N₅O₆S. Found; 574 (M + 1). |
| 893- | methyl 4-[[5-[(3-chloro-4-fluroo- | ¹H NMR (400 MHz, DMSO-d6): δ |
| ISOMER-A | phenyl)carbamoyl]-1,1-dioxo-6-(2,2,2- | 10.90 (s, 1H), 8.12 (d, J = 8.0 Hz, 1H), |
| | triflurooethyl)-1,2,6-thiadiazinane-3- | 7.89 (dd, J = 6.8, 2.4 Hz, 1H), 7.51- |
| | carbonyl]amino]piperidine-1-carboxylate. | 7.47 (m, 1H), 7.40 (t, J = 8.8 Hz, 1H), |
| | | 7.30-7.20 (m, 1H), 4.72-4.68 (m, |
| | | 1H), 4.21-4.11 (m, 2H), 3.85-3.77 |
| | | (m, 4H), 3.58 (s, 3H), 2.96-2.94 (m, |
| | | 2H), 1.76-1.67 (m, 4H), 1.33-1.27 (m, |
| | | 2H). LCMS calcd for; |
| | | C₂₀H₂₄ClF₄N₅O₆S. Found; 574.10 |
| | | (M + 1). |
| 893- | methyl 4-[[5-[(3-chloro-4-fluroo- | ¹H NMR (400 MHz, DMSO-d6): δ |
| ISOMER-B | phenyl)carbamoyl]-1,1-dioxo-6-(2,2,2- | 10.90 (s, 1H), 8.10 (d, J = 78.6 Hz, 1H), |
| | trifluoroethyl)-1,2,6-thiadiazinane-3- | 7.86-7.84 (m, 1H), 7.48-7.45 (m, 1H), |
| | carbonyl]amino]piperidine-1-carboxylate. | 7.37 (t, J = 8.8 Hz, 1H), 7.32-7.20 (m, |
| | | 1H), 4.70-4.66 (m, 1H), 4.18-4.10 (m, |
| | | 2G), 3.82-3.73 (m, 4H), 3.55 (s, 3H), |
| | | 2.95-2.90 (m, 2H), 1.73-1.64 (m, 4H), |
| | | 1.32-1.20 (m, 2H). LCMS calcd for; |
| | | C₂₀H₂₄ClF₄N₅O₆S. Found; 574.10 |
| | | (M + 1). |
| 896 | 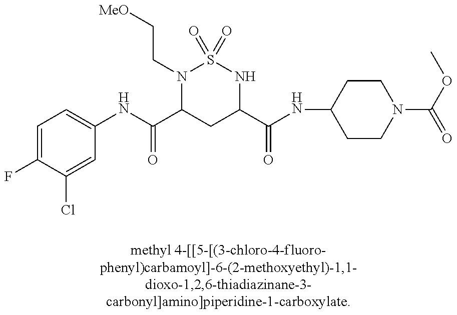 [SMILES: COCCN1C(C(=O)Nc2ccc(F)c(Cl)c2)CC(C(=O)NC2CCN(C(=O)OC)CC2)NS1(=O)=O] | ¹H NMR (400 MHz, DMSO-d6): δ 10.47 (s, 1H), 8.06 (d, J = 7.6 Hz, 1H), 7.96-7.92 (m, 1H), 7.56-7.52 (m, 1H), 7.40 (t, J = 8.8 Hz, 1H), 7.23 (s, 1H), 4.50-4.46 (m, 1H), 4.08-4.05 (m ,1H), 3.86-3.74 (m, 3H), 3.59 (s, 3H), 3.44- 3.36 (m, 3H), 3.12 (s, 3H), 3.03-2.94 (m, 3H), 1.90-1.72 (m, 4H), 1.34-1.29 (m, 2H). LCMS calcd for; C₂₁H₂₉ClFN₅O₇S. Found; 550.50 (M + 1). |
| 896- | methyl 4-[[5-[(3-chloro-4-fluoro- | ¹H NMR (400 MHz, DMSO-d6): δ |
| ISOMER-A | phenyl)carbamoyl]-6-(2-methoxyethyl)-1,1- | 10.46 (s, 1H), 8.05 (d, J = 7.6 Hz, 1H), |
| | dioxo-1,2,6-thiadiazinane-3- | 7.94-7.91 (m, 1H), 7.56-7.51 (m, 1H), |
| | carbonyl]amino]piperidine-1-carboxylate | 7.40 (t, J = 8.8 Hz, 1H), 7.25-7.20 (m, |
| | | 1H), 4.48-4.45 (m, 1H), 4.07-4.03 (m, |
| | | 1H), 3.86-3.74 (m, 3H), 3.58 (s, 3H), |
| | | 3.43-3.32 (m, 3H), 3.11 (s, 3H), 3.02- |
| | | 2.93 (m, 3H), 1.89-1.71 (m, 4H), 1.34- |
| | | 1.29 (m, 2H). LCMS calcd for; |
| | | C₂₁H₂₉ClFN₅O₇S. Found; 550.55 |
| | | (M + 1). |
| 896- | methyl 4-[[5-[(3-chloro-4-fluoro- | ¹H NMR (400 MHz, DMSO-d6): δ |
| ISOMER-B | phenyl)carbamoyl]-6-(2-methoxyethyl)-1,1- | 10.44 (s, 1H), 8.02 (d, J = 7.2 Hz, 1H), |
| | dioxo-1,2,6-thiaidazinane-3- | 7.90-7.88 (m, 1H), 7.52-7.48 (m, 1H), |
| | carbonyl]amino]piperidine-1-carboxylate | 7.36 (t, J = 8.8 Hz, 1H), 7.19-7.17 (m, |
| | | 1H), 4.46-4.43 (m, 1H), 4.06-4.01 (m, |
| | | 1H), 3.82-3.71 (m, 3H), 3.55 (s, 3H), |
| | | 3.41-3.34 (m, 3H), 3.08 (s, 3H), 2.99- |
| | | 2.90 (m, 3H), 1.86-1.68 (m, 4H), 1.31- |
| | | 1.25 (m, 2H). LCMS calcd for; |
| | | C₂₁H₂₉ClFN₅O₇S. Found; 550.40 |
| | | (M + 1). |
| 906 | 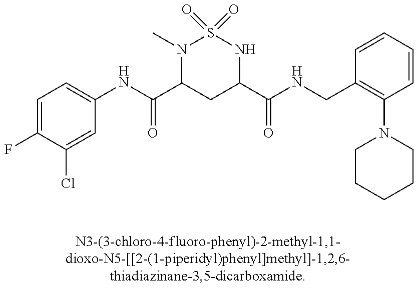 [SMILES: CN1C(C(=O)Nc2ccc(F)c(Cl)c2)CC(C(=O)NCc2ccccc2N2CCCCC2)NS1(=O)=O] | ¹H NMR (400 MHz, DMSO-d6): δ 10.54 (s, 1H), 8.52-8.50 (m, 1H), 7.97- 7.93 (m, 1H), 7.56-7.53 (m, 1H), 7.44- 7.34 (m, 2H), 7.23-7.19 (m, 2H), 7.12- 6.99 (m, 2H), 4.38-4.36 (m, 2H), 4.23- 4.15 (m, 2H), 2.78-2.76 (m, 4H), 2.58 (s, 3H), 2.00-1.95 (m, 2H), 1.66-1.64 (m, 4H), 1.52-1.50 (m, 2H). LCMS calcd for; C₂₄H₂₉ClFN₅O₄S. Found; 538.40 (M + 1). |
| 907 | 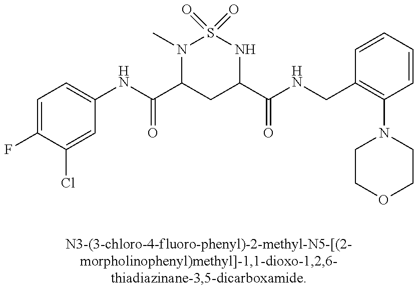 [SMILES: CN1C(C(=O)Nc2ccc(F)c(Cl)c2)CC(C(=O)NCc2ccccc2N2CCOCC2)NS1(=O)=O] | ¹H NMR (400 MHz, DMSO-d6): δ 10.52 (s, 1H), 8.53 (t, J = 5.8 Hz, 1H), 7.94 (dd, J = 6.9, 2.5 Hz, 1H), 7.55- 7.51 (m, 1H), 7.43-7.36 (m, 2H), 7.26- 7.21 (m, 2H), 7.16-7.03 (m, 2H), 4.40 (d, J = 5.7 Hz, 2H), 4.21-4.11 (m, 2H), 3.72 (t, J = 4.0 Hz, 4H), 2.82 (t, J = 4.0 Hz, 4H), 2.57 (s, 3H), 1.99-1.94 (m, 2H). LCMS calcd for; C₂₃H₂₇ClFN₅O₅S. Found; 540.1 (M + 1). |
| 909 | 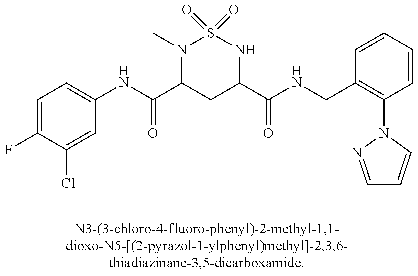 [SMILES: CN1C(C(=O)Nc2ccc(F)c(Cl)c2)CC(C(=O)NCc2ccccc2-n2cccn2)NS1(=O)=O] | ¹H NMR (400 MHz, DMSO-d6): δ 10.50 (s, 1H), 8.52-8.49 (m, 1H), 8.10 (s, 1H), 7.96-7.94 (m, 1H), 7.74 (s, 1H), 7.55-753 (m, 1H), 7.48-7.37 (m, 5H), 7.34-7.23 (m, 1H), 6.51 (s, 1H), 4.31-4.24 (m, 2H), 4.22-4.12 (m, 2H), 2.57 (s, 3H), 1.98-1.87 (m, 2H). LCMS calcd for; C₂₂H₂₂ClFN₆O₄S. Found; 521.30 (M + 1). |
| 910 | 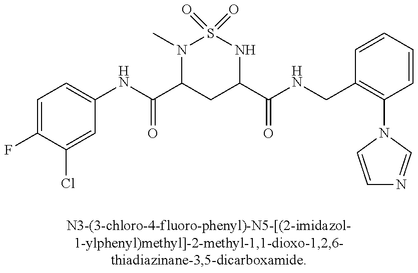 [SMILES: CN1C(C(=O)Nc2ccc(F)c(Cl)c2)CC(C(=O)NCc2ccccc2-n2ccnc2)NS1(=O)=O] | ¹H NMR (400 MHz, DMSO-d6): δ 10.53 (s, 1H), 8.62 (t, J = 5.6 Hz, 1H), 8.08 (s, 1H), 7.97-7.94 (m, 1H), 7.56-7.50 (m, 4H), 7.48-7.34 (m, 4H), 7.22 (s, 1H), 4.20-4.09 (m, 4H), 2.57 (s, 3H), 1.99-1.91 (m, 2H). LCMS calcd for; C₂₂H₂₂ClFN₆O₄S. Found; 521.30 (M + 1). |
| 910- | N3-(3-chloro-4-fluoro-phenyl)-N5-[(2-imidaozl- | ¹H NMR (400 MHz, DMSO-d6): δ |
| ISOMER-A | 1-ylphenyl)methyl]-2-methyl-1,1-dioxo-1,2,6- | 10.53 (s, 1H), 8.61 (t, J = 5.2 Hz, |
| | thiadiazinane-3,5-dicarboxamide. | 1H), 7.97-7.94 (m, 2H), 7.57-7.48 (m, |
| | | 4H), 7.45-7.39 (m, 3H), 7.37-7.32 (m, |
| | | 1H), 7.16 (s, 1H), 4.21-4.08 (m, 4H), |
| | | 2.57 (s, 3H), 1.99-1.91 (m, 2H). LCMS |
| | | calcd for; C₂₂H₂₂ClFN₆O₄S. Found; |
| | | 521.10 (M + 1). |
| 910- | N3-(3-chloro-4-fluoro-phenyl)-N5-[(2-imidazol- | ¹H NMR (400 MHz, DMSO-d6): δ |
| ISOMER-B | 1-ylphenyl)methyl]-2-methyl-1,1-dioxo-1,2,6- | 10.52 (s, 1H), 8.60 (t, J = 5.2 Hz, |
| | thiadiazinane-3,5-dicarboxamide. | 1H), 7.96-7.94 (m, 1H), 7.85 (s, 1H), |
| | | 7.55-7.53 (m, 1H), 7.50-7.47 (m, 3H), |
| | | 7.44-7.31 (m, 4H), 7.10 (s, 1H), 4.17- |
| | | 4.11 (m, 4H), 2.56 (s, 3H), 1.96-1.91 |
| | | (m, 2H). LCMS calcd for; |
| | | C₂₂H₂₂ClFN₆O₄S. Found; 521.10 |
| | | (M + 1). |
| 911 | 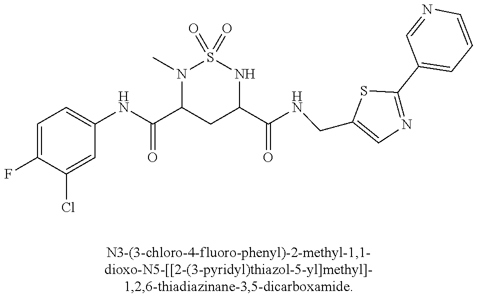 [SMILES: CN1C(C(=O)Nc2ccc(F)c(Cl)c2)CC(C(=O)NCc2cnc(-c3cccnc3)s2)NS1(=O)=O] | ¹H NMR (400 MHz, DMSO-d6): δ 10.53 (s, 1H), 9.08 (d, J = 2.4 Hz, 1H), 8.86 (t, J = 6.0 Hz, 1H), 8.66-8.64 (m, 1H), 8.27-8.23 (m, 1H), 7.94 (dd, J = 6.8, 2.4 Hz, 1H), 7.84 (s, 1H), 7.56- 7.51 (m, 2H), 7.45-7.36 (m, 2H), 4.54 (d, J = 6.4 Hz, 2H), 4.22-4.10 (m, 2H), 2.57 (s, 3H), 2.02-1.89 (m, 2H). LCMS calcd for; C₂₁H₂₀ClFN₆O₄S₂. Found; 539.1 (M + 1). |
| 911- | N3-(3-chloro-4-fluoro-phenyl)-2-methyl-1,1- | ¹H NMR (400 MHz, DMSO-d6): δ |
| ISOMER-A | dioxo-N5-[[2-(3-pyridyl)thiazol-5-yl]methyl]- | 10.40 (s, 1H), 9.08 (s, 1H), 8.70-8.63 |
| | 1,2,6-thiadiazinane-3,5-dicarboxamide. | (m, 2H), 8.26-8.24 (m, 1H), 7.97-7.94 |
| | | (m, 1H), 7.82 (s, 1H), 7.53-7.50 (m, |
| | | 3H), 7.37 (t, J = 9.2 Hz, 1H), 4.54 (d, |
| | | J = 4.8 Hz, 2H), 3.97-3.95 (m, 2H), |
| | | 2.02-1.98 (m, 1H), 1.84- |
| | | 1.74 (m, 1H). LCMS calcd for; |
| | | C₂₁H₂₀ClFN₆O₄S₂. Found; 539 (M + 1). |
| 911- | N3-(3-chloro-4-fluoro-phenyl)-2-methyl-1,1- | ¹H NMR (400 MHz, DMSO-d6): δ |
| ISOMER-B | dioxo-N5-[[2-(3-pyridyl)thiazol-5-yl]methyl]- | 10.51 (s, 1H), 9.08 (s, 1H), 8.86-8.82 |
| | 1,2,6-thiadiazinane-3,5-dicarboxmaide. | (m, 1H), 8.65-8.63 (m, 1H), 8.26-8.24 |
| | | (m, 1H), 7.97-7.93 (m, 1H), 7.84 (s, |
| | | 1H), 7.55-7.50 (m, 2H), 7.45-7.32 (m, |
| | | 2H), 4.54 (d, J = 5.6 Hz, 2H), 4.17- |
| | | 4.09 (m, 2H), 2.56 (s, 3H), 2.02-1.91 |
| | | (m, 2H). LCMS calcd for; |
| | | C₂₁H₂₀ClFN₆O₄S₂. Found; 539.1 |
| | | (M + 1). |
| 912 | 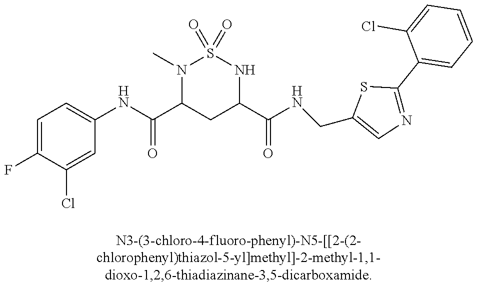 [SMILES: CN1C(C(=O)Nc2ccc(F)c(Cl)c2)CC(C(=O)NCc2cnc(-c3ccccc3Cl)s2)NS1(=O)=O] | ¹H NMR (400 MHz, DMSO-d6): δ 10.52 (s, 1H), 8.85 (t, J = 5.6 Hz, 1H), 8.15-8.12 (m, 1H), 7.96-7.93 (m, 1H), 7.87 (s, 1H), 7.64-7.62 (m, 1H), 7.56- 7.52 (m, 1H), 7.50-7.47 (m, 2H), 7.43- 7.36 (m, 2H), 4.57 (d, J = 5.6 Hz, 2H), 4.22-4.18 (m, 1H), 4.13-4.10 (m, 1H), 2.57 (s, 3H), 2.02-1.92 (m, 2H). LCMS calcd for; C₂₂H₂₀Cl₂FN₅O₄S₂. Found; 572.30 (M + 1). |
| 912- | N3-(3-chloro-4-fluoro-phenyl)-N5-[[2-(2- | .¹H NMR (400 MHz, DMSO-d6): δ |
| ISOMER-A | chlorophenyl)thiazol-5-yl]methyl]-2-methyl-1,1- | 10.45 (s, 1H), 8.79-8.77 (m, 1H), 8.11- |
| | dioxo-1,2,6-thiadiazinane-3,5-dicarboxamide | 8.09 (m, 1H), 7.93-7.90 (m, 1H), 7.83 |
| | | (s, 1H), 7.61-7.58 (m, 1H), 7.52-7.49 |
| | | (m, 1H), 7.47-7.44 (m, 3H), 7.35 (t, J = |
| | | 9.2 Hz, 1H), 4.53 (d, J = 6.0 Hz, |
| | | 2H), 4.10-3.98 (m, 2H), 2.92 (s, |
| | | 3H), 2.00-1.80 (m, 2H). LCMS calcd |
| | | for; C₂₂H₂₀Cl₂FN₅O₄S₂. Found; 572.05 |
| | | (M + 1). |
| 912- | N3-(3-chloro-4-fluoro-phenyl)-N5-[[2-(2- | ¹H NMR (400 MHz, DMSO-d6): δ |
| ISOMER-B | chlorophenyl)thiazol-5-yl]methyl]-2-methyl-1,1- | 10.48 (s, 1H), 8.81-8.79 (s, 1H), 8.15- |
| | dioxo-1,2,6-thiadiazinane-3,5-dicarboxamide. | 8.07 (m, 1H), 7.95-7.90 (m, |
| | | 1H), 7.84 (s, 1H), 7.62-7.57 (m, 1H), |
| | | 7.52-7.49 (m, 1H), 7.47-7.43 (m, |
| | | 3H), 7.36 (t, J = 9.2 Hz, 1H), 4.53 (d, |
| | | J = 5.2 Hz, 2H), 4.17-4.04 (m, 2H), 2.53 |
| | | (s, 3H), 1.99-1.82 (m, 2H). LCMS |
| | | calcd for; C₂₂H₂₀Cl₂FN₅O₄S₂. Found; |
| | | 572.05 (M + 1). |
| 913 | 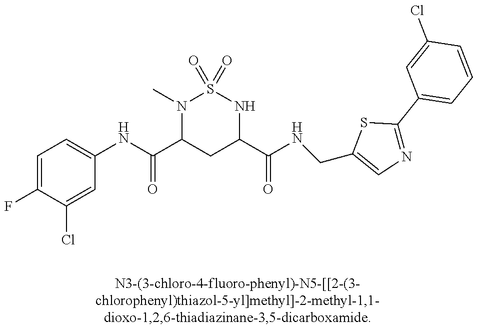 [SMILES: CN1C(C(=O)Nc2ccc(F)c(Cl)c2)CC(C(=O)NCc2cnc(-c3cccc(Cl)c3)s2)NS1(=O)=O] | ¹H NMR (400 MHz, DMSO-d6): δ 10.53 (s, 1H), 8.86-8.84 (m, 1H), 7.97- 7.94 (m, 1H), 7.92-7.90 (m, 1H), 7.86- 7.80 (m, 2H), 7.56-7.50 (m, 3H), 7.49- 7.36 (m, 2H), 4.53 (d, J = 6.0 Hz, 2H), 4.21-4.18 (m, 1H), 4.12- 4.08 (m, 1H), 2.57 (s, 3H), 2.02-1.89 (m, 2H). LCMS calcd for; C₂₂H₂₀Cl₂FN₅O₄S₂. Found; 571.9 (M + 1). |
| 913- | N3-(3-chloro-4-fluoro-phenyl)-N5-[[2-(3- | ¹H NMR (400 MHz, DMSO-d6): δ |
| ISOMER-A | chlorophenyl)thiazol-5-yl]methyl]-2-methyl-1,1- | 10.46 (s, 1H), 8.75-8.70 (m, 1H), |
| | dioxo-1,2,6-thiadiazinane-3,5-dicarboxamide. | 7.97-7.94 (m, 1H), 7.91 (s, 1H), 7.85- |
| | | 7.80 (m, 1H), 7.78 (s, 1H), 7.58-7.51 |
| | | (m, 4H), 7.37 (t, J = 9.2 Hz, 1H), 4.52 |
| | | (d, J = 5.6 Hz, 2H), 3.98-3.92 (m, |
| | | 2H), 2.46 (s, 3H), 2.02-1.96 (m, 1H), |
| | | 1.86-1.76 (m, 1H). LCMS calcd for; |
| | | C₂₂H₂₀Cl₂FN₅O₄S₂. Found; 572 |
| | | (M + 1). |
| 913- | N3-(3-chloro-4-fluoro-phenyl)-N5-[[2-(3- | ¹H NMR (400 MHz, DMSO-d6): δ |
| ISOMER-B | chlorophenyl)thiazol-5-yl]methyl]-2-methyl-1,1- | 10.52 (s, 1H), 8.86-8.82 (m, 1H), 7.97- |
| | dioxo-1,2,-thiadiazinane-3,5-dicarboxamide. | 7.94 (m, 1H), 7.91 (s, 1H), 7.85-7.83 |
| | | (m, 1H), 7.80 (s, 1H), 7.55-7.35 (m, |
| | | 5H), 4.52 (d, J = 5.2 Hz, 2H), 4.20- |
| | | 4.06 (m, 2H), 2.56 (s, 3H), 2.02-1.86 |
| | | (m, 2H). LCMS calcd for; |
| | | C₂₂H₂₀Cl₂FN₅O₄S₂. Found; 572 |
| | | (M + 1). |
| 914 | 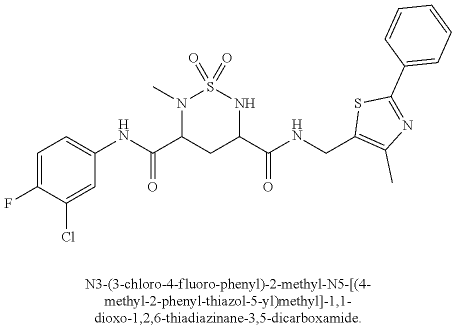 [SMILES: Cc1nc(-c2ccccc2)sc1CNC(=O)C1CC(C(=O)Nc2ccc(F)c(Cl)c2)N(C)S(=O)(=O)N1] | ¹H NMR (400 MHz, DMSO-d6): δ 105.2 (s, 1H), 8.79 (t, J = 6.0 Hz, 1H), 7.97-7.93 (m, 1H), 7.87-7.84 (m, 2H), 7.56-7.52 (m, 1H), 7.49-7.42 (m, 2H), 7.41-7.35 (m, 2H), 4.49- 4.38 (m, 2H), 4.22-4.19 (m, 1H), 4.10- 1.06 (m, 2H), 2.57 (s, 3H), 2.41 (s, 3H), 2.00-1.86 (m, 2H). LCMS calcd for; C₂₃H₂₃ClFN₅O₄S₂. Found; 552.05 (M + 1). |
| 914- | N3-(3-chloro-4-fluoro-phenyl)-2-methyl-N5-[(4- | ¹H NMR (400 MHz, DMSO-d6): δ |
| ISOMER-A | methyl-2-phenyl-thiazol-5-yl)methyl]-1,1- | 10.48 (s, 1H), 8.75-8.73 (m, 1H), 7.96- |
| | dioxo-1,2,6-thiadiazinane-3,5-dicarboxamide. | 7.94 (m, 1H), 7.87-7.84 (m, 2H), 7.55- |
| | | 7.53 (m, 12H), 7.47-7.45 (m, 3H), 7.40- |
| | | 7.36 (m, 2H), 4.44-4.42 (m, 2H), 4.15- |
| | | 4.03 (m, 2H), 2.40 (s, 3H), 1.99-1.87 |
| | | (m, 2H). LCMS calcd for; |
| | | C₂₃H₂₃ClFN₅O₄S₂. Found; 552.10 |
| | | (M + 1). |
| 914- | N3-(3-chloro-4-fluoro-phenyl)-2-methyl-N5-[(4- | ¹H NMR (400 MHz, DMSO-d6): δ |
| ISOMER-B | methyl-2-phenyl-thiazol-5-yl)methyl]-1,1- | 10.52 (s, 1H), 8.79 (t, J = 6.0 Hz, 1H), |
| | dioxo-1,2,6-thiadiazinane-3,5-dicarboxamide. | 7.96-7.93 (m, 1H), 7.87-7.84 (m, 2H), |
| | | 7.55-7.53 (m, 1H), 7.47-7.45 (m, 3H), |
| | | 7.41-7.36 (m, 2H), 4.44 (t, J = 5.2 Hz, |
| | | 2H), 4.22-4.20 (m, 1H), 4.10-4.07 (m, |
| | | 1H), 2.56 (s, 3H), 2.41 (s, 3H), 1.99- |
| | | 1.91 (m, 2H). LCMS calcd for; |
| | | C₂₃H₂₃ClFN₅O₄S₂. Found; 552.10 |
| | | (M + 1). |
| 915 | 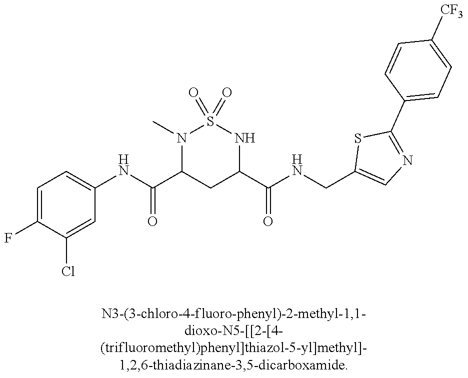 [SMILES: CN1C(C(=O)Nc2ccc(F)c(Cl)c2)CC(C(=O)NCc2cnc(-c3ccc(C(F)(F)F)cc3)s2)NS1(=O)=O] | ¹H NMR (400 MHz, DMSO-d6): δ 10.53 (s, 1H), 8.87 (t, J = 6.0 Hz, 1H), 8.11 (d, J = 8.4 Hz, 2H), 7.96-7.93 (m, 1H), 7.87-7.83 (m, 3H), 7.56-7.52 (m, 1H), 7.44-7.37 (m, 2H), 4.55 (d, J = 6.0 Hz, 2H), 4.22-4.18 (m, 1H), 4.13-4.10 (m, 1H), 2.57 (s, 3H), 2.02- 1.92 (m, 2H). LCMS calcd for; C₂₃H₂₀ClF₄N₅O₄S₂. Found; 606.30 (M + 1). |
| 916 | 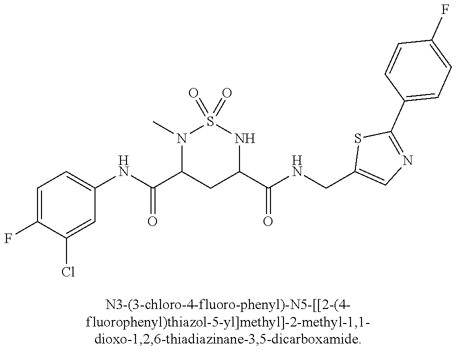 [SMILES: CN1C(C(=O)Nc2ccc(F)c(Cl)c2)CC(C(=O)NCc2cnc(-c3ccc(F)cc3)s2)NS1(=O)=O] | ¹H NMR (400 MHz, DMSO-d6): δ 10.53 (s, 1H), 8.85 (t, J = 5.6 Hz, 1H), 7.90-7.93 (m, 3H), 7.76 (s, 1H), 7.57- 7.52 (m, 1H), 7.44-.730 (m, 4H), 4.51 (d, J = 5.6 Hz, 2H), 4.23-4.19 (m, 1H), 4.11-4.08 (m, 1H), 2.57 (s, 3H), 2.02- 1.92 (m, 2H). LCMS calcd for; C₂₂H₂₀ClF₂N₅O₄S₂. Found; 556.30 (M + 1). |
| 916- | N3-(3-chloro-4-fluoro-phenyl)-N5-[[2-(4- | ¹H NMR (400 MHz, DMSO-d6): δ |
| ISOMER-A | fluorophenyl)thiazol-5-yl]methyl]-2-methyl-1,1- | 10.48 (s, 1H), 8.80-8.70 (m, 1H), 7.96- |
| | dioxo-1,2,6-thiadiazinane-3,5-dicarboxamide. | 7.92 (m, 3H), 7.74 (s, 1H), 7.57-7.52 |
| | | (m, 1H), 7.44-7.30 (m, 4H), 4.52 (d, J = 6.0 Hz, 2H), 4.11-4.03 (m, 2H), 2.53 |
| | | (s, 3H), 2.02-1.83 (m, 2H). LCMS |
| | | calcd for; C₂₂H₂₀ClF₂N₅O₄S₂. Found; |
| | | 556.05 (M + 1). |
| 916- | N3-(3-chloro-4-fluoro-phenyl)-N5-[[2-(4- | ¹H NMR (400 MHz, DMSO-d6): δ |
| ISOMER-B | fluorophenyl)thiazol-5-yl]methyl]-2-methyl-1,1- | 10.51 (s, 1H), 8.85-8.82 (m, 1H), 7.96- |
| | dioxo-1,2,6-thiadiazinane-3,5-dicarboxamide.. | 7.92 (m, 3H), 7.75 (s, 1H), 7.56-7.52 |
| | | (m, 1H), 7.42-7.30 (m, 4H), 4.51 (d, J = |
| | | 6.0 Hz, 2H), 4.21-4.07 (m, 2H), 2.57 |
| | | (s, 3H), 2.02-1.85 (m, 2H). LCMS |
| | | calcd for; C₂₂H₂₀ClF₂N₅O₄S₂. Found; |
| | | 556.05 (M + 1). |
| 917 | 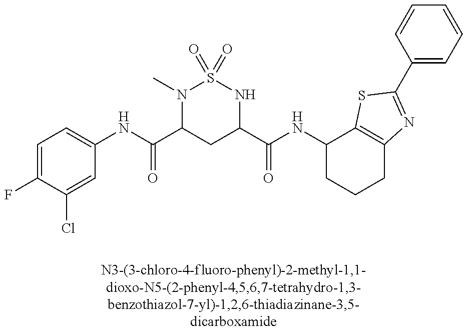 [SMILES: CN1C(C(=O)Nc2ccc(F)c(Cl)c2)CC(C(=O)NC2CCCc3nc(-c4ccccc4)sc32)NS1(=O)=O] | ¹H NMR (400 MHz, DMSO-d6): δ 10.53 (s, 1H), 8.73 (d, J = 8.0 Hz, 1H), 7.96-7.93 (m, 1H), 7.89-7.85 (m, 2H), 7.57-7.53 (m, 1H), 7.48-7.46 (m, 3H), 7.42-7.36 (m, 1H), 7.33-7.30 (m, 1H), 5.12-5.11 (m, 1H), 4.22-4.09 (m, 2H), 2.81-2.66 (m, 2H), 2.58 (s, 3H), 2.05- 1.98 (m, 4H), 1.84-1.78 (m, 2H). LCMS calcd for; C₂₅H₂₅ClFN₅O₄S₂. Found; 578.10 (M + 1). |
| 918 | 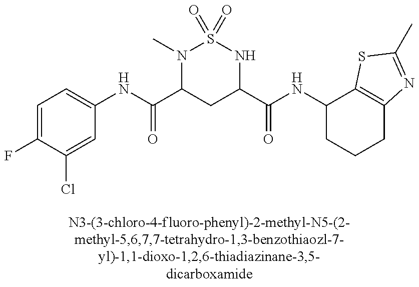 [SMILES: Cc1nc2c(s1)C(NC(=O)C1CC(C(=O)Nc3ccc(F)c(Cl)c3)N(C)S(=O)(=O)N1)CCC2] | ¹H NMR (400 MHz, DMSO-d6): δ 10.53 (s, 1H), 8.61 (d, J = 7.2 Hz, 1H), 7.95 (d, J = 6.8 Hz, 1H), 7.56- 7.52 (m, 1H), 7.39 (t, J = 9.6 Hz, 1H), 7.26-7.25 (m, 1H), 5.00-4.98 (m, 1H), 4.22-4.09 (m, 2H), 2.67-2.54 (m, 8H), 1.95-1.91 (m, 4H), 1.82-1.75 (m, 2H). LCMS calcd for; C₂₀H₂₃ClFN₅O₄S₂. Found; 516.10 (M + 1). |
| 920 | 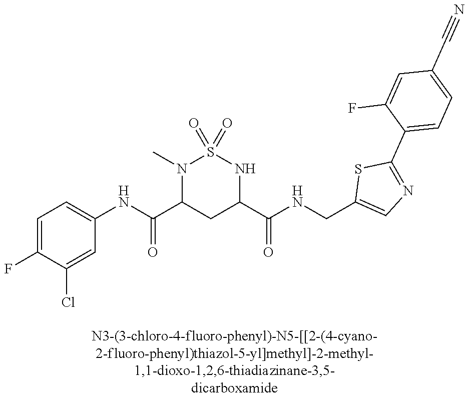 [SMILES: CN1C(C(=O)Nc2ccc(F)c(Cl)c2)CC(C(=O)NCc2cnc(-c3ccc(C#N)cc3F)s2)NS1(=O)=O] | .¹H NMR (400 MHz, DMSO-d6): δ 10.54 (s, 1H), 8.90 (t, J = 6.2 Hz, 1H), 8.36 (t, J = 8.0 Hz, 1H), 8.12 (d, J = 11.2 Hz, 1H), 7.99-7.94 (m, 2H), 7.85-7.83 (m, 1H), 7.56-7.53 (m, 1H), 7.48-7.45 (m, 1H), 7.39 (t, J = 9.2 Hz, 1H), 4.58 (d, J = 5.8 Hz, 2H), 4.25- 4.06 (m, 2H), 2.57 (s, 3H), 2.05-1.86 (m, 2H). LCMS calcd for; C₂₃H₁₉ClF₂N₆O₄S₂. Found; 581.50 (M + 1). |
| 920- | N3-(3-chloro-4-fluoro-phenyl)-N5-[[2-(4-cyano- | ¹H NMR (400 MHz, DMSO-d6): δ |
| ISOMER-A | 2-fluoro-phenyl)thiazol-5-yl]methyl]-2-methyl- | 10.50 (br.s, 1H), 8.84 (br.s, 1H), 8.36 |
| | 1,1-dioxo-1,2,6-thiadiazinane-3,5- | (t, J = 8.0 Hz, 1H), 8.12-8.09 (m, 1H), |
| | dicarboxamide. | 7.97-7.93 (m, 2H), 7.83 (d, J = 8.4 Hz, |
| | | 1H), 7.56-7.52 (m, 2H), 7.41-7.36 (m, |
| | | 1H), 4.57 (d, J = 5.6 Hz, 2H), 4.13- |
| | | 4.06 (m, 2H), 2.54 (s, 3H), 2.01-1.86 |
| | | (m, 2H). LCMS calcd for; |
| | | C₂₃H₁₉ClF₂N₆O₄S₂. Found; 581 |
| | | (M + 1). |
| 920- | N3-(3-chloro-4-fluoro-phenyl)-N5-[[2-(4-cyano- | .¹H NMR (400 MHz, DMSO-d6): δ |
| ISOMER-B | 2-fluoro-phenyl)thiazol-5-yl]methyl]-2-methyl- | 10.49 (s, 1H), 8.84-8.82 (m, 1H), 8.32 |
| | 1,1-dioxo-1,2,6-thiadiazinane-3,5- | (t, J = 8.4 Hz, 1H), 8.09-8.06 (m, 1H), |
| | dicarboxamide | 7.94-7.90 (m, 2H), 7.81-7.79 (m, 1H), |
| | | 7.53-7.49 (m, 1H), 7.48-7.33 (m, 2H), |
| | | 4.54 (d, J = 8.0 Hz, 2H), 4.17-4.05 (m, |
| | | 2H), 2.53 (s, 3H), 1.98-1.87 (m, |
| | | 2H). LCMS calcd for; |
| | | C₂₃H₁₉ClF₂N₆O₄S₂. Found; 581.1 |
| | | (M + 1). |
| 921 | 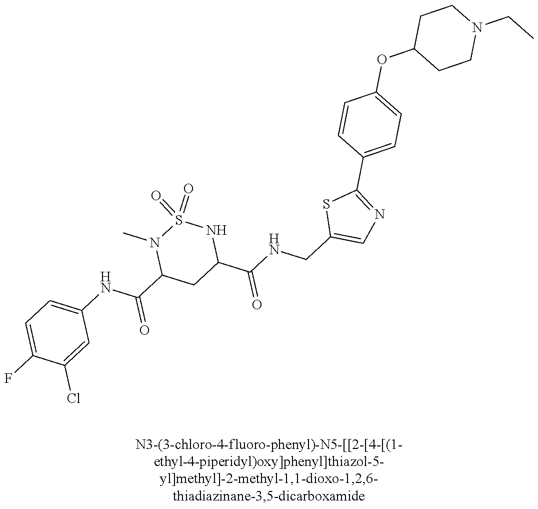 [SMILES: CCN1CCC(Oc2ccc(-c3ncc(CNC(=O)C4CC(C(=O)Nc5ccc(F)c(Cl)c5)N(C)S(=O)(=O)N4)s3)cc2)CC1] | ¹H NMR (400 MHz, DMSO-d6): δ 10.53 (s, 1H), 8.81 (t, J = 5.8 Hz, 1H), 7.95 (dd, J = 6.8, 2.6 Hz, 1H), 7.79 (d, J = 8.8 Hz, 2H), 7.67 (s, 1H), 7.56- 7.52 (m, 1H), 7.41-7.36 (m, 2H), 7.03 (d, J = 8.8 Hz, 2H), 4.49-4.46 (m, 3H), 4.22-4.18 (m, 1H), 4.14-4.05 (m, 1H), 2.74-2.66 (m, 2H), 2.57 (s, 3H), 2.39 (q, J = 7.2 Hz, 2H), 2.31-2.21 (m, 2H), 2.03-1.86 (m, 4H), 1.67-1.63 (m, 2H), 1.01 (t, J = 7.2 Hz, 3H). LCMS calcd for; C₂₉H₃₄ClFN₆O₅S₂. Found; 665.25 (M + 1). |
| 921- | N3-(3-chloro-4-fluoro-phenyl)-N5-[[2-[4-[(1- | ¹H NMR (400 MHz, DMSO-d6): δ |
| ISOMER-A | ethyl-4-piperidyl)oxy]phenyl]thiazol-5- | 10.49 (br.s., 1H), 8.75 (br.s, 1H), 7.96- |
| | yl]methyl]-2-methyl-1,1-dioxo-1,2,6- | 7.94 (m, 1H), 7.78 (d, J = 8.8 Hz, 2H), |
| | thiadiazinane-3,5-dicarboxamide. | 7.67 (s, 1H), 7.57-7.52 (m, 1H), 7.38 |
| | | (t, J = 9.6 Hz, 1H), 7.04 (d, J = 8.0 |
| | | Hz, 2H), 4.50-4.42 (m, 3H), 4.07-4.03 |
| | | (m, 2H), 2.70-2.67 (m, 2H), 2.53 (s, |
| | | 3H), 2.36-2.30 (m, 2H), 2.21-2.15 (m, |
| | | 2H), 2.00-1.87 (m, 4H), 1.66-1.58 (m, |
| | | 2H), 0.99 (t, J = 6.8 Hz, 3H). LCMS |
| | | calcd for; C₂₉H₃₄ClFN₆O₅S₂. Found; |
| | | 665.1 (M + 1). |
| 921- | N3-(3-chloro-4-fluoro-phenyl)-N5-[[2-[4-[(1- | ¹H NMR (400 MHz, DMSO-d6): δ |
| ISOMER-B | ethyl-4-piperidyl)oxy]phenyl]thiazol-5- | 10.43 (s, 1H), 8.66 (br.s, 1H), 7.93- |
| | yl]methyl]-2-methyl-1,1-dioxo-1,2,6- | 7.91 (m, 1H), 7.78 (d, J = 8.8 Hz, 2H), |
| | thiadiazinane-3,5-dicarboxamide. | 7.63 (s, 1H), 7.54-7.49 (m, 1H), 7.37- |
| | | 7.31 (m, 1H), 6.99 (d, J = 8.8 Hz, 2H), |
| | | 4.46-4.38 (m, 3H), 3.98-3.95 (m, 2H), |
| | | 2.76-2.64 (m, 2H), 2.32-2.23 (m, 2H), |
| | | 2.17-2.12 (m, 2H), 1.97-1.86 (m, 4H), |
| | | 1.62-1.54 (m, 2H), 0.96 (t, J = 7.2 Hz, |
| | | 3H). LCMS calcd for; |
| | | C₂₉H₃₄ClFN₆O₅S₂. Found; 665.1 |
| | | (M + 1). |
| 922 | 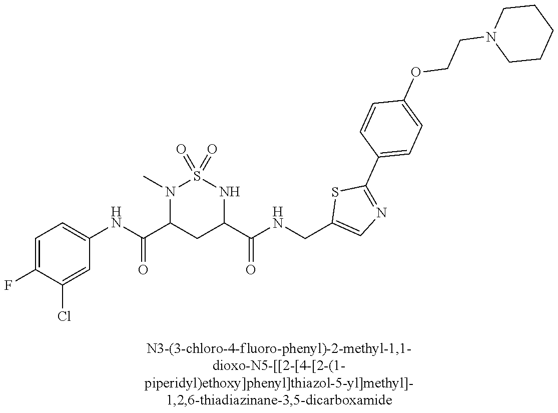 [SMILES: CN1C(C(=O)Nc2ccc(F)c(Cl)c2)CC(C(=O)NCc2cnc(-c3ccc(OCCN4CCCCC4)cc3)s2)NS1(=O)=O] | ¹H NMR (400 MHz, DMSO-d6): δ 10.53 (s, 1H), 8.82-8.80 (m, 1H), 7.96- 7.95 (m, 1H), 7.80 (d, J = 8.4 Hz, 2H), 7.69 (s, 1H), 7.54-7.52 (m, 1H), 7.42- 7.37 (m, 2H), 7.04 (d, J = 8.4 Hz, 2H), 4.50-4.48 (m, 2H), 4.23-4.19 (m, 1H), 4.13-4.11 (m, 3H), 2.67-2.65 (m, 2H), 258 (s, 3H), 2.44-2.34 (m, 4H), 1.98- 1.92 (m, 2H), 1.51-1.49 (m, 4H), 1.38-1.36 (m, 2H). LCMS calcd for; C₂₉H₃₄ClFN₆O₅S₂. Found; 665.60 (M + 1). |
| 922- | N3-(3-chloro-4-fluoro-phenyl)-2-methyl-1,1- | ¹H NMR (400 MHz, DMSO-d6): δ |
| ISOMER-A | dioxo-N5-[[2-[4-[2-(1- | 10.53 (s, 1H), 8.82-8.80 (m, 1H), 7.96- |
| | piperidyl)ethoxy]phenyl]thiazol-5-yl]methyl]- | 7.94 (m, 1H), 7.80 (d, J = 8.8 Hz, 2H), |
| | 1,2,6-thiadiazinane-3,5-dicarboxamide. | 7.68 (s, 1H), 7.55-7.52 (m, 1H), 7.42- |
| | | 7.37 (m, 2H), 7.04 (d, J = 8.8 Hz, 2H), |
| | | 4.50-4.48 (m, 2H), 4.21-4.19 (m, 1H), |
| | | 4.13-4.11 (m, 3H), 2.66 (t, J = 6.0 Hz, |
| | | 2H), 2.57 (s, 3H), 2.43-2.40 (m, 4H), |
| | | 1.98-1.94 (m, 2H), 1.52-1.48 (m, 4H), |
| | | 1.39-1.36 (m, 2H). LCMS calcd for; |
| | | C₂₉H₃₄ClFN₆O₅S₂. Found; 665.35 |
| | | (M + 1). |
| 922- | N3-(3-chloro-4-fluoro-phenyl)-2-methyl-1,1- | ¹H NMR (400 MHz, DMSO-d6): δ |
| ISOMER-B | idoxo-N5-[[2-[4-[2-(1- | 10.53 (s, 1H), 8.81 (t, J = 5.6 Hz, 1H), |
| | piperidyl)ethoxy]phenyl]thiazol-5-yl]methyl]- | 7.96-7.93 (m, 1H), 7.80 (d, J = 8.8 Hz, |
| | 1,2,6-thiadiazinane-3,5-dicarboxamide. | 2H), 7.68 (s, 1H), 7.56-7.52 (m, 1H), |
| | | 7.43-7.37 (m, 2H), 7.03 (d, J = 8.8 Hz, |
| | | 2H), 4.50-4.48 (m, 2H), 4.22-4.18 (m, |
| | | 1H), 4.13-4.10 (m, 3H), 2.68-2.64 (m, |
| | | 2H), 2.57 (s, 3H), 2.44-2.42 (m, 4H), |
| | | 1.97-1.92 (m, 2H), 1.52-1.47 (m, 4H), |
| | | 1.39-1.37 (m, 2H). LCMS calcd for; |
| | | C₂₉H₃₄ClFN₆O₅S₂. Found; 665.35 |
| | | (M + 1). |
| 923 | 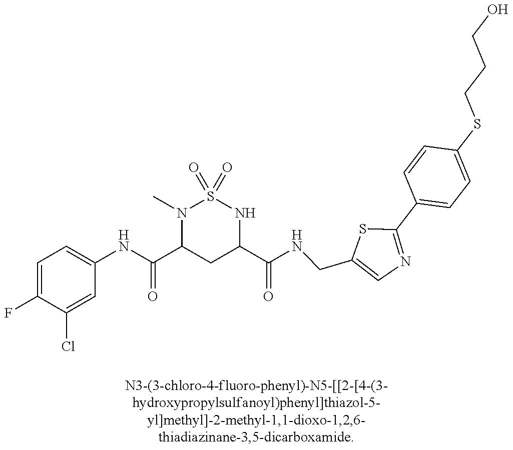 [SMILES: CN1C(C(=O)Nc2ccc(F)c(Cl)c2)CC(C(=O)NCc2cnc(-c3ccc(SCCCO)cc3)s2)NS1(=O)=O] | ¹H NMR (400 MHz, DMSO-d6): δ 10.49 (s, 1H), 8.79 (t, J = 6.0 Hz, 1H), 7.91 (dd, J = 6.8, 2.6 Hz, 1H), 7.77 (d, J = 8.4 Hz 2H), 7.70 (s, 1H), 7.52-7.49 (m, 1H), 7.38-7.34 (m, 4H), 4.56 (t, J = 5.2 Hz, 1H), 4.48 (d, J = 5.8 Hz, 2H), 4.16 (dd, J = 11.1, 3.8 Hz, 1H), 4.07-4.05 (m, 1H), 3.47 (q, J = 5.8 Hz, 2H), 3.03 (t, J = 7.3 Hz, 2H), 2.53 (s, 3H), 2.01-1.83 (m, 2H), 1.98-1.85 (m, 2H). LCMS calcd for; C₂₅H₂₇ClFN₅O₅S₃. Found; 628.1 (M + 1). |
| 924 | 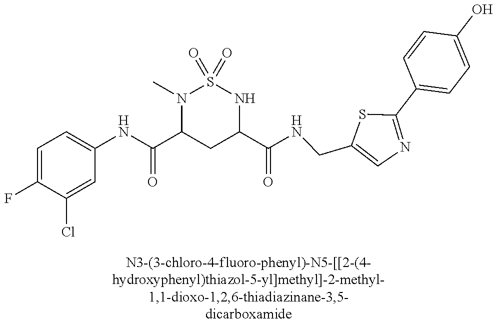 [SMILES: CN1C(C(=O)Nc2ccc(F)c(Cl)c2)CC(C(=O)NCc2cnc(-c3ccc(O)cc3)s2)NS1(=O)=O] | ¹H NMR (400 MHz, DMSO-d6): δ 10.52 (s, 1H), 9.96 (s, 1H), 8.79 (s, 1H), 8.00-7.91 (m, 1H), 7.74-7.61 (m, 3H), 7.54 (d, J = 8.3 Hz, 1H), 7.40- 7.38 (m, 2H), 6.88-6.78 (m, 2H), 4.48- 4.46 (m, 2H), 4.26-4.09 (m, 2H), 2.57 (s, 3H), 1.97-1.95 (m, 2H). LCMS calcd for; C₂₂H₂₁ClFN₅O₅S₂. Found; 554.15 (M + 1). |
| 924- | N3-(3-chloro-4-fluroo-phenyl)-N5-[[2-(4- | ¹H NMR (400 MHz, DMSO-d6): δ |
| ISOMER-A | hydroxyphenyl)thiazol-5-yl]methyl]-2-methyl- | 10.48 (s, 1H), 9.95-9.93 (m, 1H), 8.77- |
| | 1,1-dioxo-1,2,6-thiadiazinane-3,5- | 8.73 (m, 1H), 7.93-7.90 (m, 1H), 7.67 |
| | dicarboxamide. | (d, J = 9.2 Hz, 2H), 7.60 (s, 1H), |
| | | 7.53-7.50 (m, 1H), 7.38-7.33 (m, 2H), |
| | | 6.80 (d, J = 8.4 Hz, 2H), 4.45-4.44 |
| | | (m, 2H), 4.17-4.04 (m, 2H), 2.53 (s, |
| | | 3H), 1.97-1.87 (m, 2H). LCMS calcd |
| | | for; C₂₂H₂₁ClFN₅O₅S₂. Found; 554 |
| | | (M + 1). |
| 924- | N3-(3-chloro-4-fluoro-phenyl)-N5-[[2-(4- | ¹H NMR (400 MHz, DMSO-d6): δ |
| ISOMER-B | hydroxyphenyl)thiazol-5-yl]methyl]-2-methyl- | 10.49 (s, 1H), 9.94-9.92 (m, 1H), 8.76- |
| | 1,1-dioxo-1,2,6-thiadiazinane-3,5- | 8.74 (m, 1H), 7.93-7.90 (m, 1H), 7.67 |
| | dicarboxamide. | (d, J = 9.2 Hz, 2H), 7.61 (s, 1H), |
| | | 7.53-7.49 (m, 1H), 7.38-7.33 (m, 2H), |
| | | 6.80 (d, J = 8.4 Hz, 2H), 4.45-4.44 (m, |
| | | 2H), 4.17-4.04 (m, 2H), 2.53 (s, 3H), |
| | | 1.97-1.88 (m, 2H). LCMS calcd for; |
| | | C₂₂H₂₁ClFN₅O₅S₂. Found; 554.15 |
| | | (M + 1). |
| 925 | 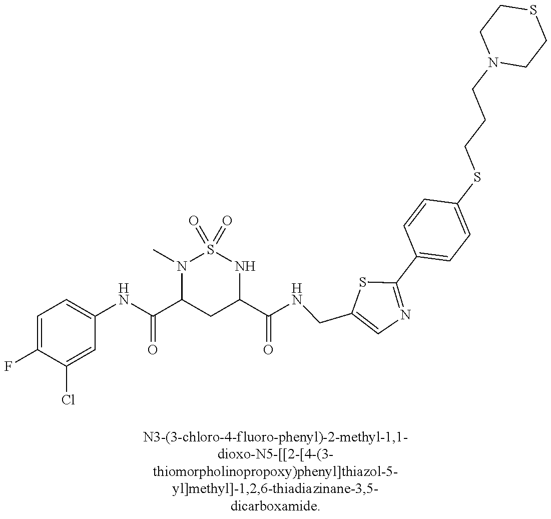 | ¹H NMR (400 MHz, DMSO-d6): δ 10.52 (s, 1H), 8.81 (t, J = 5.9 Hz, 1H), 7.96-7.93 (m, 1H), 7.80 (d, J = 8.3 Hz, 2H), 7.68 (s, 1H), 7.54-7.56 (m, 1H), 7.41-7.36 (m, 2H), 7.02 (d, J = 8.4 Hz, 2H), 4.50-4.48 (m, 2H), 4.22-4.18 (m, 1H), 4.12-4.00 (m, 3H), 2.67-2.54 (m, 10H), 2.47-2.43 (m, 3H), 2.02-1.81 (m, 4H). LCMS calcd for; C₂₉H₃₄ClFN₆O₅S₃. Found; 697.1 (M + 1). |
| 925- | N3-(3-chloro-4-fluoro-phenyl)-2-methyl-1,1- | ¹H NMR (400 MHz, DMSO-d6): δ |
| ISOMER-A | dioxo-N5-[[2-[4-(3- | 10.52 (s, 1H), 8.80-8.78 (m, 1H), 7.96- |
| | thiomorpholinopropoxy)phenyl]thiazol-5- | 7.93 (m, 1H), 7.80 (d, J = 8.8 Hz, 2H), |
| | yl]methyl]-1,2,6-thiadiazinane-3,5- | 7.68 (s, 1H), 7.56-7.52 (m, 1H), 7.41- |
| | dicarboxamide | 7.36 (m, 2H), 7.01 (d, J = 8.8 Hz, 2H), |
| | | 4.50-4.48 (m, 2H), 4.19-4.03 (m, 4H), |
| | | 2.64-2.58 (m, 8H), 2.56 (s, 3H), 2.47- |
| | | 2.43 (m, 2H), 2.00-1.83 (m, 4H). |
| | | LCMS calcd for; C₂₉H₃₄ClFN₆O₅S₃. |
| | | Found; 697.1 (M + 1). |
| 925- | N3-(3-chloro-4-fluoro-phenyl)-2-methyl-1,1- | ¹H NMR (400 MHz, DMSO-d6): δ |
| ISOMER-B | dioxo-N5-[[2-[4-(3- | 10.52 (s, 1H), 8.80-8.78 (m, 1H), 7.96- |
| | thiomorpholinopropoxy)phenyl]thiazol-5- | 7.93 (m, 1H), 7.80 (d, J = 8.0 Hz, 2H), |
| | yl]methyl]-1,2,6-thiadiazinane-3,5- | 7.68 (s, 1H), 7.56-7.53 (m, 1H), 7.41- |
| | dicarboxamide. | 7.36 (m, 2H), 7.01 (d, J = 8.8 Hz, 2H), |
| | | 4.50-4.48 (m, 2H), 4.20-4.03 (m, 4H), |
| | | 2.63-2.60 (m, 8H), 2.56 (s, 3H), 2.47- |
| | | 2.44 (m, 2H), 2.00-1.85 (m, 4H). |
| | | LCMS calcd for; C₂₉H₃₄ClFN₆O₅S₃. |
| | | Found; 697.1 (M + 1). |
| 926 | 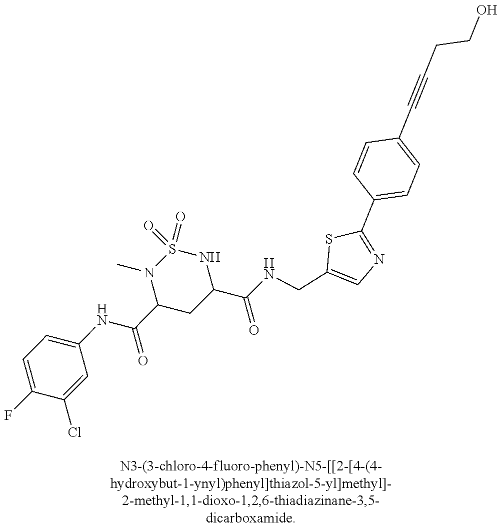 [SMILES: CN1C(C(=O)Nc2ccc(F)c(Cl)c2)CC(C(=O)NCc2cnc(-c3ccc(C#CCCO)cc3)s2)NS1(=O)=O] | ¹H NMR (400 MHz, DMSO-d6): δ 10.49 (s, 1H), 8.82-8.79 (m, 1H), 7.93- 7.90 (m, 1H), 7.82 (d, J = 8.8 Hz, 2H), 7.75 (s, 1H), 7.53-7.49 (m, 1H), 7.45 (d, J = 8.4 Hz, 2H), 7.38-7.33 (m, 2H), 4.88 (t, J = 5.2 Hz, 1H), 4.48 (d, J = 6.0 Hz, 2H), 4.19-4.15 (m, 2H), 4.09-4.06 (m, 1H), 3.56 (q, J = 12.8, 6.8 Hz, 2H), 2.57-2.55 (m, 2H), 2.54 (s, 3H), 1.98-1.85 (m, 2H). LCMS calcd for; C₂₆H₂₅ClFN₅O₅S₂. Found; 606.20 (M + 1). |
| 928 | 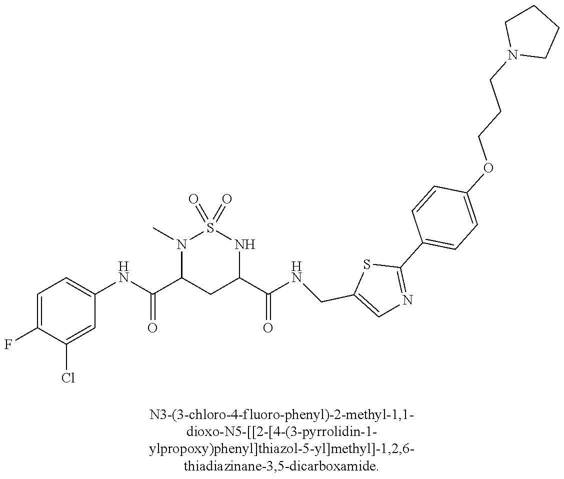 [SMILES: CN1C(C(=O)Nc2ccc(F)c(Cl)c2)CC(C(=O)NCc2cnc(-c3ccc(OCCCN4CCCC4)cc3)s2)NS1(=O)=O] | ¹H NMR (400 MHz, DMSO-d6): δ 10.50 (s, 1H), 8.79-8.71 (m, 1H), 7.93- 7.90 (m, 1H), 7.81 (d, J = 8.8 Hz, 2H), 7.65 (s, 1H), 7.53-7.48 (m, 1H), 7.39-7.32 (m, 2H), 7.01 (d, J = 8.8 Hz, 2H), 4.46 (d, J = 6.0 Hz, 2H), 4.19- 4.15 (m, 1H), 4.10-4.04 (m, 3H), 3.52- 3.48 (m, 2H), 3.10-2.80 (m, 4H), 2.54 (s, 3H), 2.10 (m, 2H), 1.94-1.80 (m, 6H). LCMS calcd for; C₂₉H₃₄ClFN₆O₅S₂. Found; 665 (M + 1). |
| 929 | 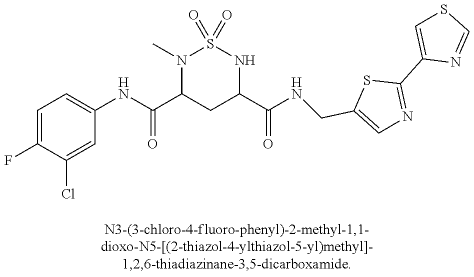 [SMILES: CN1C(C(=O)Nc2ccc(F)c(Cl)c2)CC(C(=O)NCc2cnc(-c3cscn3)s2)NS1(=O)=O] | ¹H NMR (400 MHz, DMSO-d6): δ 10.54 (s, 1H), 9.23-9.21 (m, 1H), 8.85 (t, J = 6.0 Hz, 1H), 8.26-8.25 (m, 1H), 7.99-7.92 (m, 1H), 7.76 (s, 1H), 7.56- 7.53 (m, 1H), 7.49-7.35 (m, 2H), 4.60- 4.47 (m, 2H), 4.23-4.08 (m, 2H), 2.57 (s, 3H), 2.03-1.86 (m, 2H). LCMS calcd for; C₁₉H₁₈ClFN₆O₄S₃. Found; 544.85 (M + 1). |
| 929- | N3-(3-chloro-4-fluoro-phenyl)-2-methyl-1,1- | ¹H NMR (400 MHz, DMSO-d6): δ |
| ISOMER-A | dioxo-N-[(2-thiazol-4-ylthiazol-5-yl)methyl]- | 10.51 (bs, 1H), 9.22-9.21 (m, 1H), |
| | 1,2,6-thiadiazinane-3,5-dicarboxamide. | 8.85-8.82 (m, 1H), 8.25 (s, 1H), 7.96- |
| | | 7.92 (m, 1H), 7.74 (s, 1H), 7.56-7.53 |
| | | (m, 1H), 7.49-7.35 (m, 1H), 4.52 (d, J = |
| | | 5.6 Hz, 2H), 4.23-4.08 (m, 2H), 2.56 |
| | | (s, 3H), 2.03-1.86 (m, 2H). LCMS |
| | | calcd for; C₁₉H₁₈ClFN₆O₄S₃. Found; |
| | | 544.95 (M + 1). |
| 929- | N3-(3-chloro-4-fluoro-phenyl)-2-methyl-1,1- | ¹H NMR (400 MHz, DMSO-d6): δ |
| ISOMER-B | dioxo-N5-[(2-thiazol-4-ylthiazol-5-yl)methyl]- | 10.51 (bs, 1H), 9.22-9.20 (m, 1H), |
| | 1,2,6-thiadiazinane-3,5-dicarboxamide. | 8.85-8.82 (m, 1H), 8.26-8.24 (m, 1H), |
| | | 7.96-7.94 (m, 1H), 7.74 (s, 1H), 7.56- |
| | | 7.53 (m, 1H), 7.49-7.35 (m, 2H), 4.52 |
| | | (d, J = 5.6 Hz, 2H), 4.20-4.07 (m, |
| | | 2H), 2.57 (s, 3H), 2.00-1.87 (m, |
| | | 2H). LCMS calcd for; |
| | | C₁₉H₁₈ClFN₆O₄S₃. Found; 544.95 |
| | | (M + 1). |
| 931 | 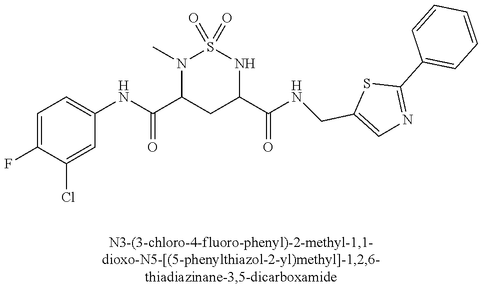 | ¹H NMR (400 MHz, DMSO-d6): δ 10.54 (s, 1H), 8.84 (t, J = 5.2 Hz, 1H), 7.96-7.94 (m, 1H), 7.90-7.88 (m, 2H), 7.77 (s, 1H), 7.56-7.53 (m, 1H), 7.50- 7.48 (m, 3H), 7.45-7.37 (m, 2H), 4.52 (d, J = 5.2 Hz, 2H), 4.23-4.20 (m, 1H), 4.12-4.10 (m, 1H), 2.58 (s, 3H), 2.02-1.93 (m, 2H). LCMS calcd for; C₂₂H₂₁ClFN₅O₄S₂. Found; 583.40 (M + 1). |
| 931- | N3-(3-chloro-4-fluoro-phenyl)-2-methyl-1,1- | ¹H NMR (400 MHz, DMSO-d6): δ |
| ISOMER-A | dioxo-N5-[(5-phenylthiazol-2-yl)methyl]-1,2,6- | 10.53 (s, 1H), 8.84 (t, J = 5.2 Hz, 1H), |
| | thiadiazinane-3,5-dicarboxamide. | 7.96-7.88 (m, 3H), 7.76 (s, 1H), 7.56- |
| | | 7.47 (m, 4H), 7.41-7.36 (m, 2H), 4.52 |
| | | (d, J = 6.0 Hz, 2H), 4.22-4.09 (m, 2H), |
| | | 2.57 (s, 3H), 1.98-1.92 (m, 2H). LCMS |
| | | calcd for; C₂₂H₂₁ClFN₅O₄S₂. Found; |
| | | 538.15 (M + 1). |
| 931- | N3-(3-chloro-4-fluoro-phenyl)-2-methyl-1,1- | ¹H NMR (400 MHz, DMSO-d6): δ |
| ISOMER-B | dioxo-N5-[(5-phenylthiazol-2-yl)methyl]-1,2,6- | 10.52 (s, 1H), 8.84 (t, J = 4.8 Hz, 1H), |
| | thiadiazinane-3,5-dicarboxamide. | 7.96-7.88 (m, 3H), 7.76 (s, 1H), 7.56- |
| | | 7.47 (m, 4H), 7.43-7.36 (m, 2H), 4.52 |
| | | (d, J = 5.6 Hz, 2H), 4.21-4.09 (m, 2H), |
| | | 2.57 (s, 3H), 2.01-1.92 (m, 2H). LCMS |
| | | calcd for; C₂₂H₂₁ClFN₅O₄S₂. Found; |
| | | 583.20 (M + 1). |
| 932 | 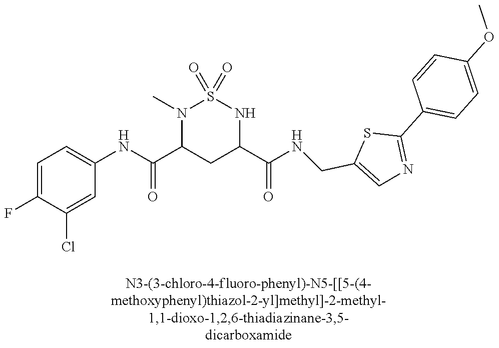 [SMILES: COc1ccc(-c2ncc(CNC(=O)C3CC(C(=O)Nc4ccc(F)c(Cl)c4)N(C)S(=O)(=O)N3)s2)cc1] | ¹H NMR (400 MHz, DMSO-d6): δ 10.53 (s, 1H), 8.84 (t, J = 5.2 Hz, 1H), 7.96-7.94 (m, 1H), 7.82 (d, J = 8.8 Hz, 2H), 7.69 (s, 1H), 7.56-7.53 (m, 1H), 7.42-7.37 (m, 2H), 7.03 (d, J = 8.4 Hz, 2H), 4.50 (d, J = 5.2 Hz, 2H), 4.23- 4.09 (m, 2H), 3.81 (s, 3H), 2.58 (s, 3H), 2.02-1.97 (m, 2H). LCMS calcd for; C₂₃H₂₃ClFN₅O₅S₂. Found; 568 (M + 1). |
| 932- | N3-(3-chloro-4-fluoro-phenyl)-N5-[[5-(4- | ¹H NMR (400 MHz, DMSO-d6): δ |
| ISOMER-A | methoxyphenyl)thiazol-2-yl]methyl]-2-methyl- | 10.45 (bs, 1H), 8.75-8.70 (m, 1H), |
| | 1,1-dioxo-1,2,6-thiadiazinane-3,5- | 7.97-7.94 (m, 1H), 7.82 (d, J = 8.8 Hz, |
| | dicarboxamide. | 2H), 7.66 (s, 1H), 7.57-7.54 (m, 2H), |
| | | 7.38 (t, J = 8.8 Hz, 1H), 7.03 (d, J = |
| | | 8.4 Hz, 2H), 4.49 (d, J = 6.0 Hz, 2H), |
| | | 4.05-4.00 (m, 2H), 3.81 (s, 3H), 2.00- |
| | | 1.85 (m, 2H). LCMS calcd for; |
| | | C₂₃H₂₃ClFN₅O₅S₂. Found; 568.20 |
| | | (M + 1). |
| 932- | N3-(3-chloro-4-fluoro-phenyl)-N5-[[5-(4- | ¹H NMR (400 MHz, DMSO-d6): |
| ISOMER-B | methoxyphenyl)thiazol-2-yl]methyl]-2-methyl- | δ10.51 (bs, 1H), 8.80-8.75 (m, 1H), |
| | 1,1-dioxo-1,2,6-thiadiazinane-3,5- | 7.96-7.94 (m, 1H), 7.82 (d, J = 8.8 |
| | dicarboxamide. | Hz, 2H), 7.68 (s, 1H), 7.56-7.52 (m, |
| | | 1H), 7.38 (t, J = 9.2 Hz, 2H), 7.03 (d, |
| | | J = 8.8 Hz, 2H), 4.49 (d, J = 5.6 Hz, |
| | | 2H), 4.17-4.06 (m, 2H), 3.81 (s, 3H), |
| | | 2.55 (s, 3H), 2.00-1.88 (m, 2H). LCMS |
| | | calcd for; C₂₃H₂₃ClFN₅O₅S₂. Found; |
| | | 568.25 (M + 1). |
| 942 | 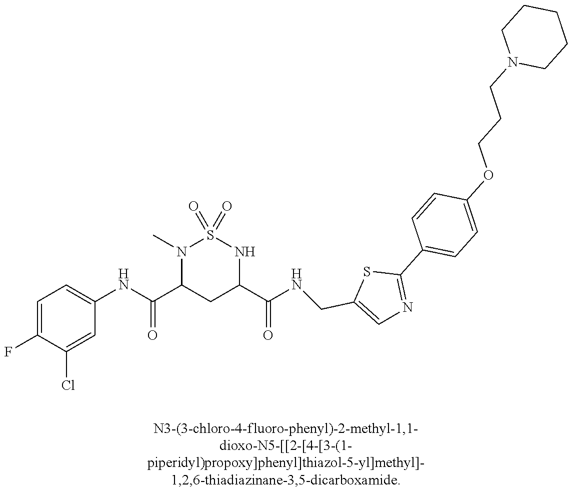 [SMILES: CN1C(C(=O)Nc2ccc(F)c(Cl)c2)CC(C(=O)NCc2cnc(-c3ccc(OCCCN4CCCCC4)cc3)s2)NS1(=O)=O] | ¹H NMR (400 MHz, DMSO-d6): δ 10.50 (s, 1H), 8.78 (t, J = 5.6 Hz, 1H), 7.93-7.90 (m, 1H), 7.77 (d, J = 8.8 Hz, 2H), 7.65 (s, 1H), 7.53-7.49 (m, 1H), 7.38-7.33 (m, 2H), 6.98 (d, J = 8.8 Hz, 2H), 4.47-4.45 (m, 2H), 4.19-4.16 (m, 1H), 4.08-4.00 (m, 3H), 2.54 (s, 3H), 2.42-1.97 (m, 6H), 1.96-1.82 (m, 4H), 1.49-1.45 (m, 4H), 1.36-1.35 (m, 2H). LCMS calcd for; C₃₀H₃₆ClFN₆O₅S₂. Found; 679.1 (M + 1). |
| 943 | 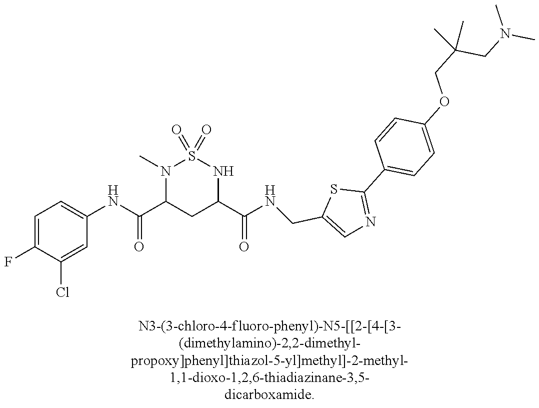 [SMILES: CN(C)CC(C)(C)COc1ccc(-c2ncc(CNC(=O)C3CC(C(=O)Nc4ccc(F)c(Cl)c4)N(C)S(=O)(=O)N3)s2)cc1] | ¹H NMR (400 MHz, DMSO-d6): δ 10.53 (bs, 1H), 8.82 (br.s, 1H), 7.96- 7.54 (m, 5H), 7.45-7.37 (m, 2H), 7.05-7.03 (m, 2H), 4.50-4.48 (m, 2H), 4.20-4.10 (m, 2H), 3.76-3.74 (m, 2H), 2.58 (s, 3H), 2.22-1.96 (m, 10H), 0.96- 0.94 (m, 6H). LCMS calcd for; C₂₉H₃₆ClFN₆O₅S₂. Found; 667.30 (M + 1). |
| 943- | N3-(3-chloro-4-fluoro-phenyl)-N5-[[2-[4-[3- | ¹H NMR (400 MHz, DMSO-d6): δ |
| ISOMER-A | (dimethylamino)-2,2-dimethyl- | 10.49 (s, 1H), 8.77-8.76 (m, 1H), 7.93- |
| | propoxy]phenyl]thiazol-5-yl]methyl]-2-methyl- | 7.90 (m, 1H), 7.76 (d, J = 8.8 Hz, 2H), |
| | 1,1-dioxo-1,2,6-thiadiazinane-3,5- | 7.64 (s, 1H), 7.52-7.49 (m, 1H), 7.38- |
| | dicarboxamide. | 7.33 (m, 2H), 7.00 (d, J = 8.8 HZ, |
| | | 2H), 4.47-4.45 (m, 2H), 4.17-4.05 (m, |
| | | 2H), 3.71 (s, 2H), 2.53 (s, 3H), 2.20- |
| | | 2.16 (m, 8H), 1.94-1.88 (m, 2H), 0.91 |
| | | (s, 6H). LCMS calcd for; |
| | | C₂₉H₃₆ClFN₆O₅S₂. Found; 667.15 |
| | | (M + 1). |
| 943- | N3-(3-chloro-4-fluoro-phenyl)-N5-[[2-[4-[3- | ¹H NMR (400 MHz, DMSO-d6): δ |
| ISOMER-B | (dimethylamino)-2,2-dimethyl- | 10.49 (s, 1H), 8.77-8.75 (m, 1H), 7.93- |
| | propoxy]phenyl]thiazol-5-yl]methyl]-2methyl- | 7.90 (m, 1H), 7.76 (d, J = 8.4 Hz, |
| | 1,1-dioxo-1,2,6-thiadiazinane-3,5- | 2H), 7.64 (s, 1H), 7.52-7.49 (m, 1H), |
| | dicarboxamide. | 7.38-7.33 (m, 2H), 7.00 (d, J = 8.4 |
| | | HZ, 2H), 4.47-4.45 (m, 2H), 4.17-4.04 |
| | | (m, 2H), 3.71 (s, 2H), 2.53 (s, 3H), |
| | | 2.20-2.16 (m, 8H), 1.94-1.89 (m, 2H), |
| | | 0.91 (s, 6H). LCMS calcd for; |
| | | C₂₉H₃₆ClFN₆O₅S₂. Found; 667.20 |
| | | (M + 1). |
| 946 | 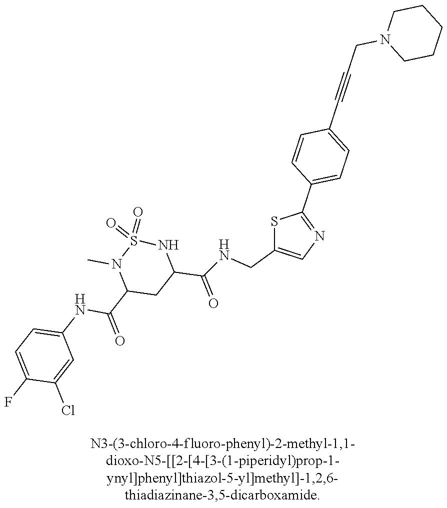 [SMILES: CN1C(C(=O)Nc2ccc(F)c(Cl)c2)CC(C(=O)NCc2cnc(-c3ccc(C#CCN4CCCCC4)cc3)s2)NS1(=O)=O] | ¹H NMR (400 MHz, DMSO-d6): δ 10.52 (s, 1H), 8.84-8.83 (m, 1H), 7.96- 7.93 (m, 1H), 7.87 (d, J = 7.6 Hz, 2H), 7.79 (s, 1H), 7.55-7.50 (m, 3H), 7.44- 7.35 (m, 2H), 4.51 (d, J = 5.6 Hz, 2H), 4.22-4.19 (m, 1H), 4.13-4.08 (m, 1H), 3.50 (s, 2H), 2.57 (s, 3H), 1.99-1.88 (m, 2H), 1.54-1.51 (m, 4H), 1.38-1.36 (m, 2H). LCMS calcd for; C₃₀H₃₂ClFN₆O₄S₂. Found; 659 (M + 1). |
| 946- | N3-(3-chloro-4-fluoro-phenyl)-2-methyl-1,1- | ¹H NMR (400 MHz, DMSO-d6): δ |
| ISOMER-A | dioxo-N5-[[2-[4-[3-(1-piperidyl)prop-1- | 10.53 (s, 1H), 8.84 (t, J = 6.0 Hz, 1H), |
| | ynyl]phenyl]thiazol-5-yl]methyl]-1,2,6- | 7.96-7.93 (m, 1H), 7.87 (d, J = 8.4 |
| | thiadiazinane-3,5-dicarboxamide. | Hz, 2H), 7.79 (s, 1H), 7.56-7.51 (m, |
| | | 3H), 7.42-7.36 (m, 2H), 4.52 (d, J = |
| | | 6.0 Hz, 2H), 4.22-4.18 (m, 1H), 4.12- |
| | | 4.08 (m, 1H), 3.49 (s, 2H), 2.56 (s, |
| | | 3H), 2.49-2.32 (m, 4H), 2.07-1.88 |
| | | (m, 2H), 1.56-1.50 (m, 4H), 1.38-1.36 |
| | | (m, 2H). LCMS calcd for; |
| | | C₃₀H₃₂ClFN₆O₄S₂. Found; 659.15 |
| | | (M + 1). |
| 946- | N3-(3-chloro-4-fluoro-phenyl)-2-methyl-1,1- | ¹H NMR (400 MHz, DMSO-d6): δ |
| ISOMER-B | dioxo-N5-[[2-[4-[3-(1-piperidyl)prop-1- | 10.53 (s, 1H), 8.84 (t, J = 5.6 Hz, 1H), |
| | ynyl]phenyl]thiazol-5-yl]methyl]-1,2,6- | 7.96-7.93 (m, 1H), 7.88 (d, J = 8.0 |
| | thiadiazinane-3,5-dicarboxamide. | Hz, 2H), 7.79 (s, 1H), 7.56-7.52 (m, |
| | | 3H), 7.44-7.36 (m, 2H), 4.52 (d, J = |
| | | 6.0 Hz, 2H), 4.22-4.18 (m, 1H), 4.13- |
| | | 4.08 (m, 1H), 3.52 (s, 2H), 2.57 (s, |
| | | 3H), 2.01-.188 (m, 2H), 1.55-1.53 (m, |
| | | 4H), 1.38-1.33 (m, 2H). LCMS calcd |
| | | for; C₃₀H₃₂ClFN₆O₄S₂. Found; 659.10 |
| | | (M + 1). |
| 948- ISOMER-A | 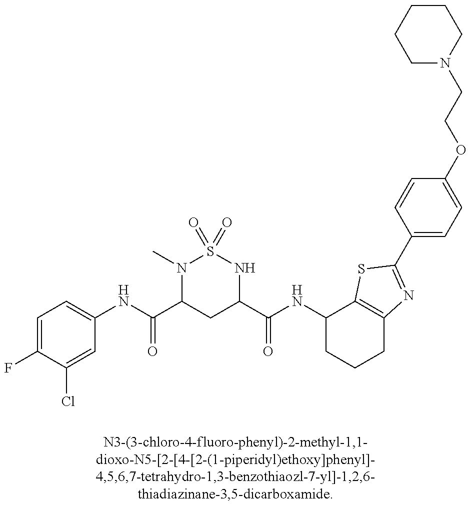 [SMILES: CN1C(C(=O)Nc2ccc(F)c(Cl)c2)CC(C(=O)NC2CCCc3nc(-c4ccc(OCCN5CCCCC5)cc4)sc32)NS1(=O)=O] | ¹H NMR (400 MHz, DMSO-d6): δ 10.51 (s, 1H), 8.68 (d, J = 8.0 Hz, 1H), 7.93-7.90 (m, 1H), 7.80-7.77 (m, 2H), 7.55-7.50 (m, 1H), 7.39-7.18 (m, 2H), 7.02-6.99 (m, 2H), 5.07-5.05 (m, 1H), 4.39-4.08 (m, 4H), 2.90-2.85 (m, 4H), 2.69-2.63 (m, 3H), 2.54 (s, 3H), 2.10-1.35 (m, 13H). LCMS calcd for; C₃₂H₃₈ClFN₆O₅S₂. Found; 704.80 (M + 1). |
| 948- | N3-(3-chloro-4-fluoro-phenyl)-2-methyl-1,1- | ¹H NMR (400 MHz, DMSO-d6): δ |
| ISOMER-B | dioxo-N5-[2-[4-[2-(1-piperidyl)ethoxy]phenyl]- | 10.50 (s, 1H), 8.67 (d, J = 8.0 Hz, 1H), |
| | 4,5,6,7-tetrahydro-1,3-benzothiazol-7-yl]-1,2,6- | 7.93-7.90 (m, 1H), 7.75 (d, J = 8.4 |
| | thiadiazinane-3,5-dicarboxamide. | Hz, 2H), 7.55-7.47 (m, 1H), 7.36 (t, J = |
| | | 9.2 Hz, 1H), 7.29-7.25 (m, 1H), 6.97 |
| | | (d, J = 9.2 Hz, 2H), 5.07-5.04 (m, |
| | | 1H), 4.20-4.17 (m, 1H), 4.10-4.04 (m, |
| | | 3H), 2.70-2.62 (m, 4H), 2.55 (s, 3H), |
| | | 2.45-2.35 (m, 5H), 2.00-1.78 (m, 5H), |
| | | 1.47-1.42 (m, 4H), 1.35-1.30 (m, |
| | | 2H). LCMS calcd for; |
| | | C₃₂H₃₈ClFN₆O₅S₂. Found; 705 (M + 1). |
| 948- | N3-(3-chloro-4-fluoro-phenyl)-2-methyl-1,1- | ¹H NMR (400 MHz, DMSO-d6): δ |
| ISOMER-D | idoxo-N5-[(2-[4-[2-(1-piperidyl)ethoxy]phenyl]- | 10.48 (s, 1H), 8.70-8.60 (m, 1H), 7.93- |
| | 4,5,6,7-tetrahydro-1,3-benzothiazol-7-yl]-1,2,6- | 7.90 (m, 1H), 7.75 (d, J = 8.0 Hz, |
| | thiadiazinane-3,5-dicarboxamide. | 2H), 7.52-7.48 (m, 1H), 7.35 (t, J = |
| | | 9.2 Hz, 1H), 7.28-7.22 (m, 1H), 6.97 |
| | | (d, J = 8.8 Hz, 2H), 5.07-5.04 (m, |
| | | 1H), 4.20-4.04 (m, 4H), 2.70-2.62 (m, |
| | | 4H), 2.53 (s, 3H), 2.45-2.37 (m, 5H), |
| | | 2.00-1.78 (m, 5H), 1.47-1.42 (m, 4H), |
| | | 1.35-1.30 (m, 2H). LCMS calcd for; |
| | | C₃₂H₃₈ClFN₆O₅S₂. Found; 705 (M + 1). |
| 957 | 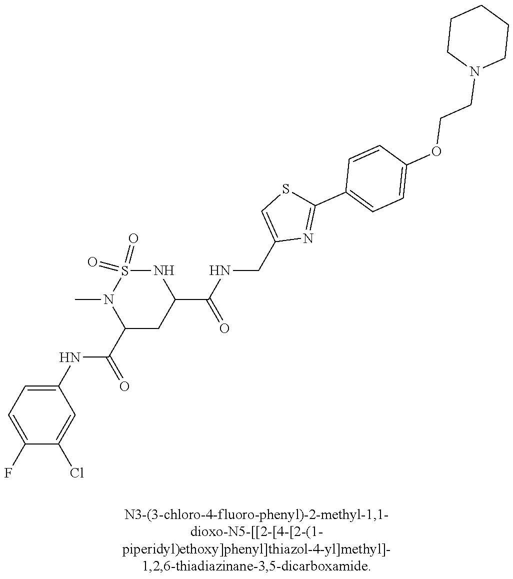 [SMILES: CN1C(C(=O)Nc2ccc(F)c(Cl)c2)CC(C(=O)NCc2csc(-c3ccc(OCCN4CCCCC4)cc3)n2)NS1(=O)=O] | ¹H NMR (400 MHz, DMSO-d6): δ 10.54 (s, 1H), 8.69-8.67 (m, 1H), 7.97- 7.95 (m, 1H), 7.83 (d, J = 8.8 Hz, 2H), 7.55-7.52 (m, 1H), 7.42-7.38 (m, 3H), 7.03 (d, J = 8.8 Hz, 2H), 4.43-4.42 (m, 2H), 4.21-4.19 (m, 2H), 4.11 (t, J = 6.0 Hz, 2H), 2.68-2.65 (m, 2H), 2.58 (s, 3H), 2.45-2.42 (m, 4H), 2.03-1.94 (m, 2H), 1.51-1.48 (m, 4H), 1.39-1.37 (m, 2H). LCMS calcd for; C₂₉H₃₄ClFN₆O₅S₂. Found; 665.15 (M + 1). |
| 957- | N3-(3-chloro-4-fluoro-phenyl)-2-methyl-1,1- | ¹H NMR (400 MHz, DMSO-d6): δ |
| ISOMER-A | dioxo-N5-[[2-[4-[2-(1- | 10.53 (s, 1H), 8.68 (t, J = 5.8 Hz, 1H), |
| | piperidyl)ethoxy]phenyl]thiazol-4-yl]methyl]- | 7.96 (dd, J = 6.8, 2.6 Hz, 1H), 7.83 (d, |
| | 1,2,6-thiadiazinane-3,5-dicarboxamide. | J = 8.8 Hz, 2H), 7.55-7.53 (m, 1H), |
| | | 7.42-7.37 (m, 3H), 7.03 (d, J = 8.0 Hz, |
| | | 2H), 4.43 (d, J = 5.7 Hz, 2H), 4.25- |
| | | 4.17 (m, 2H), 4.11 (t, J = 4.8 Hz, 2H), |
| | | 2.67 (t, J = 5.9 Hz, 2H), 2.59 (s, 3H), |
| | | 2.45-2.40 (m, 4H), 2.03-1.94 (m, 2H), |
| | | 1.52-1.47 (m, 4H), 1.39-1.36 (m, |
| | | 2H). LCMS calcd for; |
| | | C₂₉H₃₄ClFN₆O₅S₂. Found; 665.15 |
| | | (M + 1). |
| 957- | N3-(3-chloro-4-fluoro-phenyl)-2-methyl-1,1- | ¹H NMR (400 MHz, DMSO-d6): δ |
| ISOMER-B | dioxo-N5-[[2-[4-[2-(1- | 10.53 (s, 1H), 8.68 (t, J = 5.9 Hz, 1H), |
| | piperidyl)ethoxy]phenyl]thiazol-4-yl]methyl]- | 7.96 (dd, J = 6.8, 2.6 Hz, 1H), 7.83 (t, |
| | 1,2,6-thiadiazinane-3,5-dicarboxamide. | J = 8.8 Hz, 2H), 7.55-7.52 (m, 1H), |
| | | 7.44-7.35 (m, 3H), 7.03 (t, J = 8.0 Hz, |
| | | 2H), 4.43 (d, J = 6.0 Hz, 2H), 4.22- |
| | | 4.18 (m, 2H), 4.11 (t, J = 6.0 Hz, 2H), |
| | | 2.67 (t, J = 6.0 Hz, 2H), 2.59 (s, 3H), |
| | | 2.46-2.44 (m, 4H), 2.10-1.95 (m, 1H), |
| | | 1.53-1.45 (m, 4H), 1.39-1.37 (m, |
| | | 2H). LCMS calcd for; |
| | | C₂₉H₃₄ClFN₆O₅S₂. Found; 665.10 |
| | | (M + 1). |
| 961 | 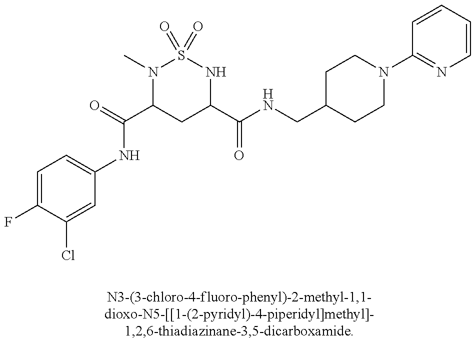 [SMILES: CN1C(C(=O)Nc2ccc(F)c(Cl)c2)CC(C(=O)NCC2CCN(c3ccccn3)CC2)NS1(=O)=O] | ¹H NMR (400 MHz, DMSO-d6): δ 10.52 (s, 1H), 8.13-8.06 (m, 2H), 7.96- 7.95 (m, 1H), 7.56-7.53 (m, 1H), 7.50- 7.46 (m, 1H), 7.40 (t, J = 8.8 Hz, 1H), 7.30-7.27 (m, 1H), 6.79 (d, J = 8.4 Hz, 1H), 6.58-6.54 (m, 1H), 4.26 (d, J = 12.8 Hz, 2H), 4.21-4.18 (m, 1H), 4.10-4.07 (m, 1H), 3.02-2.98 (m, 2H), 2.73 (d, J = 12.4 Hz, 2H), 2.57 (s, 3H), 1.95-1.92 (m, 2H), 1.71-1.67 (m, 3H), 1.11-1.07 (m, 2H). LCMS calcd for; C23H28ClFN6O4S. Found; 539.45 (M + 1). |
| 963 | 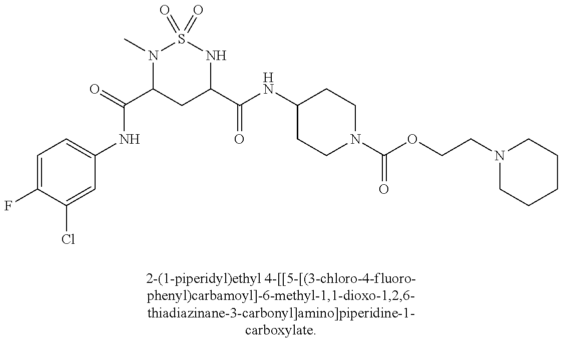 [SMILES: CN1C(C(=O)Nc2ccc(F)c(Cl)c2)CC(C(=O)NC2CCN(C(=O)OCCN3CCCCC3)CC2)NS1(=O)=O] | ¹H NMR (400 MHz, DMSO-d6): δ 10.60 (s, 1H), 8.04 (d, J = 7.6 Hz, 1H), 7.96-7.93 (m, 1H), 7.56-7.52 (m, 1H), 7.39 (t, J = 8.8 Hz, 1H), 7.27-7.24 (m, 1H), 4.20-4.18 (m, 1H), 4.17-4.07 (m, 3H), 3.86-3.80 (m, 2H), 3.77-3.74 (m, 1H), 2.92-2.90 (m, 2H), 2.56 (s, 3H), 2.38-2.36 (m, 4H), 1.98-1.87 (m, 2H), 1.72-1.70 (m, 2H), 1.47-1.45 (m, 4H), 1.37-1.23 (m, 4H). LCMS calcd for; C₂₅H₃₆ClFN₆O₆S. Found; 603.20 (M + 1). |
| 964 | 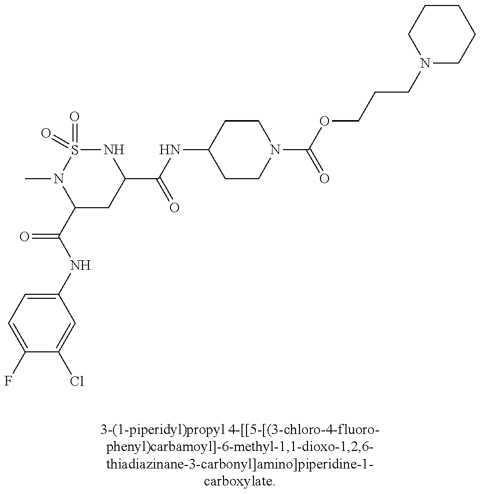 [SMILES: CN1C(C(=O)Nc2ccc(F)c(Cl)c2)CC(C(=O)NC2CCN(C(=O)OCCCN3CCCCC3)CC2)NS1(=O)=O] | ¹H NMR (400 MHz, DMSO-d6): δ 10.52 (s, 1H), 8.04 (d, J = 7.2 Hz, 1H), 7.96-7.93 (m, 1H), 7.56-7.53 (m, 1H), 7.39 (t, J = 9.2 Hz, 1H), 7.25-7.23 (m, 1H), 4.20-4.16 (m, 1H), 4.06-3.97 (m, 3H), 3.86-3.75 (m, 3H), 2.92-2.90 (m, 2H), 2.56 (s, 3H), 2.29-2.25 (m, 6H), 1.95-1.89 (m, 2H), 1.73-1.66 (m, 4H), 1.50-1.44 (m, 4H), 1.37-1.25 (m, 4H). LCMS calcd for; C₂₆H₃₈ClFN₆O₆S. Found; 617 (M + 1). |
| 971 | 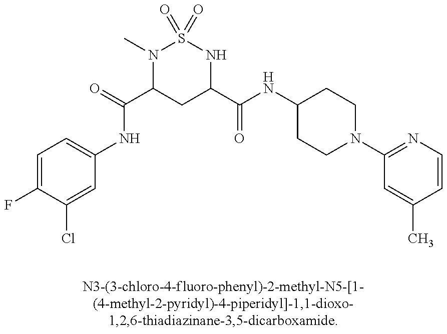 [SMILES: Cc1ccnc(N2CCC(NC(=O)C3CC(C(=O)Nc4ccc(F)c(Cl)c4)N(C)S(=O)(=O)N3)CC2)c1] | ¹H NMR (400 MHz, DMSO-d6): δ 10.54 (s, 1H), 8.10-7.92 (m, 3H), 7.60- 7.50 (m, 1H), 7.39 (d, J = 9.2 Hz, 1H), 7.25-7.15 (m, 1H), 7.75-7.68 (m, 1H), 6.50-6.45 (m, 1H), 4.20-4.15 (m, 3H), 4.07-4.03 (m, 1H), 3.90-3.82 (m, 1H), 2.98-2.90 (m, 2H), 2.56 (s, 3H), 2.21 (s, 3H), 1.96-1.90 (m, 2H), 1.80-1.75 (m, 2H), 1.50-1.38 (m, 2H). LCMS calcd for; C₂₃H₂₈ClFN₆O₄S. Found; 539.15 (M + 1). |
| 972 | 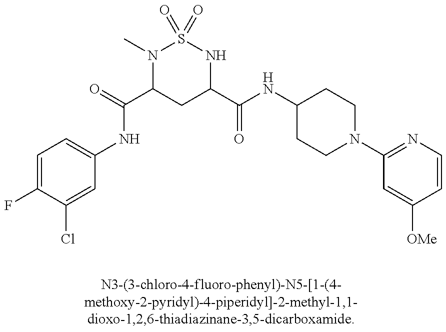 [SMILES: COc1ccnc(N2CCC(NC(=O)C3CC(C(=O)Nc4ccc(F)c(Cl)c4)N(C)S(=O)(=O)N3)CC2)c1] | ¹H NMR (400 MHz, DMSO-d6): δ 10.52 (s, 1H), 8.04 (d, J = 8.0 Hz, 1H), 7.96-7.93 (m, 1H), 7.92-7.90 (m, 1H), 7.56-7.52 (m, 1H), 7.39 (t, J = 8.8 Hz, 1H), 7.25-7.22 (m, 1H), 6.29 (m, 2H), 4.20-4.16 (m, 3H), 4.05-4.03 (m, 1H), 3.84-3.79 (m, 1H), 3.76 (s, 3H), 2.95-2.89 (m, 2H), 2.56 (s, 3H), 1.96-1.88 (m, 2H), 1.78-1.75 (m, 2H), 1.46-1.37 (m, 2H). LCMS calcd for; C₂₃H₂₈ClFN₆O₅S. Found; 555.10 (M + 1). |
| 974 | 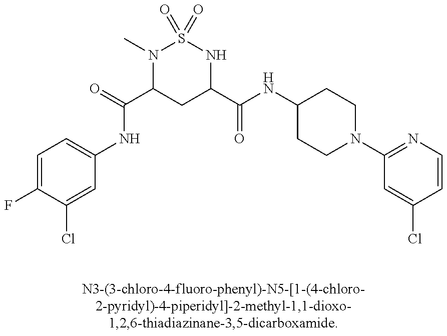 [SMILES: CN1C(C(=O)Nc2ccc(F)c(Cl)c2)CC(C(=O)NC2CCN(c3cc(Cl)ccn3)CC2)NS1(=O)=O] | ¹H NMR (400 MHz, DMSO-d6): δ 10.52 (s, 1H), 8.05 (d, J = 5.2 Hz, 2H), 7.96-7.93 (m, 1H), 7.56-7.52 (m, 1H), 7.40 (t, J = 9.6 Hz, 1H), 7.31-7.21 (m, 1H), 6.94 (s, 1H), 6.67-6.65 (m, 1H), 4.20-4.16 (m, 3H), 4.07-4.03 (m, 1H), 3.86-3.84 (m, 1H), 3.03-2.96 (m, 2H), 2.56 (s, 3H), 1.99-1.90 (m, 2H), 1.77- 1.75 (m, 2H), 1.45-1.34 (m, 2H). LCMS calcd for; C₂₂H₂₅Cl₂FN₆O₄S. Found; 559.05 (M + 1). |
| 974- | N3-(3-chloro-4-fluoro-phenyl)-N5-[1-(4-chloro- | ¹H NMR (400 MHz, DMSO-d6): δ |
| ISOMER-A | 2-pyridyl)-4-piperidyl]-2-methyl-1,1-dioxo- | 10.47 (s, 1H), 8.02-8.00 (m, 2H), 7.92- |
| | 1,2,6-thiadiazinane-3,5-dicarboxamide. | 7.90 (m, 1H), 7.51-7.49 (m, 1H), 7.36 |
| | | (t, J = 9.6 Hz, 1H), 7.21-7.19 (m, |
| | | 1H), 6.90 (s, 1H), 6.63 (d, J = 5.6 Hz, |
| | | 1H), 4.16-3.81 (m, 5H), 3.00-2.93 (m, |
| | | 2H), 2.53 (s, 3H), 1.89-1.87 (m, 2H), |
| | | 1.75-1.73 (m, 2H), 1.39-1.33 (m, 2H). |
| | | LCMS calcd for; C₂₂H₂₅Cl₂FN₆O₄S. |
| | | Found; 559.05 (M + 1). |
| 974- | N3-(3-chloro-4-fluoro-phenyl)-N5-[1-(4-chloro- | ¹H NMR (400 MHz, DMSO-d6): δ |
| ISOMER-B | 2-pyridyl)-4-piperidyl]-2-methyl-1,1-dioxo- | 10.50 (s, 1H), 8.05-8.04 (m, 2H), 7.96- |
| | 1,2,6-thiadiazinane-3,5-dicarboxamide. | 7.93 (m, 1H), 7.55-7.53 (m, 1H), 7.39 |
| | | (t, J = 8.8 Hz, 1H), 7.25-7.22 (m, |
| | | 1H), 6.93 (s, 1H), 6.66 (d, J = 5.2 Hz, |
| | | 1H), 4.17-3.81 (m, 5H), 3.03-2.97 (m, |
| | | 2H), 2.56 (s, 3H), 1.98-1.90 (m, 2H), |
| | | 1.77-1.73 (m, 2H), 1.45-1.36 (m, |
| | | 2H). LCMS calcd for; |
| | | C₂₂H₂₅Cl₂FN₆O₄S. Found; 559.05 |
| | | (M + 1). |
| 975 | 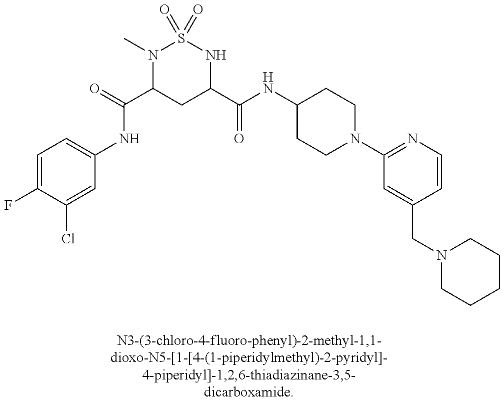 [SMILES: CN1C(C(=O)Nc2ccc(F)c(Cl)c2)CC(C(=O)NC2CCN(c3cc(CN4CCCCC4)ccn3)CC2)NS1(=O)=O] | ¹H NMR (400 MHz, DMSO-d6): δ 10.50 (s, 1H), 7.98-7.96 (m, 2H), 7.93- 7.90 (m, 1H), 7.53-7.49 (m, 1H), 7.36 (t, J = 8.0 Hz, 1H), 7.20-7.18 (m, 1H), 6.69 (s, 1H), 6.53 (d, J = 5.6 Hz, 1H), 4.13-3.77 (m, 5H), 2.93-2.86 (m, 2H), 2.51 (s, 3H), 2.27-2.25 (m, 4H), 1.89-1.73 (m, 4H), 1.47-1.34 (m, 8H). LCMS calcd for; C₂₈H₃₇ClFN₇O₄S. Found; 622.25 (M + 1). |
| 975- | N3-(3-chloro-4-fluoro-phenyl)-2-methyl-1,1- | ¹H NMR (400 MHz, DMSO-d6): δ |
| ISOMER-A | dioxo-N5-[1-[4-(1-piperidylmethyl)-2-pyridyl]- | 10.30-10.20 (m, 1H), 8.02-8.00 (m, |
| | 4-piperidyl]-1,2,6-thiadiazinane-3,5- | 2H), 7.96-7.92 (m, 1H), 7.58-7.52 |
| | dicarboxamide. | (m, 1H), 7.38 (t, J = 8.8 Hz, 1H), 6.72 |
| | | (s, 1H), 6.56 (d, J = 4.8 Hz, 1H), |
| | | 4.18-4.14 (m, 2H), 3.85-3.75 (m, 3H), |
| | | 2.96-2.85 (m, 2H), 2.40-2.35 (m, 4H), |
| | | 1.98-1.74 (m, 4H), 1.52-1.30 (m, |
| | | 8H). LCMS calcd for; |
| | | C₂₈H₃₇ClFN₇O₄S. Found; 622.20 |
| | | (M + 1).⁺. |
| 975- | N3-(3-chloro-4-fluroo-phenyl)-2-methyl-1,1- | ¹H NMR (400 MHz, DMSO-d6): δ |
| ISOMER-B | dioxo-N5-[1-[4-(1-piperidylmethyl)-2-pyridyl]- | 10.30-10.20 (m, 1H), 8.02-8.00 (m, |
| | 4-piperidyl]-1,2,6-thiadiazinane-3,5- | 2H), 7.98-7.92 (m, 1H), 7.60-7.50 |
| | dicarboxamide. | (m, 1H), 7.44-7.36 (m, 1H), 7.30-7.20 |
| | | (m, 1H), 6.72 (s, 1H), 6.56 (d, J = 4.8 |
| | | Hz, 1H), 4.20-3.80 (m, 5H), 2.98-2.80 |
| | | (m, 2H), 2.35-2.25 (m, 4H), 1.95-1.70 |
| | | (m, 4H), 1.50-1.30 (m, 8H). LCMS |
| | | calcd for; C₂₈H₃₇ClFN₇O₄S. Found; |
| | | 622.20 (M + 1).⁺. |
| 978 | 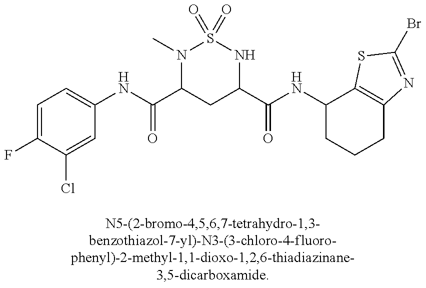 [SMILES: CN1C(C(=O)Nc2ccc(F)c(Cl)c2)CC(C(=O)NC2CCCc3nc(Br)sc32)NS1(=O)=O] | ¹H NMR (400 MHz, DMSO-d6): δ 10.54 (s, 1H), 8.75-8.69 (m, 1H), 7.96- 7.94 (m, 1H), 7.55-7.53 (m, 1H), 7.42- 7.31 (m, 2H), 4.95-4.93 (m, 1H), 4.22-4.19 (m, 1H), 4.10-4.08 (m, 1H), 2.69-2.67 (m, 2H), 2.57 (s, 3H), 1.99- 1.90 (m, 4H), 189-1.77 (m, 2H). LCMS calcd for; C₁₉H₂₀BrClFN₅O₄S₂. Found; 582.40 (M + 1). |
| 979 | 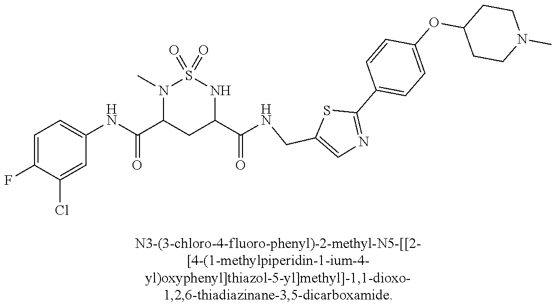 [SMILES: CN1CCC(Oc2ccc(-c3ncc(CNC(=O)C4CC(C(=O)Nc5ccc(F)c(Cl)c5)N(C)S(=O)(=O)N4)s3)cc2)CC1] | ¹H NMR (400 MHz, DMSO-d6): δ 10.53 (s, 1H), 8.81 (t, J = 6.0 Hz, 1H), 8.18 (s, 1H), 7.96-7.93 (m, 1H), 7.79 (d, J = 8.8 Hz, 2H), 7.68 (s, 1H), 7.56- 7.52 (m, 1H), 7.41-7.36 (m, 2H), 7.04 (d, J = 8.4 Hz, 2H), 4.50-4.44 (m, 3H), 4.22-4.09 (m, 2H), 2.66-2.62 (m, 2H), 2.57 (s, 3H), 2.25-2.20 (m, 4H), 2.00- 1.92 (m, 4H), 1.69-1.62 (m, 2H). LCMS calcd for; C₂₈H₃₂ClFN₆O₅S₂. Found; 651.1 (M + 1). |
| 979- | N3-(3-chloro-4-fluoro-phenyl)-2-methyl-N5-[[2- | ¹H NMR (400 MHz, DMSO-d6): δ |
| ISOMER-A | [4-[(1-methyl-4-piperidyl)oxy]phenyl]thiazol-5- | 10.53 (s, 1H), 8.83-8.00 (m, 1H), 7.96- |
| | yl]methyl]-1,1-dioxo-1,2,6-thiadiazinane-3,5- | 7.93 (m, 1H), 7.79 (d, J = 8.8 Hz, 2H), |
| | dicarboxamide. | 7.68 (s, 1H), 7.56-7.52 (m, 1H), 7.41- |
| | | 7.36 (m, 2H), 7.04 (d, J = 8.8 Hz, 2H), |
| | | 4.50-4.40 (m, 3H), 4.22-4.19 (m, 1H), |
| | | 4.11-4.08 (m, 1H), 2.62-2.59 (m, 2H), |
| | | 2.57 (s, 3H), 2.20-2.15 (m, 5H), 2.00- |
| | | 1.88 (m, 4H), 1.68-1.59 (m, |
| | | 2H). LCMS calcd for; |
| | | C₂₈H₃₂ClFN₆O₅S₂. Found; 651.15 |
| | | (M + 1). |
| 979- | N3-(3-chloro-4-fluoro-phenyl)-2-methyl-N5-[[2- | ¹H NMR (400 MHz, DMSO-d6): δ |
| ISOMER-B | [4-[(1-methyl-4-piperidyl)oxy]phenyl]thiaozl-5- | 10.52 (s, 1H), 8.82-8.79 (m, 1H), 7.96- |
| | yl]methyl]-1,1-dioxo-1,2,6-thiadiazinane-3,5- | 7.94 (m, 1H), 7.82-7.77 (m, 2H), 7.68 |
| | dicarboxamide. | (s, 1H), 7.56-7.52 (m, 1H), 7.42-7.35 |
| | | (m, 2H), 7.03 (d, J = 8.4 Hz, 2H), |
| | | 4.50-4.43 (m, 3H), 4.22-4.06 (m, 2H), |
| | | 2.60-2.55 (m, 5H), 2.20-2.15 (m, 5H), |
| | | 2.00-1.92 (m, 4H), 1.70-1.62 (m, |
| | | 2H). LCMS calcd for; |
| | | C₂₈H₃₂ClFN₆O₅S₂. Found; |
| | | 651.15 (M + 1). |
| 980 | 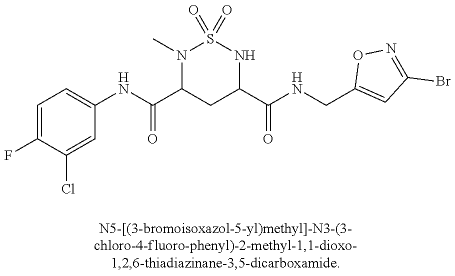 [SMILES: CN1C(C(=O)Nc2ccc(F)c(Cl)c2)CC(C(=O)NCc2cc(Br)no2)NS1(=O)=O] | ¹H NMR (400 MHz, DMSO-d6): δ 10.54 (s, 1H), 8.81 (t, J = 5.8 Hz, 1H), 7.95 (dd, J = 6.8, 2.6 Hz, 1H), 7.55- 7.53 (m, 1H), 7.49-7.47 (m, 1H), 7.40 (t, J = 9.2 Hz, 1H), 6.72 (s, 1H), 4.51- 4.38 (m, 2H), 4.19-4.16 (m, 2H), 2.58 (s, 3H), 2.05-1.92 (m, 2H). LCMS calcd for; C₁₆H₁₆BrClFN₅O₅S. Found; 526 (M + 3). |
| 987 | 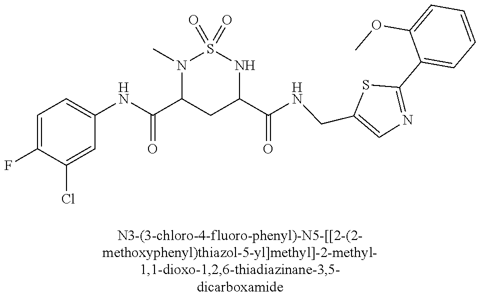 [SMILES: COc1ccccc1-c1ncc(CNC(=O)C2CC(C(=O)Nc3ccc(F)c(Cl)c3)N(C)S(=O)(=O)N2)s1] | ¹H NMR (400 MHz, DMSO-d6): δ 10.52 (s, 1H), 8.79 (t, J = 5.2 Hz, 1H), 8.22 (d, J = 7.6 Hz, 1H), 7.96-7.93 (m, 1H), 7.77 (s, 1H), 7.56-7.52 (m, 1H), 7.46-7.36 (m, 3H), 7.23 (d, J = 8.4 Hz, 1H), 7.08 (t, J = 7.2 Hz, 1H), 4.58- 4.48 (m, 2H), 4.22-4.19 (m, 2H), 4.11- 4.09 (m, 1H), 3.99 (s, 3H), 2.57 (s, 3H), 2.00-1.92 (m, 2H). LCMS calcd for; C₂₃H₂₃ClFN₅O₅S₂. Found; 568.10 (M + 1). |
| 987- | N3-(3-chloro-4-fluoro-phenyl)-N5-[[2-(2- | ¹H NMR (400 MHz, DMSO-d6): δ |
| ISOMER-A | methoxyphenyl)thiazol-5-yl]methyl]-2-methyl- | 10.52 (s, 1H), 8.79 (t, J = 4.8 Hz, 1H), |
| | 1,1-dioxo-1,2,6-thiadiazinane-3,5- | 8.22 (d, J = 7.6 Hz, 1H), 7.95-7.94 (m, |
| | dicarboxamide. | 1H), 7.77 (s, 1H), 7.55-7.53 (m, 1H), |
| | | 7.46-7.37 (m, 3H), 7.23 (d, J = 8.4 Hz, |
| | | 1H), 7.08 (t, J = 7.2 Hz, 1H), 4.55- |
| | | 4.52 (m, 2H), 4.22-4.20 (m, 1H), 4.11- |
| | | 4.09 (m, 1H), 3.99 (s, 3H), 2.57 (s, |
| | | 3H), 1.99-1.93 (m, 2H). LCMS calcd |
| | | for; C₂₃H₂₃ClFN₅O₅S₂. Found; 568.16 |
| | | (M + 1). |
| 987- | N3-(3-chloro-4-fluoro-phenyl)-N5-[[2-(2- | ¹H NMR (400 MHz, DMSO-d6): δ |
| ISOMER-B | methoxyphenyl)thiazol-5-yl]methyl]-2-methyl- | 10.52 (s, 1H), 8.79 (t, J = 5.6 Hz, 1H), |
| | 1,1-dioxo-1,2,6-thiadiazinane-3,5- | 8.22 (d, J = 8.0 Hz, 1H), 7.96-7.94 (m, |
| | dicarboxamide. | 1H), 7.77 (s, 1H), 7.55-7.53 (m, 1H), |
| | | 7.46-7.36 (m, 3H), 7.23 (d, J = 8.4 Hz, |
| | | 1H), 7.08 (t, J = 7.2 Hz, 1H), 4.55- |
| | | 4.49 (m, 2H), 4.23-4.19 (m, 1H), 4.11- |
| | | 4.09 (m, 1H), 3.99 (s, 3H), 2.57 (s, |
| | | 3H), 1.99-1.93 (m, 2H). LCMS calcd |
| | | for; C₂₃H₂₃ClFN₅O₅S₂. Found; 568.09 |
| | | (M + 1). |
| 988 | 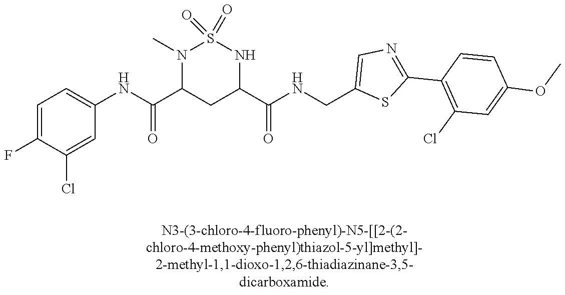 [SMILES: COc1ccc(-c2ncc(CNC(=O)C3CC(C(=O)Nc4ccc(F)c(Cl)c4)N(C)S(=O)(=O)N3)s2)c(Cl)c1] | ¹H NMR (400 MHz, DMSO-d6): δ 10.51 (s, 1H), 8.83 (t, J = 6.1 Hz, 1H), 8.08 (dd, J = 8.9, 1.6 Hz, 1H), 7.94 (dd, J = 6.8, 2.6 Hz, 1H), 7.80 (s, 1H), 7.55-7.52 (m, 1H), 7.43-7.40 (m, 2H), 7.20-7.19 (m, 1H), 7.11-7.00 (m, 1H), 4.57-4.46 (m, 2H), 4.21-4.18 (m, 1H), 4.11-4.08 (m, 1H), 3.84 (s, 3H), 2.57 (s, 3H), 2.03-1.86 (m, 2H). LCMS calcd for; C₂₃H₂₂Cl₂FN₅O₅S₂. Found; 602.05 (M + 1). |
| 991 | 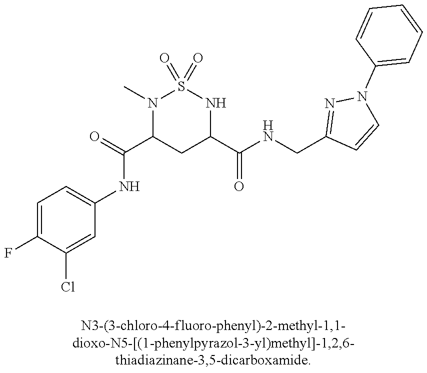 [SMILES: CN1C(C(=O)Nc2ccc(F)c(Cl)c2)CC(C(=O)NCc2ccn(-c3ccccc3)n2)NS1(=O)=O] | ¹H NMR (400 MHz, DMSO-d6): δ 10.53 (s, 1H), 8.62 (t, J = 5.2 Hz, 1H), 8.42-8.41 (m, 1H), 7.96 (dd, J = 6.8, 2.6 Hz,1H), 7.80 (d, J = 8.0 Hz, 2H), 7.59-7.23 (m, 6H), 6.45-6.44 (m, 1H), 4.36 (d, J = 5.8 Hz, 2H), 4.23-4.13 (m, 2H), 2.58 (s, 3H), 2.03-1.90 (m, 2H). LCMS calcd for; C₂₂H₂₂ClFN₆O₄S. Found; 521.15 (M + 1). |
| 991- | N3-(3-chloro-4-fluoro-phenyl)-2-methyl-1,1- | ¹H NMR (400 MHz, DMSO-d6): δ |
| ISOMER-A | dioxo-N5-[(1-phenylpyrazol-3-yl)methyl]-1,2,6- | 10.53 (s, 1H), 8.63-8.60 (m, 1H), 8.42- |
| | thiadiazinane-3,5-dicarboxamide. | 8.41 (m, 1H), 7.97-7.94 (m, 1H), 7.80 |
| | | (d, J = 8.0 Hz, 2H), 7.59-7.53 (m, 1H), |
| | | 7.50-7.45 (m, 2H), 7.42-7.36 (m, 2H), |
| | | 7.29-7.23 (m, 1H), 6.45-6.44 (m, 1H), |
| | | 4.36 (d, J = 5.6 Hz, 2H), 4.23-4.13 (m, |
| | | 2H), 2.58 (s, 3H), 2.01-1.92 (m, |
| | | 2H). LCMS calcd for; |
| | | C₂₂H₂₂ClFN₆O₄S. Found; |
| | | 521.10 (M + 1). |
| 991- | N3-(3-chloro-4-fluoro-phenyl)-2-methyl-1,1- | ¹H NMR (400 MHz, DMSO-d6): δ |
| ISOMER-B | dioxo-N5-[(1-phenylpyrazol-3-yl)methyl]-1,2,6- | 10.54 (s, 1H), 8.64-8.61 (m, 1H), 8.43- |
| | thiaidazinane-3,5-dicarboxmaide. | 8.40 (m, 1H), 7.97-7.94 (m, 1H), 7.80 |
| | | (d, J = 7.6 Hz, 2H), 7.57-7.52 (m, |
| | | 1H), 7.49-7.45 (m, 2H), 7.42-7.37 (m, |
| | | 2H), 7.29-7.25 (m, 1H), 6.44-6.42 (m, |
| | | 1H), 4.36 (d, J = 6.0 Hz, 2H), 4.23- |
| | | 4.15 (m, 2H), 2.58 (s, 3H), 2.00-1.96 |
| | | (m, 2H). LCMS calcd for; |
| | | C₂₂H₂₂ClFN₆O₄S. Found; 521.10 |
| | | (M + 1). |

### Biological Methods

### Assay Measuring Activity of Test Compounds on Viral Production from HepAD38 Cells

HepAD38 cells grown in a T-150 flask (Corning, cat #: 430825) with Growth Medium (DMEM/F12 (L:1) (Hyclone, cat #: SH30023.02), 1× Pen/Strep (Invitrogen, cat #: 15140-122), 10% FBS (Tissue Culture Biologics, cat #: 101), 250 pAg/mL G418 (Alfa Aesar, cat #: J62671), 1 μg/mL Tetracycline (Teknova, cat #: T3320)) were detached with 0.25% trypsin-EDTA (Invitrogen, cat #: 25200-056). Tetracycline-free treatment medium (15 mL DMEM/F12 (1:1) 1× Pen/step, with 2% FBS, Tet-system approved (Clontech, cat #: 631106) were then added to mix, transferred into a 50 ml conical tube (Falcon, cat #: 21008-918,) and spun at 1300 rpm for 5 min. Pelleted cells were then re-suspended/washed with 50 mL of 1×DPBS (Invitrogen, cat #: 14190-136) 2 times and 50 mL treatment medium twice. HepAD38 cells were then re-suspended with 10 mL of treatment medium, syringed and counted. Wells of 96-well clear bottom TC plate (Corning, cat #: 3904,) were seeded at 50,000 cells/well in 180 μL of treatment medium, and 20 μL of either 10% DMSO (Sigma, cat #: D4540) as controls or a 10× solution of test compounds in 10% DMSO in treatment media was added for a final compound concentration starting at 10 μM, and plates were incubated in 5% CO₂ incubator at 37° C. for 5 days.

Subsequently viral load production was assayed by quantitative PCR (qPCR) of the HBV core sequence. PCR reaction mixture containing forward primers HBV-f 5′-CTGTGCCTTGGGTGGCTIT-3′ (IDT DNA), Reverse primers HBV-r 5′-AAGGAAAGAAGTCAGAAGGCAAAA-3′ (IDT DNA), Fluorescent TaqMan™ Probes HBV-probe 5′-FAM/AGCTCCAAA/ZEN/TTCTTTATAAGGGTCGATGTC/3IABkFQ-3′ (IDT DNA), 10 μL/well of PerfeCTa® qPCR ToughMix® (Quanta Biosciences, Cat #: 95114-05K), and 6 μL/well of DEPC water (Alfa Aesar, cat #: J62087) was prepared. Four μL of supernatant was added to 16 μL of the reaction mixture in a qPCR plate (Applied Biosytems, Cat #: 4309849), sealed with a film (Applied Biosystems, Cat #: 4311971), centrifuged for a few seconds, and subsequently run on an Applied Biosystems VIIA7. The PCR mixture was incubated at 45° C. for 5 min, then 95° C. for 10 min, followed by 40 cycles of 10 seconds at 95° C. and 20 seconds at 60° C. Viral load was quantified against known HBV DNA standards by using ViiA™ 7 Software. Viral load in the supernatant from wells with treated cells were compared against viral load in supernatant from DMSO control wells (≥3 per plate). Cell viability assay was performed with CellTiter-Glo Luminescent Cell Viability Assay (Promega, cat #: G7573) with modification. Mixed appropriate amount of CellTiter-Glo (CTG) 1×DPBS in a 1:1 ratio, added 100 uL of the mixture to each well followed completely removal of all supernatant in each well without touching cell surface. Incubated the plate at room temperature for 10 min on an orbital shaker, and then read the plate with a plate reader (TECAN M1000 or Envision). EC₅₀ or CC₅₀ values were calculated through curve-fitting of the four-parameter nonlinear-logistic-regression model (GraphPad Prism or Dotmatics). CC₅₀ values were all >10 μM.

Table 4 gives the viral load lowering EC₅₀ values grouped in the following ranges: A indicates EC₅₀<0.05 μM; B indicates EC₅₀ 0.05-0.10 μM; C indicates 0.10<EC₅₀<10 μM.

TABLE 4

| Example ID | Activity |
|---|---|
| 1006 | B |
| 1007 | A |
| 1009 | A |
| 1010 | A |
| 1011 | A |
| 1011-ISOMER-A | C |
| 1011-ISOMER-B | A |
| 1012 | A |
| 1012-ISOMER-A | A |
| 1012-ISOMER-B | B |
| 1014 | A |
| 1014-ISOMER-A | A |
| 1014-ISOMER-B | C |
| 1015 | A |
| 1015-ISOMER-A | A |
| 1015-ISOMER-B | B |
| 1016 | A |
| 1016-ISOMER-A | A |
| 1016-ISOMER-B | B |
| 1017 | A |
| 1017-ISOMER-A | A |
| 1017-ISOMER-B | C |
| 1018 | A |
| 1019 | A |
| 1025 | A |
| 1025-ISOMER-A | A |
| 1025-ISOMER-B | C |
| 1028 | A |
| 1029 | C |
| 1030 | A |
| 1031 | A |
| 1033 | C |
| 1036 | A |
| 1036-ISOMER-A | A |
| 1036-ISOMER-B | C |
| 1040 | A |
| 1040-ISOMER-A | A |
| 1040-ISOMER-B | A |
| 1050 | A |
| 1050-ISOMER-A | C |
| 1050-ISOMER-B | A |
| 1051 | A |
| 1052 | A |
| 1054 | A |
| 1055 | A |
| 1055-R | A |
| 1055-R-ISOMER-A | C |
| 1055-R-ISOMER-B | A |
| 1055-S | A |
| 1055-S-ISOMER-A | C |
| 1055-S-ISOMER-B | A |
| 1056 | A |
| 1056-ISOMER-A | C |
| 1056-ISOMER-B | A |
| 1057 | A |
| 1057-ISOMER-A | A |
| 1057-ISOMER-B | A |
| 1062 | B |
| 1063 | A |
| 1064 | A |
| 1065 | A |
| 1065-ISOMER-A | C |
| 1065-ISOMER-B | A |
| 1066 | A |
| 1066-ISOMER-A | C |
| 1066-ISOMER-B | A |
| 1066-ISOMER-C | A |
| 1066-ISOMER-D | C |
| 1067 | A |
| 1067-ISOMER-A | A |
| 1067-ISOMER-B | C |
| 1067-ISOMER-C | A |
| 1067-ISOMER-D | C |
| 1068 | B |
| 1068-ISOMER-A | A |
| 1068-ISOMER-B | B |
| 1068-ISOMER-C | A |
| 1068-ISOMER-D | C |
| 1069 | A |
| 1070 | A |
| 1071 | A |
| 1072 | A |
| 1072-ISOMER-A | A |
| 1072-ISOMER-B | C |
| 1073 | A |
| 1074 | A |
| 1081 | C |
| 1082 | A |
| 1082-ISOMER-A | A |
| 1082-ISOMER-B | B |
| 1083 | A |
| 1083-ISOMER-A | A |
| 1083-ISOMER-B | C |
| 1084 | A |
| 1085 | A |
| 1086 | A |
| 1086-ISOMER-A | A |
| 1086-ISOMER-B | C |
| 1087 | A |
| 1087-ISOMER-A | A |
| 1087-ISOMER-B | B |
| 1089 | A |
| 1090 | A |
| 1093 | A |
| 1098 | A |
| 1099 | B |
| 1100 | B |
| 1100-ISOMER-A | A |
| 1100-ISOMER-B | C |
| 1100-ISOMER-C | C |
| 1100-ISOMER-D | C |
| 1101 | B |
| 1101-ISOMER-A | A |
| 1101-ISOMER-B | C |
| 1101-ISOMER-C | C |
| 1101-ISOMER-D | C |
| 1104 | A |
| 1116 | B |
| 1132 | C |
| 1132-ISOMER-A | B |
| 1132-ISOMER-B | C |
| 1134 | A |
| 1134-ISOMER-A | C |
| 1134-ISOMER-B | A |
| 1135 | A |
| 1136 | A |
| 1136-ISOMER-A | A |
| 1136-ISOMER-B | C |
| 1137 | A |
| 1141 | A |
| 1141-ISOMER-A | A |
| 1141-ISOMER-B | C |
| 1142 | A |
| 1142-ISOMER-A | A |
| 1142-ISOMER-B | C |
| 1143 | A |
| 1143-ISOMER-A | C |
| 1143-ISOMER-B | A |
| 1144 | A |
| 1144-ISOMER-A | A |
| 1144-ISOMER-B | C |
| 1145 | A |
| 1145-ISOMER-A | A |
| 1145-ISOMER-B | C |
| 1147 | B |
| 1148 | C |
| 1148-ISOMER-A | B |
| 1148-ISOMER-B | C |
| 1149 | A |
| 1149-ISOMER-A | A |
| 1149-ISOMER-B | C |
| 1150 | A |
| 1150-ISOMER-A | A |
| 1150-ISOMER-B | C |
| 1151 | A |
| 1151-ISOMER-A | A |
| 1151-ISOMER-B | C |
| 1153 | A |
| 1153-ISOMER-A | A |
| 1153-ISOMER-B | C |
| 1157 | B |
| 1158 | A |
| 1159 | C |
| 1160 | C |
| 1160-ISOMER-A | B |
| 1160-ISOMER-B | C |
| 1161 | C |
| 1162 | A |
| 1197 | C |
| 1197-ISOMER-A | C |
| 1197-ISOMER-B | B |
| 1199 | A |
| 1199-ISOMER-A | A |
| 1199-ISOMER-B | C |
| 1202 | C |
| 1202-ISOMER-A | C |
| 1202-ISOMER-B | A |
| 1205 | C |
| 476 | B |
| 477 | A |
| 477-ISOMER-A | A |
| 477-ISOMER-B | C |
| 478 | C |
| 481 | B |
| 482 | A |
| 482-ISOMER-A | A |
| 482-ISOMER-B | C |
| 485 | A |
| 489 | B |
| 493 | A |
| 493-R | A |
| 493-R-ISOMER-A | A |
| 493-R-ISOMER-B | C |
| 493-S | A |
| 493-S-ISOMER-A | A |
| 493-S-ISOMER-B | C |
| 494 | B |
| 495 | A |
| 495-ISOMER-A | C |
| 495-ISOMER-B | A |
| 498 | C |
| 498-ISOMER-A | C |
| 498-ISOMER-B | B |
| 682 | C |
| 684 | C |
| 685 | C |
| 686 | B |
| 686-ISOMER-A | C |
| 686-ISOMER-B | A |
| 687 | A |
| 687-ISOMER-A | C |
| 687-ISOMER-B | A |
| 689 | A |
| 689-ISOMER-A | C |
| 689-ISOMER-B | A |
| 691 | A |
| 691-ISOMER-A | C |
| 691-ISOMER-B | A |
| 704 | C |
| 706 | C |
| 708 | C |
| 710 | C |
| 711 | C |
| 715 | C |
| 716 | A |
| 716-ISOMER-A | A |
| 716-ISOMER-B | C |
| 717 | A |
| 717-ISOMER-A | A |
| 717-ISOMER-B | B |
| 718 | A |
| 718-ISOMER-A | A |
| 718-ISOMER-B | C |
| 719 | A |
| 719-ISOMER-A | A |
| 719-ISOMER-B | C |
| 721 | A |
| 721-ISOMER-A | A |
| 721-ISOMER-B | C |
| 724 | B |
| 725 | B |
| 726 | B |
| 728 | C |
| 729 | A |
| 729-ISOMER-A | A |
| 729-ISOMER-B | C |
| 730 | A |
| 730-ISOMER-A | A |
| 730-ISOMER-B | C |
| 731 | C |
| 732 | C |
| 734 | B |
| 736 | B |
| 738 | B |
| 739 | B |
| 740 | A |
| 740-ISOMER-A | A |
| 740-ISOMER-B | C |
| 741 | B |
| 742 | B |
| 742-ISOMER-A | C |
| 742-ISOMER-B | A |
| 744 | B |
| 745 | B |
| 755 | C |
| 766 | A |
| 766-ISOMER-A | A |
| 766-ISOMER-B | B |
| 767 | A |
| 767-ISOMER-A | A |
| 767-ISOMER-B | C |
| 769 | A |
| 769-ISOMER-A | A |
| 769-ISOMER-B | C |
| 770 | B |
| 772 | A |
| 773 | C |
| 774 | C |
| 776 | C |
| 817 | A |
| 818 | A |
| 819 | A |
| 820 | A |
| 821 | A |
| 822 | A |
| 823 | A |
| 823-ISOMER-A | A |
| 823-ISOMER-B | C |
| 826 | C |
| 828 | C |
| 829 | C |
| 830 | B |
| 831 | C |
| 832 | B |
| 833 | C |
| 834 | C |
| 835 | B |
| 836 | B |
| 837 | A |
| 838 | C |
| 851 | C |
| 852 | C |
| 854 | A |
| 854-ISOMER-A | A |
| 854-ISOMER-B | C |
| 855 | A |
| 855-ISOMER-A | A |
| 855-ISOMER-B | C |
| 856 | C |
| 858 | C |
| 859 | C |
| 862 | A |
| 862-ISOMER-A | C |
| 862-ISOMER-B | A |
| 866 | C |
| 868 | A |
| 888 | C |
| 888-ISOMER-A | C |
| 888-ISOMER-B | A |
| 890 | A |
| 890-ISOMER-A | A |
| 890-ISOMER-B | C |
| 891 | C |
| 891-ISOMER-A | C |
| 891-ISOMER-B | C |
| 893 | A |
| 893-ISOMER-A | A |
| 893-ISOMER-B | C |
| 896 | C |
| 896-ISOMER-A | B |
| 896-ISOMER-B | C |
| 906 | C |
| 907 | B |
| 909 | A |
| 910 | B |
| 910-ISOMER-A | A |
| 910-ISOMER-B | C |
| 911 | B |
| 911-ISOMER-A | C |
| 911-ISOMER-B | A |
| 912 | A |
| 912-ISOMER-A | C |
| 912-ISOMER-B | A |
| 913 | B |
| 913-ISOMER-A | C |
| 913-ISOMER-B | A |
| 914 | A |
| 914-ISOMER-A | A |
| 914-ISOMER-B | B |
| 915 | C |
| 916 | A |
| 916-ISOMER-A | C |
| 916-ISOMER-B | A |
| 917 | A |
| 918 | A |
| 920 | A |
| 920-ISOMER-A | C |
| 920-ISOMER-B | A |
| 921 | B |
| 921-ISOMER-A | A |
| 921-ISOMER-B | B |
| 922 | A |
| 922-ISOMER-A | C |
| 922-ISOMER-B | A |
| 923 | B |
| 924 | A |
| 924-ISOMER-A | C |
| 924-ISOMER-B | A |
| 925 | A |
| 925-ISOMER-A | A |
| 925-ISOMER-B | C |
| 926 | A |
| 928 | B |
| 929 | A |
| 929-ISOMER-A | C |
| 929-ISOMER-B | A |
| 931 | A |
| 931-ISOMER-A | C |
| 931-ISOMER-B | A |
| 932 | A |
| 932-ISOMER-A | C |
| 932-ISOMER-B | A |
| 942 | B |
| 943 | A |
| 943-ISOMER-A | A |
| 943-ISOMER-B | C |
| 946 | B |
| 946-ISOMER-A | A |
| 946-ISOMER-B | C |
| 948-ISOMER-A | A |
| 948-ISOMER-B | C |
| 948-ISOMER-D | A |
| 957 | A |
| 957-ISOMER-A | A |
| 957-ISOMER-B | C |
| 961 | C |
| 963 | C |
| 964 | C |
| 971 | A |
| 972 | A |
| 974 | A |
| 974-ISOMER-A | A |
| 974-ISOMER-B | B |
| 975 | A |
| 975-ISOMER-A | A |
| 975-ISOMER-B | C |
| 978 | A |
| 979 | A |
| 979-ISOMER-A | C |
| 979-ISOMER-B | A |
| 980 | C |
| 987 | A |
| 987-ISOMER-A | A |
| 987-ISOMER-B | C |
| 988 | A |
| 991 | A |
| 991-ISOMER-A | C |
| 991-ISOMER-B | A |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound of Formula I: 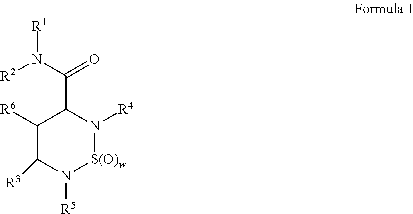
or a pharmaceutically acceptable salt thereof, wherein:
R¹ is a phenyl, naphthyl or heteroaryl, wherein: the phenyl, naphthyl or heteroaryl is optionally substituted with one, two, or three independently selected R³² groups;
R² is hydrogen or C₁₋₆alkyl;
R³ is -L-R⁷;
L is —C(O)—, —C(O)C₁₋₆alkyl-, —C₁₋₆alkylC(O)—, —C(O)NR^{c}—, —NR^{c}C(O)—, —C(O)NR^{c}C₁₋₆alkyl-, —NR^{c}C(O)C₁₋₆alkyl-, —C₁₋₆alkylC(O)NR^{c}—, or —C₁₋₆alkylNR^{c}C(O)—;
R⁷ is hydrogen, cycloalkyl, cycloalkenyl, carbocyclyl, heterocycloalkyl, heterocycloalkenyl, heterocyclyl, phenyl or heteroaryl, wherein: the cycloalkyl, cycloalkenyl, carbocyclyl. heterocycloalkyl, heterocycloalkenyl, heterocyclyl, phenyl or heteroaryl is optionally substituted with one, two or three substituents independently selected from the group consisting of R³², R³⁴ and R^{7a};
R^{7a} is a phenyl or heteroaryl, wherein: the phenyl or heteroaryl is optionally substituted with one, two or three independently selected R³² groups;
R⁴ is hydrogen or C₁₋₆alkyl optionally substituted with one, two, or three substituents independently selected from the group consisting of halogen, —OH, —CN, —S(O)_{q}—C₁₋₆alkyl, —NR^{a}R^{b}, —NR^{c}—S(O)ₜ—C₁₋₆alkyl, —S(O)ₜ—NR^{a}R^{b}, C₂₋₆alkenyl, C₂₋₆alkynyl, haloC₁₋₆alkyl, C₁₋₆alkoxy, haloC₁₋₆alkoxy, —C(O)NR^{a}R^{b}, —C(O)—C₁₋₆alkyl, formyl, —C(O)OH, a-C(O)O—C₁₋₆alkyl, benzyloxy, C₁₋₄alkoxyphenyl, pyrrolidinyl, morpholinyl, tetrahydrofuranyl and triazolyl;
R⁵ is hydrogen or C₁₋₆alkyl optionally substituted with a substituent selected from the group consisting of halogen, —OH, C₁₋₆alkoxy, —NR^{a}R^{b}, and R^{a}R^{b}N—C₁₋₆alkyl;
R⁶ is hydrogen or C₁₋₆alkyl;
R³² is halo, —OH, —CN, —NO₂, oxo, hydrazino, formyl, azido, silyl, siloxy, —S(O)_{q}—C₁₋₆alkyl, —NR^{a}R^{b}, —NR^{c}—S(O)ₜ—C₁₋₆alkyl, —S(O)ₜ—NR^{a}R^{b}, C₁₋₆alkyl, C₂₋₆alkenyl, C₂₋₆alkynyl, C₃₋₆cycloalkyl, haloC₁₋₆alkyl, hydroxyC₁₋₆alkyl, R^{a}R^{b}N—C₁₋₆alkyl-, C₁₋₆alkoxy, haloC₁₋₆alkoxy, hydroxyC₁₋₆alkoxy-, R^{a}R^{b}N—C₁₋₆alkoxy-, C₁₋₆alkoxyC₁₋₆alkyl, —C(O)NR^{a}R^{b}, —C(O)—C₁₋₆alkyl, —C(O)OH, or —C(O)O—C₁₋₆alkyl;
R³⁴ is hydrogen or C₁₋₄alkyl;
R^{a} and R^{b} are independently selected for each occurrence from the group consisting of hydrogen and C₁₋₆alkyl; or
R^{a} and R^{b} may be taken together with the nitrogen to which R^{a} and R^{b} are attached to form: 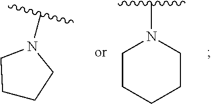 [SMILES: *N1CCCC1 ; *N1CCCCC1]
R^{c} is independently selected for each occurrence from the group consisting of hydrogen and C₁₋₆alkyl;
for each occurrence, q is independently 0, 1 or 2;
for each occurrence, t is independently 1 or 2; and
w is 0, 1 or 2.

2. The compound of claim 1, wherein the compound of Formula I is of Formula II: 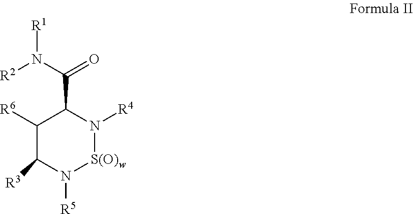
or a pharmaceutically acceptable salt thereof.

3. The compound of claim 1, or a pharmaceutically acceptable salt thereof, wherein R² is hydrogen.

4. The compound of claim 1, or a pharmaceutically acceptable salt thereof, wherein R⁴ is methyl or methoxyethyl.

5. The compound of claim 4, or a pharmaceutically acceptable salt thereof, wherein R⁴ is methyl.

6. The compound of claim 1, or a pharmaceutically acceptable salt thereof, wherein R⁵ is hydrogen.

7. The compound of claim 1, or a pharmaceutically acceptable salt thereof, wherein R⁶ is hydrogen.

8. The compound of claim 1, or a pharmaceutically acceptable salt thereof, wherein: L is —C(O)—, —C(O)C₁₋₆alkyl- or —C₁₋₆alkylC(O)—.

9. The compound of claim 1, or a pharmaceutically acceptable salt thereof, wherein: L is —C₁₋₆alkylC(O)NR^{c}— or —C₁₋₆alkylNR^{c}C(O)—;

10. The compound of claim 1, or a pharmaceutically acceptable salt thereof, wherein: L is —C(O)NR^{c}—, —C(O)NR^{c}C₁₋₆alkyl- or —NR^{c}C(O)C₁₋₆alkyl-.

11. The compound of claim 1, or a pharmaceutically acceptable salt thereof, wherein: L is —C(O)NR^{c}— or —C(O)NR^{c}C₁₋₆alkyl-.

12. The compound of claim 1, or a pharmaceutically acceptable salt thereof, wherein L is —C(O)NR^{c}C₁₋₆alkyl-.

13. The compound of claim 1, or a pharmaceutically acceptable salt thereof, wherein L is —C(O)NHC₁₋₆alkyl-.

14. The compound of claim 1, or a pharmaceutically acceptable salt thereof, wherein L is —C(O)NR^{c}—.

15. The compound of claim 1, or a pharmaceutically acceptable salt thereof, wherein L is —C(O)NH—.

16. The compound of claim 1, or a pharmaceutically acceptable salt thereof, wherein R⁷ is cycloalkyl, cycloalkenyl or carbocyclyl, wherein: cycloalkyl, cycloalkenyl, carbocyclyl the is optionally substituted with one, two or three substituents independently selected from the group consisting of R³², R³⁴ and R^{7a}.

17. The compound of claim 1, or a pharmaceutically acceptable salt thereof, wherein R⁷ is heterocycloalkyl, heterocycloalkenyl or heterocyclyl, wherein: the heterocycloalkyl, heterocycloalkenyl or heterocyclyl is optionally substituted with one, two or three substituents independently selected from the group consisting of R³², R³⁴ and R^{7a}.

18. The compound of claim 1, or a pharmaceutically acceptable salt thereof, wherein: R⁷ is phenyl optionally substituted with one, two or three substituents independently selected from the group consisting of R³², R³⁴ and R^{7a}.

19. The compound of claim 1, or a pharmaceutically acceptable salt thereof, wherein: R⁷ is heteroaryl optionally substituted with one, two or three substituents independently selected from the group consisting of R³², R³⁴ and R^{7a}.

20. The compound of claim 1, or a pharmaceutically acceptable salt thereof, wherein R¹ is phenyl optionally substituted with one, two or three substituents independently selected from halo, cyano, methyl and trifluoromethyl.

21. The compound of claim 20, or a pharmaceutically acceptable salt thereof, wherein R¹ is 3-chloro-4-fluorophenyl.

22. A pharmaceutical composition comprising a compound according to claim 1, or a pharmaceutically acceptable salt thereof, and a pharmaceutically acceptable excipient.

23. A method of treating a Hepatitis B (HBV) infection in a patient, the method comprising: administering an effective amount of the compound according to claim 1, or a pharmaceutically acceptable salt thereof, to a patient in need thereof.

24. A method of treating a Hepatitis B (HBV) infection in a patient, the method comprising: administering an effective amount of a pharmaceutical composition of claim 22 to a patient in need thereof.

25. A pharmaceutical composition comprising a compound according to claim 2, or a pharmaceutically acceptable salt thereof, and a pharmaceutically acceptable excipient.

26. A method of treating a Hepatitis B (HBV) infection in a patient, the method comprising: administering an effective amount of the compound according to claim 2, or a pharmaceutically acceptable salt thereof, to a patient in need thereof.

27. A method of treating a Hepatitis B (HBV) infection in a patient, the method comprising: administering an effective amount of a pharmaceutical composition of claim 25 to a patient in need thereof.
